(19) United States
(12) Patent Application Publication
(10) Pub. No.: US 2025/0235409 A1
(43) Pub. Date: Jul. 24, 2025

(54) IMPLANTABLE PARTICLES AND RELATED METHODS

(71) Applicant: SIGILON THERAPEUTICS, INC., Cambridge, MA (US)

(72) Inventors: Lauren Emily Barney, Cambridge, MA (US); Michael Beauregard, Boston, MA (US); Guillaume Carmona, Cambridge, MA (US); Francisco Caballero Gonzalez, Brookline, MA (US); Richard Heidebrecht, Somerville, MA (US); Erika Ellen Johnston, Cambridge, MA (US); Robert James Miller, East Bridgewater, MA (US); Matthias Alexander Oberli, Cambridge, MA (US); Owen O'Connor, Raynham, MA (US); David Peritt, Skokie, IL (US); Jared A. Sewell, Somerville, MA (US); Devyn McKinley Smith, Barrington, RI (US); Omid Veiseh, Bellaire, TX (US); Paul Kevin Wotton, Boston, MA (US)

(21) Appl. No.: 18/932,580

(22) Filed: Oct. 30, 2024

Related U.S. Application Data
(62) Division of application No. 17/045,127, filed on Oct. 2, 2020, now Pat. No. 12,161,760.
(60) Provisional application No. 62/812,568, filed on Mar. 1, 2019.
(60) Provisional application No. 62/737,838, filed on Sep. 27, 2018.
(60) Provisional application No. 62/652,880, filed on Apr. 4, 2018.

(51) Int. Cl. A61K 9/50; A61K 35/28; A61K 35/30; C07K 14/62; C07K 14/755; C12N 9/64

(52) U.S. Cl. CPC A61K 9/5031; A61K 9/5036; A61K 9/5073; A61K 9/5089; A61K 35/28; A61K 35/30; C07K 14/62; C07K 14/755; C12N 9/644; C12Y 304/21022

(57) ABSTRACT

Described herein are particles comprising a first compartment, a second compartment, and a compound of Formula (I), as well as compositions and methods of making and using the same. The particles may comprise a cell capable of expressing a therapeutic agent useful for the treatment of a disease, disorder, or condition described herein.

### CLAIM OF PRIORITY

This application is a Continuation of U.S. application Ser. No. 17/045,127, filed Oct. 2, 2020, which is a U.S. National Phase application under 35 U.S.C. § 371 of International Application No. PCT/US2019/024371, filed Mar. 27, 2019, which claims priority to U.S. Provisional Application No. 62/652,880, filed Apr. 4, 2018; U.S. Application No. 62/737,838, filed Sep. 27, 2018; and U.S. Application No. 62/812,568, filed Mar. 1, 2019. The disclosure of each of the foregoing applications is incorporated herein by reference in its entirety.

### SEQUENCE LISTING

The instant application contains a Sequence Listing which has been submitted electronically in ASCII format and is hereby incorporated by reference in its entirety. Said ASCII copy, created on Sep. 26, 2018, is named S2225-7022WO_SL.txt and is 205,145 bytes in size.

### BACKGROUND

The function of implanted particles comprising engineered cells depends on numerous factors including the ability to provide a product and the biological immune response pathway of the recipient (Anderson et al., Semin Immunol (2008) 20:86-100; Langer, Adv Mater (2009) 21:3235-3236). The performance of such particles after implant will depend to a large extent on their biocompatibility, including the degree to which they are afibrotic, e.g., are able to avoid or mitigate the foreign-body response. Several publications have reported that the foreign body response (FBR) to implanted hydrogel capsules in rodents and non-human primates can be significantly reduced by using spherical capsules that have a size of at least 1 mm in diameter, e.g., millicapsules (Veiseh, O., et al, Nature Materials 14:643-652 (2015); WO2014/153126; WO2016/187225) and/or that are prepared using hydrogel-forming polymers that are chemically modified with certain compounds that mitigate the FBR (Vegas, A., et al., Nature Medicine 22(3):306-311 (2016), Vegas, A., et al., Nature Biotechnology 34(3):345-352 (2016); WO 2012/167223; WO 2017/075631).

### SUMMARY

Described herein are particles comprising a first compartment, a second compartment, and a compound of Formula (I) (e.g., as described herein), as well as compositions and methods of making and using the same. In some embodiments, the particle comprises a cell (e.g., a cell described herein). In some embodiments, the cell produces a therapeutic agent useful, e.g., for the treatment of a disease, disorder or condition in a subject, e.g., a blood clotting disorder or a lysosomal storage disease. In some embodiments, the particle is capable of modulating the immune response (e.g., FBR) or the effect of an immune response (e.g., FBR) in a subject.

In one aspect, the present disclosure features a particle comprising a) a first compartment; b) a second compartment; and c) a compound of Formula (I):

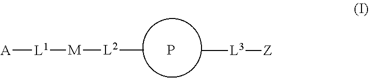

or a pharmaceutically acceptable salt thereof, wherein the variables A, L¹, M, L², P, L³, and Z, as well as related subvariables, are defined herein. In some embodiments, the first compartment is surrounded by the second compartment. In some embodiments, the second compartment forms a barrier around the first compartment. In some embodiments, the first compartment comprises a compound of Formula (I). In some embodiments, the second compartment comprises a compound of Formula (I). In some embodiments, each of the first and second compartments independently comprise a compound of Formula (I). In some embodiments, a compound of Formula (I) is disposed on the exterior surface of the particle.

In some embodiments, the compound of Formula (I) or a pharmaceutically acceptable salt thereof (e.g., Formulas (I-a), (I-b), (I-c), (I-d), (I-e), (I-f), (II), (II-a), (III), (III-a), (III-b), (III-c), or (III-d)) is a compound described herein. In some embodiments, the compound of Formula (I) is one of the compounds shown in Table 2 herein.

In some embodiments, at least one of the compartments in the particle comprises a polymer. In some embodiments, both the first compartment and the second compartment of the particle comprise a polymer (e.g., a polysaccharide, e.g., alginate). In some embodiments, the first compartment and the second compartment of the particle comprise the same polymer. In some embodiments, the first compartment and the second compartment of the particle comprise a different polymer.

In some embodiments, the polymer is a polysaccharide or other hydrogel-forming polymer (e.g., alginate, hyaluronate or chondroitin). In some embodiments, the polymer is an alginate. In some embodiments, the particle comprises an alginate that is chemically modified with a compound of Formula (I). In some embodiments, the chemically modified alginate has a low molecular weight (e.g., approximate molecular weight of <75 kD). In some embodiments, the particle comprises a mixture of chemically modified alginate and unmodified alginate. In some embodiments, the particle is a hydrogel capsule. In some embodiments, the particle is a millicapsule or a microcapsule (e.g., a hydrogel millicapsule or a hydrogel microcapsule).

In some embodiments, the particle is spherical. In some embodiments, the total volume (as defined herein) of the second compartment is greater than (e.g. >1.5×, 2×, 3×, or 5×) the volume of the first compartment. In some embodiments, the differential volume (as defined herein) of the second compartment is less than (e.g. <1.5×, 2×, 3×, or 5×) the volume of the first compartment. In some embodiments, the total volume of the second compartment is about 1%, 2%, 5%, 7.5%, 10%, 15%, 20%, 25%, 30%, 35%, 40%, 45%, 50%, 55%, 60%, 65%, or 75% greater than the volume of the first compartment. In some embodiments, the differential volume of the first compartment is greater than (e.g., >1.5×, 2×, 3×, or 5×) the volume of the second compartment. In some embodiments, the total volume of the first compartment is about 1%, 2%, 5%, 7.5%, 10%, 15%, 20%, 25%, 30%, 35%, 40%, 45%, 50%, 55%, 60%, 65%, or 75% greater than the volume of the second compartment.

In some embodiments, the particle has a largest linear dimension (LLD), e.g., diameter, of between about 20 nanometers to about 10 millimeters. In some embodiments, the largest linear dimension (LLD), e.g., diameter, of the particle is between about 500 nanometers to about 10 millimeters, between about 1 millimeter to 10 millimeters, between about 1 millimeter to 5 millimeters, between about 1 millimeter to 4 millimeters, between about 1 millimeter to 3 millimeters, between about 1 millimeter to 2 millimeters, or between about 1.5 millimeters to 2 millimeters or about 1.5 millimeters.

In some embodiments, the average distance between the outer boundary of the second (outer) compartment and the interface is between about 1 nanometers and 1 millimeter, e.g., between about 100 nanometers and 1 millimeter, between about 500 nanometers and about 1 millimeter, or between about 500 nanometers and 500 micrometers.

In some embodiments, the particle comprises a cell. In some embodiments, the first compartment comprises a cell and/or the second compartment comprises a cell. In some embodiments, the first compartment and the second compartment both comprise the same type of cell or different types of cells. In some embodiments, the first compartment comprises a cell and the second compartment does not comprise a cell. A particle described herein may comprise a plurality of cells. The cell or plurality of cells may be present in the particle as single cells, cell clusters (e.g., as spheroids), or attached to a microcarrier. In some embodiments, the particle is formed from a polymer solution and comprises at least any of 5, 10, 15, 20, 30, 40, 50, 75, 100, 150, 200, 250 or 400 million cells/ml of the polymer solution or any number between these values. In some embodiments, the particle comprises an epithelial cell, endothelial cell, fibroblast cell, mesenchymal stem cell, keratinocyte cell or an islet cell or a cell derived from any of the foregoing cell types. In some embodiments, the particle comprises a retinal pigment epithelial (RPE cell) or a mesenchymal stem cell (MSC). In some embodiments, the particle comprises an engineered cell (e.g., an engineered RPE cell or an engineered MSC).

In some embodiments, the particle comprises a cell that expresses a therapeutic agent, such as a nucleic acid (e.g., a nucleotide, DNA, or RNA), a polypeptide, a lipid, a sugar (e.g., a monosaccharide, disaccharide, oligosaccharide, or polysaccharide), or a small molecule. In some embodiments, the therapeutic agent is a replacement therapy or a replacement protein, e.g., useful for the treatment of a blood clotting disorder or a lysosomal storage disease in a subject. In some embodiments, the therapeutic agent is a polypeptide, e.g., a Factor VIII protein or variant thereof of a Factor IX protein or variant thereof.

In another aspect, the present disclosure features a preparation of a plurality of particles, wherein one or more of the particles in the plurality comprises: a) a first compartment; b) a second compartment; and c) a compound of Formula (I) as described herein. In some embodiments, each particle in the plurality comprises the first and second compartments and a compound of Formula (I). In some embodiments, at least 75%, 80%, 85%, 90%, 95%, 99%, or more of the particles in the plurality are spherical particles. In some embodiments, the preparation is a pharmaceutically acceptable preparation.

In another aspect, the present disclosure features a method of making a particle described herein. In some embodiments, the first compartment of the particle is formed at the same time as the second compartment of the particle. In some embodiments, the method comprises use of an electrostatic droplet generator equipped with a coaxial needle to form multiple droplets from first and second polymer solutions that comprise a hydrogel forming polymer or a mixture of hydrogel forming polymers. In some embodiments, the polymer or mixture of polymers is modified with a compound of Formula (I). In some embodiments, the polymer is an alginate. In some embodiments, the method further comprises contacting the droplets with a cross-linking solution comprising multivalent cations to cross-link each droplet into a particle (e.g., a hydrogel capsule with an inner compartment and an outer compartment). In some embodiments, the cross-linking solution comprises a cross-linking agent, a buffer, and an osmolarity-adjusting agent. In some embodiments, the cross-linking solution further comprises a surfactant.

In another aspect, the present disclosure features a method of implanting a particle described herein into a subject. In another aspect, the present disclosure features a method of providing a substance (e.g., a therapeutic agent, e.g., a polypeptide) to a subject comprising administering to the subject a particle described herein, wherein the particle comprises, or has the ability to produce, the substance. In another aspect, the present disclosure features a method of treating a subject in need of a substance (e.g., a therapeutic agent, e.g., a polypeptide) comprising administering to the subject a particle described herein, wherein the particle comprises, or has the ability to produce, the substance. In some embodiments, the administering step comprises implanting in the subject a pharmaceutically acceptable preparation comprising a plurality of particles, each of which comprises, or has the ability to produce, the substance. In some embodiments, the subject is a mammal (e.g., a human).

In another aspect, the present disclosure features a method of evaluating a particle described herein. In some embodiments, the method comprises providing a particle described herein and evaluating a structural or functional parameter of the particle. In some embodiments, the method comprises evaluating the particle or a plurality of particles described herein for one or more of: a) structural integrity; b) cell viability; c) the production of a therapeutic agent (e.g., a polypeptide); d) the uptake of a nutrient or oxygen; e) the production of a waste product; and f) fibrosis. In some embodiments, the evaluation is performed at least 1, 5, 10, 20, 30, 60, 90 or 120 days after formation of the particle or administration of the particle to a subject. In some embodiments, the subject is a mammal (e.g., a human).

The details of one or more embodiments of the disclosure are set forth herein. Other features, objects, and advantages of the disclosure will be apparent from the Detailed Description, the Figures, the Examples, and the Claims.

### BRIEF DESCRIPTION OF THE DRAWINGS

FIG. 1 illustrates an exemplary spherical particle of the disclosure, with lines indicating: a first, inner compartment and cells encapsulated therein; a second, outer compartment with an outer boundary; and the interface between the first and second compartments.

FIGS. 2A-2B shows exemplary amino acid sequences encoded by exemplary engineered cells, with FIG. 2A showing the amino acid sequence (SEQ ID NO:1) of a Factor VIII-BDD protein encoded by an exemplary engineered cell and FIG. 2B showing the amino acid sequence (SEQ ID NO:2) of a human wild-type Factor IX protein.

FIG. 3 is a graph comparing quality of single-compartment and two-compartment hydrogel capsules as a function of equivalent cell loading (million cells/ml alginate), where ml alginate is the sum of alginate used to make the first (inner) compartment and second (outer) compartment of the two-compartment capsules.

FIGS. 4A-4B show the effect of altering the flow rate of extruded alginate on the thickness of the second (outer) compartment of an exemplary particle of the disclosure (i.e., a two-compartment hydrogel millicapsule). FIG. 4A is a graph showing the mean second (outer) compartment thickness for particles (about 1.5 millimeter (mm) diameter) produced by varying flow rates of the polymer solutions used to form the first (inner) compartment and second (outer) compartments. FIG. 4B is a table of first (inner) compartment and second (outer) compartment volume percentages and the resulting compartment thicknesses achieved.

FIG. 5 is a graph showing the initial fracture of exemplary particles of the disclosure (i.e., two-compartment hydrogel millicapsules) with varying ratios of inner:outer flow rates (ml/h). The polymer in the first compartment (Inner) is an unmodified high molecular weight alginate and the polymer in the second compartment (Outer) is a mixture of a chemically modified low molecular weight alginate and an unmodified high molecular weight alginate at a 70:30 ratio of chemically modified to unmodified alginate.

FIGS. 6A-6D are brightfield images of exemplary particles (i.e., two-compartment hydrogel millicapsules) with a 50:50 volume ratio of inner:outer compartments. Second (outer) compartments contain low, medium or high conjugation alginate or a control (unmodified), alginate. Exemplary RPE cells engineered to express an exogenous protein were encapsulated in the first (inner) compartment for visualization of the two-compartment architecture.

FIGS. 7A-7F illustrate the effect on fibrosis in vivo of varying the level of chemical modification on the alginate comprising the second (outer) compartment of exemplary particles (i.e., two-compartment hydrogel millicapsules). FIGS. 7A-7E are brightfield images of particles retrieved from C57/BL6 mice 1 week after implantation. Particles containing the engineered RPE cells within the first (inner) compartment had second (outer) compartments composed of: (i) low, medium or high levels of a compound of Formula (I) conjugated to an alginate, (ii) an unmodified alginate, or (iii) empty capsules composed of medium levels of a compound of Formula (I) conjugated to an alginate. FIG. 7F is a graph comparing the mean initial fracture of particles prior to implantation in a mouse model (initial, black bars) and after retrieval following 7 days implantation in C57/BL6 mice (retrieval, gray bars).

FIGS. 8A-8E are images comparing various hydrogel millicapsules and their effect on the fibrotic response. Schematics are shown of the millicapsules retrieved from C57/BL6 mice after a 2-week implantation. FIG. 8A: empty capsules comprising no cells. FIG. 8B: one-compartment capsules with 5000 cells/capsule; FIG. 8C: two-compartment capsules with 5000 cells/particle: FIG. 8D: two-compartment capsules with 2500 cells/capsule; FIG. 8E: two-compartment capsules with 2500 cells/capsule and a thicker second (outer) compartment.

FIGS. 9A-9K are immunofluorescent staining images comparing the level of macrophage adhesion in vivo on exemplary particles (i.e., two-compartment hydrogel millicapsules) with varying (low, medium, or high) amounts of chemically modified alginate in the second (outer) compartment at 1, 2, and 4 weeks post-implantation in C57/BL6 mice. A positive control (SLG20: unmodified medium MW alginate) and a negative control (empty capsule) were included in these experiments.

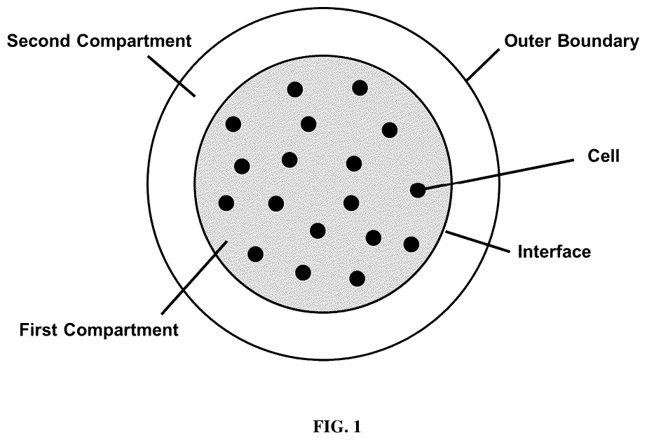

FIGS. 1A-10E are brightfield images indicating the level of fibrotic response on exemplary particles (i.e., two-compartment hydrogel millicapsules) 2 weeks post-implantation in C57/BL6 mice. The particles comprised varying (medium, medium high, high, or double high) amounts of chemically modified alginate in the second (outer) compartment. A negative control (empty) capsule with a medium amount of chemically modified alginate in the second (outer) compartment was also included.

FIG. 11 is a graph comparing the mean initial fracture of particles prior to implantation in a mouse model (initial, black bars) and after retrieval following 2 weeks of implantation in C57/BL6 mice (retrieval, gray bars). The particles comprise varying (medium, medium high, high, or double high) amounts of chemically modified alginate in the second (outer) compartment. A negative control (empty) capsule with a medium amount of chemically modified alginate in the second (outer) compartment was included.

FIGS. 12A-12C are brightfield images indicating the level of fibrotic response in vivo on exemplary particles (i.e., two-compartment hydrogel millicapsules) with either varying (medium or high) amounts of chemically modified alginate in the second (outer) compartment, or non-conjugated afibrotic small molecules (e.g., a compound of Formula (I)) in the second (outer) compartment (“amine added back” capsules), 2 weeks post-implantation in C57/BL6 mice.

FIGS. 13A-13F are immunofluorescent staining images comparing the level of macrophage adhesion in vivo on exemplary particles (i.e., two-compartment hydrogel millicapsules) with differing second (outer) compartments. The second (outer) compartments were prepared from either 70:30 or 60:40 ratio blends of chemically modified low-molecular weight (CM-LMW) alginate to unmodified high-molecular weight (U-HMW) alginate, and also with varying (medium, medium high, or high) amounts of chemically modified alginate in the second (outer) compartment.

FIGS. 14A-14D are brightfield images of encapsulated HEK293F cells in one-compartment or two-compartment hydrogel millicapsules that were cultured for 1 week after encapsulation. FIGS. 14A-14B correspond to images of one-compartment or two-compartment capsules. FIGS. 14C-14D correspond to images of the culture surface to identify cells not contained in the capsules following a 1-week incubation at 37° C.

FIGS. 15A-15C illustrate a correlation between expression levels in vivo of FIX by two-compartment hydrogel millicapsules and concentration of ARPE-19:FIX cells in the inner compartment of the capsules. FIG. 15A shows cell numbers in capsules prepared with different cell loading concentration prior to implant into the IP space mice (Initial) and upon retrieval five days after implant (Retrieval). FIGS. 15B and 15C show FIX levels in plasma and IP fluid produced by the implanted capsules, respectively.

FIGS. 16A-16C illustrate a correlation between expression levels in vivo of FIX by two-compartment hydrogel millicapsules, concentration of ARPE-19:FIX cells in the inner compartment of the capsules and capsule integrity. FIG. 16A shows FIX levels in IP fluid of mice implanted with the capsules. FIG. 16B are brightfield images of the capsules prepared with 646M/ml cells at the pre-implantation (initial) and retrieval time points.

FIG. 17 shows in Tables 4-8 exemplary amino acid sequences and coding sequences for therapeutic polypeptides and nucleotide sequences within an exemplary expression vector useful for engineering RPE cells.

FIG. 18A-18B are brightfield images of exemplary particles (i.e., two-compartment hydrogel capsules about 0.75 mm in diameter (FIG. 18A) or about 1.0 mm in diameter (FIG. 18B) with a 50:50 volume ratio of inner:outer compartments. Each of the first (inner) and second (outer) compartments contain medium conjugation alginate. Exemplary RPE cells engineered to express an exogenous protein were encapsulated in the first (inner) compartment for visualization of the two-compartment architecture.

### DETAILED DESCRIPTION

The present disclosure features a particle comprising a first compartment, a second compartment, and a compound of Formula (I) (e.g., as described herein), as well as compositions and methods of making and using the same. In some embodiments, the particles and compositions thereof are useful for the prevention or treatment of a disease, disorder, or condition. In some embodiments, particles configured as hydrogel millicapsules comprising a first hydrogel compartment and a second hydrogel compartment and a compound of Formula (I) exhibit advantageous properties, e.g., they are more afibrotic than similar millicapsules lacking a compound of Formula (I) but comprised of the same type of polymer, and substantially the same size, and can hold a greater number of cells with minimal detrimental effect on capsule quality compared with millicapsules containing a single compartment. In some embodiments, the particles described herein comprise a cell (e.g., an engineered cell) that produces a therapeutic agent (e.g., a polypeptide) suitable for treating a disease, disorder, or condition in a subject.

### Abbreviations and Definitions

Throughout the detailed description and examples of the disclosure the following abbreviations will be used.
CM-Alg chemically modified alginate
CM-LMW-Alg chemically modified, low molecular weight alginate
CM-LMW-Alg-101 low molecular weight alginate, chemically modified with Compound 101 shown in Table 2
CM-HMW-Alg chemically modified, high molecular weight alginate
CM-HMW-Alg-101 high molecular weight alginate, chemically modified with Compound 101 shown in Table 2
CM-MMW-Alg chemically modified, medium molecular weight alginate
CM-MMW-Alg-101 medium molecular weight alginate, chemically modified with Compound 101 shown in Table 2
HMW-Alg high molecular weight alginate
MMW-Alg medium molecular weight alginate
U-Alg unmodified alginate
U-HMW-Alg unmodified high molecular weight alginate
U-LMW-Alg unmodified low molecular weight alginate
U-MMW-Alg unmodified medium molecular weight alginate
70:30 CM-Alg:U-Alg 70:30 mixture (V:V) of a chemically modified alginate and an unmodified alginate

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About”, when used herein to modify a numerically defined parameter (e.g., a physical description of a hydrogel capsule such as diameter, sphericity, number of cells in a particle, the number of particles in a preparation), means that the parameter may vary by as much as 15% above or below the stated numerical value for that parameter. For example, a hydrogel capsule defined as having a diameter of about 1.5 millimeters (mm) and encapsulating about 5 million (M) cells may have a diameter of 1.275 to 1.725 mm and may encapsulate about 4.25 M to 5.75 M cells. In some embodiments, about means that the parameter may vary by as much as 10% above or below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., fluorescence microscope to acquire fluorescence microscopy data.

“Administer”, “administering”, or “administration”, as used herein, refer to implanting, absorbing, ingesting, injecting, or otherwise introducing an entity described herein (e.g., a particle comprising a first compartment, a second compartment, and a compound of Formula (I) (including particles encapsulating cells, e.g., engineered RPE cells), or a composition comprising said particles), or providing the same to a subject.

“Afibrotic”, as used herein, refers to a compound or material that mitigates the foreign body response (FBR). For example, the amount of FBR in a biological tissue that is induced by implant into that tissue of a particle (e.g., a hydrogel capsule) comprising an afibrotic compound (e.g., a compound of Formula (I), e.g., a compound listed in Table 2) is lower than the FBR induced by implantation of an afibrotic-null reference particle, i.e., a particle that lacks the afibrotic compound or material, but is of substantially the same composition (e.g., same cell type(s)) and structure (e.g., size, shape, no. of compartments, same encapsulating polymers, etc.). In an embodiment, the degree of the FBR is assessed by the immunological response in the tissue containing the implanted particle (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myfibroblasts (alpha-muscle actin, SMA) or general cellular deposition, quantification of 79 RNA sequences of known inflammation factors and immune cell markers, or FACS analysis for macrophage and neutrophil cells on retrieved particles (e.g., capsules) after a set time period (e.g., 14 days) in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a particle described herein (e.g., a two-compartment hydrogel capsule comprising an afibrotic compound disposed in and/or on the surface of the outer compartment), is at least about 80%, about 85%, about 90%, about 95%, about 99%, or about 100% lower than the FBR induced by an FBR-null reference particle, e.g., a particle that is substantially identical to the claimed particle except for lacking the afibrotic compound or material but is otherwise substantially identical to the claimed particle. In some embodiments, the FBR (e.g., FBR biomarker level(s)) induced by an implanted particle is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Cell,” as used herein, refers to an engineered cell or a cell that is not engineered. In an embodiment, a cell is an immortalized cell.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 1 below.

TABLE 1
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface Exposure | Asp, Glu, Asn, Aln, His, Arg, Lys |
| | Cys, Ser, Tyr, Pro, Ala, Gly, Trp, Tyr |
| | Met, Ile, Leu, Val, Phe |
| Secondary Structure Propensity | Ala, Glu, Aln, His, Lys, Met, Leu, Arg |
| | Cys, Thr, Ile, Val, Phe, Tyr, Trp |
| | Ser, Gly, Pro, Asp, Asn |
| Evolutionary Conservation | Asp, Glu |
| | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, particle, or method. As a non-limiting example, a therapeutic protein that consists essentially of a recited amino acid sequence may also include one or more amino acids, including substitutions in the recited amino acid sequence, of one or more amino acid residues, which do not materially affect the relevant biological activity of the therapeutic protein, respectively. As another non-limiting example, a polypeptide that consists essentially of a recited amino acid sequence may contain one or more covalently attached moieties (e.g., a radioactive or fluorescent label) that do not materially change the relevant biological activity of the polypeptide.

“Derived from”, as used herein with respect to a cell or cells, refers to a cell or cells obtained from tissue, a cell line, or other cells, which optionally are then cultured, passaged, immortalized, differentiated and/or induced, to produce the derived cell(s).

“Differential volume,” as used herein, refers to a volume of one compartment within a particle that excludes the space occupied by another compartment(s). For example, the differential volume of the second compartment in a 2-compartment particle refers to a volume within the second (e.g., outer) compartment that excludes space occupied by the first compartment.

“Effective amount” as used herein refers to an amount of a composition of particles (e.g., a particle composition) or a particle component, e.g, a cell, e.g., an engineered cell, or an agent, e.g., a therapeutic agent, produced by a cell, e.g., an engineered cell, sufficient to elicit a biological response, e.g., to treat a disease, disorder, or condition. In some embodiments, the term “effective amount” refers to the amount of a particle component, e.g., number of cells in the particle, the concentration or density of an afibrotic compound disposed on the particle surface and/or in the outer compartment. As will be appreciated by those of ordinary skill in this art, the effective amount may vary depending on such factors as the desired biological endpoint, the pharmacokinetics of the therapeutic agent, composition or particle, the condition being treated, the mode of administration, and the age and health of the subject. An effective amount encompasses therapeutic and prophylactic treatment. For example, to mitigate the FBR induced by a particle, an afibrotic-effective amount of a compound of Formula (I) may reduce the fibrosis or stop the growth or spread of fibrotic tissue on or near the implanted particle. An afibrotic-effective amount of a particle, composition or component thereof (e.g., an afibrotic compound, e.g., an afibrotic polymer) may be determined by any technique known in the art or described herein.

An “endogenous nucleic acid” as used herein, is a nucleic acid that occurs naturally in a subject cell.

An “endogenous polypeptide,” as used herein, is a polypeptide that occurs naturally in a subject cell.

“Engineered cell,” as used herein, is a cell (e.g., an RPE cell) having a non-naturally occurring alteration, and typically comprises a nucleic acid sequence (e.g., DNA or RNA) or a polypeptide not present (or present at a different level than) in an otherwise similar cell under similar conditions that is not engineered (an exogenous nucleic acid sequence). In an embodiment, an engineered cell comprises an exogenous nucleic acid (e.g., a vector or an altered chromosomal sequence). In an embodiment, an engineered cell comprises an exogenous polypeptide. In an embodiment, an engineered cell comprises an exogenous nucleic acid sequence, e.g., a sequence, e.g., DNA or RNA, not present in a similar cell that is not engineered. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a chromosomal or extra-chromosomal nucleic acid sequence, which comprises a sequence that encodes a polypeptide, or which is expressed as a polypeptide. In an embodiment, the exogenous nucleic acid sequence comprises a first chromosomal or extra-chromosomal exogenous nucleic acid sequence that modulates the conformation or expression of a second nucleic acid sequence, wherein the second amino acid sequence can be exogenous or endogenous. For example, an engineered cell can comprise an exogenous nucleic acid that controls the expression of an endogenous sequence. In an embodiment, an engineered cell comprises a polypeptide present at a level or distribution which differs from the level found in a similar cell that has not been engineered. In an embodiment, an engineered cell comprises an RPE cell engineered to provide an RNA or a polypeptide. For example, an engineered cell may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, an engineered cell (e.g., an RPE cell) comprises an exogenous nucleic acid sequence that comprises a chromosomal or extra-chromosomal nucleic acid sequence comprising a sequence that encodes a polypeptide, or which is expressed as a polypeptide. In an embodiment, an engineered cell (e.g., an RPE cell) comprises an exogenous nucleic acid sequence that modulates the conformation or expression of an endogenous sequence.

“An “exogenous nucleic acid,” as used herein, is a nucleic acid that does not occur naturally in a subject cell.

An “exogenous polypeptide,” as used herein, is polypeptide that does not occur naturally in a subject cell.

“Factor VII protein” or “FVII protein” as used herein, means a polypeptide that comprises the amino acid sequence of a naturally-occurring factor VII protein or variant thereof that has a FVII biological activity, e.g., promoting blood clotting, as determined by an art-recognized assay, unless otherwise specified. Naturally-occurring FVII exists as a single chain zymogen, a zymogen-like two-chain polypeptide and a fully activated two-chain form (FVIIa). In some embodiments, reference to FVII includes single-chain and two-chain forms thereof, including zymogen-like and FVIIa. FVII proteins that may be produced by a particle described herein (e.g., a two-compartment hydrogel capsule containing engineered RPE cells), include wild-type primate (e.g., human), porcine, canine, and murine proteins, as well as variants of such wild-type proteins, including fragments, mutants, variants with one or more amino acid substitutions and/or deletions. In some embodiments, a variant FVII protein is capable of being activated to the fully activated two-chain form (Factor Vila) that has at least 50%, 75%, 90% or more (including >100%) of the activity of wild-type Factor VIIa. Variants of FVII and FVIIa are known, e.g., marzeptacog alfa (activated) (MarzAA) and the variants described in European Patent No. 1373493, U.S. Pat. Nos. 7,771,996, 9,476,037 and US published application No. US20080058255.

FVII biological activity may be quantified by an art recognized assay, unless otherwise specified. For example, FVII biological activity in a sample of a biological fluid, e.g., plasma, may be quantified by (i) measuring the amount of Factor Xa produced in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to tissue factor (TF) using an instrument based on surface plasmon resonance (Persson, FEBS Letts. 413:359-363, 1997); or (iv) measuring hydrolysis of a synthetic substrate; and/or (v) measuring generation of thrombin in a TF-independent in vitro system. In an embodiment, FVII activity is assessed by a commercially available chromogenic assay (BIOPHEN FVII, HYPHEN BioMed Neuville sur Oise, France), in which the biological sample containing FVII is mixed with thromboplastin calcium, Factor X and SXa-11 (a chromogenic substrate specific for Factor Xa.

“Factor VIII protein” or “FVIII protein” as used herein, means a polypeptide that comprises the amino acid sequence of a naturally occurring factor VIII polypeptide or variant thereof that has an FVIII biological activity, e.g., coagulation activity, as determined by an art-recognized assay, unless otherwise specified. FVIII proteins that may be expressed by a particle described herein, e.g., a two-compartment hydrogel capsule containing engineered RPE cells, include wild-type primate (e.g., human), porcine, canine, and murine proteins, as well as variants of such wild-type proteins, including fragments, mutants, variants with one or more amino acid substitutions and/or deletions, B-domain deletion (BDD) variants, single chain variants and fusions of any of the foregoing wild-type or variants with a half-life extending polypeptide. In an embodiment, the cells are engineered to encode a precursor factor VIII polypeptide (e.g., with the signal sequence) with a full or partial deletion of the B domain. In an embodiment, the cells are engineered to encode a single chain factor VIII polypeptide. A variant FVIII protein preferably has at least 50%, 75%, 90% or more (including >100%) of the coagulation activity of the corresponding wild-type factor VIII. Assays for measuring the coagulation activity of FVIII proteins include the one stage or two stage coagulation assay (Rizza et al., 1982, Coagulation assay of FVIII:C and FIXa in Bloom ed. The Hemophelias. NY Churchill Livingston 1992) or the chromogenic substrate FVIII:C assay (Rosen, S. 1984. Scand J Haematol 33:139-145, suppl.)

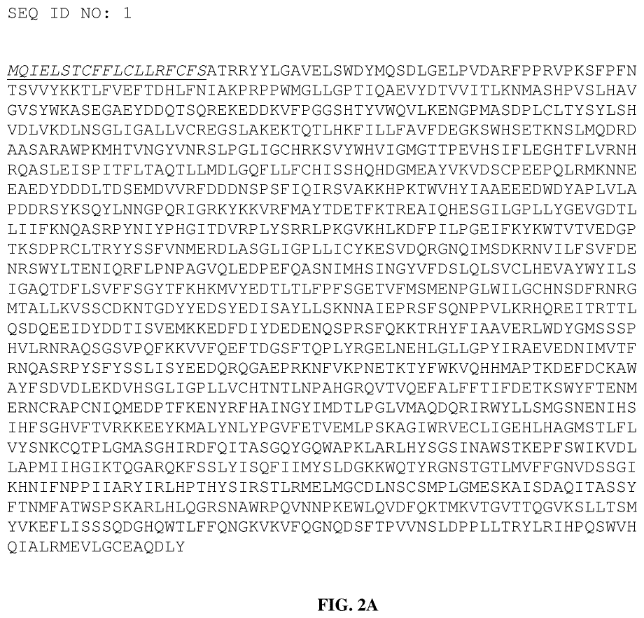

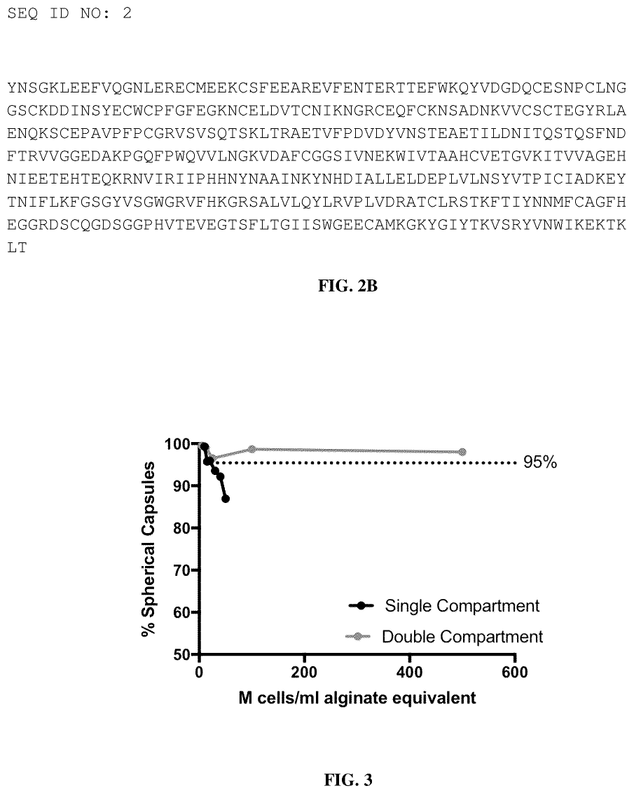

A number of FVIII-BDD variants are known, and include, e.g., variants with the full or partial B-domain deletions disclosed in any of the following U.S. Pat. No. 4,868,112 (e.g., col. 2, line 2 to col. 19, line 21 and table 2); U.S. Pat. No. 5,112,950 (e.g., col. 2, lines 55-68, FIG. 2, and example 1); U.S. Pat. No. 5,171,844 (e.g., col. 4, line 1 22 to col. 5, line 36); U.S. Pat. No. 5,543,502 (e.g., col. 2, lines 17-46); U.S. Pat. Nos. 5,595,886; 5,610,278; 5,789,203 (e.g., col. 2, lines 26-51 and examples 5-8); U.S. Pat. No. 5,972,885 (e.g., col. 1, lines 25 to col. 2, line 40); U.S. Pat. No. 6,048,720 (e.g., col. 6, lines 1-22 and example 1); U.S. Pat. Nos. 6,060,447; 6,228,620; 6,316,226 (e.g., col. 4, line 4 to col. 5, line 28 and examples 1-5); U.S. Pat. Nos. 6,346,513; 6,458,563 (e.g., col. 4, lines 25-53) and U.S. Pat. No. 7,041,635 (e.g., col. 2, line 1 to col. 3, line 19, col. 3, line 40 to col. 4, line 67, col. 7, line 43 to col. 8, line 26, and col. 11, line 5 to col. 13, line 39).

In some embodiments, a FVIII-BDD protein produced by a particle described herein (e.g., expressed by engineered cells contained in the particle) has one or more of the following deletions of amino acids in the B-domain: (i) most of the B domain except for amino-terminal B-domain sequences essential for intracellular processing of the primary translation product into two polypeptide chains (WO 91/09122); (ii) a deletion of amino acids 747-1638 (Hoeben R. C., et al. J. Biol. Chem. 265 (13): 7318-7323 (1990)); amino acids 771-1666 or amino acids 868-1562 (Meulien P., et al. Protein Eng. 2(4):301-6 (1988); amino acids 982-1562 or 760-1639 (Toole et al., Proc. Natl. Acad. Sci. U.S.A. 83:5939-5942 (1986)); amino acids 797-1562 (Eaton et al., Biochemistry 25:8343-8347 (1986)); 741-1646 (Kaufman, WO 87/04187)), 747-1560 (Sarver et al., DNA 6:553-564 (1987)); amino acids 741-1648 (Pasek, WO 88/00831)), amino acids 816-1598 or 741-1689 (Lagner (Behring Inst. Mitt. (1988) No 82:16-25, EP 295597); a deletion that includes one or more residues in a furin protease recognition sequence, e.g., LKRHQR at amino acids 1643-1648, including any of the specific deletions recited in U.S. Pat. No. 9,956,269 at col. 10, line 65 to col. 11, line 36.

In other embodiments, a FVIII-BDD protein retains any of the following B-domain amino acids or amino acid sequences: (i) one or more N-linked glycosylation sites in the B-domain, e.g., residues 757, 784, 828, 900, 963, or optionally 943, first 226 amino acids or first 163 amino acids (Miao, H. Z., et al., Blood 103(a): 3412-3419 (2004), Kasuda, A., et al., J. Thromb. Haemost. 6: 1352-1359 (2008), and Pipe, S. W., et al., J. Thromb. Haemost. 9: 2235-2242 (2011).

In some embodiments, the FVIII-BDD protein is a single-chain variant generated by substitution of one or more amino acids in the furin protease recognition sequence (LKRHQR at amino acids 1643-1648) that prevents proteolytic cleavage at this site, including any of the substitutions at the R1645 and/or R1648 positions described in U.S. Pat. Nos. 10,023,628, 9,394,353 and 9,670,267.

In some embodiments, any of the above FVIII-BDD proteins may further comprise one or more of the following variations: a F309S substitution to improve expression of the FVIII-BDD protein (Miao, H. Z., et al., Blood 103(a): 3412-3419 (2004); albumin fusions (WO 2011/020866); and Fc fusions (WO 04/101740).

All FVIII-BDD amino acid positions referenced herein refer to the positions in full-length human FVIII, unless otherwise specified.

“Factor IX protein” or “FIX protein”, as used herein, means a polypeptide that comprises the amino acid sequence of a naturally occurring factor IX protein or variant thereof that has a FIX biological activity, e.g., coagulation activity, as determined by an art-recognized assay, unless otherwise specified. FIX is produced as an inactive zymogen, which is converted to an active form by factor XIa excision of the activation peptide to produce a heavy chain and a light chain held together by one or more disulfide bonds. FIX proteins that may be produced by a particle described herein (e.g., expressed by engineered RPE cells contained in the particle) include wild-type primate (e.g., human), porcine, canine, and murine proteins, as well as variants of such wild-type proteins, including fragments, mutants, variants with one or more amino acid substitutions and/or deletions and fusions of any of the foregoing wild-type or variant proteins with a half-life extending polypeptide. In an embodiment, cells are engineered to encode a full-length wild-type human factor IX polypeptide (e.g., with the signal sequence) or a functional variant thereof. A variant FIX protein preferably has at least 50%, 75%, 90% or more (including >100%) of the coagulation activity of wild-type factor VIX. Assays for measuring the coagulation activity of FIX proteins include the Biophen Factor IX assay (Hyphen BioMed) and the one stage clotting assay (activated partial thromboplastin time (aPTT), e.g., as described in EP 2 032 607 B2, thrombin generation time assay (TGA) and rotational thromboelastometry, e.g., as described in WO 2012/006624.

A number of functional FIX variants are known and may be expressed by engineered cells encapsulated in a particle described herein, including any of the functional FIX variants described in the following international patent publications: WO 02/040544 A3 at page 4, lines 9-30 and page 15, lines 6-31; WO 03/020764 A2 in Tables 2 and 3 at pages 14-24, and at page 12, lines 1-27; WO 2007/149406 A2 at page 4, line 1 to page 19, line 11; WO 2007/149406 A2 at page 19, line 12 to page 20, line 9; WO 08/118507 A2 at page 5, line 14 to page 6, line 5; WO 09/051717 A2 at page 9, line 11 to page 20, line 2; WO 09/137254 A2 at page 2, paragraph [006] to page 5, paragraph [011] and page 16, paragraph [044] to page 24, paragraph [057]; WO 09/130198 A2 at page 4, line 26 to page 12, line 6; WO 09/140015 A2 at page 11, paragraph [0043] to page 13, paragraph [0053]; WO 2012/006624; WO 2015/086406.

In certain embodiments, the FIX polypeptide comprises a wild-type or variant sequence fused to a heterologous polypeptide or non-polypeptide moiety extending the half-life of the FIX protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof. An exemplary FIX polypeptide is the rFIXFc protein described in WO 2012/006624, which is an FIXFc single chain (FIXF c-sc) and an Fc single chain (Fc-sc) bound together through two disulfide bonds in the hinge region of Fc.

FIX variants also include gain and loss of function variants. An example of a gain of function variant is the “Padua” variant of human FIX, which has a L (leucine) at position 338 of the mature protein instead of an R (arginine) (corresponding to amino acid position 384 of SEQ ID NO:2), and has greater catalytic and coagulant activity compared to wild-type human FIX (Chang et al., J. Biol. Chem., 273:12089-94 (1998)). An example of a loss of function variant is an alanine substituted for lysine in the fifth amino acid position from the beginning of the mature protein, which results in a protein with reduced binding to collagen IV (e.g., loss of function).

“Interleukin-2 protein” or “IL-2 protein”, as used herein means a polypeptide comprising the amino acid sequence of a naturally occurring IL-2 protein or variant thereof that has an IL-2 biological activity, e.g., activate IL-2 receptor signaling in Treg cells, as determined by an art-recognized assay, unless otherwise specified. IL-2 proteins that may be produced by a particle described herein, e.g., a particle containing engineered RPE cells, include wild-type primate (e.g., human), porcine, canine, and murine proteins, as well as variants of such wild-type proteins. A variant IL-2 protein preferably has at least 50%, 75%, 90% or more (including >100%) of the biological activity of the corresponding wild-type IL-2. Biological activity assays for IL-2 proteins are described in U.S. Pat. No. 10,035,836, and include, e.g., measuring the levels of phosphorylated STAT5 protein in Treg cells compared to CD4+CD25−/low T cells or NK cells. Variant IL-2 proteins that may be produced by a particle of the present disclosure (e.g., a particle containing engineered RPE cells) include proteins with one or more of the following amino acid substitutions: N88R, N88I, N88G, D20H, Q126L, Q126F, and C125S or C125A.

“Islet cell” as used herein means a cell that comprises any naturally occurring or any synthetically created, or modified, cell that is intended to recapitulate, mimic or otherwise express, in part or in whole, the functions, in part or in whole, of the cells of the pancreatic islets of Langerhans. The term “islet cells” includes glucose-responsive, insulin producing cells derived from stem cells, e.g., from an induced pluripotent stem cell line.

“Mannitol”, as used herein, refers to D-mannitol unless otherwise explicitly stated.

“Mesenchymal stem function cell” or “MSFC,” as those terms are used herein, refers to a cell derived from, or having at least one characteristic specific to a cell of, mesodermal lineage, and wherein the MSFC is i) not in a terminal state of differentiation and ii) can terminally differentiate into one or more cell types. An MSFC does not comprise a cell of endodermal origin, e.g., a gut cell, or of ectodermal origin, e.g., a cell derived from skin, CNS, or a neural cell. In an embodiment, the MSFC is multipotent. In an embodiment, the MSFC is not totipotent. In an embodiment, an MSFC comprises one or more of the following characteristics:
a) it comprises a mesenchymal stem cell (MSC) or a cell derived therefrom, including a cell derived from a primary cell culture of MSCs, a cell isolated directly (without long term culturing, e.g., less than 5 or 10 passages or rounds of cell division since isolation) from naturally occurring MSCs, e.g., from a human or other mammal, a cell derived from a transformed, a pluripotent, an immortalized, or a long term (e.g., more than 5 or 10 passages or rounds of cell division) MSC culture. In an embodiment, the MSFC is derived from a human source, e.g., the blood (e.g., peripheral blood), bone marrow (e.g., the iliac crest, femora, tibiae, spine, rib, or knee), synovial tissue, adipose tissue, skin, fetal tissue, umbilical cord, or the placenta;
b) it comprises a cell that has been obtained from a less differentiated cell, e.g., a cell developed, programmed, or reprogramed (e.g., in vitro) into an MSC or a cell that is, except for any genetic engineering, substantially similar to one or more of a naturally occurring MSC or a cell from a primary or long term culture of MSCs, or a cell described in a) above. Examples of less differentiated cells from which MSFC can be derived include IPS cells, embryonic stem cells, or other totipotent or pluripotent cells; see, e.g., Chen, Y. S. et al (2012) Stem Cells Transl Med 1(83-95); Frobel, J et al (2014) Stem Cell Reports 3(3):414-422; Zou, L et al (2013) Sci Rep 3:2243;
c) it is multipotent, e.g., as measured by any assay capable of providing information about cell multipotency, e.g., microscopy;
d) it exhibits a characteristic mononuclear ovoid, stellate shape or spindle shape, with a round to oval nucleus. The oval elongate nucleus may have prominent nucleoli and a mix of heterochromatin and euchromatin. An MSFC (e.g., an MSC) may have little cytoplasm, but many thin processes that appear to extend from the nucleus;
e) it is capable of cell division, e.g., as measured any assay capable of providing information about cell division, e.g., microscopy. In an embodiment, an MSFC is capable of cell division in culture (e.g., prior to being encapsulated or incorporated into a particle described herein). In an embodiment, it is capable of cell division after being encapsulated, e.g., encapsulated as described herein, or incorporated into a particle (e.g., a 2-compartment capsule described herein). In an embodiment, it is incapable of cell division after reaching confluence;
f) it is capable of differentiating into a mesenchymal cell lineage, e.g., an osteoblast, a chrondoblast, an adipocyte, or a fibroblast;
g) it expresses a mesenchymal cell marker, e.g., one, two, three, four, five or all of CD105, CD106, CD73, CD90, Stro-1, CD49a, CD29, CD44, CD146, CD166, TNAP+, THY-1+, Stro-2, Stro-4, and alkaline phosphatase;
h) it does not express significant levels of one, two, three, or any of CD34, CD31, VE-cadherin, CD45, HLA-DR, CD11b and a glycophorin or leukocyte differentiation antigen, e.g. CD14, CD33, CD3 and CD19;
i) it expresses one, two, or all of CD75, CD90, and CD105 and does not express one, two, or any of CD45, CD34, and CD14;
j) it is anti-inflammatory or immune-dampening, e.g., as measured by any method capable of providing information regarding inflammation, e.g., in vivo inhibition of T cell proliferation;
k) it is capable of being adherent, e.g., plastic adherent, e.g., as determined by, e.g., visual inspection; or
l) can grow in three dimensions, e.g., as determined by, e.g., visual inspection.

“Parathyroid hormone” or “PTH” as used herein means a polypeptide or peptide that comprises the amino acid sequence of a naturally occurring parathyroid hormone polypeptide or peptide or variant thereof that has a PTH biological activity, e.g., as determined by an art recognized assay. PTH polypeptides and peptides that may be expressed by encapsulated cells described herein include wild-type primate (e.g., human), porcine, canine, and murine proteins, as well as variants of such wild-type proteins. Such PTH polypeptides and peptides may consist essentially of the wild-type human sequence for pre-pro-PTH polypeptide (115 amino acids), pro-PTH polypeptide (90 amino acids), the mature 84-amino acid peptide (PTH(1-84)), and biologically active variants thereof, such as the truncated variant peptide PTH(1-34). PTH peptide variants with/one or more amino acid substitutions in the human wild-type sequence have been described, e.g., in U.S. Pat. Nos. 7,410,948 and 8,563,513 and in US published patent application US20130217630. A PTH variant preferably has at least 50%, 75%, 90% or more (including >100%) of a biological activity of the corresponding wild-type PTH. An assay to detect certain PTH variants by tandem mass spectrometry is described in U.S. Pat. No. 8,383,417. A biological activity assay for PTH peptide variants—stimulation of adenylate cyclase as determined by measuring cAMP levels—is described in U.S. Pat. No. 7,410,948.

“Poloxamer”, as used herein, refers to the standard generic term for a class of nonionic triblock linear copolymers composed of a central hydrophobic chain of polyoxypropylene (poly(propylene oxide)) flanked by two polyoxyethylene (poly(ethylene oxide)) moieties.

“Poloxamer 188” or “P 188”, as used herein, refers to a poloxamer with an approximate molecular mass of 1800 g/mole for the polyoxypropylene core and an oxyethylene content of about 80% weight percent, e.g., 79.0 to 83.7 percent. In an embodiment, poloxamer 188 has an average molecular weight of 8350 g/mole. In an embodiment, poloxamer 188 has an average molecular weight of 7680 g/mole to 9510 g/mole, e.g., as determined by size exclusion chromatography, and an oxyethylene content of 81.8±1.9% weight percent. In an embodiment, each polyoxyethylene chain in poloxamer 188 has 75-85 (e.g., 80) ethylene oxide monomers and the polyoxypropylene core has 25-30 (e.g., 27) propylene oxide monomers. In an embodiment, poloxamer 188 used in a process described herein substantially meets the specifications set forth in a poloxamer monograph published by the United States Pharmacopeia-National Formulary (USP-NF) or the European Pharmacopoeia (Ph. Eur.) that is official at the time the process is performed.

“Poloxamer 407” or “P 407”, as used herein, means a poloxamer with an approximate molecular mass of 4000 g/mole for the polypropylene core and an oxyethylene content of about 70% by weight. In an embodiment, poloxamer 407 has an average molecular weight of 9,840 g/mole to 14,600 g/mole and an oxyethylene content of 73.2±1.7% by weight. In an embodiment, each polyoxyethylene chain in poloxamer 407 has 95-105 (e.g., 101) ethylene oxide monomers (e.g., and the polyoxypropylene core has 54-60 (e.g., 56) propylene oxide monomers.

“Polypeptide”, as used herein, refers to a polymer comprising amino acid residues linked through peptide bonds and having at least two, and in embodiments, at least 10, 50, 75, 100, 150 or 200 amino acid residues.

“Prevention,” “prevent,” and “preventing” as used herein refers to a treatment that comprises administering or applying a therapy, e.g., administering a composition of particles encapsulating cells (e.g., as described herein), prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “replacement therapy” or “replacement protein” is a therapeutic protein or functional fragment thereof that replaces or augments a protein that is diminished, present in insufficient quantity, altered (e.g., mutated) or lacking in a subject having a disease or condition related to the diminished, altered or lacking protein. Examples are certain blood clotting factors in certain blood clotting disorders or certain lysosomal enzymes in certain lysosomal storage diseases. In an embodiment, a replacement therapy or replacement protein provides the function of an endogenous protein. In an embodiment, a replacement therapy or replacement protein has the same amino acid sequence of a naturally occurring variant, e.g., a wild type allele or an allele not associated with a disorder, of the replaced protein. In an embodiment, or replacement therapy or a replacement protein differs in amino acid sequence from a naturally occurring variant, e.g., a wild type allele or an allele not associated with a disorder, e.g., the allele carried by a subject, at no more than about 1, 2, 3, 4, 5, 10, 15 or 20% of the amino acid residues.

“RPE cell” as used herein refers to a cell having one or more of the following characteristics: a) it comprises a retinal pigment epithelial cell (RPE) (e.g., cultured using the ARPE-19 cell line (ATCC® CRL-2302™)) or a cell derived therefrom, including a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BEST1, or αB-crystallin; iii) it is naturally found in the retina and forms a monolayer above the choroidal blood vessels in the Bruch's membrane; iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC® CRL-2302™)). In an embodiment, an RPE cell described herein is engineered, e.g., to have a new property, e.g., the cell is engineered to express a therapeutic protein. In other embodiments, an RPE cell is not engineered.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Publication No. 2017/02334455. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein, refers to a particle having a curved surface that forms a sphere (e.g., a completely round ball) or sphere-like shape. Spheres and sphere-like objects can be mathematically defined by rotation of circles, ellipses, or a combination around each of the three perpendicular axes, a, b, and c. For a sphere, the three axes a, b, and c are the same length. Generally, a sphere-like shape is an ellipsoid (for its averaged surface) with semi-principal axes a, b, and c that are within 10%, or 5%, or 2.5% of each other. The diameter of a sphere or sphere-like shape is the average diameter, such as the average of the semi-principal axes.

“Subject” as used herein refers to a human or non-human animal. In an embodiment, the subject is a human (i.e., a male or female), e.g., of any age group, a pediatric subject (e.g., infant, child, adolescent) or adult subject (e.g., young adult, middle-aged adult, or senior adult). In an embodiment, the subject is a non-human animal, for example, a mammal (e.g., a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Total volume,” as used herein, refers to a volume within one compartment of a particle that includes the space occupied by another compartment. For example, the total volume of the second (e.g., outer) compartment of a two-compartment particle refers to a volume within the second compartment that includes space occupied by the first compartment.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not.

“Von Willebrand factor protein” or “VWF protein”, as used herein, means a polypeptide that comprises the amino acid sequence of a naturally occurring VWF polypeptide or variant thereof that has VWF biological activity, e.g., FVIII binding activity, as determined by an art-recognized assay, unless otherwise specified. VWF proteins that may be produced by a particle described herein (e.g., expressed by engineered cells contained in the particle) include wild-type primate (e.g., human), porcine, canine, and murine proteins, as well as variants of such wild-type proteins. The encapsulated cells may be engineered to encode any of the following VWF polypeptides: precursor VWF of 2813 amino acids, a VWF lacking the signal peptide of 22 amino acids and optionally the prepropeptide of 741 amino acids, mature VWF protein of 2050 amino acids, and truncated variants thereof, such as a VWF fragment sufficient to stabilize endogenous FVIII levels in VWF-deficient mice, e.g, a truncated variant containing the D′D3 region (amino acids 764-1247) or the D1D2D′D3 region; and VWF variants with one or more amino acid substitutions, e.g., in the D′ region as described in U.S. Pat. No. 9,458,223. A variant VWF protein preferably has at least 50%, 75%, 90% or more (including >100%) of a biological activity of the corresponding wild-type VWF protein. Art-recognized assays for determining the biological activity of a VWF include ristocetin co-factor activity (Federici A B et al. 2004. Haematologica 89:77-85), binding of VWF to GP Ibα of the platelet glycoprotein complex Ib-V-IX (Sucker et al. 2006. Clin Appl Thromb Hemost. 12:305-310), and collagen binding (Kallas & Talpsep. 2001. Annals of Hematology 80:466-471).

In some embodiments, the VWF protein produced by a particle of the disclosure comprises a naturally-occurring or variant VWF amino acid sequence fused to a heterologous polypeptide or non-polypeptide moiety extending the half-life of the VWF protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 12 carbon atoms (“C₂-C₁₂ alkenyl”), 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 12 carbon atoms (“C₂-C₁₀ alkynyl”), 2 to 10 carbon atoms (“C₂-C₁₀ alkynyl”), 2 to 8 carbon atoms (“C₂-C₅ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds used in the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

The present disclosure may employ compounds of Formula (I) in a prodrug form. Prodrugs are those compounds that readily undergo chemical changes under physiological conditions to provide the compounds useful in the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of a electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

The symbol “ [symbol] ” as used herein refers to a connection to an entity, e.g., a polymer (e.g., hydrogel-forming polymer such as alginate) or an implantable element (e.g., a particle, device or material). The connection represented by “ [symbol] ” may refer to direct attachment to the entity, e.g., a polymer or an implantable element, or may refer to linkage to the entity through an attachment group. An “attachment group,” as described herein, refers to a moiety for linkage of a compound of Formula (I) to an entity (e.g., a polymer or an implantable element as described herein), and may comprise any attachment chemistry known in the art. A listing of exemplary attachment groups is outlined in Bioconjugate Techniques (3^{rd} ed, Greg T. Hermanson, Waltham, MA: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl, alkenyl, alkynyl, or thiol. In some embodiments, an attachment group is a cross-linker. In some embodiments, the attachment group is —C(O)(C₁-C₆-alkylene)-, wherein alkylene is substituted with R¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Features of Particles

The present disclosure features particles comprising a first compartment, a second compartment, and a compound of Formula (I), e.g., a described herein. The particle may be spherical (e.g., a hydrogel capsule) or any other shape. The particle may comprise materials such as metals, metallic alloys, ceramics, polymers, fibers, inert materials, and combinations thereof. A particle may be completely made up of one type of material, or may comprise numerous other materials within the second compartment and any first compartments.

In some embodiments, the first compartment is modified with a compound of Formula (I). In some embodiments, the second compartment is modified with a compound of Formula (I). In some embodiments, both the first compartment and the second compartment are independently modified with a compound of Formula (I).

In some embodiments, a particle has a largest linear dimension (LLD), e.g., mean diameter, or size that is greater than 1 millimeter (mm), preferably 1.5 mm or greater. In some embodiments, a particle can be as large as 10 mm in diameter or size. For example, a particle described herein is in a size range of 0.5 mm to 10 mm, 1 mm to 10 mm, 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm. In some embodiments, the particle has a mean diameter or size between 1 mm to 8 mm. In some embodiments, the particle has a mean diameter or size between 1 mm to 4 mm. In some embodiments, the particle has a mean diameter or size between 1 mm to 2 mm. In some embodiments, the particle has a mean diameter or size between 1.5 mm to 2 mm.

In some embodiments, a particle has a largest linear dimension (LLD), e.g., mean diameter, or size that is 1 millimeter (mm) or smaller. In some embodiments, the particle is in a size range of 0.3 mm to 1 mm, 0.4 mm to 1 mm, 0.5 mm to 1 mm, 0.6 mm to 1 mm, 0.7 mm to 1 mm, 0.8 mm to 1 mm or 0.9 mm to 1 mm.

In some embodiments, the second (outer) compartment completely surrounds the first (inner) compartment, and the inner boundary of the second compartment forms an interface with the outer boundary of the first compartment, e.g., as illustrated in FIG. 1. In such embodiments, the thickness of the second (outer) compartment means the average distance between the outer boundary of the second compartment and the interface between the two compartments. In some embodiments, the thickness of the outer compartment is greater than about 10 nanometers (nm), preferably 100 nm or greater and can be as large as 1 mm. For example, the thickness of the outer compartment in a particle described herein may be 10 nanometers to 1 millimeter, 100 nanometers to 1 millimeter, 500 nanometers to 1 millimeter, 1 micrometer (μm) to 1 millimeter, 1 μm to 1 mm, 1 μm to 500 μm, 1 μm to 250 μm, 1 μm to 1 mm, 5 μm to 500 μm, 5 μm to 250 μm, 10 μm to 1 mm, 10 μm to 500 μm, or 10 μm to 250 μm. In some embodiments, the thickness of the outer compartment is 100 nanometers to 1 millimeters, between 1 μm and 1 mm, between 1 μm and 500 μm or between 5 μm and 1 mm.

In some embodiments, a particle comprises at least one pore or opening, e.g., to allow for the free flow of materials. In some embodiments, the mean pore size of a particle is between about 0.1 μm to about 10 μm. For example, the mean pore size may be between 0.1 μm to 10 μm, 0.1 μm to 5 μm, 0.1 μm to 2 μm, 0.1 μm to 10 μm, 0.15 μm to 5 μm, 0.15 μm to 2 μm, 0.2 μm to 10 μm, 0.2 μm to 5 μm, 0.25 μm to 10 μm, 0.25 μm to 5 μm, 0.5 μm to 10 μm, 0.75 μm to 10 μm, 1 μm to 10 μm, 1 μm to 5 μm, 1 μm to 2 μm, 2 μm to 10 μm, 2 μm to 5 μm, or 5 μm to 10 μm. In some embodiments, the mean pore size of a particle is between about 0.1 μm to 10 μm. In some embodiments, the mean pore size of a particle is between about 0.1 μm to 5 μm. In some embodiments, the mean pore size of a particle is between about 0.1 μm to 1 μm. In some embodiments, the mean pore size of the first compartment and the second compartment of the particle is substantially the same. In some embodiments, the mean pore size of the first compartment and the second compartment of the particle differ by about 1.5%, 2%, 5%, 7.5%, 10%, 15%, 20%, 25%, 30%, 35%, 40%, 45%, 50%, 55%, 60%, 65%, 70%, 75%, or more. In some embodiments, the mean pore size of the particle (e.g., mean pore size of the first compartment and/or mean pore size of the second compartment) is dependent on a number of factors, such as the material(s) within each compartment and the presence and density of a compound of Formula (I).

In some embodiments, the particle comprises a metal or a metallic alloy. The first compartment, the second compartment, or both compartments may comprise a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys, e.g., ELGILOY® and PHYNOX®). For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S). In metal-containing particles, the amount of metal (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the particle comprises a ceramic. The first compartment, the second compartment, or both compartments may comprise a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used. In ceramic-containing particles, the amount of ceramic (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the particle comprises a polymer. The first compartment, the second compartment, or both compartments may comprise a polymer. A polymer may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, ladders, and dendrimers. A polymer may be a thermoresponsive polymer, e.g., gel (e.g., becomes a solid or liquid upon exposure to heat or a certain temperature) or a photocrosslinkable polymer. Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, PEEK®, Teflon® (polytetrafluoroethylene, PTFE), PEEK, silicones, epoxy resins, Kevlar®, Dacron® (a condensation polymer obtained from ethylene glycol and terephthalic acid), polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), fluoroplastics, carbon fiber, agarose, alginate, chitosan, and blends or copolymers thereof. In polymer-containing particles, the amount of a polymer (e.g., by % weight of the particle, actual weight of the polymer) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the polymer comprises a polyethylene. The first compartment, the second compartment, or both compartments may comprise a polyethylene. Exemplary polyethylenes include ultra-low-density polyethylene (ULDPE) (e.g., with polymers with densities ranging from 0.890 to 0.905 g/cm³, containing comonomer); very-low-density polyethylene (VLDPE) (e.g., with polymers with densities ranging from 0.905 to 0.915 g/cm³, containing comonomer); linear low-density polyethylene (LLDPE) (e.g., with polymers with densities ranging from 0.915 to 0.935 g/cm³, contains comonomer); low-density polyethylene (LDPE) (e.g., with polymers with densities ranging from about 0.915 to 0.935 g/m³); medium density polyethylene (MDPE) (e.g., with polymers with densities ranging from 0.926 to 0.940 g/cm³, may or may not contain comonomer); high-density polyethylene (HDPE) (e.g., with polymers with densities ranging from 0.940 to 0.970 g/cm³, may or may not contain comonomer).

In some embodiments, the particle comprises a polypropylene. The first compartment, the second compartment, or both compartments may comprise a polypropylene. Exemplary polypropylenes include homopolymers, random copolymers (homophasic copolymers), and impact copolymers (heterophasic copolymers), e.g., as described in McKeen, Handbook of Polymer Applications in Medicine and Medical Devices, 3—Plastics Used in Medical Devices, (2014):21-53, which is incorporated herein by reference in its entirety.

In some embodiments, the particle comprises a polystyrene. The first compartment, the second compartment, or both compartments may comprise a polystyrene. Exemplary polystyrenes include general purpose or crystal (PS or GPPS), high impact (HIPS), and syndiotactic (SPS) polystyrene.

In some embodiments, the particle comprises a thermoplastic elastomer (TPE). The first compartment, the second compartment, or both compartments may comprise a TPE. Exemplary TPEs include (i) TPA—polyamide TPE, comprising a block copolymer of alternating hard and soft segments with amide chemical linkages in the hard blocks and ether and/or ester linkages in the soft blocks; (ii) TPC—co-polyester TPE, consisting of a block copolymer of alternating hard segments and soft segments, the chemical linkages in the main chain being ester and/or ether; (iii) TPO—olefinic TPE, consisting of a blend of a polyolefin and a conventional rubber, the rubber phase in the blend having little or no cross-linking; (iv) TPS—styrenic TPE, consisting of at least a triblock copolymer of styrene and a specific diene, where the two end blocks (hard blocks) are polystyrene and the internal block (soft block or blocks) is a polydiene or hydrogenated polydiene; (v) TPU—urethane TPE, consisting of a block copolymer of alternating hard and soft segments with urethane chemical linkages in the hard blocks and ether, ester or carbonate linkages or mixtures of them in the soft blocks; (vi) TPV—thermoplastic rubber vulcanizate consisting of a blend of a thermoplastic material and a conventional rubber in which the rubber has been cross-linked by the process of dynamic vulcanization during the blending and mixing step; and (vii) TPZ—unclassified TPE comprising any composition or structure other than those grouped in TPA, TPC, TPO, TPS, TPU, and TPV.

In some embodiments, the particle comprises a polysaccharide, and the polysaccharide is an alginate. Alginate is a polysaccharide made up of P-D-mannuronic acid (M) and α-L-guluronic acid (G). In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In alginate-containing particles, the amount of alginate (e.g., by % weight of the particle, actual weight of the alginate) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, both the first compartment and the second compartment comprise the same polymer. In some embodiments, the first compartment and the second compartment comprise different polymers. In some embodiments, the first compartment comprises an alginate. In some embodiments, the second compartment comprises an alginate. In some embodiments, both the first compartment and the second compartment comprise an alginate. In some embodiments, the alginate in the first compartment is different than the alginate in the second compartment. In some embodiments, the first compartment comprises an alginate and the second compartment comprises a different polymer (e.g., a polysaccharide, e.g., hyaluronate or chitosan). In some embodiments, the second compartment comprises an alginate and the first compartment comprises a different polymer (e.g., a polysaccharide, e.g., hyaluronate or chitosan).

Both the first compartment and the second compartment may include a single component (e.g., one polymer) or more than one component (e.g., a blend of polymers). In some embodiments, the first compartment comprises only alginate (e.g., chemically modified alginate, or a blend of an unmodified alginate and a chemically modified alginate). In some embodiments, the second compartment comprises only alginate (e.g., chemically modified alginate or a blend of an unmodified alginate and a chemically modified alginate). In some embodiments, both the first and the second compartment independently comprise only alginate (e.g., chemically modified alginate or blend of an unmodified alginate and a chemically modified alginate).

In some embodiments, the polymer in one or both of the first and second compartments is (i) a low-molecular weight alginate, e.g., has an approximate MW<75 kDa and G:M ratio ≥1.5, (ii) a medium molecular weight alginate, e.g., has approximate molecular weight of 75-150 kDa and G:M ratio ≥1.5, (iii) a high molecular weight alginate, e.g., has an approximate MW of 150 kDa-250 kDa and G:M ratio ≥1.5, (iv) or a blend of two or more of these alginates. In an embodiment, the polymer in the first (inner) compartment is an unmodified, high molecular weight alginate or an unmodified, medium molecular weight alginate and the polymer in the second (outer) compartment is a blend of a chemically-modified alginate (e.g., alginate modified with Compound 101 shown in Table 2) and an unmodified alginate, e.g. a 70:30 blend or a 60:40 blend of CM-LMW-Alg-101:U-HMW-Alg, which may be prepared as described in the Examples below.

In some embodiments, the particle comprises alginate, and the compound of Formula (I) is covalently attached to some or all the monomers in the alginate. In some embodiments, some or all the monomers in the alginate are modified with the same compound of Formula (I). In some embodiments, some or all the monomers in the alginate are modified with different compounds of Formula (I).

In some embodiments, a polymer of the first compartment of the particle is modified with one compound of Formula (I), and a polymer of the second compartment of the particle is modified with a different compound of Formula (I). In some embodiments, the particle comprises a mixture of polymers modified with a compound of Formula (I) and unmodified polymers (e.g., polymers not modified with a compound of Formula (I)). In some embodiments, the first compartment comprises a mixture of polymers modified with a compound of Formula (I) and unmodified polymers (e.g., polymers not modified with a compound of Formula (I)). In some embodiments, the second compartment comprises a mixture of polymers modified with a compound of Formula (I) and unmodified polymers (e.g., polymers not modified with a compound of Formula (I)).

A polymer of a particle described herein may be modified with a compound of Formula (I) or a pharmaceutically acceptable salt thereof on one or more monomers of the polymer. The modified polymer of the particle may be present in the first (inner) compartment of the particle, the second (outer) compartment of the particle, or both the first (inner) and second (outer) compartments of the particle. In some embodiments, the modified polymer is present only in the second compartment (which includes the exterior particle surface). In some embodiments, at least 0.5% of the monomers of a polymer are modified with a compound of Formula (I) (e.g., at least 1%, 2.5%, 5%, 10%, 15%, 20%, 25%, 30%, 35%, 40%, 45%, 50%, 55%, 60%, 65%, 70%, 75%, 80%, 85%, 90%, 95%, 99%, or more of the monomers of a polymer are modified with a compound of Formula (I)). In some embodiments, 0.5% to 50%, 10% to 90%, 10% to 50%, or 25-75%, of the monomers of a polymer are modified with a compound of Formula (I). In some embodiments, 1% to 20% of the monomers of a polymer are modified with a compound of Formula (I). In some embodiments, 1% to 10% of the monomers of a polymer are modified with a compound of Formula (I).

In some embodiments, the polymer (e.g., alginate) (when modified with a compound of Formula (I), e.g., Compound 101 of Table 2) comprises an increase in % N (as compared with unmodified polymer, e.g., alginate) of any of the following values: (i) at least 0.1%, 0.2%, 0.5%, 1.0%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% N by weight; (ii) 0.1% to 10% by weight, (iii) 0.1% to 2% N by weight; (iv) 2% to 4% N by weight; (v) 4% to 8% N by weight; (vi) 5% to 9% N by weight; (vii) 6% to 9% N by weight, (viii) 6% to 8% N by weight; (ix) 7% to 9% N by weight; and (x) 8% to 9% N by weight where, in each case, % N is determined by combustion analysis (e.g., as described in Example 2 herein) and corresponds to the amount of compound of Formula (I) in the modified polymer.

A particle (e.g., a first compartment or second compartment therein) may comprise a compound of Formula (I) in an amount that confers a specific feature to the particle. For example, the particle surface (e.g., the exterior of the outer compartment) may comprise a concentration or density of a compound of Formula (I) such that the particle is afibrotic (i.e., mitigates the foreign body response) in a subject. In an embodiment, the particle surface comprises an alginate chemically modified with an afibrotic-effective amount of Compound 101. In an embodiment, the afibrotic-effective amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis (e.g., as described in Example 2 herein) and corresponds to the amount of Compound 101 in the modified alginate.

As described in the examples below, certain higher concentrations of a compound of Formula (I) in the outer-compartment of two-compartment alginate hydrogel capsules compromised the mechanical strength of the capsules, possibly due to a reduction in sites on the alginate molecules that are available for cross-linking. Thus, in an embodiment, the particle surface (e.g., the exterior of the outer compartment) may comprise a concentration or density of a Formula (I) compound that is high enough to render the particle afibrotic but is lower than a threshold at which a desired mechanical strength is not achieved. In an embodiment, a desired mechanical strength refers to the ability of the particle to maintain its shape and/or remain intact when subjected to any one or more of the following stressors: (i) compression (e.g., at a constant rate); (ii) during administration (e.g., implantation) to a subject; and (iii) after a desired implantation period. The mechanical strength of a particle may be measured prior to implantation of the particle in a subject and/or after retrieval of the implanted particle (e.g., after 1 day, 2 days, 3 days, 4 days, 5 days, 6 days, 1 week, 2 weeks, 3 weeks, 4 weeks, 6 weeks, 8 weeks, 12 weeks, or longer after implant). In an embodiment, the desired mechanical strength of a particle (e.g., a hydrogel capsule) determined after manufacture but before implantation is determined by performing a fracture test using a texture analyzer.

In an embodiment, mechanical testing of hydrogel capsules is performed on a TA.XT plus Texture Analyzer (Stable Micro Systems, Surrey, United Kingdom) using a 5 mm probe attached to a 5 kg load cell. Individual capsules are placed on a platform and are compressed from above by the probe at a fixed rate of 0.5 mm/sec. Contact between the probe and capsule is detected when a repulsive force of 1 g is measured. The probe continues to travel 90% of the distance between the contact height of the probe and the platform, compressing the capsule to the point of bursting. The resistance to the compressive force of the probe is measured and can be plotted as a function of probe travel (force v. displacement curve). Typically, before a capsule bursts completely it will fracture slightly and the force exerted against the probe will decrease a small amount. An analysis macro can be programmed to detect the first time a decrease of 0.25-0.5 g occurs in the force v. displacement curve. The force applied by the probe when this occurs is termed the initial fracture force. In an embodiment, the desired mechanical strength of a particle described herein (e.g., a two-compartment hydrogel capsule) has an initial fracture force of greater than 1, 1.5, 2, 2.5 or 3 grams or at least 2 grams.

In an embodiment, the desired mechanical strength of a particle is the ability to remain intact at a desired timepoint after implantation in a subject, e.g., both the outer and inner compartments of a hydrogel capsule removed from a subject are visibly intact after retrieval from an immune competent mouse when observed by optical microscopy, e.g., by brightfield imaging as described in the Examples herein.

In an embodiment, the particle surface comprises an alginate chemically modified with Compound 101 in an amount that provides the particle with both an afibrotic property and a desired mechanical strength, e.g., a concentration or density of Compound 101 in the modified alginate that produces an increase in % N (as compared with the unmodified alginate) of any of the following values: (i) 1% to 3% by weight, (ii) 2% to 4% N by weight; (iii) 4% to 8% N by weight; (iv) 5% to 9% N by weight; (v) 6% to 9% N by weight, (vi) 6% to 8% N by weight; (vii) 7% to 9% N by weight; and (ix) 8% to 9% N by weight; where, in each case, % N is determined by combustion analysis (e.g., as described in Example 2 herein) and corresponds to the amount of compound of Formula (I) in the modified alginate.

When a particle (e.g., a first compartment or second compartment therein) comprises alginate, the alginate can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with 2-chloro-4,6-dimethoxy-1,3,5-triazine (0.5 eq) and N-methylmorpholine (1 eq). To this mixture may be added a solution of the compound of Formula (I) dissolved in a buffer or solvent, such as acetonitrile (0.3 M). The reaction may be warmed, e.g., to 55° C. for 16 h, then cooled to room temperature and concentrated via rotary evaporation. The residue may then be dissolved in a buffer or solvent, e.g., water. The mixture may then be filtered, e.g., through a bed of cyano-modified silica gel (Silicycle) and the filter cake washed with water. The resulting solution may then be dialyzed (10,000 MWCO membrane) against a buffer or water for 24 hours, e.g., replacing the buffer or water at least one time, at least two times, at least three times, or more. The resulting solution can be concentrated, e.g., via lyophilization, to afford the desired chemically modified alginate.

In some embodiments, a particle described herein comprises a cell. In some embodiments, the cell is engineered to produce a therapeutic agent (e.g., a protein or polypeptide, e.g., an antibody, protein, enzyme, or growth factor). In some embodiments, the cell is disposed with the first compartment. In some embodiments, the cell is disposed within the second compartment. In some embodiments, the cell is disposed in the first compartment and the second compartment does not comprise a cell. A particle may comprise an active or inactive fragment of a protein or polypeptide, such as glucose oxidase (e.g., for glucose sensor), kinase, phosphatase, oxygenase, hydrogenase, reductase.

In some embodiments, a particle is capable of preventing materials over a certain size from passing through a pore or opening. In some embodiments, a particle is capable of preventing materials greater than 50 kD, 75 kD, 100 kD, 125 kD, 150 kD, 175 kD, 200 kD, 250 kD, 300 kD, 400 kD, 500 kD, 750 kD, or 1,000 kD from passing through.

A particle described herein may be configured to release a therapeutic agent, e.g., an exogenous substance, e.g., a therapeutic agent described herein. In some embodiments, the therapeutic agent is a compound of Formula (I) or a pharmaceutically acceptable salt thereof. In some embodiments, the therapeutic agent is a biological material. In some embodiments, the therapeutic agent is a nucleic acid (e.g., an RNA or DNA), protein (e.g., a hormone, enzyme, antibody, antibody fragment, antigen, or epitope), small molecule, lipid, drug, vaccine, or any derivative thereof.

A particle (e.g., as described herein) may be provided as a preparation or composition for implantation or administration to a subject. In some embodiments, at least 20%, 30%, 35%, 40%, 45%, 50%, 55%, 60%, 65%, 70%, 75%, 80%, 85%, 90%, 95% or 100% of the particles in a preparation or composition have a characteristic as described herein, e.g., mean diameter or mean pore size.

In some embodiments, a particle targets or is designed for a certain system of the body, e.g. the nervous system (e.g., peripheral nervous system (PNS) or central nervous system (CNS)), vascular system, skeletal system, respiratory system, endocrine system, lymph system, reproductive system, or gastrointestinal tract. In some embodiments, a particle is targeted to the CNS. In some embodiments, a particle targets or is designed for a certain part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

A particle may be configured for implantation, or implanted or disposed into or onto any site of the body. In some embodiments, a particle is configured for implantation, implanted or disposed into the omentum of a subject, into the subcutaneous fat of a subject, or into the muscle tissue of a subject. A particle can be configured for implantation, or implanted, or disposed on or in the skin; a mucosal surface, a body cavity, the peritoneal cavity (e.g., the lesser sac); the central nervous system, e.g., the brain or spinal cord; an organ, e.g., the heart, liver, kidney, spleen, lung, lymphatic system, vasculature, the oral cavity, the nasal cavity, the teeth, the gums, the GI tract; bone; hip; fat tissue; muscle tissue; circulating blood; the eye (e.g., intraocular); breast, vagina; uterus, a joint, e.g., the knee or hip joint, or the spine.

In some embodiments, the particle is configured for implantation or implanted or disposed into the peritoneal cavity (e.g., the omentum). In some embodiments, the particle is configured for implantation or implanted or disposed into or onto the lesser sac, also known as the omental bursa or bursalis omentum. The lesser sac refers to a cavity located in the abdomen formed by the omentum, and is in close proximity to, for example, the greater omentum, lesser omentum, stomach, small intestine, large intestine, liver, spleen, gastrosplenic ligament, adrenal glands, and pancreas. Typically, the lesser sac is connected to the greater sac via the omental foramen (i.e., the Foramen of Winslow). In some embodiments, the lesser sac comprises a high concentration of adipose tissue. A particle may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of a particle into the omentum (e.g., the lesser sac) are provided in M. Pellicciaro et al. (2017) CellR4 5(3):e2410, which is incorporated herein by reference in its entirety.

In some embodiments, the particle is configured for implantation or implanted or disposed into the central nervous system (CNS), e.g., the brain or spinal cord and their corresponding tissues and cavities. In vertebrates, the CNS is contained within the dorsal body cavity, including the cranial cavity and the spinal canal. In some embodiments, the particle is configured for implantation or implanted or disposed into an intracerebral space, e.g., the intraparenchymal space, the intraventricular space, or the subdural space. A particle may be implanted in the CNS or disposed on a surface within the CNS through a hole made in the skull and delivered via injection or catheter.

In some embodiments, the particle is configured for implantation or implanted in or disposed into the eye. Exemplary regions suitable for implantation or disposition of the particle include any surface or cavity within the eye, such as the retina, cornea, epithelium, aqueous humor, or vitreal space. In some embodiments, the particle is configured for implantation or implanted or disposed into the vitreal space. A particle may be implanted in the eye or disposed on a surface within the eye through incision and/or injection.

In some embodiments, the particle is easily retrievable from a subject, e.g., without causing injury to the subject or without causing significant disruption of the surrounding tissue.

In an embodiment, the particle can be retrieved with minimal or no surgical separation of the particle from surrounding tissue, e.g., via minimally invasive surgical approach, extraction, or resection.

A particle can be configured for limited exposure (e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). A particle can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more) A particle can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

In some embodiments, the particle is not a particle disclosed in any of WO2012/112982, WO2012/167223, WO2014/153126, WO2016/019391, WO2016/187225, US2012-0213708, US 2016-0030359, and US 2016-0030360.

### Compounds

In some embodiments, the particles described herein comprise a compound of Formula (I). In some embodiments, the first compartment and/or second compartment of the particle comprise a compound of Formula (I):

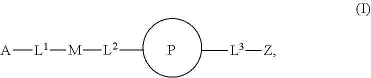

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₂-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocycyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶.
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, the compound of Formula (I) is a compound of Formula (I-a):

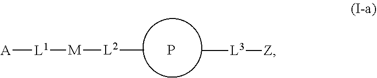

or a salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —N(R^{C})C(O)(C₁-C₆-alkylene)-, —N(R^{C})C(O)(C₂-C₆-alkenylene)-, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶.
or R^{C} and R^{D}, taken together with the nitrogen atom to which they are attached, form a ring (e.g., a 5-7 membered ring), optionally substituted with one or more R⁶.
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, for Formulas (I) or (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{C})—, and R^{C} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) or (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) or (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) or (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂-)z, wherein z is an integer selected from 1 to 10. In some embodiments, z is an integer selected from 1 to 5. In some embodiments, M is —OCH₂CH₂—, (—OCH₂CH₂—)₂, (—OCH₂CH₂—)₃, (—OCH₂CH₂—)₄, or (—OCH₂CH₂—)₅. In some embodiments, M is —OCH₂CH₂—, (—OCH₂CH₂—)₂, (—OCH₂CH₂—)₃, or (—OCH₂CH₂—)₄. In some embodiments, M is (—OCH₂CH₂—)₃.

In some embodiments, M is aryl. In some embodiments, M is phenyl. In some embodiments, M is unsubstituted phenyl. In some embodiments, M is

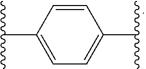 [SMILES: *c1ccc(*)cc1]

In some embodiments, M is phenyl substituted with R⁷ (e.g., 1 R⁷). In some embodiments, M is

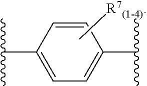

In some embodiments, R⁷ is CF₃.

In some embodiments, for Formulas (I) or (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, pyrrolyl, oxazolyl, or thiazolyl. In some embodiments, P is tetrazolyl, imidazolyl, pyrazolyl, or triazolyl, or pyrrolyl. In some embodiments, P is imidazolyl. In some embodiments, P is

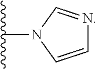 [SMILES: *n1ccnc1]

In some embodiments, P is triazolyl. In some embodiments, P is 1,2,3-triazolyl. In some embodiments, P is

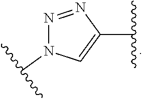 [SMILES: *c1cn(*)nn1]

In some embodiments, P is heterocyclyl. In some embodiments, P is a 5-membered heterocyclyl or a 6-membered heterocyclyl. In some embodiments, P is imidazolidinonyl. In some embodiments, P is

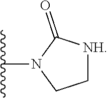 [SMILES: *N1CCNC1=O]

In some embodiments, P is thiomorpholinyl-1,1-dioxidyl. In some embodiments, P is

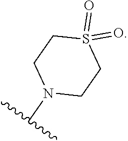 [SMILES: *N1CCS(=O)(=O)CC1]

In some embodiments, for Formulas (I) or (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl. In some embodiments, Z is an oxygen-containing heterocyclyl. In some embodiments, Z is a 4-membered heterocyclyl, 5-membered heterocyclyl, or 6-membered heterocyclyl. In some embodiments, Z is a 6-membered heterocyclyl. In some embodiments, Z is a 6-membered oxygen-containing heterocyclyl. In some embodiments, Z is tetrahydropyranyl. In some embodiments, Z is

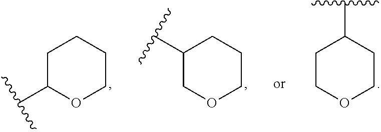 [SMILES: *C1CCCCO1 ; *C1CCCOC1 ; *C1CCOCC1]

In some embodiments, Z is a 4-membered oxygen-containing heterocyclyl. In some embodiments, Z is

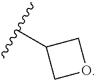 [SMILES: *C1COC1]

In some embodiments, Z is a bicyclic oxygen-containing heterocyclyl. In some embodiments, Z is phthalic anhydridyl. In some embodiments, Z is a sulfur-containing heterocyclyl. In some embodiments, Z is a 6-membered sulfur-containing heterocyclyl. In some embodiments, Z is a 6-membered heterocyclyl containing a nitrogen atom and a sulfur atom. In some embodiments, Z is thiomorpholinyl-1,1-dioxidyl. In some embodiments, Z is

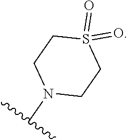 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

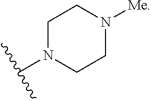 [SMILES: *N1CCN(C)CC1]

In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl. In some embodiments, Z is

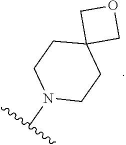 [SMILES: *N1CCC2(CC1)COC2]

In some embodiments, Z is 1-oxa-3,8-diazaspiro[4.5]decan-2-one. In some embodiments, Z is

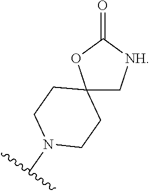 [SMILES: *N1CCC2(CC1)CNC(=O)O2]

In some embodiments, for Formulas (I) or (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) or (I-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) or (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A}(e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

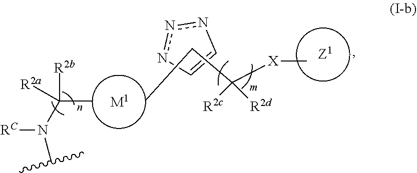

or a salt thereof, wherein Ring M¹ is cycloalkyl, heterocyclyl, aryl, or heteroaryl, each of which is optionally substituted with 1-5 R³; Ring Z¹ is cycloalkyl, heterocyclyl, aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵, and R⁶ is independently alkyl, alkenyl, alkynyl, heteroalkyl, halogen, cyano, azido, oxo, —OR^{A}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

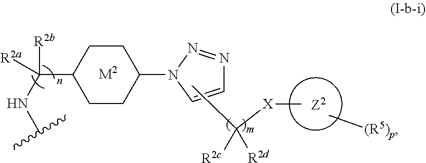

or a pharmaceutically acceptable salt thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I-b-i) is a compound of Formula (I-b-ii):

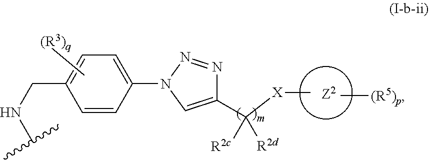

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c):

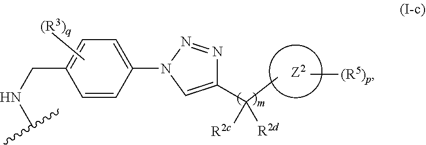

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

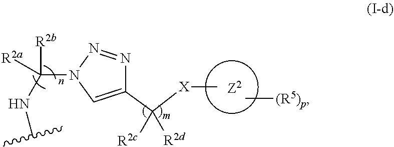

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

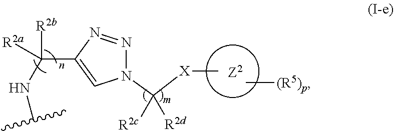

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

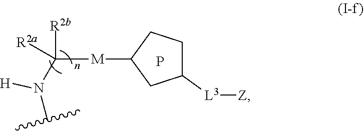

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

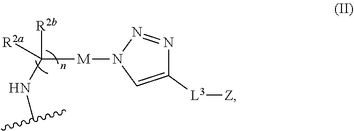

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR^{A}, wherein alkyl, heteroalkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

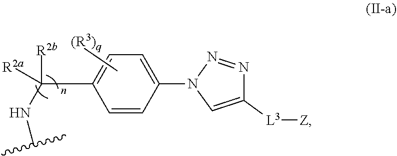

or a pharmaceutically acceptable salt thereof, wherein L³ is alkyl or heteroalkyl, each of which is optionally substituted with one or more R²; Z is hydrogen, alkyl, heteroalkyl, or —OR^{A}, wherein alkyl and heteroalkyl are optionally substituted with one or more R⁵; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; R^{A} is hydrogen; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (III):

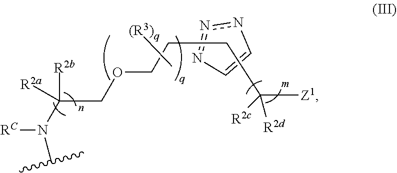

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2c} is independently hydrogen, alkyl, alkenyl, alkynyl, heteroalkyl, halo, cyano, nitro, amino, cycloalkyl, heterocyclyl, aryl, or heteroaryl; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; and “ [symbol] ” refers to a connection to an attachment group or a polymer described herein.

In some embodiments, the compound of Formula (III) is a compound of Formula (III-a):

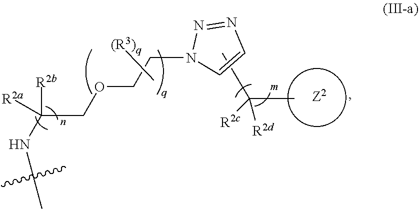

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-b):

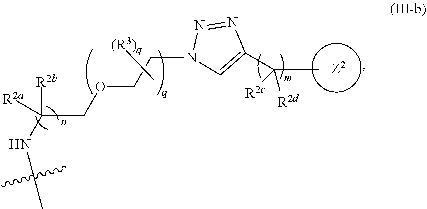

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-c):

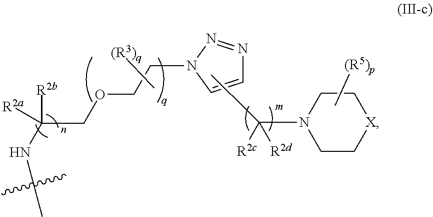

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound of Formula (III-c) is a compound of Formula (III-d):

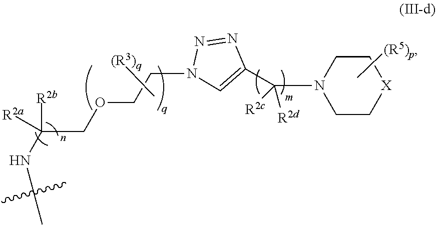

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein. [symbol] In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is O, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is O, p is 0, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-d) is Compound 110 or Compound 114.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃. In some embodiments, the compound of Formula (II-a) is Compound 112.

In some embodiments of Formula (II-a), each of R¹ and R² is independently hydrogen, n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., an nitrogen-containing heterocyclyl, e.g., a nitrogen-containing spiro heterocyclyl, e.t., 2-oxa-7-azaspiro[3.5]nonanyl). In some embodiments, the compound of Formula (II-b) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂. In some embodiments, the compound of Formula (III-d) is Compound 101, Compound 117, Compound 118, or Compound 119.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound of Formula (I) is not a compound disclosed in WO20121112982, WO2012/167223, WO2014/153126, WO2016/019391, WO 2017/075630, US2012-0213708, US 2016-0030359 or US 2016-0030360.

In some embodiments, the compound of Formula (I) comprises a compound shown in Table 2, or a pharmaceutically acceptable salt thereof. In some embodiments, a particle described herein comprises a compound shown in Table 2, or a pharmaceutically acceptable salt thereof.

TABLE 2
Exemplary compounds

| Compound No. | Structure |
|---|---|
| 100 | 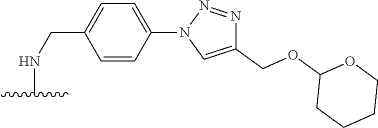 [SMILES: *NCc1ccc(-n2cc(COC3CCCCO3)nn2)cc1] |
| 101 | 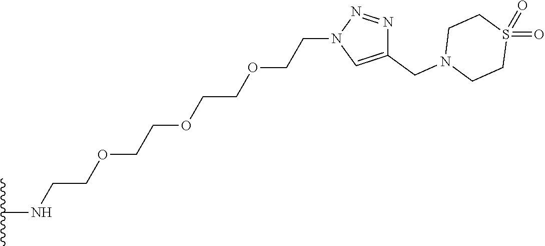 [SMILES: *NCCOCCOCCOCCn1cc(CN2CCS(=O)(=O)CC2)nn1] |
| 102 | 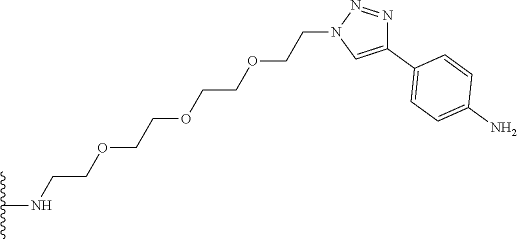 [SMILES: *NCCOCCOCCOCCn1cc(-c2ccc(N)cc2)nn1] |
| 103 | 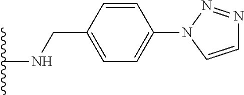 [SMILES: *NCc1ccc(-n2ccnn2)cc1] |
| 104 | 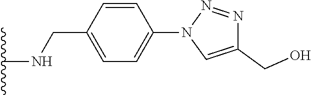 [SMILES: *NCc1ccc(-n2cc(CO)nn2)cc1] |
| 105 | 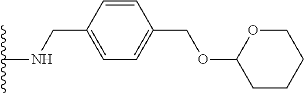 [SMILES: *NCc1ccc(COC2CCCCO2)cc1] |
| 106 | 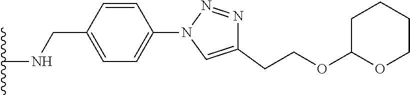 [SMILES: *NCc1ccc(-n2cc(CCOC3CCCCO3)nn2)cc1] |
| 107 | 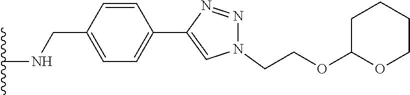 [SMILES: *NCc1ccc(-c2cn(CCOC3CCCCO3)nn2)cc1] |
| 108 | 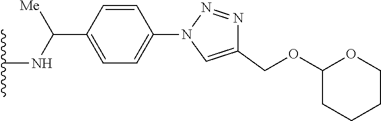 [SMILES: *NC(C)c1ccc(-n2cc(COC3CCCCO3)nn2)cc1] |
| 109 | 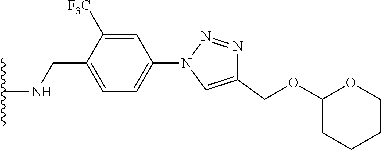 [SMILES: *NCc1ccc(-n2cc(COC3CCCCO3)nn2)cc1C(F)(F)F] |
| 110 | 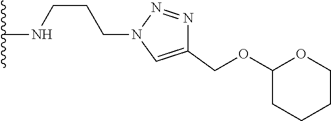 [SMILES: *NCCCn1cc(COC2CCCCO2)nn1] |
| 111 | 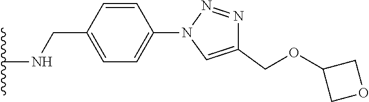 [SMILES: *NCc1ccc(-n2cc(COC3COC3)nn2)cc1] |
| 112 | 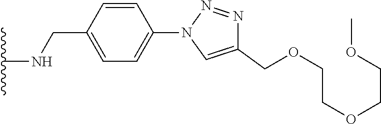 [SMILES: *NCc1ccc(-n2cc(COCCOCCOC)nn2)cc1] |
| 113 | 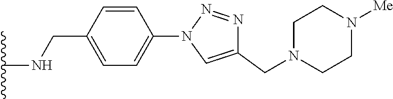 [SMILES: *NCc1ccc(-n2cc(CN3CCN(C)CC3)nn2)cc1] |
| 114 | 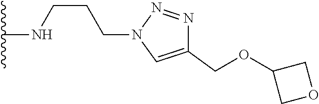 [SMILES: *NCCCn1cc(COC2COC2)nn1] |
| 115 | 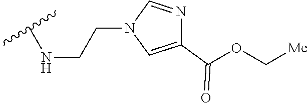 [SMILES: *NCCn1cnc(C(=O)OCC)c1] |
| 116 | 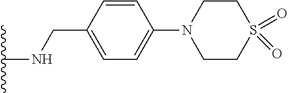 [SMILES: *NCc1ccc(N2CCS(=O)(=O)CC2)cc1] |
| 117 | 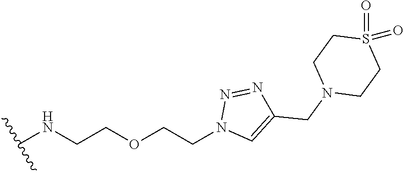 [SMILES: *NCCOCCn1cc(CN2CCS(=O)(=O)CC2)nn1] |
| 118 | 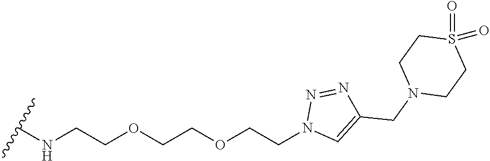 [SMILES: *NCCOCCOCCn1cc(CN2CCS(=O)(=O)CC2)nn1] |
| 119 | 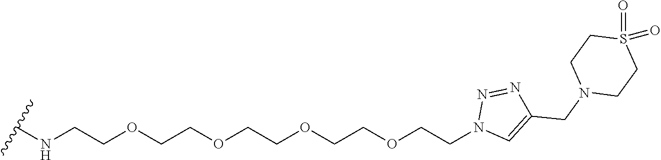 [SMILES: *NCCOCCOCCOCCOCCn1cc(CN2CCS(=O)(=O)CC2)nn1] |
| 120 | 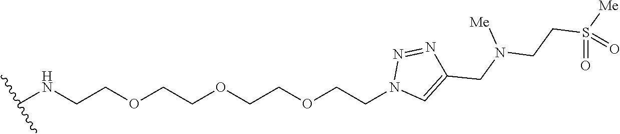 [SMILES: *NCCOCCOCCOCCn1cc(CN(C)CCS(C)(=O)=O)nn1] |
| 121 | 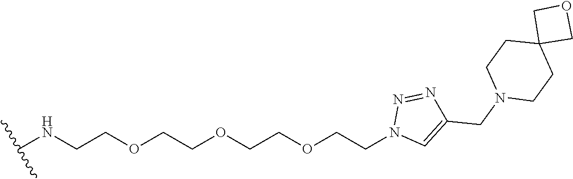 [SMILES: *NCCOCCOCCOCCn1cc(CN2CCC3(CC2)COC3)nn1] |

In some embodiments, the compound is a compound of Formula (I) (e.g., Formulas (I-a), (I-b), (I-c), (I-d), (I-e), (I-f), (II), (II-a), (III), (III-a), (III-b), (III-c), or (III-d), or a pharmaceutically acceptable salt thereof, and is selected from:

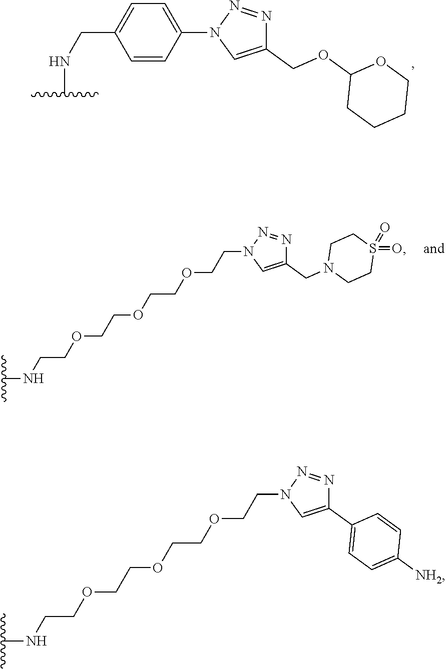 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt thereof.

In some embodiments, the article described herein comprises the compound of

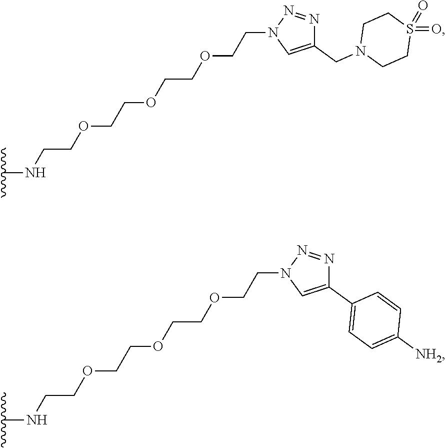 [SMILES: *NCCOCCOCCOCCn1cc(CN2CCS(=O)(=O)CC2)nn1 ; *NCCOCCOCCOCCn1cc(-c2ccc(N)cc2)nn1]

pharmaceutically acceptable salt thereof.

In an embodiment, a particle described herein comprises a compound of Formula (I) (e.g., a compound shown in Table 2) covalently bound to an alginate polymer. In an embodiment, a particle described herein comprises a compound of Formula (I) (e.g., a compound shown in Table 2, e.g., Compound 101) covalently bound to one or more guluronic acid and/or mannuronic acid monomers in an alginate polymer, e.g., by an amide bond.

In some embodiments, a compound of Formula (I) (e.g., Compound 101 in Table 2) is covalently attached to an alginate (e.g., an alginate with approximate MW<75 kDa, G:M ratio ≥1.5) at a conjugation density of at least 2.0% and less than 9.0% nitrogen, or 2.0% to 5% nitrogen, 3.0% to 8.0% nitrogen, 5% to 8.0% nitrogen, 4.0% to 7.0% nitrogen, 5.0% to 7.0% nitrogen, or 6.0% to 7.0% nitrogen or about 6.8% nitrogen as determined by combustion analysis for percent nitrogen as described in the Examples below.

### Cells

The particles of the present disclosure may comprise a wide variety of different cell types (e.g., human cells), including epithelial cells, endothelial cells, fibroblast cells, mesenchymal stem cells, keratinocyte cells, islet cells, and cells derived from any of the foregoing cell types.

The cells may be derived from stem cells or induced pluripotent stem cells. Exemplary cell types include the cell types recited in WO 2017/075631. In some embodiments, the cells are derived from a cell-line shown in Table 3 below.

TABLE 3
Exemplary cell lines

| | | Germ | |
|---|---|---|---|
| Cell Line | Cell Type | Layer | Commercial Source |
| ARPE-19 | Epithelial (Retinal) | Ectoderm | ATCC (CRL-2302) |
| BJ | Fibroblast (Foreskin) | Ectoderm | ATCC (CRL-2522) |
| CCD-841- | Epithelial (Colon) | Endoderm | ATCC (CRL-1790) |
| CoN | | | |
| HaCat | Keratinocyte | Ectoderm | Addexbio (T0020001) |
| HHSEC | Endothelial (Hepatic Sinusoidal) | Endoderm | Sciencellonline.com (#5000) |
| Huv-EC-C | Endothelial (Embryonic umbilical) | Mesoderm | ATCC (CRL-1730) |
| MCF-10A | Epithelial (Mammary Gland) | Ectoderm | ATCC (CRL-10317) |
| MRC-5 | Fibroblast (Lung) | Mesoderm | ATCC (CCL-171) |
| MSC, human | Mesenchyme (Bone Marrow) | Mesoderm | ATCC (PCS-500-012) |
| MSC, mouse | Mesenchyme (Bone Marrow) | Mesoderm | Cyagen (MU BMX-01001) |
| WS-1 | Fibroblast (Skin) | Ectoderm | ATCC (CRL-1502) |
| 293F | Epithelial (Embryonic Kidney) | Mesoderm | Thermo Fisher (R790007) |

In some embodiments, the particle does not comprise any islet cells, as defined herein. In an embodiment, cells contained in a particle of the disclosure, e.g., RPE cells, MSFCs, including engineered RPE cells and MSFCs, have one or more of the following characteristics: (i) are not capable of producing insulin (e.g., insulin A-chain, insulin B-chain, or proinsulin) in an amount effective to treat diabetes or another disease or condition that may be treated with insulin; (ii) not capable of producing insulin in a glucose-responsive manner; or (iii) not an induced pluripotent cell that is engineered into a differentiated insulin-producing pancreatic beta cell.

In an embodiment, the particles described herein comprise a plurality of cells. In an embodiment, the plurality of cells is in the form of a cell suspension prior to being encapsulated within a particle described herein. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In some embodiments, the cells have been engineered to produce a therapeutic agent for the prevention or treatment of a disease, disorder, or condition described, e.g., in WO 2017/075631. The therapeutic agent may be any biological substance, such as a nucleic acid (e.g., a nucleotide, DNA, or RNA), a polypeptide, a lipid, a sugar (e.g., a monosaccharide, disaccharide, oligosaccharide, or polysaccharide), or a small molecule. Exemplary therapeutic agents include the agents listed in WO 2017/075631.

In some embodiments, the therapeutic agent is a peptide or polypeptide (e.g., a protein), such as a hormone, enzyme, cytokine (e.g., a pro-inflammatory cytokine or an anti-inflammatory cytokine), growth factor, clotting factor, or lipoprotein. A peptide or polypeptide (e.g., a protein, e.g., a hormone, growth factor, clotting factor or coagulation factor, antibody molecule, enzyme, cytokine, cytokine receptor, or a chimeric protein including cytokines or a cytokine receptor) produced by an engineered cell can have a naturally occurring amino acid sequence, or may contain a variant of the naturally occurring sequence. The variant can be a naturally occurring or non-naturally occurring amino acid substitution, mutation, deletion or addition relative to the reference naturally occurring sequence. The naturally occurring amino acid sequence may be a polymorphic variant. The naturally occurring amino acid sequence can be a human or a non-human amino acid sequence. In some embodiments, the naturally occurring amino acid sequence or naturally occurring variant thereof is a human sequence. In addition, a peptide or polypeptide (e.g., a protein) for use with the present disclosure may be modified in some way, e.g., via chemical or enzymatic modification (e.g., glycosylation, phosphorylation). In some embodiments, the peptide has about 2, 3, 4, 5, 6, 7, 8, 9, 10, 12, 14, 16, 18, 20, 25, 30, 35, 40, 45, or 50 amino acids. In some embodiments, the protein has an average molecular weight of 5 kD, 10 kD, 25 kD, 50 kD, 100 kD, 150 kD, 200 kD, 250 kD, 500 kD, or more.

In some embodiments, the protein is a hormone. Exemplary hormones include anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone, aldosterone, cortisol, epinephrine, glucagon, insulin, estrogen, progesterone, and testosterone. In some embodiments, the protein is insulin (e.g., insulin A-chain, insulin B-chain, or proinsulin). In some embodiments, the protein is a growth hormone, such as human growth hormone (hGH), recombinant human growth hormone (rhGH), bovine growth hormone, methionine-human growth hormone, des-phenylalanine human growth hormone, and porcine growth hormone.

In some embodiments, the protein is a growth factor, e.g., vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the protein is a clotting factor or a coagulation factor, e.g., a blood clotting factor or a blood coagulation factor. In some embodiments, the protein is a protein involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor, prekallikrein, heparin cofactor II, high molecular weight kininogen (e.g., Fitzgerald factor), antithrombin III, and fibronectin. In some embodiments, the protein is an anti-clotting factor, such as Protein C.

In some embodiments, the protein is an antibody molecule. As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full-length antibody which has an immunoglobulin Fc region). In an embodiment, an antibody molecule comprises a full-length antibody, or a full-length immunoglobulin chain. In an embodiment, an antibody molecule comprises an antigen binding or functional fragment of a full-length antibody, or a full-length immunoglobulin chain. In an embodiment, an antibody molecule is a monospecific antibody molecule and binds a single epitope, e.g., a monospecific antibody molecule having a plurality of immunoglobulin variable domain sequences, each of which binds the same epitope. In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

Various types of antibody molecules may be produced by the encapsulated engineered cells, including whole immunoglobulins of any class, fragments thereof, and synthetic proteins containing at least the antigen binding variable domain of an antibody. The antibody molecule can be an antibody, e.g., an IgG antibody, such as IgG₁, IgG₂, IgG₃, or IgG₄. An antibody molecule can be in the form of an antigen binding fragment including a Fab fragment, F(ab′)2 fragment, a single chain variable region, and the like. Antibodies can be polyclonal or monoclonal (mAb). Monoclonal antibodies may include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity. In some embodiments, the antibody molecule is a single-domain antibody (e.g., a nanobody). The described antibodies can also be modified by recombinant means, for example by deletions, additions or substitutions of amino acids, to increase efficacy of the antibody in mediating the desired function. Exemplary antibodies include anti-beta-galactosidase, anti-collagen, anti-CD14, anti-CD20, anti-CD40, anti-HER2, anti-IL-1, anti-IL-4, anti-IL6, anti-IL-13, anti-IL17, anti-IL18, anti-IL-23, anti-IL-28, anti-IL-29, anti-IL-33, anti-EGFR, anti-VEGF, anti-CDF, anti-flagellin, anti-IFN-α, anti-IFN-β, anti-IFN-γ, anti-mannose receptor, anti-VEGF, anti-TLR1, anti-TLR2, anti-TLR3, anti-TLR4, anti-TLR5, anti-TLR6, anti-TLR9, anti-PDF, anti-PD1, anti-PDL-1, or anti-nerve growth factor antibody. In some embodiments, the antibody is an anti-nerve growth factor antibody (e.g., fulranumab, fasinumab, tanezumab).

In some embodiments, the protein is a cytokine or a cytokine receptor, or a chimeric protein including cytokines or their receptors, including, for example tumor necrosis factor alpha and beta, their receptors and their derivatives, renin; lipoproteins; colchicine; corticotrophin; vasopressin; somatostatin; lypressin; pancreozymin; leuprolide; alpha-i-antitrypsin; atrial natriuretic factor; lung surfactant; a plasminogen activator other than a tissue-type plasminogen activator (t-PA), for example a urokinase; bombesin; thrombin; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues. Suitable proteins or peptides may be native or recombinant and include, e.g., fusion proteins.

Examples of a polypeptide (e.g., a protein) produced by particle described herein also include CCL1, CCL2 (MCP-1), CCL3 (MIP-1α), CCL4 (MIP-1β), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL 11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL5, IL4, IL13, IL7, IL9, IL21, IL3, IL5, IL6, IL11, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, IL1A (IL1IF1), IL1B (IL1F2), IL1Ra (IL1F3), IL1F5 (IL36RN), IL1F6 (IL36A), IL1F7 (IL37), IL1F8 (IL36B), IL1F9 (IL36G), IL IF10 (IL38), IL33 (IL IF11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, C1qtnf4, Ccl21a, Ccl27a, Cd70, Cer1, Cklf, Clcf1, Cmtm2a, Cmtm2b, Cmtm3, Cmtm4, Cmtm5, Cmtm6, Cmtm7, Cmtm8, Crlf1, Ctf2, Ebi3, Edn1, Fam3b, Fas1, Fgf2, Flt31, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Gm, Hmgb1, Ifna11, Ifna12, Ifna9, Ifnab, Ifne, Il17a, Il23a, Il25, Il31, Iltifb, Inhba, Lefty1, Lefty2, Mstn, Nampt, Ndp, Nodal, Pf4, Pglyrp1, Prl7d1, Scg2, Scgb3a1, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcl1, epinephrine, melatonin, triiodothyronine, a prostaglandin, a leukotriene, prostacyclin, thromboxane, islet amyloid polypeptide, müllerian inhibiting factor or hormone, adiponectin, corticotropin, angiotensin, vasopressin, arginine vasopressin, atriopeptin, brain natriuretic peptide, calcitonin, cholecystokinin, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, inhibin, somatomedin, leptin, lipotropin, melanocyte stimulating hormone, motilin, orexin, oxytocin, pancreatic polypeptide, pituitary adenylate cyclase-activating peptide, relaxin, renin, secretin, somatostatin, thrombopoietin, thyrotropin, thyrotropin-releasing hormone, vasoactive intestinal peptide, androgen, alpha-glucosidase (also known as acid maltase), glycogen phosphorylase, glycogen debrancher enzyme, phosphofructokinase, phosphoglycerate kinase, phosphoglycerate mutase, lactate dehydrogenase, carnitine palymityl transferase, camitine, and myoadenylate deaminase.

In some embodiments, the protein is a replacement therapy or a replacement protein. In some embodiments, the replacement therapy or replacement protein is a clotting factor or a coagulation factor, e.g., Factor VIII (e.g., comprises a naturally occurring human Factor VIII amino acid sequence or a variant thereof) or Factor IX (e.g., comprises a naturally occurring human Factor IX amino acid sequence or a variant thereof).

In some embodiments, the cell is engineered to express a human Factor VIII protein, e.g., a recombinant Factor VIII. In some embodiments, the recombinant Factor VIII is a B-domain-deleted recombinant Factor VIII (FVIII-BDD). In some embodiments, the cell is derived from a human RPE cell line and comprises an exogenous nucleic acid sequence which encodes the FVIII-BDD amino acid sequence shown in FIG. 2A (SEQ ID NO: 1).

In some embodiments, the cell is engineered to express a FIX, e.g., a wild-type human F IX, such as that shown in FIG. 2B (SEQ ID NO: 2) or a polymorphic variant thereof (e.g., alanine substituted for threonine at amino acid position 148 of SEQ ID NO: 2). In some embodiments, the cell is engineered to express a gain-in-function (GIF) variant of a wild-type FIX protein (FIX-GIF), wherein the GIF variant has higher specific activity than the corresponding wild-type FIX. In some embodiments, the cell is derived from a human RPE cell line and comprises an exogenous nucleic acid sequence which encodes SEQ ID NO: 2, except for having an amino acid substituted for arginine at a position corresponding to amino acid position 338 of SEQ ID NO: 2. In some embodiments, the substituting amino acid at a position corresponding to amino acid position 338 of SEQ ID NO: 2 is alanine, asparagine, aspartic acid, cysteine, glutamic acid, glutamine, histidine, leucine, lysine, or tyrosine. In some embodiments, the FIX protein encoded by cells contained in a particle described herein is a FIX-padua protein and comprises, consists essentially of, or consists of SEQ ID NO:36 (FIG. 17).

In some embodiments, the encapsulated cells are derived from a human RPE cell line and comprise an exogenous nucleic acid sequence which comprises a promoter sequence (e.g., nucleotides 337-2069 of SEQ ID NO:26) operably linked to a coding sequence for a polypeptide. In an embodiment, the coding sequence is a codon-optimized FVIII-BDD coding sequence shown in FIG. 17 (SEQ ID NO: 9, 10, 11, 12, 13, 14, 15, 16 or 17) or a codon-optimized FIX-padua coding sequence shown in FIG. 17 (SEQ ID NO:19, 20 or 21).

In some embodiments, the encapsulated cells are derived from a human RPE cell line and comprise a promoter sequence (e.g., SEQ ID NO:23 or a nucleotide sequence that is at least 95%, 96%, 97%, 98% or 99% identical to SEQ ID NO:23) operably linked to a nucleotide sequence that encodes an amino acid sequence selected from the group consisting of SEQ ID NO:1, 2, 3, 4, 5, 6, 7, 29, 30, 31, 32, 33, 34, 35 and 36.

In some embodiments, the particle is a two-compartment hydrogel capsule, in which the inner compartment was formed from a polymer solution comprising about 20 million cells/ml to about 40 million cells/ml, wherein the cells are derived from the ARPE-19 cell line and comprise nucleotides 337-2069 of SEQ ID NO:26 operably linked to a codon-optimized FVIII-BDD coding sequence shown in FIG. 17. In an embodiment, the FVIII-BDD coding sequence is SEQ ID NO:15.

In some embodiments, the replacement therapy or replacement protein is an enzyme, e.g., alpha-galactosidase, alpha-L-iduronidase (IDUA), or N-sulfoglucosamine sulfohydrolase (SGSH). In some embodiments, the replacement therapy or replacement protein is an enzyme, e.g., an alpha-galactosidase A (e.g., comprises a naturally occurring human alpha-galactosidase A amino acid sequence or a variant thereof). In some embodiments, the replacement therapy or replacement protein is a cytokine or an antibody.

In some embodiments, the therapeutic agent is a sugar, e.g., monosaccharide, disaccharide, oligosaccharide, or polysaccharide. In some embodiments, a sugar comprises a triose, tetrose, pentose, hexose, or heptose moiety. In some embodiments, the sugar comprises a linear monosaccharide or a cyclized monosaccharide. In some embodiments, the sugar comprises a glucose, galactose, fructose, rhamnose, mannose, arabinose, glucosamine, galactosamine, sialic acid, mannosamine, glucuronic acid, galactosuronic acid, mannuronic acid, or guluronic acid moiety. In some embodiments, the sugar is attached to a protein (e.g., an N-linked glycan or an O-linked glycan). Exemplary sugars include glucose, galactose, fructose, mannose, rhamnose, sucrose, ribose, xylose, sialic acid, maltose, amylose, inulin, a fructooligosaccharide, galactooligosaccharide, a mannan, a lectin, a pectin, a starch, cellulose, heparin, hyaluronic acid, chitin, amylopectin, or glycogen. In some embodiments, the therapeutic agent is a sugar alcohol.

In some embodiments, the therapeutic agent is a lipid. A lipid may be hydrophobic or amphiphilic, and may form a tertiary structure such as a liposome, vesicle, or membrane or insert into a liposome, vesicle, or membrane. A lipid may comprise a fatty acid, glycerolipid, glycerophospholipid, sterol lipid, prenol lipid, sphingolipid, saccharolipid, polyketide, or sphingolipid. Examples of lipids produced by the encapsulated cells include anandamide, docosahexaenoic acid, a prostaglandin, a leukotriene, a thromboxane, an eicosanoid, a triglyceride, a cannabinoid, phosphatidylcholine, phosphatidylethanolamine, a phosphatidylinositol, a phosohatidic acid, a ceramide, a sphingomyelin, a cerebroside, a ganglioside, estrogen, androsterone, testosterone, cholesterol, a carotenoid, a quinone, a hydroquinone, or a ubiquinone.

In some embodiments, the therapeutic agent is a small molecule. A small molecule may include a natural product produced by a cell. In some embodiments, the small molecule has poor availability or does not comply with the Lipinski rule of five (a set of guidelines used to estimate whether a small molecule will likely be an orally active drug in a human; see, e.g., Lipinski, C. A. et al (2001) Adv Drug Deliv 46:2-36). Exemplary small molecule natural products include an anti-bacterial drug (e.g., carumonam, daptomycin, fidaxomicin, fosfomycin, ispamicin, micronomicin sulfate, miocamycin, mupiocin, netilmicin sulfate, teicoplanin, thienamycin, rifamycin, erythromycin, vancomycin), an anti-parasitic drug (e.g., artemisinin, ivermectin), an anticancer drug (e.g., doxorubicin, aclarubicin, aminolaevulinic acid, arglabin, omacetaxine mepesuccinate, paclitaxel, pentostatin, peplomycin, romidepsin, trabectdin, actinomycin D, bleomycin, chromomycin A, daunorubicin, leucovorin, neocarzinostatin, streptozocin, trabectedin, vinblastine, vincristine), anti-diabetic drug (e.g., voglibose), a central nervous system drug (e.g., L-dopa, galantamine, zicontide), a statin (e.g., mevastatin), an anti-fungal drug (e.g., fumagillin, cyclosporin), 1-deoxynojirimycin, and theophylline, sterols (cholesterol, estrogen, testerone). Additional small molecule natural products are described in Newman, D. J. and Cragg, M. (2016) J Nat Prod 79:629-661 and Butler, M. S. et al (2014) Nat Prod Rep 31:1612-1661, which are incorporated herein by reference in their entirety.

In some embodiments, the cells are engineered to synthesize a non-protein or non-peptide small molecule. For example, in an embodiment an engineered cell can produce a statin (e.g., taurostatin, pravastatin, fluvastatin, or atorvastatin).

In some embodiments, the therapeutic agent is an antigen (e.g., a viral antigen, a bacterial antigen, a fungal antigen, a plant antigen, an environmental antigen, or a tumor antigen). An antigen is recognized by those skilled in the art as being immunostimulatory, i.e., capable of stimulating an immune response or providing effective immunity to the organism or molecule from which it derives. An antigen may be a nucleic acid, peptide, protein, sugar, lipid, or a combination thereof.

The particles comprising a cell may produce a single therapeutic agent or a plurality of therapeutic agents. The plurality of therapeutic agents may be related or may form a complex. In some embodiments, the therapeutic agent secreted or released from a particle comprising a cell is in an active form. In some embodiments, the therapeutic agent is secreted or released from a particle comprising a cell an inactive form, e.g., as a prodrug. In the latter instance, the therapeutic agent may be activated by a downstream agent, such as an enzyme.

### Methods of Treatment

Described herein are methods for preventing or treating a disease, disorder, or condition in a subject through administration or implantation of particles comprising a first compartment, a second compartment, and a compound of Formula (I) (e.g., as described herein), or a composition comprising the same. In some embodiments, the methods described herein directly or indirectly reduce or alleviate at least one symptom of a disease, disorder, or condition. In some embodiments, the methods described herein prevent or slow the onset of a disease, disorder, or condition. In some embodiments, the subject is a human.

In some embodiments, the disease, disorder, or condition affects a system of the body, e.g. the nervous system (e.g., peripheral nervous system (PNS) or central nervous system (CNS)), vascular system, skeletal system, respiratory system, endocrine system, lymph system, reproductive system, or gastrointestinal tract. In some embodiments, the disease, disorder, or condition affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

In some embodiments, the disease, disorder or condition is a neurodegenerative disease, diabetes, a heart disease, an autoimmune disease, a cancer, a liver disease, a lysosomal storage disease, a blood clotting disorder or a coagulation disorder, an orthopedic condition, an amino acid metabolism disorder.

In some embodiments, the disease, disorder or condition is a neurodegenerative disease. Exemplary neurodegenerative diseases include Alzheimer's disease, Huntington's disease, Parkinson's disease (PD) amyotrophic lateral sclerosis (ALS), multiple sclerosis (MS) and cerebral palsy (CP), dentatorubro-pallidoluysian atrophy (DRPLA), neuronal intranuclear hyaline inclusion disease (NIHID), dementia with Lewy bodies, Down's syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Jakob-Creutzfeldt disease, Niemann-Pick disease type 3, progressive supranuclear palsy, subacute sclerosing panencephalitis, spinocerebellar ataxias, Pick's disease, and dentatorubral-pallidoluysian atrophy.

In some embodiments, the disease, disorder, or condition is an autoimmune disease, e.g., scleroderma, multiple sclerosis, lupus, or allergies.

In some embodiments, the disease is a liver disease, e.g., hepatitis B, hepatitis C, cirrhosis, NASH.

In some embodiments, the disease, disorder, or condition is cancer. Exemplary cancers include leukemia, lymphoma, melanoma, lung cancer, brain cancer (e.g., glioblastoma), sarcoma, pancreatic cancer, renal cancer, liver cancer, testicular cancer, prostate cancer, or uterine cancer.

In some embodiments, the disease, disorder, or condition is an orthopedic condition. Exemplary orthopedic conditions include osteoporosis, osteonecrosis, Paget's disease, or a fracture.

In some embodiments, the disease, disorder or condition is a lysosomal storage disease. Exemplary lysosomal storage diseases include Gaucher disease (e.g., Type I, Type II, Type III), Tay-Sachs disease, Fabry disease, Farber disease, Hurler syndrome (also known as mucopolysaccharidosis type I (MPS I)), Hunter syndrome, lysosomal acid lipase deficiency, Niemann-Pick disease, Salla disease, Sanfilippo syndrome (also known as mucopolysaccharidosis type IIIA (MPS3A)), multiple sulfatase deficiency, Maroteaux-Lamy syndrome, metachromatic leukodystrophy, Krabbe disease, Scheie syndrome, Hurler-Scheie syndrome, Sly syndrome, hyaluronidase deficiency, Pompe disease, Danon disease, gangliosidosis, or Morquio syndrome.

In some embodiments, the disease, disorder, or condition is a blood clotting disorder or a coagulation disorder. Exemplary blood clotting disorders or coagulation disorders include hemophilia (e.g., hemophilia A or hemophilia B), Von Willebrand disease, thrombocytopenia, uremia, Bernard-Soulier syndrome, Factor XII deficiency, vitamin K deficiency, or congenital afibrinogenimia.

In some embodiments, the disease, disorder, or condition is an amino acid metabolism disorder, e.g., phenylketonuria, tyrosinemia (e.g., Type 1 or Type 2), alkaptonuria, homocystinuria, hyperhomocysteinemia, maple syrup urine disease.

In some embodiments, the disease, disorder, or condition is a fatty acid metabolism disorder, e.g., hyperlipidemia, hypercholesterolemia, galactosemia.

In some embodiments, the disease, disorder, or condition is a purine or pyrimidine metabolism disorder, e.g., Lesch-Nyhan syndrome.

In some embodiments, the disease, disorder, or condition is diabetes (e.g., Type I or Type II diabetes).

The present disclosure further comprises methods for identifying a subject having or suspected of having a disease, disorder, or condition described herein, and upon such identification, administering to the subject particles comprising a first compartment, a second compartment, and a compound of Formula (I) (e.g., as described herein), or a composition comprising such particles. In an embodiment, the subject is a human.

### Pharmaceutical Compositions, Kits, and Administration

The present disclosure further comprises pharmaceutical compositions comprising the particles described herein, as well as kits thereof.

In some embodiments, a pharmaceutical composition comprises a particle comprising a first compartment, a second compartment, and a compound of Formula (I), as well as a pharmaceutically acceptable excipient. In some embodiments, the particles in the pharmaceutical composition comprise a cell (e.g., a human cell, e.g., an engineered human cell) and a pharmaceutically acceptable excipient. In some embodiments, the particles are provided in an effective amount in the pharmaceutical composition. In some embodiments, the effective amount is a therapeutically effective amount. In some embodiments, the effective amount is a prophylactically effective amount.

Pharmaceutical compositions described herein can be prepared by any method known in the art of pharmacology. In general, such preparatory methods include the steps of bringing the particles (e.g., particles, i.e., “the active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient (i.e., number of particles). The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the disclosure are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The particles described herein may be administered orally, parenterally (including subcutaneous, intramuscular, and intradermal), topically, rectally, nasally, intratumorally, intrathecally, buccally, vaginally or via an implanted reservoir. In some embodiments, provided particles or compositions are administrable subcutaneously or by implant.

In some embodiments, the particles and related compositions described herein may be administered or implanted in or on a certain region of the body, such as a mucosal surface or a body cavity. Exemplary sites of administration or implantation include the peritoneal cavity (e.g., lesser sac), adipose tissue, heart, eye, muscle, spleen, lymph node, esophagus, nose, sinus, teeth, gums, tongue, mouth, throat, small intestine, large intestine, thyroid, bone (e.g. hip or a joint), breast, cartilage, vagina, uterus, fallopian tube, ovary, penis, testicles, blood vessel, liver, kidney, central nervous system (e.g., brain, spinal cord, nerve), or ear (e.g., cochlea).

In some embodiments, the particles and related compositions described herein are administered or implanted at a site other than the central nervous system, e.g., the brain, spinal cord, nerve. In some embodiments, the particles and related compositions described herein are administered or implanted at a site other than the eye (e.g., retina).

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For ophthalmic use, provided pharmaceutically acceptable compositions may be formulated as micronized suspensions or in an ointment such as petrolatum.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The particles and related compositions described herein may be formulated in dosage unit form, e.g., single unit dosage form, for ease of administration and uniformity of dosage. It will be understood, however, that the total dosage and usage regimens of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a treatment required to achieve an effective amount will vary from subject to subject, depending, for example, on species, age, and general condition of a subject, severity of the side effects or disorder, identity of the particular particle(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

It will be appreciated that the particles and related compositions, as described herein, can be administered in combination with one or more additional pharmaceutical agents. The particles or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

Also encompassed by the disclosure are kits (e.g., pharmaceutical packs). The inventive kits may be useful for preventing and/or treating any of the diseases, disorders or conditions described herein. The kits provided may comprise an inventive pharmaceutical composition or particle as described herein and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or particle described herein. In some embodiments, the inventive pharmaceutical composition or particle described herein provided in the container and the second container are combined to form one unit dosage form.

### Methods of Making Particles

The present disclosure further comprises methods for making a particle described herein, e.g., a particle comprising a first compartment, a second compartment, and a compound of Formula (I). In some embodiments where the particle is a hydrogel capsule, the method of making the particle comprises contacting a plurality of droplets comprising first and second polymer solutions (e.g., each comprising a hydrogel-forming polymer) with an aqueous cross-linking solution. The droplets can be formed using any technique known in the art.

Each compartment of a particle described herein may comprise an unmodified polymer, a polymer modified with a compound of Formula (I), or a blend thereof. Briefly, in performing a method of preparing a particle configured as a two-compartment hydrogel capsule, a volume of a first polymer solution (e.g., comprising an unmodified polymer, a polymer modified with a compound of Formula (I), or a blend thereof, and optionally containing cells,) is loaded into a first syringe connected to the inner lumen of a coaxial needle. The first syringe may then be connected to a syringe pump oriented vertically above a vessel containing an aqueous cross-linking solution which comprises a cross-linking agent, a buffer, and an osmolarity-adjusting agent. A volume of the second polymer solution (e.g., comprising an unmodified polymer, a polymer modified with a compound of Formula (I), or a blend thereof, and optionally containing cells) is loaded into a second syringe connected to the outer lumen of the coaxial needle. The second syringe may then be connected to a syringe pump oriented horizontally with respect to the vessel containing the cross-linking solution. A high voltage power generator may then be connected to the top and bottom of the needle. The syringe pumps and power generator can then be used to extrude the first and second polymer solutions through the syringes with settings determined to achieve a desired droplet rate of polymer solution into the cross-linking solution. The skilled artisan may readily determine various combinations of needle lumen sizes, voltage range, flow rates, droplet rate and drop distance to create 2-compartment hydrogel capsule compositions in which the majority (e.g., at least 80%, 85%, 90% or more) of the capsules are within 10% of the target size and have a sphere-like in shape. After exhausting the first and second volumes of polymer solution, the droplets may be allowed to cross-link in the cross-linking solution for certain amount of time, e.g., about five minutes.

Exemplary process parameters for preparing a composition of millicapsules (e.g., 1.5 mm diameter millicapsules) include the following. A coaxial needle is disposed above the surface of the cross-linking solution at a distance sufficient to provide a drop distance from the needle tip to the solution surface. In an embodiment, the distance between the needle tip and the solution surface is between 1 to 5 cm. In an embodiment, the first and second polymer solutions are extruded through the needle with a total flow rate of between 0.05 mL/min to 5 mL/min, or 0.05 mL/min to 2.5 mL/min, or 0.05 mL/min to about 1 mL/min, or 0.05 mL/min to 0.5 mL/min, or 0.1 mL/min to 0.5 mL/min. In an embodiment, the first and second polymer solutions are extruded through the needle with a total flow rate of about 0.05 mL/min, 0.1 mL/min, 0.15 mL/min, 0.2 mL/min, 0.25 mL/min, 0.3 mL/min, 0.35 mL/min, 0.4 mL/min, 0.45 mL/min, or 0.5 mL/min. In an embodiment, the flow rate of the first and second polymer solutions through the needle are substantially the same. In an embodiment, the flow rate of the first and second polymer solutions through the needle are different.

In an embodiment, the voltage of the instrument is between 1 kV to 20 kV, or 1 to 15 kV, or 1 kV to 10 kV, or 5 kV to 10 kV. The voltage may be adjusted until a desired droplet rate is reached. In an embodiment, the droplet rate of the instrument is between 1 droplet/10 seconds to 50 droplets/10 seconds, or 1 droplet/10 seconds to 25 droplets/10 seconds.

In an embodiment, the number of non-particle debris on the surface of the cross-linking solution is determined. Particles that have fallen to the bottom of the cross-linking vessel may then be collected, e.g., by transferring cross-linking solution containing the particles to a separate container, leaving behind any non-particle debris on the solution surface in the original cross-linking vessel. The removed particles may then be allowed to settle, the cross-linking solution can be removed, and the particles may then be washed one or more times with a buffer (e.g., a HEPES buffer). In an embodiment, one or more aliquots of the resulting particle composition (e.g., preparation of particles) is inspected by microscopy to assess the quality of the composition, e.g., the number of particle defects and satellite particles.

In some embodiments, the cross-linking solution further comprises a process additive (e.g., a hydrophilic, non-ionic surfactant). A process additive may reduce surface tension of the cross-linking solution. Agents useful as the process additive in the present disclosure include polysorbate-type surfactants, copolymer of polyethyleneoxide (PEO) and polypropyleneoxide (PPO), poly(ethylene oxide)-poly(propylene oxide)-poly(ethylene oxide) (PEO-PPO-PEO) triblock copolymers, and non-ionic surfactants, such as Tween® 20, Tween® 80, Triton™ X-100, IGEPAL® CA-630, poloxamer 188, or poloxamer 407, or surfactants with substantially the same chemical and physical properties listed in the Exemplary Surfactant Table immediately below.

Exemplary Surfactant Table

| | | Approximate | |
|---|---|---|---|
| | | Average | |
| Brand or Generic | Commercial | Molecular Weight | Hydrophilicity |
| Name | Supplier | (g/mole) | HLB^{a} |
| Tween ® 20^{b} | Millipore Sigma | 1228 | 16.7 |
| Tween ® 80^{c} | Millipore Sigma | 1310 | 15 |
| Triton ™ X-100^{d} | Millipore Sigma | 625 | 13.4 |
| IGEPAL ® CA-630^{e} | Millipore Sigma | 603 | 13 |
| poloxamer 188^{f} | Millipore Sigma | 8400 | >24 |
| poloxamer 407^{g} | Millipore Sigma | 12,500 | 18-23 |

^{a}hydrophilic-lipophilic balance
^{b}Chemical names and synonyms: polyethylene glycol sorbitan monolaurate, polyoxyethylene (20) sorbitan monolaurate, polysorbate 20, polyoxyethylene 20 sorbitan monododecanoate
^{c}Chemical names and synonyms: polyethylene glycol sorbitan monooleate, polyoxyethylene (20) sorbitan monooleate, polysorbate 80, (x)-sorbitan mono-9-octadecenoate poly(oxy-1,2-ethanediyl)
^{d}Chemical names and synonyms: 4-(1,1,3,3-Tetramethylbutyl)phenyl-polyethylene glycol, t-octylphenoxypolyethoxyethanol, polyethylene glycol tert-octylphenyl ether; octylphenol ethoxylate, octylphenol ethylene oxide condensate
^{e}Chemical names and synonyms: octylphenoxypolyethoxyethanol, octylphenoxy poly(ethyleneoxy)ethanol, branched
^{f}Chemical name: Poly(ethylene glycol)-block-poly(propylene glycol)-block-poly(ethylene glycol)
^{g}Chemical name: Poly(ethylene glycol)-block-poly(propylene glycol)-block-poly(ethylene glycol)

In some embodiments, the process additive is a non-ionic surfactant. In an embodiment, the process additive comprises more than one surfactant, e.g., more than one hydrophilic surfactant. In some embodiments, the process additive does not contain Tween® 20 (polysorbate 20) or Triton™ X-100. In an embodiment, the process additive is IGEPAL® CA-630 (polyethylene glycol sorbitan monooleate). In some embodiments, the process additive is poloxamer 188.

In some embodiments, the process additive (e.g., surfactant) is present in the cross-linking solution at a concentration of at least 0.0001% or more. In some embodiments, the cross-linking solution comprises at least 0.001%, 0.01%, or 0.1% of the process additive. In some embodiments, the process additive is present at a concentration selected from about 0.001% to about 0.1%, about 0.005% to about 0.05%, about 0.005% to about 0.01%, and about 0.01% to about 0.5%. In an embodiment, the process additive is a surfactant and is present at a concentration that is below the critical micelle concentration for the surfactant.

In some embodiments, the cross-linking agent comprises divalent cations of a single type or a mixture of different types, e.g., one or more of Ba²⁺, Ca²⁺, Sr²⁺. In some embodiments, the cross-linking agent is BaCl₂, e.g., at a concentration of 1 mM to 100 mM or 7.5 mM to 20 mM. In some embodiments, the cross-linking agent is CaCl₂, e.g., at a concentration of 50 mM to 100 mM. In some embodiments, the cross-linking agent is SrCl₂, e.g., at a concentration of 37.5 mM to 100 mM. In some embodiments, the cross-linking agent is a mixture of BaCl₂ (e.g., 5 mM to 20 mM) and CaCl₂)(e.g., 37.5 mM to 12.5 mM) or a mixture of BaCl₂ (e.g., 5 mM to 20 mM) and SrCl₂ (e.g., 37.5 mM to 12.5 mM).

In some embodiments, the cross-linking agent is SrCl₂, and the process additive is Tween® 80 (or a surfactant with substantially the same chemical and physical properties listed in the Exemplary Surfactant Table) at a concentration of less than 0.1%, e.g., about 0.005% to 0.05%, about 0.005% to about 0.01%. In some embodiments, the concentration of SrCl₂ is about 50 mM. In some embodiments, the cross-linking agent is SrCl₂ and the process additive is poloxamer 188 at a concentration of 1%.

The type and concentration of buffer in the aqueous cross-linking solution is selected to maintain the solution pH at approximately neutral, e.g., from about 6.5 to about 7.5, about 7.0 to about 7.5, or about 7.0. In an embodiment, the buffer is compatible with a biological material to be encapsulated in the particle, e.g., cells. In some embodiments, the buffer in the aqueous cross-linking solution comprises HEPES (4-(2-hydroxyethyl)-1-piperazineethanesulfonic acid).

The osmolarity-adjusting agent in the aqueous cross-linking solution is selected to maintain the solution osmolarity at a value similar to the osmolarity of the polymer solution (which in some embodiments comprises a suspension of cells), e.g., an osmolarity that has a higher or lower variance of up to 20%, 10% or 5%. In some embodiments, the osmolarity agent is mannitol at a concentration of 0.1 M to 0.3 M.

In some embodiments, the cross-linking solution comprises 25 mM HEPES buffer, 20 mM BaCl₂, 0.2 M mannitol and 0.01% poloxamer 188.

In some embodiments, the cross-linking solution comprises 50 mM strontium chloride hexahydrate, 0.165 M mannitol, 25 mM HEPES and 0.01% of a surfactant with substantially the same chemical and physical properties listed in the Exemplary Surfactant Table for Tween 80.

In an embodiment, the process additive is poloxamer 188, which is present in the particle composition (e.g., preparation of particles) in a detectable amount after the wash steps. Poloxamer 188 may be detected by any technique known in the art, e.g., by partially or completely dissolving the particles in an aliquot of the composition by sodium sulfate precipitation and analyzing the supernatant by LC/MS.

Reduction in the surface tension of the cross-linking solution may be assessed by any method known in the art, for example, through the use of a contact angle goniometer or a tensiometer, e.g., via the du Nouy ring method (see, e.g., Davarci et al (2017) Food Hydrocolloids 62:119-127).

### Enumerated Exemplary Embodiments

1. A particle comprising:
a) a first compartment;
b) a second compartment; and
c) a compound of Formula (I-a):

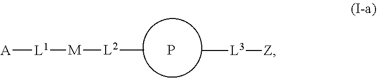

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆-alkylene)-, —N(R^{C})C(O)(C₂-C₆-alkenylene)-, —C(═N(R^{C})(R^{D}))O—, —S—, —S(O)ₓ—, —OS(O)ₓ—, —N(R^{C})S(O)ₓ—, —S(O).N(R^{C})—, —P(R^{F})—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

2. The particle of embodiment 1, wherein the first compartment is surrounded by the second compartment.

3. The particle of any one of embodiments 1-2, wherein the first compartment is disposed within the second compartment.

4. The particle of any one of embodiments 1-3, wherein the second compartment forms a barrier around the first compartment.

5. The particle of any one of embodiments 1-4, wherein the total volume of the second compartment is greater than, e.g. 1.5×, 2×, 3×, or 5×, the volume of the first compartment.

6. The particle of any one of embodiments 1-4, wherein the differential volume of the second compartment is greater than, e.g. 1.5×, 2×, 3×, or 5×, the volume of the first compartment.

7. The particle of any one of embodiments 1-4, wherein the total volume of the second compartment is about 1%, 2%, 5%, 7.5%, 10%, 15%, 20%, 25%, 30%, 35%, 40%, 45%, 50%, 55%, 60%, 65%, or 75% greater than the volume of the first compartment.

8. The particle of any one of embodiments 1-4, wherein the differential volume of the second compartment is about 1%, 2%, 5%, 7.5%, 10%, 15%, 20%, 25%, 30%, 35%, 40%, 45%, 50%, 55%, 60%, 65%, or 75% greater than the volume of the first compartment.

9. The particle of any one of embodiments 1-4, wherein the differential volume of the second compartment is less than, e.g. 1.5×, 2×, 3×, or 5×, the volume of the first compartment.

10. The particle of any one of embodiments 1-4, wherein the total volume of the second compartment is about 1%, 2%, 5%, 7.5%, 10%, 15%, 20%, 25%, 30%, 35%, 40%, 45%, 50%, 55%, 60%, 65%, or 75% less than the volume of the first compartment.

11. The particle of any one of embodiments 1-4, wherein the differential volume of the second compartment is about 1%, 2%, 5%, 7.5%, 10%, 15%, 20%, 25%, 30%, 35%, 40%, 45%, 50%, 55%, 60%, 65%, or 75% less than the volume of the first compartment.

12. The particle of embodiment 1, comprising a property selected from the following:
a) the first compartment comprises a compound of Formula (I-a);
b) the second compartment comprises a compound of Formula (I-a);
c) a compound of Formula (I-a) is disposed on the exterior surface of the particle; and/or
d) the particle comprises an interface between the first and second compartments and a compound of Formula (I-a) is disposed at the interface.

13. The particle of embodiment 12, comprising property a.

14. The particle of any one of embodiments 12-13, comprising property b.

15. The particle of any one of embodiments 12-14, comprising property c.

16. The particle of any one of embodiments 12-15, comprising property d.

17. The particle of embodiment 1, wherein the first compartment or the second compartment is substantially free of a compound of Formula (I-a).

18. The particle of embodiment 1, wherein the outer surface of the particle is substantially free of a compound of Formula (I-a).

19. The particle of embodiment 1, comprising a property selected from the following:
a) the first compartment is substantially free of a compound of Formula (I-a);
b) the second compartment is substantially free of a compound of Formula (I-a);
c) the outer surface of the particle is substantially free of a compound of Formula (I-a); or
d) the particle comprises an interface between the first and second compartment and the interface is substantially free of a compound of Formula (I-a).

20. The particle of embodiment 19, comprising property a.

21. The particle of any one of embodiments 19-20, comprising property b.

22. The particle of any one of embodiments 19-21, comprising property c.

23. The particle of any one of embodiments 19-22, comprising property d.

24. The particle of embodiment 19, comprising properties a and b.

25. The particle of any one of embodiments 1-24, wherein the particle has a largest linear dimension (LLD), e.g., diameter, of between 20 nanometers to 10 millimeters.

26. The particle of any one of embodiments 1-25, wherein the particle has a largest linear dimension (LLD), e.g., diameter, of between 500 nanometers to 10 millimeters.

27. The particle of any one of embodiments 1-26, wherein the particle has a largest linear dimension (LLD), e.g., diameter, of between 1 millimeter to 5 millimeters, e.g., between 1 millimeter to 4 millimeters, 1 millimeter to 3 millimeters, 1 millimeter to 2 millimeters, about 1.5 millimeters to 2 millimeters, or about 1.5 millimeters.

28. The particle of any one of embodiments 1-27, wherein the particle is configured as a hydrogel capsule with the first compartment surrounded by the second compartment.

29. The particle of embodiment 28, wherein the thickness of the second compartment is selected from the group consisting of:
(a) 1 nanometers and 1 millimeter;
(b) 100 nanometers and 1 millimeter; and
(c) 500 nanometers and 500 micrometers.

30. The particle of embodiment 29, wherein the thickness of the second compartment is at least about 2.5%, 5%, 10%, 15%, 20%, 25%, 30%, 35%, 40%, 50%, 60%, 70%, or 80% of the diameter of the particle.

31. The particle of any one of embodiments 1-30, wherein the particle comprises a cell.

32. The particle of any one of embodiments 1-31, wherein the first compartment comprises a cell.

33. The particle of any one of embodiments 1-32, wherein the second compartment comprises a cell.

34. The particle of any one of embodiments 1-33, wherein the first compartment comprises a cell and the second compartment does not comprise a cell.

35. The particle of any one of embodiments 1-34, wherein the first compartment comprises a cell and the second compartment comprises a cell.

36. The particle of embodiment 35, wherein the first compartment and the second compartment comprise the same type of cell.

37. The particle of embodiment 35, wherein the cell in the first compartment is a different type of cell than the cell in the second compartment.

38. The particle of any one of embodiments 31-36, wherein the particle comprises an interface between the first compartment and the second compartment and a cell is disposed at the interface, e.g., a cell contacts both the first and second compartments.

39. The particle of any one of embodiments 31-37, wherein the number or density of cells in the second compartment is less than the number or density of cells in the first compartment.

40. The particle of any one of embodiments 31-39, wherein the first compartment is formed from a polymer solution comprising at least 0.5×10⁶, 1×10⁶, 5×10⁶, 10×10⁶, 15×10⁶ or 20×10⁶ cells per mL.

41. The particle of any one of embodiments 31-40, wherein the first compartment is formed from a polymer solution comprising at least 0.5×10⁶, 1×10⁶, 5×10⁶, 10×10⁶, 15×10⁶, 20×10⁶ or 25×10⁶ cells per mL or from a polymer solution comprising 100 to 300 million cells per mL.

42. The particle of any one of embodiments 31-41, wherein the particle comprises at least 100; 250; 500; 750; 1,000; 2,500; 5,000; 10,000; 25,000; or 50,000 cells.

43. The particle of any one of embodiments 31-42, wherein the first compartment comprises at least 100; 250; 500; 750; 1,000; 2,500; 5,000; 10,000; 25,000; or 50,000 cells.

44. The particle of any one of embodiments 31-43, wherein the cells are present as single cells, one or more spheroids, or bound to one or more microcarriers.

45. The particle of any one of embodiments 31-44, wherein the exterior surface of the particle is substantially free of cells.

46. The particle of any one of embodiments 31-45, wherein:
a) one or a plurality of cells is disposed within the first compartment;
b) the number or density of cells in the second compartment is at least 2, 5, 10, 10², 10³, or 10⁴ times less than the number of density of cells in the first compartment;
c) the first compartment (e.g., the outer boundary of the first compartment) comprises a compound of Formula (I-a); or
d) the second compartment (e.g., the outer boundary of the second compartment) comprises a compound of Formula (I-a).

47. The particle of embodiment 46, comprising property a.

48. The particle of any one of embodiments 46-47, comprising property b.

49. The particle of any one of embodiments 46-48, comprising property c.

50. The particle of any one of embodiments 46-49, comprising property d.

51. The particle of embodiment 50, comprising properties a and b.

52. The particle of embodiment 50, comprising properties a, b, and c.

53. The particle of embodiment 50, comprising properties a, b, and d.

54. The particle of embodiment 50, comprising properties a, b, c, and d.

55. The particle of any one of embodiments 31-54, wherein the second compartment is substantially free of cells.

56. The particle of any one of embodiments 31-55, wherein the cell is an epithelial cell, endothelial cell, fibroblast cell, mesenchymal stem cell, or keratinocyte cell.

57. The particle of any one of embodiments 31-56, wherein the cell is an RPE (e.g., ARPE-19) cell or an MSC.

58. The particle of any one of embodiments 31-56, wherein the cell is an islet cell.

59. The particle of any one of embodiments 31-58, wherein the cell expresses a therapeutic agent (e.g., a polypeptide).

60. The particle of embodiment 59, wherein the polypeptide is a Factor VIII protein or a variant thereof or a Factor IX protein or a variant thereof.

61. The particle of any one of embodiments 59-60, wherein the polypeptide comprises SEQ ID NO:1 or a variant thereof.

62. The particle of any one of embodiments 59-60, wherein the polypeptide comprises SEQ ID NO: 2 or a variant thereof, e.g., an alanine substituted for threonine at amino acid position 148 of SEQ ID NO:2 or a leucine substituted for arginine at amino acid position 338 of SEQ ID NO:2.

63. The particle of embodiment 59, wherein the polypeptide is insulin (e.g., insulin A-chain, insulin B-chain, or proinsulin).

64. The particle of any one of embodiments 1-63, wherein the particle comprises a polymer.

65. The particle of embodiment 64, wherein the polymer is a polysaccharide.

66. The particle of any one of embodiments 64-65, wherein the polymer is selected from alginate, chitosan, hyaluronate, gelatin, poly(L-lactic acid) (PLLA), or poly(lactic glycolic acid) (PLGA).

67. The particle of any one of embodiments 64-66, wherein the first compartment comprises a polymer (e.g., a polysaccharide, e.g., an alginate).

68. The particle of any one of embodiments 64-67, wherein the second compartment comprises a polymer (e.g., a polysaccharide, e.g., an alginate).

69. The particle of any one of embodiments 64-68, wherein both the first compartment and the second compartment comprise a polymer (e.g., a polysaccharide, e.g., an alginate).

70. The particle of any one of embodiments 64-69, wherein the first compartment and the second compartment comprise the same polymer.

71. The particle of any one of embodiments 64-70, wherein the first compartment and the second compartment comprise a different polymer.

72. The particle of any one of embodiments 64-71, wherein the first compartment does not comprise alginate and the second compartment comprises alginate.

73. The particle of any one of embodiments 64-72, wherein first compartment comprises an alginate and the second compartment comprises a polymer other than alginate.

74. The particle of any one of embodiments 64-73, wherein second compartment comprises an alginate and the first compartment comprises a polymer other than alginate.

75. The particle of embodiment 74, wherein the first compartment comprises hyaluronate or chondroitin and the second compartment comprises an alginate.

76. The particle of any one of embodiments 73-75, wherein the polymer of the first compartment is modified with a compound of Formula (I-a).

77. The particle of any one of embodiments 73-76, wherein the polymer of the second compartment is modified with a compound of Formula (I-a).

78. The particle of any one of embodiments 1-77, wherein the exterior surface of the particle and interior of the second compartment comprise a compound of Formula (I-a).

79. The particle of any one of embodiments 64-78, wherein the polymers of both the first compartment and second compartment are modified with a compound of Formula (I-a).

80. The particle of any one of embodiments 1-79, wherein the compound of Formula (I-a) is a compound of any one of Formulas (I-b), (I-c), (I-d), (I-e), (I-f), (II), (II-a), (III), (III-a), (III-b), (III-c), or (III-d), or a pharmaceutically acceptable salt thereof.

81. The particle of any one of embodiments 76-80, wherein the compound of Formula (I-a) is selected from Compound 110, Compound 112, Compound 113 or Compound 114 shown in Table 2.

82. The particle of any one of embodiments 76-80, wherein the compound of Formula (I-a) is Compound 112 shown in Table 2.

83. The particle of any one of embodiments 76-80, wherein the compound of Formula (I-a) is Compound 113 shown in Table 2.

84. The particle of any one of embodiments 76-80, wherein the compound of Formula (I-a) is Compound 114 shown in Table 2.

85. The particle of any one of embodiments 76-80, wherein the compound of Formula (I-a) is not Compound 100 shown in Table 2.

86. The particle of any one of embodiments 76-80, wherein the compound of Formula (I-a) is Compound 101 shown in Table 2.

87. The particle of any one of embodiments 76-86, wherein at least 0.5% of the monomers of a polymer are modified with a compound of Formula (I-a) (e.g., at least 1%, 2.5%, 5%, 10%, 15%, 20%, 25%, 30%, 35%, 40%, 45%, 50%, 55%, 60%, 65%, 70%, 75%, 80%, 85%, 90%, 95%, 99%, or more of the monomers of a polymer are modified with a compound of Formula (I-a)).

88. The particle of any one of embodiments 76-86, wherein at least 0.5% of the monomers of a polymer in the first (inner) compartment of the particle are modified with a compound of Formula (I-a) (e.g., at least 1%, 2.5%, 5%, 10%, 15%, 20%, 25%, 30%, 35%, 40%, 45%, 50%, 55%, 60%, 65%, 70%, 75%, 80%, 85%, 90%, 95%, 99%, or more of the monomers of a polymer in the first (inner) compartment of the particle are modified with a compound of Formula (I-a)).

89. The particle of any one of embodiments 76-86, wherein at least 0.5% of the monomers of a polymer in the second (outer) compartment of the particle are modified with a compound of Formula (I-a) (e.g., at least 1%, 2.5%, 5%, 10%, 15%, 20%, 25%, 30%, 35%, 40%, 45%, 50%, 55%, 60%, 65%, 70%, 75%, 80%, 85%, 90%, 95%, 99%, or more of the monomers of a polymer in the second (outer) compartment of the particle are modified with a compound of Formula (I-a)).

90. The particle of any one of embodiments 76-86, wherein the polymer (when modified with a compound of Formula (I-a)) comprises an increase in % N (as compared with unmodified polymer) of 0.1% to 10% N by weight (e.g., 0.1% to 2% N, 2% to 4%, or 4% to 8% N by weight), where % N is determined by combustion analysis and corresponds to the amount of compound of Formula (I-a) in the modified polymer.

91. The particle of any one of embodiments 76-86, wherein the first (inner) compartment of the particle comprises a polymer (when modified with a compound of Formula (I-a)) that comprises an increase in % N (as compared with unmodified polymer) of 0.1% to 10% N by weight (e.g., 0.1% to 2% N, 2% to 4%, or 4% to 8% N by weight), where % N is determined by combustion analysis and corresponds to the amount of compound of Formula (I-a) in the modified polymer.

92. The particle of any one of embodiments 76-84, wherein the second (outer) compartment of the particle comprises a polymer (when modified with a compound of Formula (I-a)) that comprises increase in % N (as compared with unmodified polymer) of 0.1% to 10% N by weight (e.g., 0.1% to 2% N, 2% to 4%, or 4% to 8% N by weight), where % N is determined by combustion analysis and corresponds to the amount of compound of Formula (I-a) in the modified polymer.

93. The particle of any one of embodiments 76-84, wherein the particle is a hydrogel capsule and the second (outer) compartment of the capsule is formed using a mixture of an unmodified alginate and an alginate modified with a compound of Formula (I-a) (e.g., Compound 101) at a conjugation density of at least 2.0% and less than 9.0% nitrogen (N) as determined by combustion analysis for percent nitrogen as described in the Examples hereinbelow, or is 3.0% to 8.0%, 4.0% to 7.0%, 5.0% to 7.0%, or 6.0% to 7.0% or about 6.8%.

94. The particle of any one of embodiments 1-93, wherein the particle is a spherical particle.

95. The particle of any one of embodiments 1-94, wherein the particle is made by a method wherein the second compartment is formed around the first compartment.

96. The particle of any of embodiments 1-95, made by a method comprising contacting a plurality of droplets of a polymer solution with an aqueous cross-linking solution for a period of time sufficient to produce a particle, wherein the cross-linking solution comprises a cross-linking agent, a buffer, and an osmolarity-adjusting agent.

97. The particle of embodiment 96, wherein the cross-linking solution further comprises a process additive.

98. The particle of embodiment 97, wherein the process additive is a surfactant.

99. The particle of embodiment 98, wherein the surfactant is selected from a polysorbate-type surfactant, a copolymer of polyethyleneoxide (PEO) and polypropyleneoxide (PPO), a poly(ethylene oxide)-poly(propylene oxide)-poly(ethylene oxide) (PEO-PPO-PEO) triblock copolymer, polysorbate 20, polysorbate 80, 4-(1,1,3,3-Tetramethylbutyl)phenyl-polyethylene glycol, octylphenoxypolyethoxyethanol, poloxamer 188 and poloxamer 407.

100. The particle of any one of embodiments 98-99, wherein the surfactant has a hydrophilic-lipophilic balance (HLB) of at least 18 or at least 24, and optionally wherein the surfactant is poloxamer 188.

101. The particle of any one of embodiments 97-99, wherein the process additive is present in the cross-linking solution at a concentration of at least about 0.001% to about 0.1%, about 0.005% to about 0.05%, about 0.005% to about 0.01%, or about 0.01% to about 0.05%.

102. The particle of any one of embodiments 96-101, wherein the cross-linking agent comprises divalent cations of a single type or a mixture of different types, optionally wherein the cross-linking agent comprises one or more of Ba²⁺, Ca²⁺ and Sr²⁺.

103. The particle of any one of embodiments 96-102, wherein the cross-linking agent is selected from the group consisting of:
a. BaCl₂ at a concentration of 1 mM to 100 mM or 7.5 mM to 20 mM;
b. CaCl₂ at a concentration of 50 mM to 100 mM;
c. SrCl₂ at a concentration of 37.5 mM to 100 mM;
d. a mixture of BaCl₂ at a concentration of 5 mM to 20 mM and CaCl₂ at a concentration of 37.5 mM to 12.5 mM; and
e. a mixture of BaCl₂ at a concentration of 5 mM to 20 mM and SrCl₂ at a concentration of 37.5 mM to 12.5 mM.

104. The particle of any one of embodiments 96-103, wherein the buffer comprises 4-(2-hydroxyethyl)-1-piperazineethanesulfonic acid) (HEPES).

105. The particle of any one of embodiments 96-104, wherein the osmolarity-adjusting agent comprises mannitol at a concentration of 0.1 M to 0.3 M.

106. The particle of any of embodiments 96-105, wherein the cross-linking agent is not SrCl₂.

107. The particle of any one of embodiments 96-106, wherein the cross-linking agent is BaCl₂.

108. The particle of any one of embodiments 96-107, wherein the cross-linking solution comprises 25 mM HEPES buffer, 20 mM BaCl₂, 0.2 M mannitol and 0.01% poloxamer 188.

109. The particle of any one of embodiments 96-108, wherein the cross-linking agent is SrCl₂ and the process additive is a surfactant at a concentration of about 0.01%, wherein the surfactant is polysorbate 80.

110. The particle of embodiment 109, wherein the cross-linking solution comprises 50 mM strontium chloride hexahydrate, 0.165 M mannitol, 25 mM HEPES and 0.01% of polysorbate 80.

111. The particle of any one of embodiments 1 to 110, wherein the particle is a hydrogel millicapsule comprising a hydrogel forming polymer in each of the first and second compartments.

112. The particle of embodiment 111, wherein the only hydrogel forming polymer in the first compartment is a high molecular weight alginate and the hydrogel forming polymer in the second compartment is a mixture of a chemically modified low molecular weight alginate and an unmodified high molecular weight alginate.

113. A preparation of a plurality of particles, wherein the plurality comprises a particle of any one of embodiments 1-112

114. The preparation of embodiment 113, wherein at least 75%, 80%, 85%, 90%, 95%, 99%, or more of the particles in the plurality are spherical particles, and optionally wherein the preparation comprises a detectable amount of the process additive.

115. The preparation of embodiment 113 to 114, wherein the preparation is a pharmaceutically acceptable preparation.

116. A method of making a particle described herein, e.g., a particle of any of embodiments 1-115.

117. The method of embodiment 116, comprising forming the first compartment prior to formation of the second compartment.

118. The method of embodiment 117, comprising forming the first compartment at the same time as the formation of the second compartment.

119. The method of any one of embodiments 116-118, comprising contacting a plurality of droplets of first and second polymer solutions with an aqueous cross-linking solution for a period of time sufficient to produce a hydrogel capsule with first and second compartments, wherein the cross-linking solution comprises a cross-linking agent, a buffer, and an osmolarity-adjusting agent.

120. The method of any one of embodiments 116-119, wherein the method comprises use of a coaxial needle.

121. The method of any one of embodiments 116-120, wherein the first polymer solution comprises cells.

122. A method of implanting a particle in a subject comprising:
providing a particle described herein, e.g., in any of embodiments 1 to 112; and
disposing the particle in the body of the subject.

123. A method of providing a substance, e.g., a therapeutic substance, e.g., a polypeptide, to a subject comprising:
providing a particle described herein, e.g., in any of embodiments 1-112; which comprises or has the ability to produce the substance; and
disposing the particle in the body of the subject.

124. A method of evaluating a particle, e.g., in a subject comprising:
providing a particle described herein, e.g., in any of embodiments 1-112; and
disposing the particle in the body of the subject.

125. A method of treating a subject in need of a substance, e.g., a polypeptide, to a subject comprising:
providing a particle described herein, e.g., in any of embodiments 1-112; which comprises or has the ability to produce the substance; and
disposing the particle in the body of the subject.

126. A composition of particles for use in treating a subject in need of a substance, e.g., a polypeptide, to a subject comprising:
providing a particle described herein, e.g., in any of embodiments 1-112; which comprises or has the ability to produce the substance; and
disposing the particle in the body of the subject.

### EXAMPLES

In order that the disclosure described herein may be more fully understood, the following examples are set forth. The examples described in this application are offered to illustrate the particles, chemical modifications, compositions and methods provided herein and are not to be construed in any way as limiting their scope.

### Example 1: Synthesis of Exemplary Compounds for Preparation of Chemically Modified Implantable Elements

### General Protocols

The procedures below describe methods of preparing exemplary compounds for preparation of chemically modified implantable elements. The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Huisgen Cycloaddition to Afford 1,4-Substituted Triazoles

The copper-catalyzed Huisgen [3+2]cycloaddition was used to prepare triazole-based compounds and compositions, devices, and materials thereof. The scope and typical protocols have been the subject of many reviews (e.g., Meldal, M. and Tornoe, C. W. Chem. Rev. (2008) 108:2952-3015; Hein, J. E. and Fokin, V. V. Chem. Soc. Rev. (2010) 39(4):1302-1315; both of which are incorporated herein by reference).

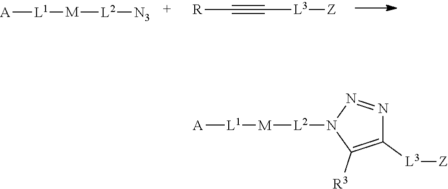

In the example shown above, the azide is the reactive moiety in the fragment containing the connective element A, while the alkyne is the reactive component of the pendant group Z. As depicted below, these functional handles can be exchanged to produce a structurally related triazole product. The preparation of these alternatives is similar, and do not require special considerations.

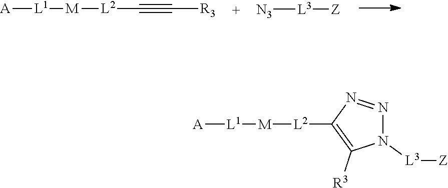

A typical Huisgen cycloaddition procedure starting with an iodide is outlined below. In some instances, iodides are transformed into azides during the course of the reaction for safety.

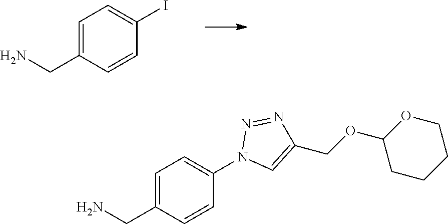 [SMILES: NCc1ccc(I)cc1 ; NCc1ccc(-n2cc(COC3CCCCO3)nn2)cc1]

A solution of sodium azide (1.1 eq), sodium ascorbate, (0.1 eq) trans-N,N′-dimethylcyclohexane-1,2-diamine (0.25 eq), copper (I) iodide in methanol (1.0 M, limiting reagent) was degassed with bubbling nitrogen and treated with the acetylene (1 eq) and the aryl iodide (1.2 eq). This mixture was stirred at room temperature for 5 minutes, then warmed to 55° C. for 16 h. The reaction was then cooled to room temperature, filtered through a funnel, and the filter cake washed with methanol. The combined filtrates were concentrated and purified via flash chromatography on silica gel (120 g silica, gradient of 0 to 40% (3% aqueous ammonium hydroxide, 22% methanol, remainder dichloromethane) in dichloromethane to afford the desired target material.

A typical Huisgen cycloaddition procedure starting with an azide is outlined below.

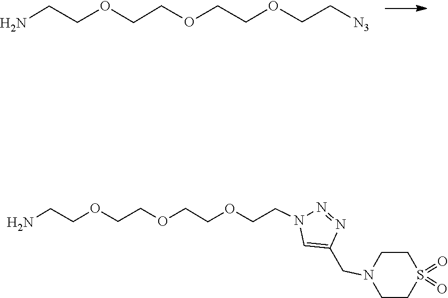 [SMILES: [N-]=[N+]=NCCOCCOCCOCCN ; NCCOCCOCCOCCn1cc(CN2CCS(=O)(=O)CC2)nn1]

A solution of tris[(1-benzyl-1H-1,2,3-triazol-4-yl)methyl]amine (0.2 eq), triethylamine (0.5 eq), copper (I) iodide (0.06 eq) in methanol (0.4 M, limiting reagent) was treated with the acetylene (1.0 eq) and cooled to 0° C. The reaction was allowed to warm to room temperature over 30 minutes, then heated to 55° C. for 16 h. The reaction was cooled to room temperature, concentrated, and purified with HPLC (C18 column, gradient of 0 to 100% (3% aqueous ammonium hydroxide, 22% methanol remainder dichloromethane) in dichloromethane to afford the desired target material.

### Huisgen Cycloaddition to Afford 1,5-Substituted Triazoles

The Huisgen [3+2]cycloaddition was also performed with ruthenium catalysts to obtain 1,5-disubstituted products preferentially (e.g., as described in Zhang et al, J. Am. Chem. Soc., 2005, 127, 15998-15999; Boren et al, J. Am. Chem. Soc., 2008, 130, 8923-8930, each of which is incorporated herein by reference in its entirety).

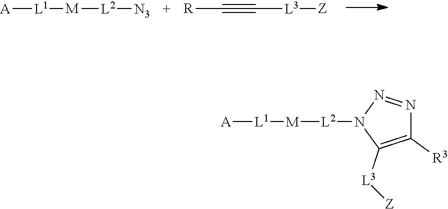

As described previously, the azide and alkyne groups may be exchanged to form similar triazoles as depicted below.

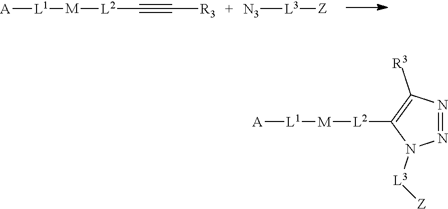

A typical procedure is described as follows: a solution of the alkyne (1 eq) and the azide (1 eq) in dioxane (0.8M) were added dropwise to a solution of pentamethylcyclo-pentadienylbis(triphenylphosphine) ruthenium(II) chloride (0.02 eq) in dioxane (0.16M). The vial was purged with nitrogen, sealed and the mixture heated to 60° C. for 12 h. The resulting mixture was concentrated and purified via flash chromatography on silica gel to afford the requisite compound.

### Experimental Procedure for (4-(4-((4-methylpiperazin-1-yl)methyl)-1H-1,2,3-triazol-1-yl)phenyl)methanamine (3)

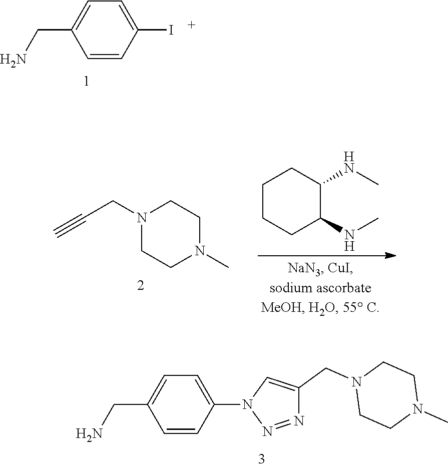

A mixture of (4-iodophenyl)methanamine (1, 843 mg, 3.62 mmol, 1.0 eq), (1S,2S)—N1,N2-dimethylcyclohexane-1,2-diamine (74 μL, 0.47 mmol, 0.13 eq), Sodium ascorbate (72 mg, 0.36 mmol, 0.1 eq), Copper Iodide (69 mg, 0.36 mmol, 0.1 eq), Sodium azide (470 mg, 7.24 mmol, 2.0 eq), and 1-methyl-4-(prop-2-yn-1-yl)piperazine (2, 0.5 g, 3.62 mmol, 1.0 eq) in Methanol (9 mL) and water (1 mL) were purged with nitrogen for 5 minutes and heated to 55° C. for over night. The reaction mixture was cooled to room temperature, concentrated under reduced pressure, and the brownish slurry was extracted with dichloromethane. Celite was added to the combined dichloromethane phases and the solvent was removed under reduced pressure. The crude product was purified over silica gel (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 7.5% to afford (4-(4-((4-methylpiperazin-1-yl)methyl)-1H-1,2,3-triazol-1-yl)phenyl)methanamine (3, 0.45 g, 43%). LCMS m/z: [M+H]⁺ Calcd for C₁₅H₂₂N₆, 287.2. Found 287.1.

### Experimental Procedure for N-(4-(4-((4-methylpiperazin-1-yl)methyl)-1H-1,2,3-triazol-1-yl)benzyl)methacrylamide (4)

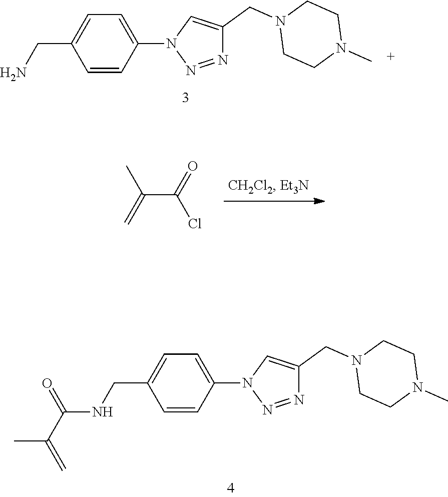

A solution of (4-(4-((4-methylpiperazin-1-yl)methyl)-1H-1,2,3-triazol-1-yl)phenyl)methanamine (3, 1.2 g, 4.19 mmol, 1.0 eq) and triethylamine (0.70 mL, 5.03 mmol, 1.2 eq) in CH₂Cl₂ (50 mL) was cooled to 0° C. with an ice-bath and methacryloyl chloride (0.43 mL, 4.40 mmol, 1.05 eq in 5 mL of CH₂Cl₂) was added. The reaction was stirred for a day while cooled with an ice-bath. 10 grams of Celite were added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 7.5%. The solvent was removed under reduced pressure and the resulting solid was triturated with diethyl ether, filtered and washed multiple times with diethyl ether to afford N-(4-(4-((4-methylpiperazin-1-yl)methyl)-1H-1,2,3-triazol-1-yl)benzyl)methacrylamide (4, 0.41 g, 28% yield) as a white solid. LCMS m/z: [M+H]⁺ Calcd for C₁₉H₂₆N₆O, 355.2. Found 355.2.

### Experimental Procedure for (4-(4-((2-(2-methoxyethoxy)ethoxy)methyl)-1H-1,2,3-triazol-1-yl)phenyl)methanamine (6)

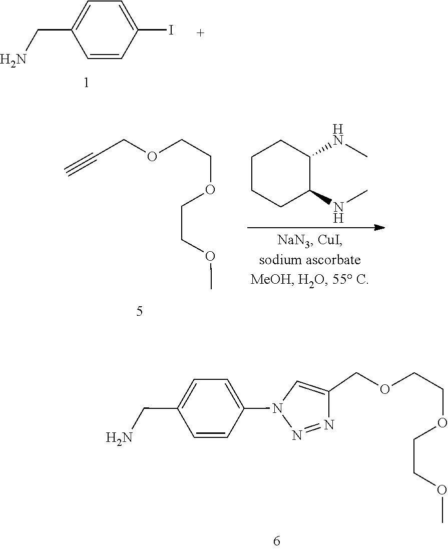

A mixture of (4-iodophenyl)methanamine (1, 2.95 g, 12.64 mmol, 1.0 eq), (1S,2S)—N1,N2-dimethylcyclohexane-1,2-diamine (259 μL, 1.64 mmol, 0.13 eq), sodium ascorbate (250 mg, 1.26 mmol, 0.1 eq), copper iodide (241 mg, 1.26 mmol, 0.1 eq), sodium azide (1.64 g, 25.29 mmol, 2.0 eq), and 3-(2-(2-methoxyethoxy)ethoxy)prop-1-yne (5, 2.0 g, 12.64 mmol, 1.0 eq) in methanol (40 mL) and water (4 mL) was purged with nitrogen for 5 minutes and then heated to 55° C. overnight. The reaction mixture was cooled to room temperature and concentrated under reduced pressure. The residue was dissolved in dichloromethane, filtered, and concentrated with Celite® (10 g). The crude product was purified on silica gel (220 g) using dichloromethane/(methanol containing 12% (v/v) aqueous ammonium hydroxide) as the eluent. The concentration of (methanol containing 12% (v/v) aqueous ammonium hydroxide) was gradually increased from 0% to 6.25% to afford (4-(4-((2-(2-methoxyethoxy)ethoxy)methyl)-1H-1,2,3-triazol-1-yl)phenyl)methanamine (6, 1.37 g, 35%). LCMS m/z: [M+H]⁺ Calcd for C₁₅H₂₂N₄O₃, 307.2. Found 307.0.

### Experimental Procedure for N-(4-(4-((2-(2-methoxyethoxy)ethoxy)methyl)-1H-1,2,3-triazol-1-yl)benzyl)methacrylamide (7)

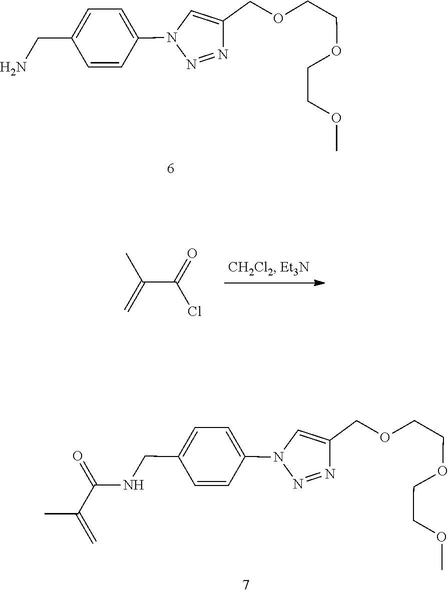

A solution of 4-(4-((2-(2-methoxyethoxy)ethoxy)methyl)-1H-1,2,3-triazol-1-yl)phenyl)methanamine (6, 1.69 g, 5.52 mmol, 1.0 eq) and triethylamine (0.92 mL, 6.62 mmol, 1.2 eq) in CH₂Cl₂ (50 mL) was cooled to 0° C. with an ice-bath and methacryloyl chloride (0.57 mL, 5.79 mmol, 1.05 eq) was added in a dropwise fashion. The reaction was stirred for 4 h at room temperature. 10 grams of Celite were added and the solvent was removed under reduced pressure. The residue was purified by silica gel (80 g) chromatography using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(4-(4-((2-(2-methoxyethoxy)ethoxy)methyl)-1H-1,2,3-triazol-1-yl)benzyl)methacrylamide (7, 1.76 g, 85% yield) as a white solid. LCMS m/z: [M+H]⁺ Calcd for C₁₉H₂₆N₄O₄, 375.2. Found 375.0.

### Experimental Procedure for 3-(prop-2-yn-1-yloxy)oxetane (9)

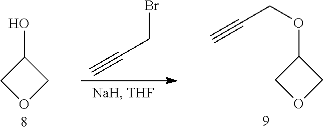

A suspension of sodium hydride (27.0 g, 675 mmol, 60% purity) in THF (200 mL) was cooled with an ice bath. Oexetan-3-ol (8, 25 g, 337 mmol) was added in a dropwise fashion and stirred for 30 minutes at 0° C. 3-Bromoprop1-yne (9, 41.2 mL, 371 mmol, 80% purity) was then added in a dropwise fashion. The mixture was stirred over night while allowed to warm to room temperature. The mixture was filtered over Celite, washed with THF, and concentrated with Celite under reduced pressure. The crude product was purified over silica gel (220 g) and eluted with Hexanes/EtOAc. The concentration of EtOAc in the mobile phase was increased from 0 to 25% to afford a yellow oil of (9, 18.25 g 48%).

### Experimental Procedure for 3-(4-((oxetan-3-yloxy)methyl)-1H-1,2,3-triazol-1-yl)propan-1-amine (11)

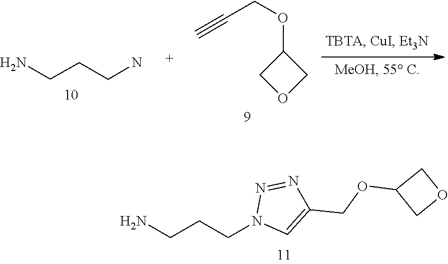

A mixture of 3-(prop-2-yn-1-yloxy)oxetane (9, 7.96 g, 71 mmol, 1.0 eq), 3-azidopropan-1-amine (10, 7.82 g, 78 mmol, 1.1 eq), Tris[(1-benzyl-1H-1,2,3-triazol-4-yl)methyl]-amine (8.29 g, 15.6 mmol, 0.22 eq), Copper Iodide (1.35 g, 7.1 mmol, 0.1 eq), and Triethylamine (2.47 mL, 17.8 mmol, 0.25 eq) in Methanol (80 mL) was warmed to 55° C. and stirred over night under Nitrogen atmosphere. The reaction mixture was cooled to room temperature, Celite (20 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 15% to afford 3-(4-((oxetan-3-yloxy)methyl)-1H-1,2,3-triazol-1-yl)propan-1-amine (11, 11.85 g, 79%) as a yellow oil. LCMS m/z: [M+H]⁺ Calcd for C₉H₁₆N₄O₂, 213.1. Found 213.0.

### Experimental Procedure for N-(3-(4-((oxetan-3-yloxy)methyl)-1H-1,2,3-triazol-1-yl)propyl)methacrylamide (12)

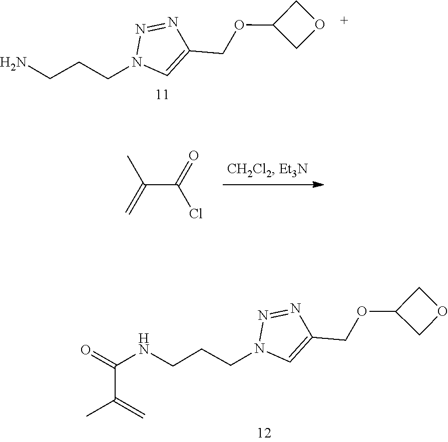

A solution of 3-(4-((oxetan-3-yloxy)methyl)-1H-1,2,3-triazol-1-yl)propan-1-amine (11, 3.94 g, 18.56 mmol, 1.0 eq) and triethylamine (3.1 mL, 22.28 mmol, 1.2 eq) in CH₂Cl₂ (100 mL) was cooled to 0° C. with an ice-bath and methacryloyl chloride (1.99 mL, 20.42 mmol, 1.1 eq) was added in a dropwise fashion. The reaction was stirred over night while allowed to warm to room temperature. 20 grams of Celite were added and the solvent was removed under reduced pressure. The residue was purified by silica gel chromatography (220 g) using dichloromethane/methanol as mobile phase. The concentration of methanol was gradually increased from 0% to 5% to afford N-(3-(4-((oxetan-3-yloxy)methyl)-1H-1,2,3-triazol-1-yl)propyl)methacrylamide (12, 3.22 g, 62% yield) as a solid. LCMS m/z: [M+H]⁺ Calcd for C₁₃H₂₀N₄O₃, 281.2. Found 281.0.

### Experimental Procedure for N-(4-(1H-1,2,3-triazol-1-yl)benzyl) methacrylamide (14)

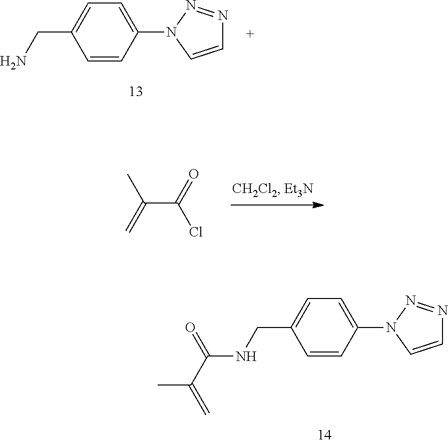

To a solution of (4-(1H-1,2,3-triazol-1-yl)phenyl)methanamine (13, obtained from WuXi, 1.2 g, 5.70 mmol, 1.0 eq) and triethylamine (15 mL, 107.55 mmol, 18.9 eq) in CH₂Cl₂ (100 mL) was slowly added methacryloyl chloride (893 mg, 8.54 mmol, 1.5 eq) in a dropwise fashion. The reaction was stirred over night. 20 grams of Celite were added and the solvent was removed under reduced pressure. The residue was purified by silica gel chromatography using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(4-(1H-1,2,3-triazol-1-yl)benzyl) methacrylamide (14, 1.38 g, 40% yield).

### Experimental Procedure for (4-(4-(((tetrahydro-2H-pyran-2-yl)oxy)methyl)-1H-1,2,3-triazol-1-yl)phenyl)methanamine (15)

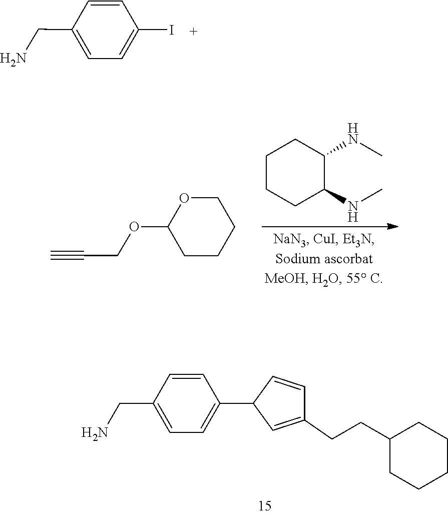

A mixture of (4-iodophenyl)methanamine hydrochloride (5.0 g, 18.55 mmol, 1.0 eq), (1S,2S)—N1,N2-dimethylcyclohexane-1,2-diamine (0.59 mL 3.71 mmol, 0.2 eq), Sodium ascorbate (368 mg, 1.86 mmol, 0.1 eq), Copper Iodide (530 mg, 2.78 mmol, 0.15 eq), Sodium azide (2.41 g, 37.1 mmol, 2.0 eq), Et₃N (3.11 mL, 22.26 mmol, 1.2 eq) and 2-(prop-2-yn-1-yloxy)tetrahydro-2H-pyran (2.6 g, 18.55 mmol, 1.0 eq) in Methanol (50 mL) and water (12 mL) were purged with Nitrogen for 5 minutes and heated to 55° C. for over night. The reaction mixture was cooled to room temperature and filtered through 413 filter paper. Celite was added and the solvent was removed under reduced pressure and the residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 6.25% to afford (4-(4-(((tetrahydro-2H-pyran-2-yl)oxy)methyl)-1H-1,2,3-triazol-1-yl)phenyl)methanamine (15, 3.54 g, 66%) as a white solid. LCMS m/z: [M+H]⁺ Calcd for C₁₅H₂₀N₄O₂, 289.2. Found 289.2.

### Experimental Procedure for N-(4-(4-(((tetrahydro-2H-pyran-2-yl)oxy)methyl)-1H-1,2,3-triazol-1-yl)benzyl)methacrylamide (16)

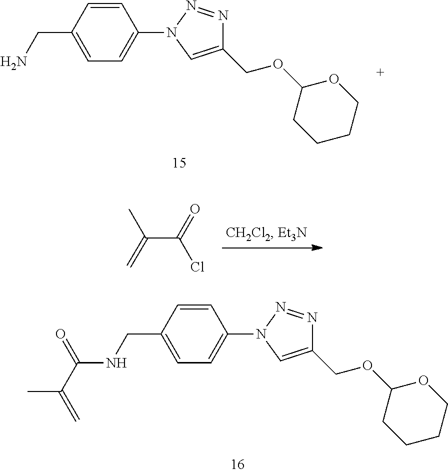

A solution of (4-(4-(((tetrahydro-2H-pyran-2-yl)oxy)methyl)-1H-1,2,3-triazol-1-yl)phenyl)methanamin (15, 3.46 g, 12.00 mmol, 1.0 eq) and triethylamine (2.01 mL, 14.40 mmol, 1.2 eq) in CH₂Cl₂ (40 mL) was cooled to 0° C. with an ice-bath and methacryloyl chloride (1.23 mL, 12.60 mmol, 1.05 eq, diluted in 5 mL of CH₂Cl₂) was added in a dropwise fashion. The cooling bath was removed and the reaction was stirred for 4 h. 20 grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 3.75% to afford N-(4-(4-(((tetrahydro-2H-pyran-2-yl)oxy)methyl)-1H-1,2,3-triazol-1-yl)benzyl)methacrylamide (16, 2.74 g, 64% yield) as a white solid. LCMS m/z: [M+H]⁺ Calcd for C₁₉H₂₄N₄O₃, 357.2. Found 357.3.

### Experimental Procedure for N-(4-(4-(hydroxymethyl)-1H-1,2,3-triazol-1-yl)benzyl)methacrylamide (17)

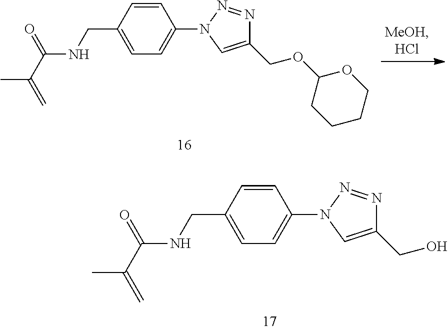 [SMILES: C=C(C)C(=O)NCc1ccc(-n2cc(COC3CCCCO3)nn2)cc1 ; C=C(C)C(=O)NCc1ccc(-n2cc(CO)nn2)cc1]

A solution of N-(4-(4-(hydroxymethyl)-1H-1,2,3-triazol-1-yl)benzyl)methacrylamide (16, 1.2 g, 3.37 mmol, 1.0 eq) was dissolved in Methanol (6 mL) and HCl (1N, aq., 9 mL) for over night at room temperature. Celite was added and the solvent was removed under reduced pressure. The crude product was purified over silica gel chromatography (24 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 12.5% to afford N-(4-(4-(hydroxymethyl)-1H-1,2,3-triazol-1-yl)benzyl)methacrylamide (17, 0.85 g, 92% yield) as a white solid. LCMS m/z: [M+H]⁺ Calcd for C₁₄H₁₆N₄O₂, 273.1. Found 273.1.

### Experimental Procedure for (4-(((tetrahydro-2H-pyran-2-yl)oxy)methyl)benzyl)carbamate (19)

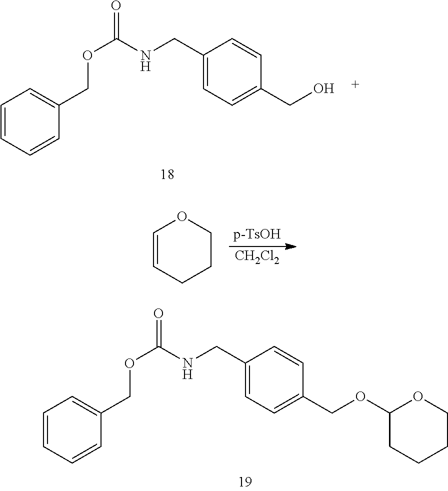

Benzyl (4-(hydroxymethyl)benzyl)carbamate (2.71 g, 10 mmol, 1 eq), 3,4-dihydro-2H-pyran (1.81 mL, 20 mmol, 2 eq), p-Toluenesulfonic acid monohydrate (285 mg, 1.5 mmol, 0.15 eq) in dichloromethane (100 mL) were stirred at room temperature over night. Celite was added and the solvent was removed under reduced pressure. The crude product was purified over silica gel (24 g) using Hexanes/EtOAc as eluent starting at 100% Hexanes and increasing the concentration of EtOAc gradually to 100% to afford benzyl (4-(((tetrahydro-2H-pyran-2-yl)oxy)methyl)benzyl)-carbamate (19, 2.4 g, 68%) as a colorless oil. LCMS m/z: [M+Na]⁺ Calcd for C₂₁H₂₅NO₄, 378.17. Found 378.17.

### Experimental Procedure for (4-(((tetrahydro-2H-pyran-2-yl)oxy)methyl)-phenyl)methanamine (20)

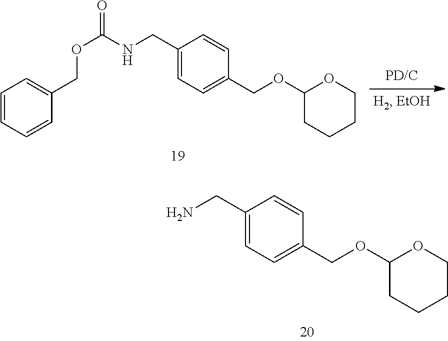 [SMILES: O=C(NCc1ccc(COC2CCCCO2)cc1)OCc1ccccc1 ; NCc1ccc(COC2CCCCO2)cc1]

(4-(((tetrahydro-2H-pyran-2-yl)oxy)methyl)benzyl)carbamate (19, 1.5 g, 4.2 mmol, 1 eq), Palladium on carbon (160 mg, 10 wt. %) in EtOH was briefly evacuated and then Hydrogen was added via a balloon and the mixture was stirred for 1 hour at room temperature. Celite was added and the solvent was removed under reduced pressure. The crude product was purified over silica gel (12 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(((tetrahydro-2H-pyran-2-yl)oxy)methyl)phenyl)methanamine (20, 890 mg, 95%) as a colorless oil. LCMS m/z: [M+H]⁺ Calcd for C₁₃H₁₉NO₂, 222.15. Found 222.14.

### Experimental Procedure for N-(4-(((tetrahydro-2H-pyran-2-yl)oxy)methyl)benzyl)-methacrylamide (21)

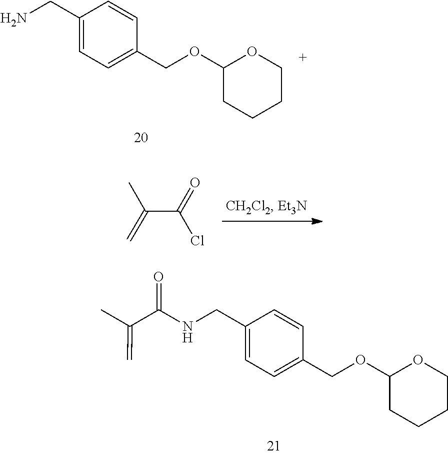

A solution of (4-(((tetrahydro-2H-pyran-2-yl)oxy)methyl)phenyl)methanamine (20, 0.5 g, 2.26 mmol, 1.0 eq) and triethylamine (0.47 mL, 3.39 mmol, 1.5 eq) in CH₂Cl₂ (10 mL) were briefly evacuated and flushed with Nitrogen. Methacryloyl chloride (0.33 mL, 3.39 mmol, 1.5 eq) was added in a dropwise fashion. The reaction mixture was stirred over night at room temperature. 10 grams of Celite was added and the solvent was removed under reduced pressure. The residue was purified by silica gel chromatography (12 g) using Hexanes/EtOAc as eluent starting at 100% Hexanes and increasing the concentration of EtOAc gradually to 100% to afford N-(4-(((tetrahydro-2H-pyran-2-yl)oxy)methyl)benzyl)methacrylamide (21, 0.47 g, 72% yield) as a colorless solid. LCMS m/z: [M+Na]⁺ Calcd for C₁₇H₂₃NO₃, 312.16. Found 312.17.

### Experimental Procedure (4-(4-(2-((tetrahydro-2H-pyran-2-yl)oxy)ethyl)-1H-1,2,3-triazol-1-yl)phenyl)methanamine (22)

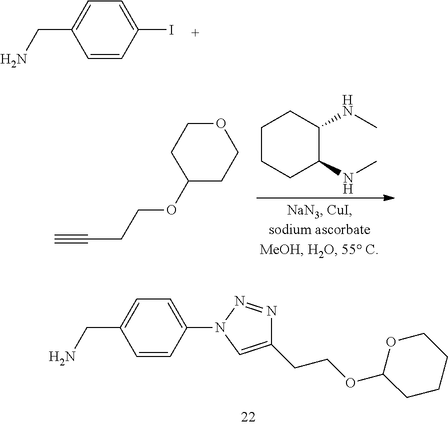

A mixture of (4-iodophenyl)methanamine (5.0 g, 21.45 mmol, 1.0 eq), (1S,2S)—N1,N2-dimethylcyclohexane-1,2-diamine (0.44 mL 2.79 mmol, 0.13 eq), Sodium ascorbate (425 mg, 2.15 mmol, 0.1 eq), Copper Iodide (409 mg, 2.15 mmol, 0.1 eq), Sodium azide (2.79 g, 42.91 mmol, 2.0 eq), and 2-(but-3-yn-1-yloxy)tetrahydro-2H-pyran (3.36 mL, 21.45 mmol, 1.0 eq) in Methanol (20 mL) and water (5 mL) were purged with Nitrogen for 5 minutes and heated to 55° C. for over night. The reaction mixture was cooled to room temperature and filtered through 413 filter paper. Celite (10 g) was added and the solvent was removed under reduced pressure and the residue was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 5% to afford (4-(4-(2-((tetrahydro-2H-pyran-2-yl)oxy)ethyl)-1H-1,2,3-triazol-1-yl)phenyl)methanamine (22, 3.15 g, 49%) as a solid. LCMS m/z: [M+H]⁺ Calcd for C₁₆H₂₂N₄O₂, 303.18. Found 303.18.

### Experimental Procedure for N-(4-(4-(2-((tetrahydro-2H-pyran-2-yl)oxy)ethyl)-1H-1,2,3-triazol-1-yl)benzyl)methacrylamide (23)

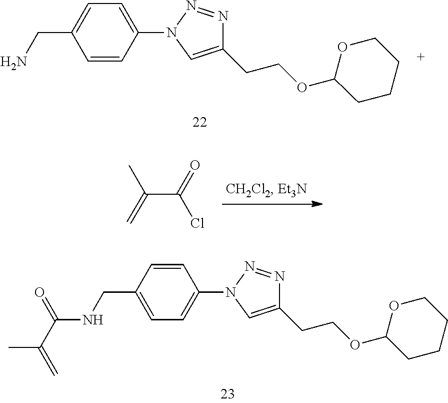

A solution of (4-(4-(2-((tetrahydro-2H-pyran-2-yl)oxy)ethyl)-1H-1,2,3-triazol-1-yl)phenyl)methanamine (22, 3.10 g, 10.25 mmol, 1.0 eq) and triethylamine (1.71 mL, 12.30 mmol, 1.2 eq) in CH₂Cl₂ (55 mL) was cooled to 0° C. with an ice-bath and methacryloyl chloride (1.05 mL, 12.30 mmol, 1.2 eq, diluted in 5 mL of CH₂Cl₂) was added in a dropwise fashion. The cooling bath was removed and the reaction was stirred for 4 h. 8 grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 2.5% to afford N-(4-(4-(2-((tetrahydro-2H-pyran-2-yl)oxy)ethyl)-1H-1,2,3-triazol-1-yl)benzyl)methacrylamide (23, 2.06 g, 54% yield) as a white solid. LCMS m/z: [M+H]⁺ Calcd for C₂₀H₂₆N₄O₃, 371.2078. Found 371.2085.

### Experimental Procedure (4-(1-(2-((tetrahydro-2H-pyran-2-yl)oxy)ethyl)-1H-1,2,3-triazol-4-yl)phenyl)methanamine (24)

A mixture of (4-ethynylphenyl)methanamine (2.36 g, 18.00 mmol, 1.0 eq), (1S,2S)—N1,N2-dimethylcyclohexane-1,2-diamine (0.56 mL, 3.60 mmol, 0.2 eq), Sodium ascorbate (357 mg, 1.80 mmol, 0.1 eq), Copper Iodide (514 mg, 2.70 mmol, 0.15 eq), and 2-(2-azidoethoxy)tetrahydro-2H-pyran (3.08, 18.00 mmol, 1.0 eq) in Methanol (24 mL) and water (6 mL) were purged with Nitrogen for 5 minutes and heated to 55° C. for over night. The reaction mixture was cooled to room temperature and filtered over Celite and rinsed with MeOH (3×50 mL). The solvent was removed under reduced pressure and the residue was redissolved in dichloromethane, Celite (20 g) was added and the solvent was removed under reduced pressure and the residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(1-(2-((tetrahydro-2H-pyran-2-yl)oxy)ethyl)-1H-1,2,3-triazol-4-yl)phenyl)methanamine (24, 3.51 g, 64%) as a yellowish oil. LCMS m/z: [M+H]⁺ Calcd for C₁₆H₂₂N₄O₂, 303.1816. Found 303.1814.

### Experimental Procedure for N-(4-(1-(2-((tetrahydro-2H-pyran-2-yl)oxy)ethyl)-1H-1,2,3-triazol-4-yl)benzyl)methacrylamide (25)

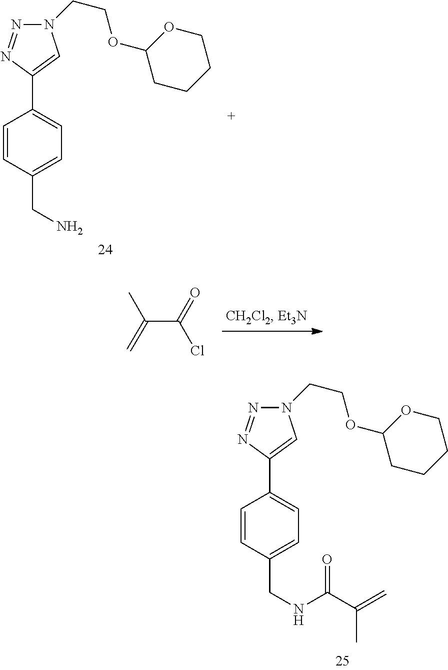

A solution of (4-(1-(2-((tetrahydro-2H-pyran-2-yl)oxy)ethyl)-1H-1,2,3-triazol-4-yl)phenyl)methanamine (24, 1.5 g, 4.96 mmol, 1.0 eq) and triethylamine (1.04 mL, 7.44 mmol, 1.5 eq) in CH₂Cl₂ (30 mL) were briefly evacuated and flushed with Nitrogen. Methacryloyl chloride (0.72 mL, 7.44 mmol, 1.5 eq) was added in a dropwise fashion. The reaction mixture was stirred for 2 h at room temperature. 10 grams of Celite was added and the solvent was removed under reduced pressure. The residue was purified by silica gel chromatography (40 g) using Hexanes/EtOAc as eluent starting at 100% Hexanes and increasing the concentration of EtOAc gradually to 100% to afford N-(4-(1-(2-((tetrahydro-2H-pyran-2-yl)oxy)ethyl)-1H-1,2,3-triazol-4-yl)benzyl)methacrylamide (25, 0.9 g, 49% yield) as a colorless solid. LCMS m/z: [M+Na]⁺ Calcd for C₂₀H₂₆N₄O₃, 371.2078. Found 371.2076.

### Experimental Procedure for 1-(4-(4-(((tetrahydro-2H-pyran-2-yl)oxy)methyl)-1H-1,2,3-triazol-1-yl)phenyl)ethan-1-amine (26)

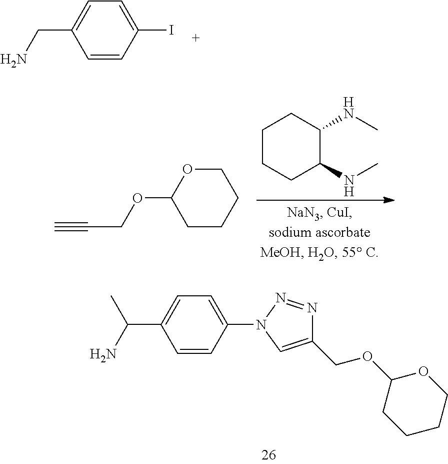

A mixture of 1-(4-iodophenyl)ethan-1-amine hydrochloride (1.0 g, 4.05 mmol, 1.0 eq), (1S,2S)—N1,N2-dimethylcyclohexane-1,2-diamine (0.08 mL 0.53 mmol, 0.13 eq), Sodium ascorbate (80 mg, 0.40 mmol, 0.1 eq), Copper Iodide (77 mg, 0.40 mmol, 0.1 eq), Sodium azide (526 g, 8.09 mmol, 2.0 eq), and 2-(prop-2-yn-1-yloxy)tetrahydro-2H-pyran (0.57 g, 4.05 mmol, 1.0 eq) in Methanol (9 mL) and water (1 mL) were purged with Nitrogen for 5 minutes and heated to 55° C. for over night. The reaction mixture was cooled to room temperature and the solvent was removed under reduced pressure. The residue was redissolved in dichloromethane and filtered over a plug of Celite. Celite was added to the filtrate and the solvent was removed under reduced pressure. The residue was purified over silica gel (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 5% to afford 1-(4-(4-(((tetrahydro-2H-pyran-2-yl)oxy)methyl)-1H-1,2,3-triazol-1-yl)phenyl)ethan-1-amine (26, 0.62 g, 51%) as a yellowish solid. LCMS m/z: [M+H]⁺ Calcd for C₁₆H₂₂N₄O₂, 303.2. Found 303.2.

### Experimental Procedure for N-(1-(4-(4-(((tetrahydro-2H-pyran-2-yl)oxy) methyl)-1H-1,2,3-triazol-1-yl)phenyl)ethyl)methacrylamide (27)

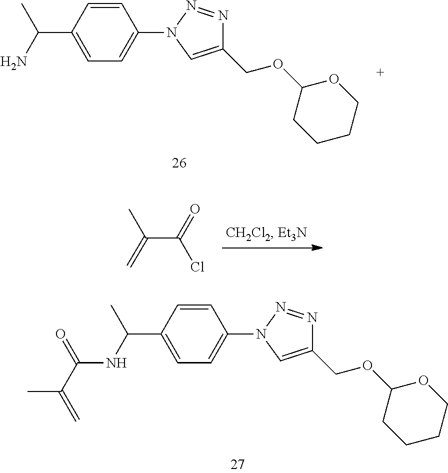

A solution of 1-(4-(4-(((tetrahydro-2H-pyran-2-yl)oxy)methyl)-1H-1,2,3-triazol-1-yl)phenyl)ethan-1-amine (26, 0.52 g, 1.7 mmol, 1.0 eq) and triethylamine (0.29 mL, 2.1 mmol, 1.2 eq) in CH₂Cl₂ (11 mL) was cooled to 0° C. with an ice-bath and methacryloyl chloride (0.18 mL, 1.8 mmol, 1.05 eq, diluted in 11 mL of CH₂Cl₂) was added in a dropwise fashion. The cooling bath was removed and the reaction was stirred for 4 h. 5 grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 2.5% to afford N-(1-(4-(4-(((tetrahydro-2H-pyran-2-yl)oxy) methyl)-1H-1,2,3-triazol-1-yl)phenyl)ethyl)methacrylamide (27, 0.49 g, 76% yield) as a white solid. LCMS m/z: [M+H]⁺ Calcd for C₂₀H₂₆N₄O₃, 371.2078. Found 371.2087.

### Experimental Procedure for (4-(4-(((tetrahydro-2H-pyran-2-yl)oxy)methyl)-1H-1,2,3-triazol-1-yl)-2-(trifluoromethyl)phenyl)methanamine (28)

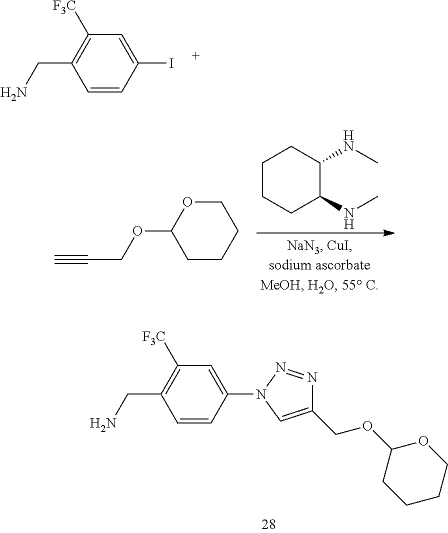

A mixture of (4-iodo-2-(trifluoromethyl)phenyl)methanamine (3.0 g, 9.97 mmol, 1.0 eq), (1S,2S)—N1,N2-dimethylcyclohexane-1,2-diamine (0.31 mL 1.99 mmol, 0.2 eq), Sodium ascorbate (197 mg, 1.00 mmol, 0.1 eq), Copper Iodide (285 mg, 1.49 mmol, 0.15 eq), Sodium azide (1.30 g, 19.93 mmol, 2.0 eq), Et₃N (1.67 mL, 11.96 mmol, 1.2 eq) and 2-(prop-2-yn-1-yloxy)tetrahydro-2H-pyran (1.40 g, 9.97 mmol, 1.0 eq) in Methanol (24 mL) and water (6 mL) were purged with Nitrogen for 5 minutes and heated to 55° C. for over night. The reaction mixture was cooled to room temperature and filtered through a plug of Celite and rinsed with Methanol (3×50 mL). Celite was added to the filtrate and the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(4-(((tetrahydro-2H-pyran-2-yl)oxy)methyl)-1H-1,2,3-triazol-1-yl)-2-(trifluoromethyl)phenyl)methanamine (28, 2.53 g, 71%) as a green oil. LCMS m/z: [M+H]⁺ Calcd for C₁₆H₁₉N₄O₂F₃, 357.2. Found 357.1.

### Experimental Procedure for N-(4-(4-(((tetrahydro-2H-pyran-2-yl)oxy)methyl)-1H-1,2,3-triazol-1-yl)-2(trifluoromethyl)benzyl) methacrylamide (29)

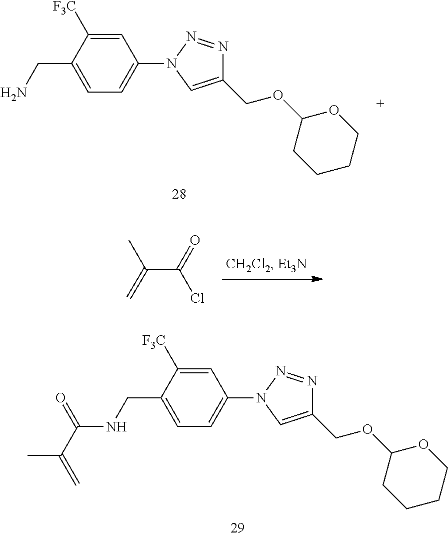

A solution of (4-(4-(((tetrahydro-2H-pyran-2-yl)oxy)methyl)-1H-1,2,3-triazol-1-yl)-2-(trifluoromethyl)phenyl) methanamine (28, 1.0 g, 2.81 mmol, 1.0 eq) and triethylamine (0.59 mL, 4.21 mmol, 1.5 eq) in CH₂Cl₂ (25 mL) were briefly evacuated and flushed with Nitrogen. Methacryloyl chloride (0.41 mL, 4.21 mmol, 1.5 eq) was added in a dropwise fashion. The reaction mixture was stirred for 6 h at room temperature. 10 grams of Celite was added and the solvent was removed under reduced pressure. The residue was purified by silica gel chromatography (40 g) using Hexanes/EtOAc as eluent starting at 100% Hexanes and increasing the concentration of EtOAc gradually to 100% to afford N-(4-(4-(((tetrahydro-2H-pyran-2-yl)oxy)methyl)-1H-1,2,3-triazol-1-yl)-2(trifluoromethyl)benzyl) methacrylamide (29, 0.65 g, 55% yield) as a colorless solid. LCMS m/z: [M+H]⁺ Calcd for C₂₀H₂₃N₄O₃F₃, 425.2. Found 425.1.

### Experimental Procedure for 3-(4-(((tetrahydro-2H-pyran-2-yl)oxy)methyl)-1H-1,2,3-triazol-1-yl)propan-1-amine (30)

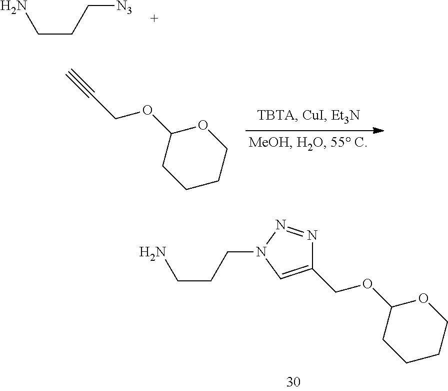

A mixture of 3-azidopropan-1-amine hydrochloride (1.5 g, 14.98 mmol, 1.0 eq), Tris[(1-benzyl-1H-1,2,3-triazol-4-yl)methyl]-amine (1.99 g, 3.75 mmol, 0.25 eq), Copper Iodide (0.29 g, 1.50 mmol, 0.1 eq), and Triethylamine (0.52 mL, 3.75 mmol, 0.25 eq) in Methanol (50 mL) and water (6 mL) were purged with Nitrogen for 5 minutes and cooled to 0 C. 2-(prop-2-yn-1-yloxy)tetrahydro-2H-pyran (2.10 g, 14.98 mmol, 1.0 eq) was added and the reaction mixture was warmed to 55° C. and stirred over night under Nitrogen atmosphere. The reaction mixture was cooled to room temperature, filtered over a plug of Celite and rinsed with Methanol (3×50 mL). Celite (20 g) was added to the filtrate the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 20% to afford 3-(4-(((tetrahydro-2H-pyran-2-yl)oxy)methyl)-1H-1,2,3-triazol-1-yl)propan-1-amine (30, 2.36 g, 66%). LCMS m/z: [M+H]⁺ Calcd for C₁₁H₂₀N₄O₂, 241.2. Found 241.2.

### Experimental Procedure for N-(3-(4-(((tetrahydro-2H-pyran-2-yl)oxy)methyl)-1H-1,2,3-triazol-1-yl)propyl)methacrylamide (31)

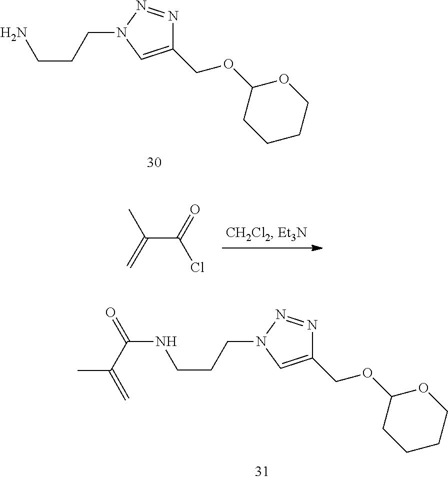

A solution of 3-(4-(((tetrahydro-2H-pyran-2-yl)oxy)methyl)-1H-1,2,3-triazol-1-yl)propan-1-amine (30, 1.0 g, 4.16 mmol, 1.0 eq) and triethylamine (0.58 mL, 4.16 mmol, 1.0 eq) in CH₂Cl₂ (20 mL) were briefly evacuated and flushed with Nitrogen. Methacryloyl chloride (0.40 mL, 4.16 mmol, 1.0 eq) was added in a dropwise fashion. The reaction mixture was stirred at room temperature over night. 10 grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 20% to afford N-(3-(4-(((tetrahydro-2H-pyran-2-yl)oxy)methyl)-1H-1,2,3-triazol-1-yl)propyl)methacrylamide (31, 0.96 g, 75% yield) as a colorless oil. LCMS m/z: [M+H]⁺ Calcd for C₁₅H₂₄N₄O₃, 309.2. Found 309.4.

### Experimental Procedure for (4-(4-((oxetan-3-yloxy)methyl)-1H-1,2,3-triazol-1-yl)phenyl)methanamine (32)

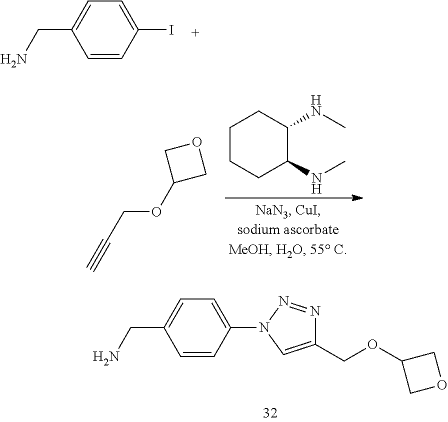

A mixture of (4-iodophenyl)methanamine hydrochloride (2.64 g, 9.80 mmol, 1.0 eq), (1S,2S)—N1,N2-dimethylcyclohexane-1,2-diamine (0.31 mL 1.96 mmol, 0.2 eq), Sodium ascorbate (198 mg, 0.98 mmol, 0.1 eq), Copper Iodide (279 mg, 1.47 mmol, 0.15 eq), Sodium azide (1.27 g, 19.59 mmol, 2.0 eq), Et₃N (1.64 mL, 11.75 mmol, 1.2 eq) and 3-(prop-2-yn-1-yloxy)oxetane (9, 1.10 g, 9.80 mmol, 1.0 eq) in Methanol (24 mL) and water (6 mL) were purged with Nitrogen for 5 minutes and heated to 55° C. for over night. The reaction mixture was cooled to room temperature and filtered through a plug of Celite and rinsed with Methanol (3×50 mL). Celite was added to the filtrate and the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(4-((oxetan-3-yloxy)methyl)-1H-1,2,3-triazol-1-yl)phenyl)methanamine (32, 1.43 g, 56%) as an oil. LCMS m/z: [M+H]⁺ Calcd for C₁₃H₁₆N₄O₂, 261.1346. Found 261.1342.

### Experimental Procedure for N-(4-(4-((oxetan-3-yloxy)methyl)-1H-1,2,3-triazol-1-yl)benzyl)methacrylamide (33)

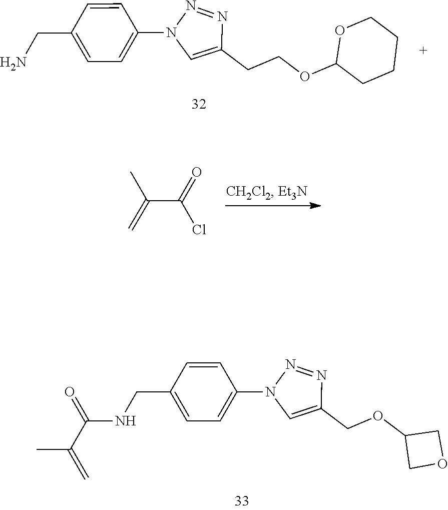

A solution of (4-(4-((oxetan-3-yloxy)methyl)-1H-1,2,3-triazol-1-yl)phenyl)methanamine (32, 0.58 g, 2.23 mmol, 1.0 eq) and triethylamine (0.47 mL, 3.34 mmol, 1.5 eq) in CH₂Cl₂ (20 mL) were briefly evacuated and flushed with Nitrogen. Methacryloyl chloride (0.32 mL, 3.34 mmol, 1.5 eq) was added in a dropwise fashion. The reaction mixture was stirred for 6 h at room temperature. 10 grams of Celite was added and the solvent was removed under reduced pressure. The residue was purified by silica gel chromatography (24 g) using Hexanes/EtOAc as eluent starting at 100% Hexanes and increasing the concentration of EtOAc gradually to 100% to afford N-(4-(4-((oxetan-3-yloxy)methyl)-1H-1,2,3-triazol-1-yl)benzyl)methacrylamide (33, 0.48 g, 66% yield) as a colorless solid. LCMS m/z: [M+H]⁺ Calcd for C₁₇H₂₀N₄O₃, 329.1608. Found 329.1611.

### Experimental Procedure for ethyl 1-(2-methacrylamidoethyl)-1H-imidazole-4-carboxylate (35)

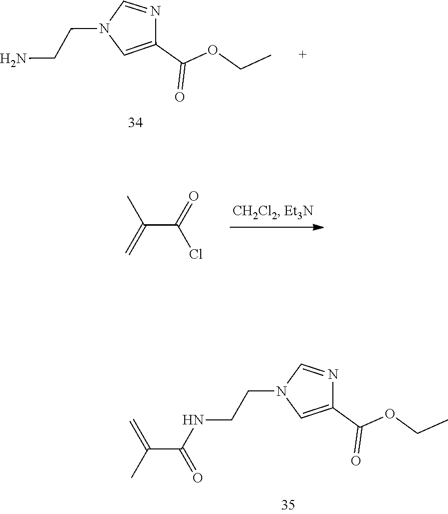

A solution of ethyl 1-(2-aminoethyl)-1H-imidazole-4-carboxylate (34, 2.0 g, 10.91 mmol, 1.0 eq) and triethylamine (3.80 mL, 27.29 mmol, 2.5 eq) in CH₂Cl₂ (20 mL) were briefly evacuated and flushed with Nitrogen. Methacryloyl chloride (1.60 mL, 16.37 mmol, 1.5 eq) was added in a dropwise fashion. The reaction mixture was stirred for 3 h at room temperature. 15 grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford ethyl 1-(2-methacrylamidoethyl)-1H-imidazole-4-carboxylate (35, 1.28 g, 47% yield) as a colorless solid. LCMS m/z: [M+H]⁺ Calcd for C₁₂H₁₇N₃O₃, 252.1. Found 252.1.

### Experimental Procedure for N-(4-(1,1-dioxidothiomorpholino)benzyl) methacrylamide (37)

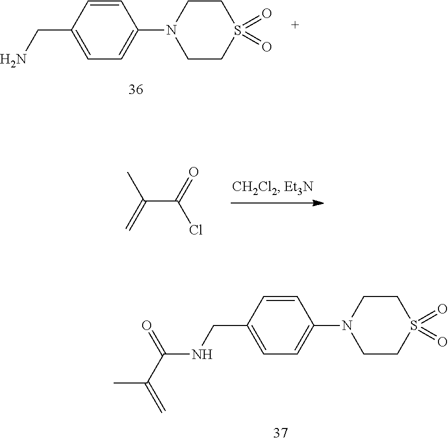

To a solution of 4-(4-(aminomethyl)phenyl)thiomorpholine 1,1-dioxide hydrochloride (36, 1.15 g, 4.15 mmol, 1.0 eq) and triethylamine (1.39 mL, 9.97 mmol, 2.4 eq) in CH₂Cl₂ (80 mL) was added a solution of methacryloyl chloride (0.43 mL, 4.36 mmol, 1.05 eq, in CH₂Cl₂, 5 mL) in a dropwise fashion. The reaction mixture was stirred for 22 h at room temperature. 8 grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 3.75% to afford N-(4-(1,1-dioxidothiomorpholino)benzyl) methacrylamide (37, 0.32 g, 25% yield) as a solid.

### Experimental Procedure for N-methyl-N-(2-(methylsulfonyl)ethyl)prop-2-yn-1-amine (38)

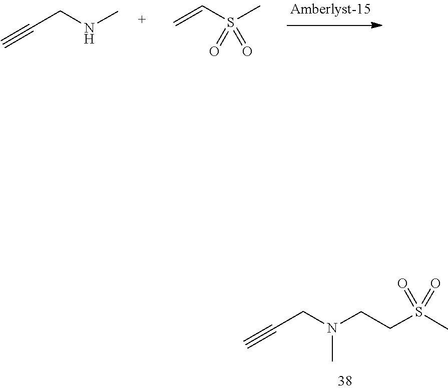

To a mixture of 1-methylsulfonylethylene (4.99 g, 47.03 mmol, 4.13 mL) and Amberlyst-15 ((30% w/w)), N-methylprop-2-yn-1-amine (2.6 g, 37.62 mmol) was added in a dropwise fashion.

The mixture was stirred at room temperature for 12 hours. The catalyst was removed by filtration and the filtrate was concentrated under reduced pressure to afford: N-methyl-N-(2-(methylsulfonyl)ethyl)prop-2-yn-1-amine (38, 6.43 g, 98%) as an oil. LCMS m/z: [M+H]⁺ Calcd for C₇H₁₃NSO₂, 176.11. Found 176.1.

### Experimental Procedure for N-((1-(2-(2-(2-(2-aminoethoxy)ethoxy)ethoxy) ethyl)-1H-1,2,3-triazol-4-yl)methyl)-N-methyl-2-(methylsulfonyl)ethan-1-amine (40)

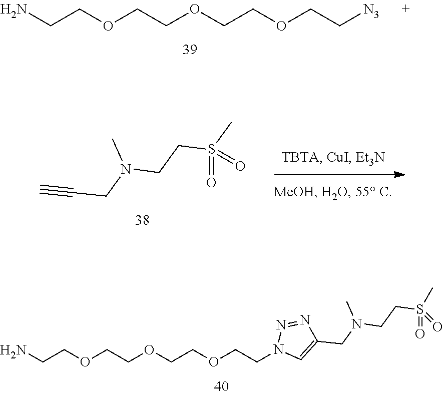 [SMILES: [N-]=[N+]=NCCOCCOCCOCCN ; C#CCN(C)CCS(C)(=O)=O ; CN(CCS(C)(=O)=O)Cc1cn(CCOCCOCCOCCN)nn1]

A mixture of N-methyl-N-(2-(methylsulfonyl)ethyl)prop-2-yn-1-amine (38, 5.02 g, 28.64 mmol, 1.25 eq), Tris[(1-benzyl-1H-1,2,3-triazol-4-yl)methyl]-amine (3.04 g, 5.73 mmol, 0.25 eq), Copper Iodide (436 mg, 2.29 mmol, 0.1 eq), and Triethylamine (0.8 mL, 5.7 mmol, 0.25 eq) in Methanol (50 mL) and water (6 mL) was evacuated and flushed with Nitrogen (3 times) and cooled with an ice bath. 2-(2-(2-(2-azidoethoxy)ethoxy)ethoxy)ethan-1-amine (39, 5.02 g, 22.91 mmol, 1.0 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite (20 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford N-((1-(2-(2-(2-(2-aminoethoxy)ethoxy)ethoxy)ethyl)-1H-1,2,3-triazol-4-yl)methyl)-N-methyl-2-(methylsulfonyl)ethan-1-amine (40, 4.98 g, 55%) as an oil. LCMS m/z: [M+H]⁺ Calcd for C₁₅H₃₁N₅O₅S, 394.2. Found 394.2.

### Experimental Procedure N-(2-(2-(2-(2-(4-((methyl(2-(methylsulfonyl)ethyl) amino)methyl)-1H-1,2,3-triazol-1-yl)ethoxy)ethoxy)ethoxy) ethyl)methacrylamide (41)

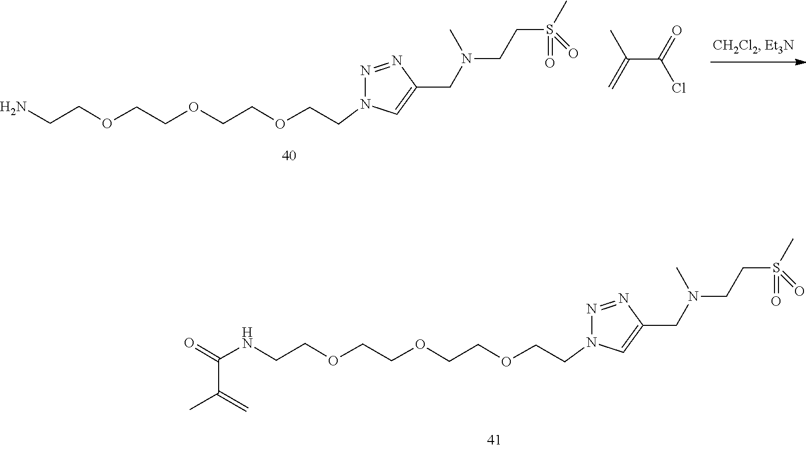

To a solution of N-((1-(2-(2-(2-(2-aminoethoxy)ethoxy)ethoxy)ethyl)-1H-1,2,3-triazol-4-yl)methyl)-N-methyl-2-(methylsulfonyl)ethan-1-amine (40, 1.0 g, 2.54 mmol, 1.0 eq) and triethylamine (0.43 mL, 3.05 mmol, 1.2 eq) in CH₂Cl₂ (15 mL) was added a solution of methacryloyl chloride (0.30 mL, 3.05 mmol, 1.5 eq) in a dropwise fashion. The reaction mixture was stirred for 5 h at room temperature. Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 12.5% to afford N-(2-(2-(2-(2-(4-((methyl(2-(methylsulfonyl)ethyl) amino)methyl)-1H-1,2,3-triazol-1-yl)ethoxy)ethoxy)ethoxy) ethyl)methacrylamide (41, 0.86 g, 73% yield) as an oil. LCMS m/z: [M+H]⁺ Calcd for C₁₉H₃₅N₅O₆S, 462.2. Found 462.2.

### Experimental Procedure for 7-(prop-2-yn-1-yl)-2-oxa-7-azaspiro[3.5]nonane (42)

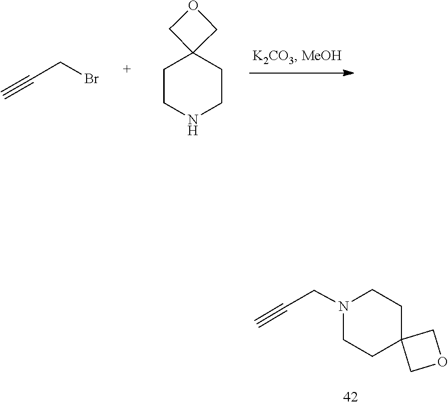

3-Bromoprop-1-yne (4.4 mL, 39.32 mmol 1.0 eq) was added to a mixture of 2-oxa-7-azaspiro[3.5]nonane (8.54 g, 39.32 mmol, 1.0 eq), potassium carbonate (17.9 g, 129.7 mmol, 3.3 eq) in Methanol (200 mL) and stirred over night at room temperature. The mixture was filtered, Celite was added and the solvent was removed under reduced pressure. The residue was purified by silica gel chromatography (220 g) using dichloromethane/methanol as mobile phase. The concentration of methanol was gradually increased from 0% to 5% to afford 7-(prop-2-yn-1-yl)-2-oxa-7-azaspiro[3.5]nonane (42, 4.44 g, 68%) as an oil. Experimental Procedure for 2-(2-(2-(2-(4-((2-oxa-7-azaspiro[3.5]nonan-7-yl) methyl)-1H-1,2,3-triazol-1-yl)ethoxy)ethoxy)ethoxy)ethan-1-amine (43)

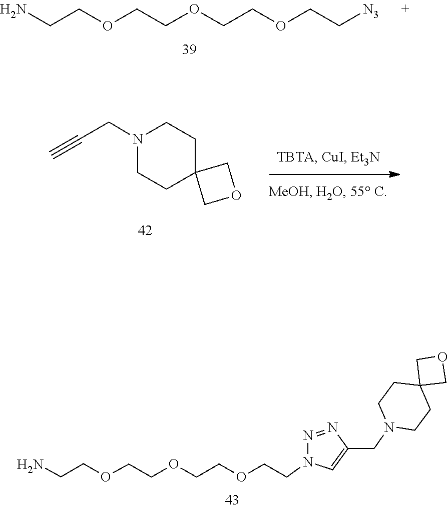 [SMILES: [N-]=[N+]=NCCOCCOCCOCCN ; C#CCN1CCC2(CC1)COC2 ; NCCOCCOCCOCCn1cc(CN2CCC3(CC2)COC3)nn1]

A mixture of 7-(prop-2-yn-1-yl)-2-oxa-7-azaspiro[3.5]nonane (42, 2.5 g, 15.13 mmol, 1.0 eq), Tris[(1-benzyl-1H-1,2,3-triazol-4-yl)methyl]-amine (1.77 g, 3.33 mmol, 0.22 eq), Copper Iodide (288 mg, 1.51 mmol, 0.1 eq), and Triethylamine (0.53 mL, 3.8 mmol, 0.25 eq) in Methanol (50 mL) was cooled with an ice bath. 2-(2-(2-(2-azidoethoxy)ethoxy)ethoxy)ethan-1-amine (39, 3.86 g, 17.70 mmol, 1.17 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite (10 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 2-(2-(2-(2-(4-((2-oxa-7-azaspiro[3.5]nonan-7-yl) methyl)-1H-1,2,3-triazol-1-yl)ethoxy)ethoxy)ethoxy)ethan-1-amine (43, 4.76 g, 82%) as an oil. LCMS m/z: [M+H]⁺ Calcd for C₁₈H₃₃N₅O₄, 384.3. Found 384.2.

### Experimental Procedure for N-(2-(2-(2-(2-(4-((2-oxa-7-azaspiro[3.5]nonan-7-yl)methyl)-1H-1,2,3-triazol-1-yl)ethoxy)ethoxy)ethoxy)ethyl)methacrylamide (44)

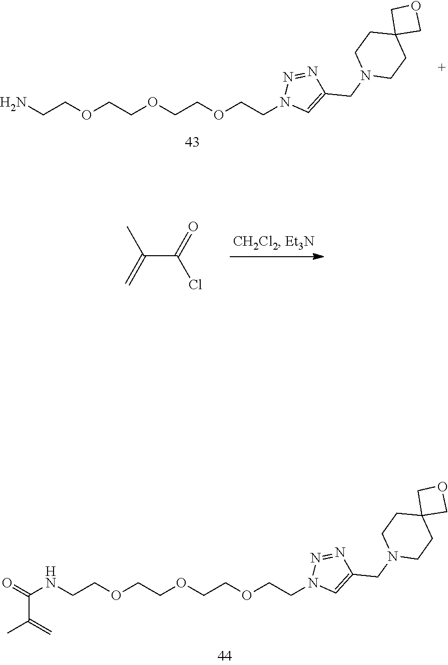

A solution of 2-(2-(2-(2-(4-((2-oxa-7-azaspiro[3.5]nonan-7-yl) methyl)-1H-1,2,3-triazol-1-yl)ethoxy)ethoxy)ethoxy)ethan-1-amine (43, 2.65 g, 6.91 mmol, 1.0 eq) and triethylamine (1.16 mL, 8.29 mmol, 1.2 eq) in CH₂Cl₂ (100 mL) was cooled with an ice-bath under Nitrogen atmosphere. Methacryloyl chloride (0.74 mL, 7.6 mmol, 1.1 eq) was added in a dropwise fashion. The cooling bath was removed and the reaction mixture was stirred for 4 h at room temperature. 10 grams of Celite was added and the solvent was removed under reduced pressure. The residue was purified by silica gel chromatography (120 g) using dichloromethane/methanol as mobile phase. The concentration of methanol was gradually increased from 0% to 10% to afford N-(2-(2-(2-(2-(4-((2-oxa-7-azaspiro[3.5]nonan-7-yl)methyl)-1H-1,2,3-triazol-1-yl)ethoxy)ethoxy)ethoxy)ethyl)methacrylamide (44, 1.50 g, 48% yield) as a colorless oil. LCMS m/z: [M+H]⁺ Calcd for C₂₂H₃₇N₅O₅, 452.29. Found 452.25.

### Experimental Procedure for 4-((1-(2-(2-aminoethoxy)ethyl)-1H-1,2,3-triazol-4-yl)methyl)thiomorpholine 1,1-dioxide (45)

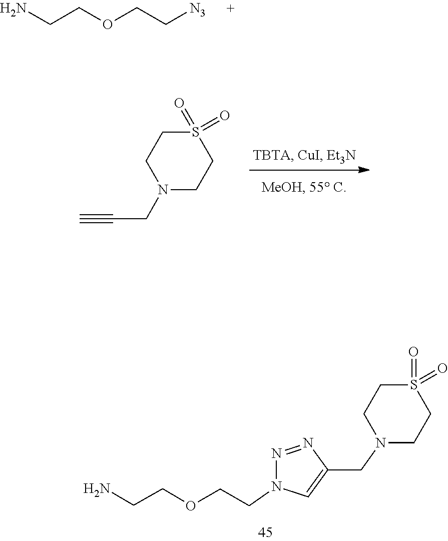 [SMILES: [N-]=[N+]=NCCOCCN ; C#CCN1CCS(=O)(=O)CC1 ; NCCOCCn1cc(CN2CCS(=O)(=O)CC2)nn1]

A mixture of 4-(prop-2-yn-1-yl)thiomorpholine 1,1-dioxide (1.14 g, 6.58 mmol, 1.0 eq), Tris[(1-benzyl-1H-1,2,3-triazol-4-yl)methyl]-amine (768 mg, 1.45 mmol, 0.22 eq), Copper Iodide (125 mg, 0.66 mmol, 0.1 eq), and Triethylamine (0.23 mL, 1.65 mmol, 0.25 eq) in Methanol (20 mL) was cooled with an ice bath. 2-(2-azidoethoxy)ethan-1-amine (1.00 g, 7.70 mmol, 1.17 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite (10 g) was added, and concentrated under reduced pressure. The crude product was purified over silica gel (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 9.5% to afford for 4-((1-(2-(2-aminoethoxy)ethyl)-1H-1,2,3-triazol-4-yl)methyl)thiomorpholine 1,1-dioxide (45, 1.86 g, 93%) as a white solid. LCMS m/z: [M+H]⁺ Calcd for C₁₁H₂₁N₅O₄S, 304.1438. Found 304.1445.

### Experimental Procedure for N-(2-(2-(4-((1,1-dioxidothiomorpholino)methyl)-1H-1,2,3-triazol-1-yl)ethoxy)ethyl)methacrylamide (46)

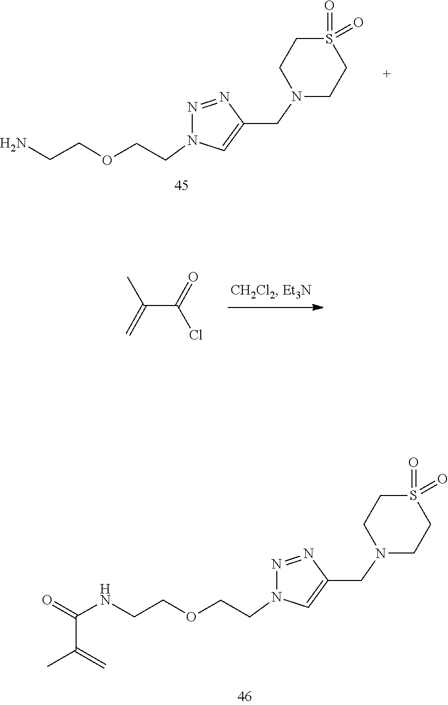

A solution of 4-((1-(2-(2-aminoethoxy)ethyl)-1H-1,2,3-triazol-4-yl)methyl)thiomorpholine 1,1-dioxide (45, 1.32 g, 4.35 mmol, 1.0 eq) and triethylamine (0.73 mL, 5.22 mmol, 1.2 eq) in CH₂Cl₂ (100 mL) was cooled with an ice-bath under Nitrogen atmosphere. Methacryloyl chloride (0.47 mL, 4.8 mmol, 1.1 eq) was added in a dropwise fashion. The cooling bath was removed and the reaction mixture was stirred for 4 h at room temperature. 10 grams of Celite was added and the solvent was removed under reduced pressure. The residue was purified by silica gel chromatography (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(2-(2-(4-((1,1-dioxidothiomorpholino)methyl)-1H-1,2,3-triazol-1-yl)ethoxy)ethyl)-methacrylamide (46, 0.90 g, 56% yield) as a colorless oil. LCMS m/z: [M+H]⁺ Calcd for C₁₅H₂₅N₅O₄S, 372.17. Found 372.15.

### Experimental Procedure for 4-((1-(2-(2-(2-aminoethoxy)ethoxy)ethyl)-1H-1,2,3-triazol-4-yl)methyl)thiomorpholine 1,1-dioxide (47)

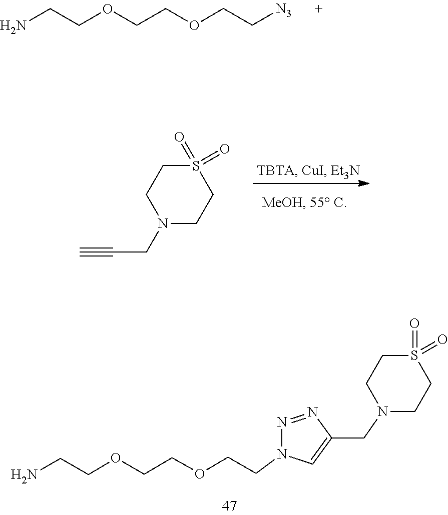 [SMILES: [N-]=[N+]=NCCOCCOCCN ; C#CCN1CCS(=O)(=O)CC1 ; NCCOCCOCCn1cc(CN2CCS(=O)(=O)CC2)nn1]

A mixture of 4-(prop-2-yn-1-yl)thiomorpholine 1,1-dioxide (4.6 g, 26.55 mmol, 1.0 eq), Tris[(1-benzyl-1H-1,2,3-triazol-4-yl)methyl]-amine (3.1 g, 5.84 mmol, 0.22 eq), Copper Iodide (506 mg, 2.66 mmol, 0.1 eq), and Triethylamine (0.93 mL, 6.64 mmol, 0.25 eq) in Methanol (80 mL) was cooled with an ice bath. 2-(2-(2-azidoethoxy)ethoxy)ethan-1-amine (5.00 g, 28.68 mmol, 1.08 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 4-((1-(2-(2-(2-aminoethoxy)ethoxy)ethyl)-1H-1,2,3-triazol-4-yl)methyl)thiomorpholine 1,1-dioxide (47, 5.26 g, 57%) as a yellowish oil. LCMS m/z: [M+H]⁺ Calcd for C₁₃H₂₅N₅O₄S, 348.1700. Found 348.1700.

### Experimental Procedure N-(2-(2-(2-(4-((1,1-dioxidothiomorpholino)methyl)-1H-1,2,3-triazol-1-yl)ethoxy)ethoxy)ethyl)methacrylamide (48)

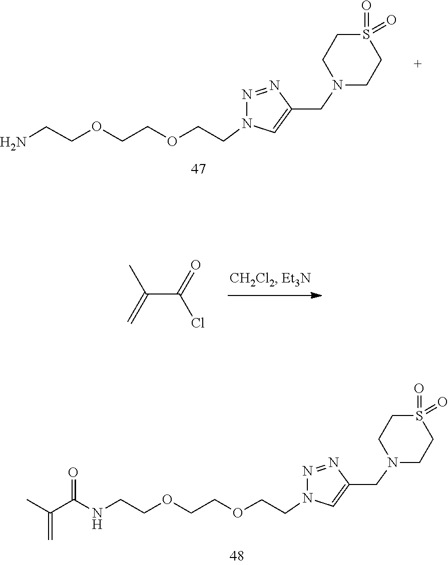

A solution of 4-((1-(2-(2-(2-aminoethoxy)ethoxy)ethyl)-1H-1,2,3-triazol-4-yl)methyl)thiomorpholine 1,1-dioxide (47, 1.49 g, 4.29 mmol, 1.0 eq) and triethylamine (0.72 mL, 5.15 mmol, 1.2 eq) in CH₂Cl₂ (50 mL) was cooled with an ice-bath under Nitrogen atmosphere. Methacryloyl chloride (0.46 mL, 4.7 mmol, 1.1 eq) was added in a dropwise fashion. The cooling bath was removed and the reaction mixture was stirred for 4 h at room temperature. 10 grams of Celite was added and the solvent was removed under reduced pressure. The residue was purified by silica gel chromatography (80 g) using dichloromethane/methanol as mobile phase. The concentration of methanol was gradually increased from 0% to 5% to afford N-(2-(2-(2-(4-((1,1-dioxidothiomorpholino)methyl)-1H-1,2,3-triazol-1-yl)ethoxy)ethoxy)ethyl)-methacrylamide (48, 0.67 g, 38% yield) as a colorless oil. LCMS m/z: [M+H]⁺ Calcd for C₁₇H₂₉N₅O₅S, 416.20. Found 416.20.

### Experimental Procedure for 4-((1-(14-amino-3,6,9,12-tetraoxatetradecyl)-1H-1,2,3-triazol-4-yl)methyl)thiomorpholine 1,1-dioxide (49)

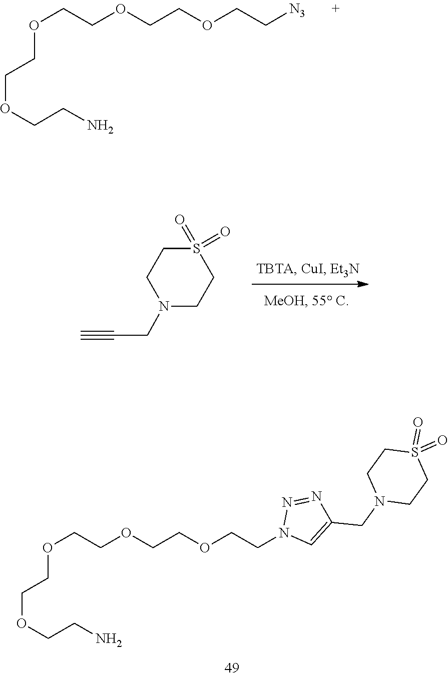 [SMILES: [N-]=[N+]=NCCOCCOCCOCCOCCN ; C#CCN1CCS(=O)(=O)CC1 ; NCCOCCOCCOCCOCCn1cc(CN2CCS(=O)(=O)CC2)nn1]

A mixture of 4-(prop-2-yn-1-yl)thiomorpholine 1,1-dioxide (5.0 g, 28.86 mmol, 1.0 eq), Tris[(1-benzyl-1H-1,2,3-triazol-4-yl)methyl]-amine (3.37 g, 6.35 mmol, 0.22 eq), Copper Iodide (550 mg, 2.89 mmol, 0.1 eq), and Triethylamine (1.01 mL, 7.22 mmol, 0.25 eq) in Methanol (90 mL) was cooled with an ice bath. 14-azido-3,6,9,12-tetraoxatetradecan-1-amine (8.86 g, 33.77 mmol, 1.17 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite (15 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 4-((1-(14-amino-3,6,9,12-tetraoxatetradecyl)-1H-1,2,3-triazol-4-yl)methyl)thiomorpholine 1,1-dioxide (49, 7.56 g, 60%) as an oil. LCMS m/z: [M+H]⁺ Calcd for C₁₇H₃₃N₅O₆S, 436.2224. Found 436.2228.

### Experimental Procedure N-(14-(4-((1,1-dioxidothiomorpholino)methyl)-1H-1,2,3-triazol-1-yl)-3,6,9,12-tetraoxatetradecyl)methacrylamide (50)

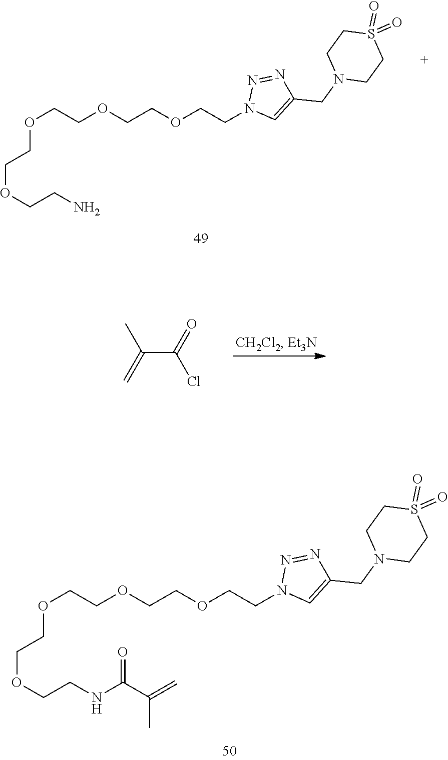

A solution of 4-((1-(14-amino-3,6,9,12-tetraoxatetradecyl)-1H-1,2,3-triazol-4-yl)methyl)thiomorpholine 1,1-dioxide (49, 1.95 g, 4.79 mmol, 1.0 eq) and triethylamine (0.80 mL, 5.74 mmol, 1.2 eq) in CH₂Cl₂ (50 mL) was cooled with an ice-bath under Nitrogen atmosphere. Methacryloyl chloride (0.51 mL, 5.26 mmol, 1.1 eq) was added in a dropwise fashion. The cooling bath was removed and the reaction mixture was stirred for 4 h at room temperature. 10 grams of Celite was added and the solvent was removed under reduced pressure. The residue was purified by silica gel chromatography (80 g) using dichloromethane/methanol as mobile phase. The concentration of methanol was gradually increased from 0% to 5% to afford N-(14-(4-((1,1-dioxidothiomorpholino)methyl)-1H-1,2,3-triazol-1-yl)-3,6,9,12-tetraoxatetradecyl)methacrylamide (50, 0.76 g, 32% yield) as a colorless oil. LCMS m/z: [M+H]⁺ Calcd for C₂₁H₃₇N₅O₇S, 504.25. Found 504.20.

### Example 2: Chemical Modification of Exemplary Polymers

A polymeric material may be chemically modified with a compound of Formula (I) (or pharmaceutically acceptable salt thereof) prior to formation of a particle (e.g., a hydrogel capsule described herein). Synthetic protocols of exemplary compounds for modification of polymeric materials are outlined above in Example 1. These compounds, or others, may be used to chemically modify any polymeric material.

For example, in the case of alginate, the alginate carboxylic acid is activated for coupling to one or more amine-functionalized compounds to achieve an alginate modified with an afibrotic compound, e.g., a compound of Formula (I). The alginate polymer is dissolved in water (30 mL/gram polymer) and treated with 2-chloro-4,6-dimethoxy-1,3,5-triazine (0.5 eq) and N-methylmorpholine (1 eq). To this mixture is added a solution of the compound of interest (e.g., Compound 101 shown in Table 2) in acetonitrile (0.3M).

The amounts of the compound and coupling reagent added depends on the desired concentration of the compound bound to the alginate, e.g., conjugation density. A medium conjugation density of Compound 101 typically ranges from 2% to 5% N, while a high conjugation density of Compound 101 typically ranges from 5.1% to 8% N. To prepare a CM-LMW-Alg-101-Medium polymer solution, the dissolved unmodified low molecular weight alginate (approximate MW<75 kDa, G:M ratio ≥1.5) is treated with 2-chloro-4,6-dimethoxy-1,3,5-triazine (5.1 mmol/g alginate) and N-methylmorpholine (10.2 mmol/g alginate) and Compound 101 (5.4 mmol/g alginate). To prepare a CM-LMW-Alg-101-High polymer solution, the dissolved unmodified low-molecular weight alginate (approximate MW<75 kDa, G:M ratio ≥1.5) is treated with 2-chloro-4,6-dimethoxy-1,3,5-triazine (10.2 mmol/g alginate) and N-methylmorpholine (20.4 mmol/g alginate) and Compound 101 (10.8 mmol/g alginate).

The reaction is warmed to 55° C. for 16 h, then cooled to room temperature and gently concentrated via rotary evaporation, then the residue is dissolved in water. The mixture is filtered through a bed of cyano-modified silica gel (Silicycle) and the filter cake is washed with water. The resulting solution is then extensively dialyzed (10,000 MWCO membrane) and the alginate solution is concentrated via lyophilization to provide the desired chemically-modified alginate as a solid or is concentrated using any technique suitable to produce a chemically modified alginate solution with a viscosity of 25 cP to 35 cP.

The conjugation density of a chemically modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

### Example 3: Preparation of Exemplary Alginate Solutions

70:30 mixture of chemically-modified and unmodified alginate. A low molecular weight alginate (PRONOVA™ VLVG alginate, NovaMatrix, Sandvika, Norway, cat. #4200506, approximate molecular weight <75 kDa; G:M ratio ≥1.5) was chemically modified with Compound 101 in Table 2 to produce chemically modified low molecular weight alginate (CM-LMW-Alg-101) solution with a viscosity of 25 cp to 35 cP. A solution of high molecular weight unmodified alginate (U-HMW-Alg) was prepared by dissolving unmodified alginate (PRONOVA™ SLG100, NovaMatrix, Sandvika, Norway, cat. #4202106, approximate molecular weight of 150 kDa-250 kDa) at 3% weight to volume in 0.9% saline. The CM-LMW-Alg solution was blended with the U-HMW-Alg solution at a volume ratio of 70% CM-LMW-Alg to 30% U-HMW-Alg (referred to herein as a 70:30 CM-Alg:UM-Alg solution).

Unmodified alginate control solution. An unmodified medium molecular weight alginate (SLG20, NovaMatrix, Sandvika, Norway, cat. #4202006, approximate molecular weight of 75-150 kDa), was dissolved at 1.4% weight to volume in 0.9% saline to prepare a U-MMW-Alg solution.

### Example 4: Culturing Exemplary Cells for Encapsulation as Single Cells

4A. ARPE-19 cells. These RPE cells were cultured and subsequently encapsulated in one-compartment or two-compartment hydrogel millicapsules. ARPE-19 cells may be cultured according to any method known in the art, such as according to the following protocol. ARPE-19 cells in a 75 cm² culture flask were aspirated to remove culture medium, and the cell layer was briefly rinsed with 0.05% (w/v) trypsin/0.53 mM EDTA solution (“TrypsinEDTA”) to remove all traces of serum containing a trypsin inhibitor. 2-3 mL Trypsin/EDTA solution was added to the flask, and the cells were observed under an inverted microscope until the cell layer was dispersed, usually between 5-15 minutes. To avoid clumping, cells were handled with care and hitting or shaking the flask during the dispersion period was minimized. If the cells did not detach, the flasks were placed at 37° C. to facilitate dispersal. Once the cells dispersed, 6-8 mL complete growth medium was added and the cells were aspirated by gentle pipetting. The cell suspension was transferred to a centrifuge tube and spun down at approximately 125×g for 5-10 minutes to remove TrypsinEDTA. The supernatant was discarded, and the cells were resuspended in fresh growth medium. Appropriate aliquots of cell suspension was added to new culture vessels, which were incubated at 37° C. The medium was renewed 2-3 times weekly.

4B. HEK293F cells. These cells, marketed as FreeStyle™ 293 F (Thermo Fisher Scientific, Waltham, MA, USA) were grown in suspension using a 125 ml Erlenmeyer flask with a working volume of 25 ml of FreeStyle 293 Expression Medium. Flasks were incubated at 37° C. on a shaker plate set for 125 RPM. Cells were grown to a density between 2 and 3×10⁶ cells/ml at which time the cells are re-seeded to a density between 2 and 3×10⁵ cells/ml, typically every 3-4 days. To avoid clumping, cells were handled with care, placed into a 50 ml falcon tube and vortexed for 5-10 seconds to maximize cell homogeneity. After counting the cell density, appropriate aliquots of cell suspension was added to new culture vessels.

### Example 5: Preparation of Cell Clusters for Encapsulation

Spheroid clusters of exemplary cells (e.g., ARPE-19 cells) are prepared using AggreWell™ spheroid plates (STEMCELL Technologies) and the protocol outlined herein. On Day 1, rinsing solution (4 mL) is added to each plate, and the plates is spun down for 5 minutes at 3,000 RPM in a large centrifuge. The rinsing solution is removed by pipet, and 4 mL of the complete growth medium is added. The ARPE-19 cells are seeded into the plates at the desired cell density and pipetted immediately to prevent aggregation, with the general rule of thumb that 3.9 million cells per well will generate 150 μm diameter clusters. The plate is spun down for 3 minutes at 800 RPM, and the plate is placed into an incubator overnight.

On Day 2, the plate is removed from incubation. Using wide bore pipet tips, the cells are gently pipetted to dislodge the spheroid clusters. The clusters are filtered through a 40 μm or 80 μm cell strainer to remove extraneous detached single cells and then spun down in a centrifuge for 2×1 minute. The clusters are resuspended gently using wide bore pipet tips and are gently stirred to distribute them throughout the medium or another material (e.g., alginate).

Alternatively, ARPE-19 spheroids are prepared using the following protocol. On Day 1, AggreWell™ plates are removed from the packaging in a sterile tissue culture hood. 2 mL of Aggrewell™ Rinsing solution is added to each well. The plate is centrifuged at 2,000 g for 5 minutes to remove air bubbles, and the AggreWell™ Rinsing Solution is removed from the wells. Each well is rinsed with 2 mL of the complete growth medium, and 2 million ARPE-19 cells in 3.9 mL of the complete growth medium is added to each well. The plate is centrifuged at 100 g for 3 minutes, then the cells are incubated the cells at 37° C. for 48 hours. On Day 3, the same protocol described above is used to dislodge the spheroid clusters.

### Example 6: Formation of One-Compartment and Two-Compartment Hydrogel Capsules

Suspensions of single cells (ARPE-19 cells or HEK293F cells as described in Example 4) were encapsulated in one-compartment or two-compartment hydrogel capsules according to the protocols described below.

Immediately before encapsulation, single cells were centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, pH≈7.4, ≈290 mOsm). After washing, the cells were centrifuged again and all of the supernatant was aspirated. In some experiments, the cell pellet was then resuspended in the 70:30 CM-Alg:UM-Alg solution described in Example 3 at a range of densities of suspended single cells per ml alginate solution. In some experiments, cells were used directly without suspension (e.g, dilution) in the alginate solution (“undiluted cells”).

Prior to fabrication of one-compartment and two-compartment hydrogel capsules, buffers and alginate solutions were sterilized by filtration through a 0.2-μm filter using aseptic processes.

To prepare particles configured as two-compartment hydrogel millicapsules of about 1.5 mm diameter, an electrostatic droplet generator was set up as follows: an ES series 0-100-kV, 20-watt high-voltage power generator (EQ series, Matsusada, NC, USA) was connected to the top and bottom of a coaxial needle (inner lumen of 22 G, outer lumen of 18 G, Ramé-Hart Instrument Co., Succasunna, NJ, USA). The inner lumen was attached to a first 5-ml Luer-lock syringe (BD, NJ, USA), which was connected to a syringe pump (Pump 11 Pico Plus, Harvard Apparatus, Holliston, MA, USA) that was oriented vertically. The outer lumen was connected via a luer coupling to a second 5-ml Luer-lock syringe which was connected to a second syringe pump (Pump 11 Pico Plus) that was oriented horizontally. When preparing two-compartment capsules that encapsulate cells only in the inner compartment, a first alginate solution comprising the cells (as single cell suspension) was placed in the first syringe and a second alginate solution lacking cells was placed in the second syringe. The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump were 12.06 mm diameter and the flow rates of each pump were adjusted to achieve various test flow rates in the Examples below, but keeping the total flow rate set at 10 ml/h.

For fabrication of one-compartment hydrogel capsules of about 1.5 mm diameter, the 70:30 CM-Alg:UM-Alg solution described in Example 3 (with or without a suspension of single cells) was loaded into a syringe and capped with an 18-gauge blunt tipped needle (SAI Infusion Technologies). The syringe was placed into a syringe pump oriented vertically above a dish containing the crosslinking buffer. A high voltage power generator was connected to the needle and grounded to the biosafety cabinet. The syringe pump and power generator were turned on to extrude the alginate solution through the needle with a flow-rate of 0.16 mL/min or 10 ml/hr and adjusting the voltage in a range of 5-9 kV until there was a droplet rate of 12 droplets per 10 seconds.

For fabrication of both the two-compartment and one-compartment millicapsules, after extrusion of the desired volumes of alginate solutions, the alginate droplets were crosslinked for five minutes in a cross-linking solution which contained 25 mM HEPES buffer, 20 mM BaCl₂, and 0.2M mannitol. In some experiments, the cross-linking solution also contained 0.01% of poloxamer 188. Capsules that had fallen to the bottom of the crosslinking vessel were collected by pipetting into a conical tube. After the capsules settled in the tube, the crosslinking buffer was removed, and capsules were washed. Capsules without cells were washed four times with HEPES buffer (NaCl 15.428 g, KCl 0.70 g, MgCl₂·6H₂O 0.488 g, 0 ml of HEPES (1 M) buffer solution (Gibco, Life Technologies, California, USA) in 2 liters of deionized water) and stored at 4° C. until use. Capsules encapsulating cells were washed four times in HEPES buffer, two times in 0.9% saline, and two times in culture media and stored in an incubator at 37° C.

In some experiments, the quality of capsules in a composition of two-compartment or one-compartment capsules was examined. An aliquot containing at least 200 capsules was taken from the composition and transferred to a well plate and the entire aliquot examined by optical microscopy for quality by counting the number of spherical capsules out of the total.

In some experiments, the mechanical strength of capsules in a composition of two-compartment capsules was examined using a texture analyzer to determine the initial fracture force as described herein above.

### Example 7. Assessing the Effect of Cell Loading on Capsule Quality

Capsule compositions (comprising two-compartment capsules or one-compartment capsules) were prepared as described in Example 6 using the 70:30 CM-Alg:UM-Alg solution described in Example 3 and various loading amounts of cells in the alginate solution used to form the first (inner) compartment. The cross-linking solution included 0.01% poloxamer 188. Two-compartment capsules (1.5 mm diameter) with equal volume first and second compartments formed from the 70:30 alginate solution were prepared using a flow rate of 5 ml/hour for the alginate solutions in each of the first and second syringes. In addition, a composition of two-compartment, 1.5 mm capsules was prepared in substantially the same manner except the inner compartment was formed using undiluted cells (concentration equiv. to 500 million cells/ml) in the first syringe (e.g., no alginate solution.) Engineered ARPE19 cells expressing Factor VIII were encapsulated at 10-50 million cells/ml alginate solution in one-compartment capsules or 10-500 million cells/ml alginate solution (concentration equiv. to one-compartment capsules) in 2-compartment capsules. The different capsule compositions were examined for quality and the results are shown in FIG. 3.

For the one-compartment millicapsules, it was generally observed that capsule quality (i.e., spherical shape) decreased as cell loading increased. Quality of compositions comprising one-compartment millicapsules was below the acceptable threshold of 95% spherical particles in the examined aliquot at cell loadings greater than 20 million cells/ml alginate. Compositions comprising two-compartment millicapsules had very high spherical quality up to a loading cell amount of undiluted cells equivalent to 500 million cells/ml alginate solution, which is about a 25× higher cell loading capacity than the highest acceptable cell loading capacity for the one-compartment particles. For this particular configuration of 70:30 CM-Alg:UM-Alg and capsule size, a cell loading equivalent to 500 million cells/ml alginate solution appears to be the upper loading limit for the first (inner) compartment. Therefore, the two-compartment millicapsules permitted encapsulation of a significantly greater number of cells without affecting the spherical morphology of the capsules.

### Example 8: Altering the Effect of Flow Rate Ratio on the Thickness of the Second Compartment

Compositions containing two-compartment hydrogel millicapsules (about 1.5 mm in diameter) were prepared with a 70:30 mixture of CM-Alg:U-Alg in both compartments. The combined flow rates of alginate solutions through the outer and inner lumens was held constant at 10 ml/h, while the ratio of these flow rates was varied to prepare capsules with varying compartment thicknesses. To visualize the compartments in the resulting capsules, ARPE19-FVIII cells were encapsulated at 20 million cells/ml alginate solution in the first (inner) compartment. Compartment thickness was measured via image analysis.

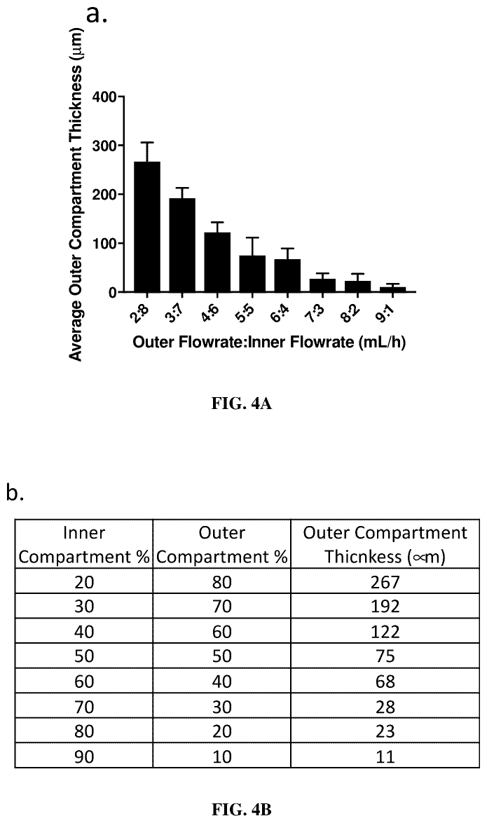

By changing the outer:inner lumen flow rate ratio, the mean thickness of the second (outer) compartment of a 1.5 mm two-compartment capsule was varied from 11-267 microns as shown in FIGS. 4A-4B. In all cases, spherical capsules of about 1.5 mm were formed. The smallest second (outer) compartment created was about 11 microns in diameter, and increasing the ratio of outer:inner lumen flow rates increased the second (outer) compartment thickness from about 11 microns to a maximum of 267 microns. Therefore, the second (outer) compartment thickness of hydrogel capsules may be altered by varying inner and outer flow rates to generate a composition of uniform spherical millicapsules.

### Example 9: Effect of Varying the Composition and Size of the First Compartment on the Mechanical Properties of Particles

Compositions of two-compartment hydrogel millicapsules were prepared by extruding first and second alginate solutions through a coaxial needle as described in Example 6. The second (outer) compartment was prepared using the 70:30 CM-Alg:U-Alg solution described in Example 3 and the first (inner) compartment was prepared using the U-HMW-Alg solution described in Example 3. While keeping the total (e.g., combined) flow rate at 10 ml/h, the inner outer flow rate ratios (I:O) were varied from 1 ml to 9 ml per hour and 9 ml to 1 ml per hour to produce millicapsules with different inner and outer compartment thicknesses. Control capsule compositions were also prepared. One control contained one-compartment capsules made from the same 70:30 CM-Alg:U-Alg solution. A second control composition contained one-compartment capsules made from the same U-HMW-Alg solution. Mechanical testing was performed on aliquots from each of the compositions using a texture analyzer to measure initial fracture of individual capsules.

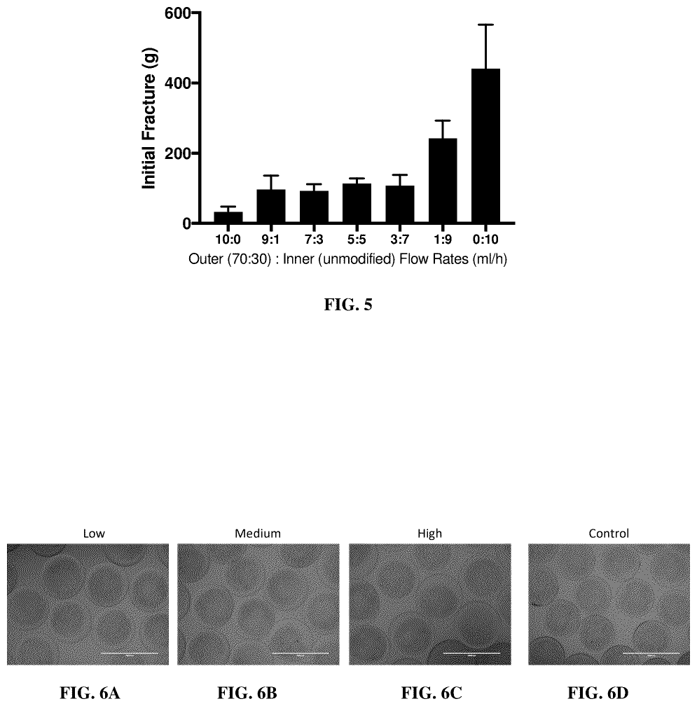

Two-compartment capsules of about 1.5 mm in diameter were created in all conditions. One-compartment capsules prepared from the 70:30 mixture had the lowest initial fracture compared to all of the two-compartment capsule configurations. The mechanical strength of two-compartment capsules with an inner compartment of U-HMW-Alg increased with increasing volume fraction of the inner compartment to the entire capsule (see FIG. 5). Capsules in the second control composition (one-compartment U-HMW-Alg capsules) had higher initial fracture than all two-compartment particles with inner and outer compartments composed of U-HMW-Alg and 70:30 CM-Alg:U-Alg, respectively. As the volume fraction of the inner compartment (U-HMW-Alg) increased, mean initial fracture increased. Therefore, changing the configuration of the inner compartment (identity of the alginate and/or thickness) of a 2-compartment millicapsule can alter its mechanical properties. Thus, 2-compartment hydrogel millicapsules can present the same capsule surface with respect to chemical modification (e.g., to mitigate FBR) but have stronger mechanical strength by changing the alginate composition in the first (inner) compartment.

### Example 10: Preparation of Two-Compartment Hydrogel Capsules with Varying Amounts of Chemical Modification in the 2^{nd} (Outer) Compartment

Chemically modified alginate solutions were prepared with varying amounts of conjugation of compound 101. Polymers were prepared with low (2.03% N), medium (4.42% N), or high (6.72% N) levels of compound 101 conjugation, where % nitrogen is determined by combustion analysis and corresponds to the amount of small molecule conjugated to the polymer. Compositions of two-compartment hydrogel millicapsules were prepared as follows. The outer compartment was formed using (i) a solution containing one of these conjugated polymers blended with U-HMW-Alg at a 70:30 ratio of CM-Alg-101 to U-HMW-Alg or (ii) the U-MMW-Alg solution described in Example 3 as a control. The inner compartment was formed using a solution containing the medium CM-Alg-101 conjugation blended with U-HMW-Alg. The inner and outer flow rates were both 5 ml/h. To visualize the first (inner) compartment, 20 million ARPE19-FVIII cells/ml alginate solution were encapsulated in the first (inner) compartment of the capsule.

FIGS. 6A-6D show the capsules created with low, medium, high conjugation CM-Alg polymers or the control (unmodified) polymer in the second (outer) compartment. Compositions of spherical, uniform millicapsules of about 1.5 mm in diameter were formed regardless of which CM-A polymer solution was used to form the outer compartment, demonstrating that high quality capsules can be prepared with the same polymer used in the first (inner) compartment and varying amounts of chemical modification in the second (outer) compartment.

### Example 11: Effect of Varying the Level of Chemical Modification in the Second Compartment on Biocompatibility

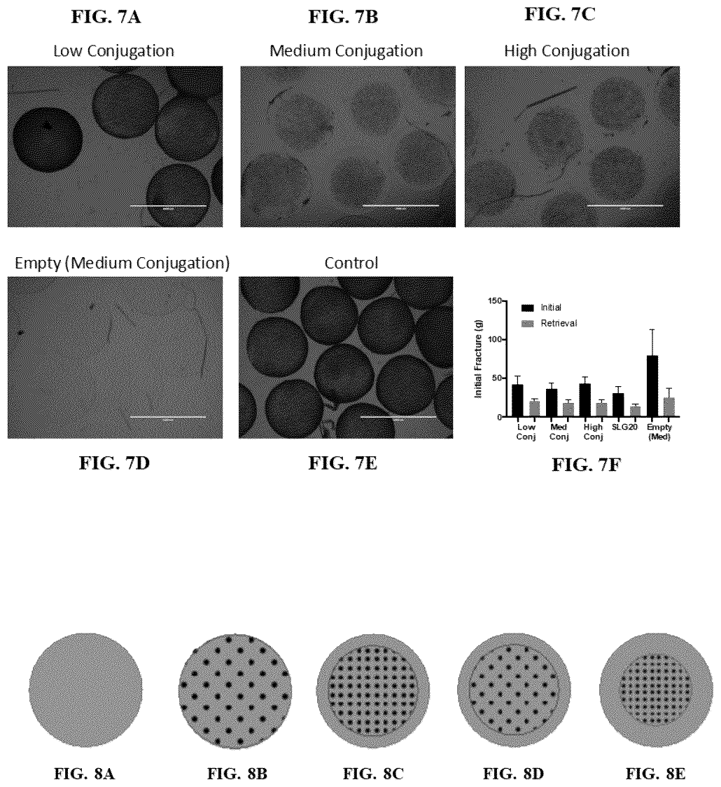

The hydrogel capsules prepared in Example 10 were examined for fibrosis in vivo by implanting the capsules into the IP space of C57/BL6 mice for one week. In this mouse model, encapsulated xenogeneic cell lines, such as human RPE cells, generally induce a fibrotic response. At retrieval, particles were imaged for the presence of fibrosis, and initial fracture measured using a texture analyzer. The results are shown in FIGS. 7A-7F.

Upon retrieval, the empty capsules (no cells) had no visible fibrosis (FIG. 7D). Control capsules (U-MMW-Alg outer compartment) had fibrotic buildup surrounding the particles (FIG. 7E). For capsules with a CM-Alg-101 in the outer compartment, a fibrotic response was only observed on the low conjugation CM-Alg capsules (FIG. 7A), with minimal fibrosis observed on the medium and high conjugation CM-Alg capsules (FIGS. 7B-7C). Initial fracture was measured prior to implantation and at retrieval, and all 2-compartment capsules had similar initial fracture at each time point (FIG. 7F). Therefore, altering the chemical modification profile only on the second (outer) compartment of the capsule may be used to modulate an afibrotic property, i.e., increasing the concentration of a compound of Formula I in the outer compartment can substantially reduce fibrosis without affecting mechanical strength.

### Example 12: Effect of Varying the Level of Chemical Modification in the Second Compartment on Macrophage Adhesion Over Time

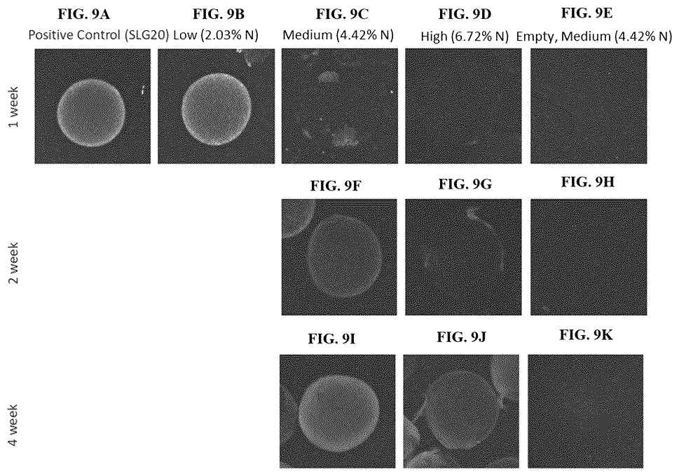

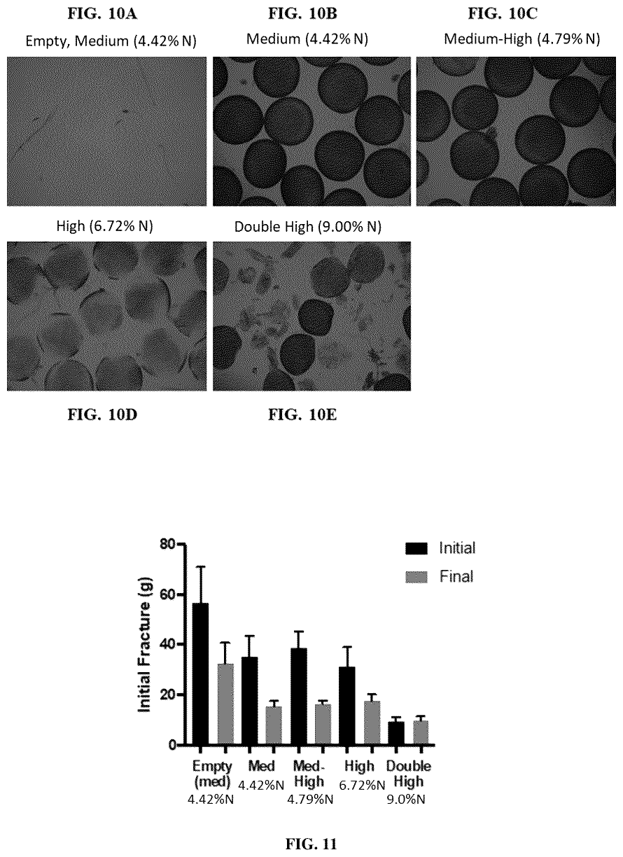

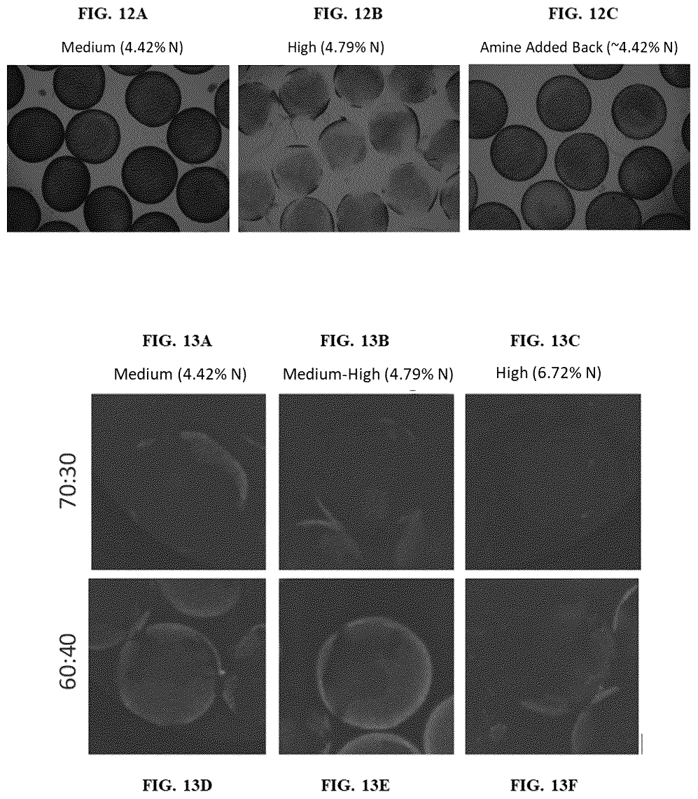

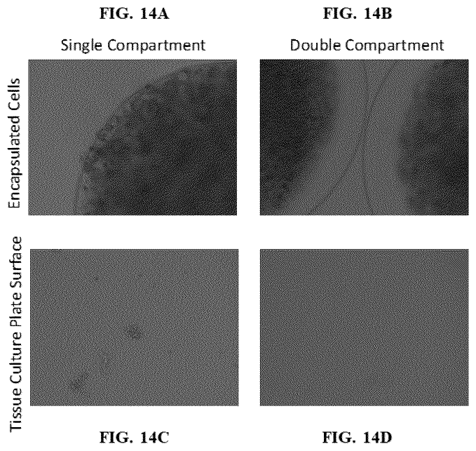

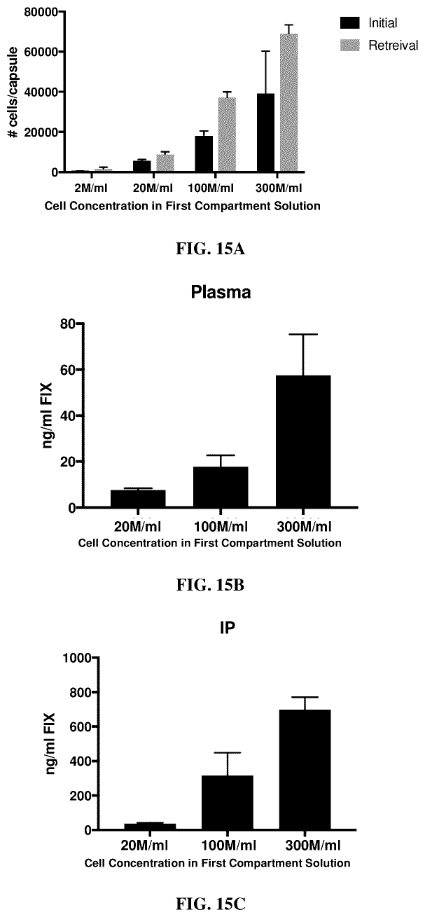

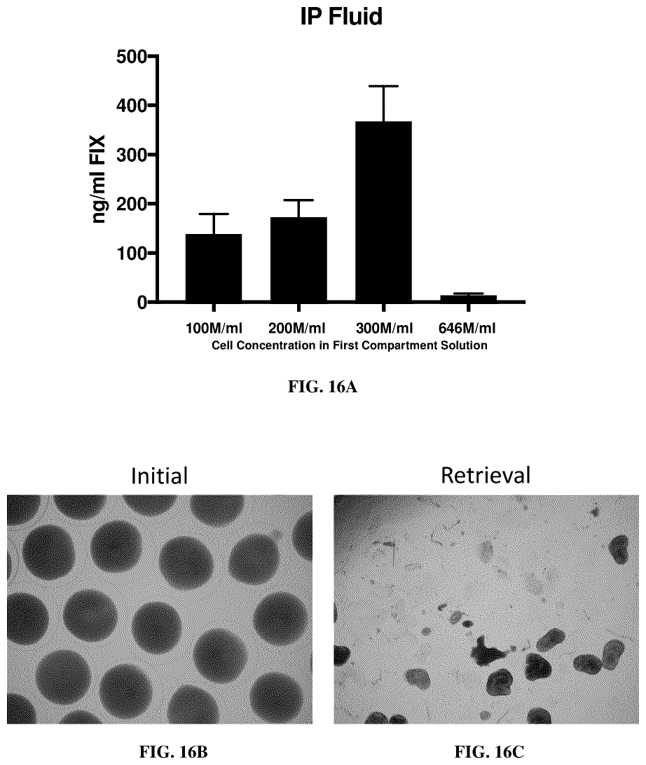

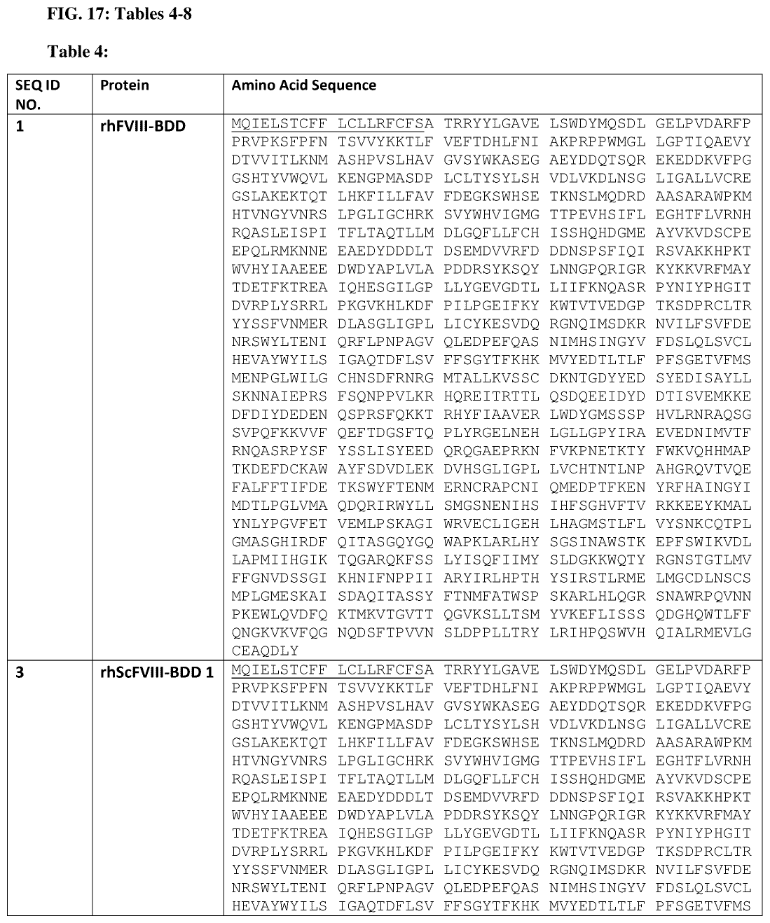

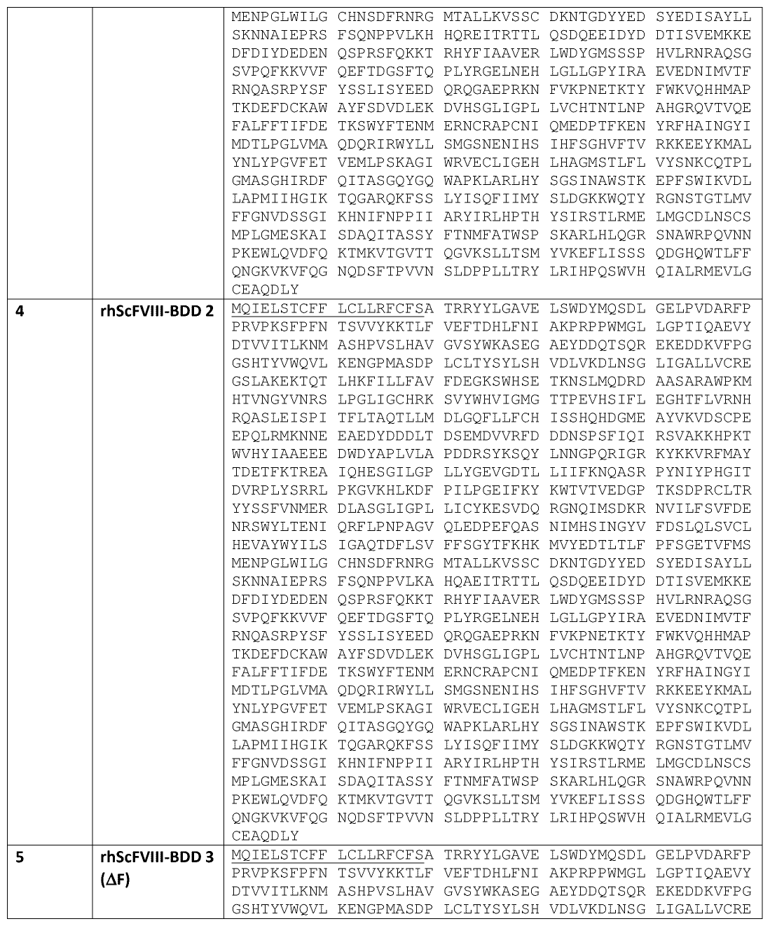

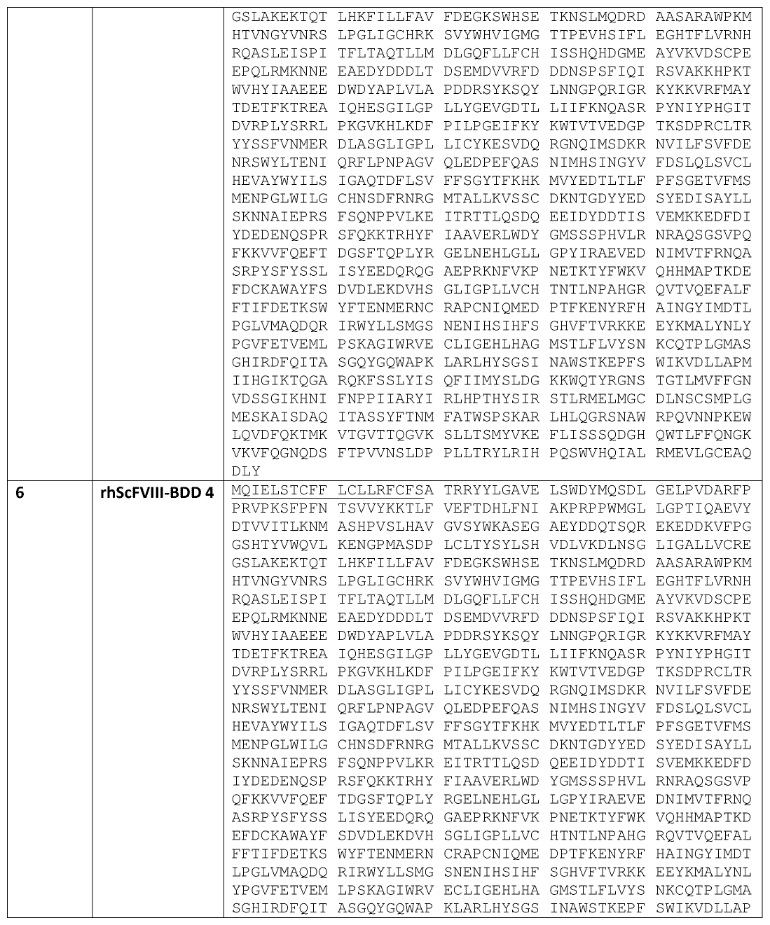

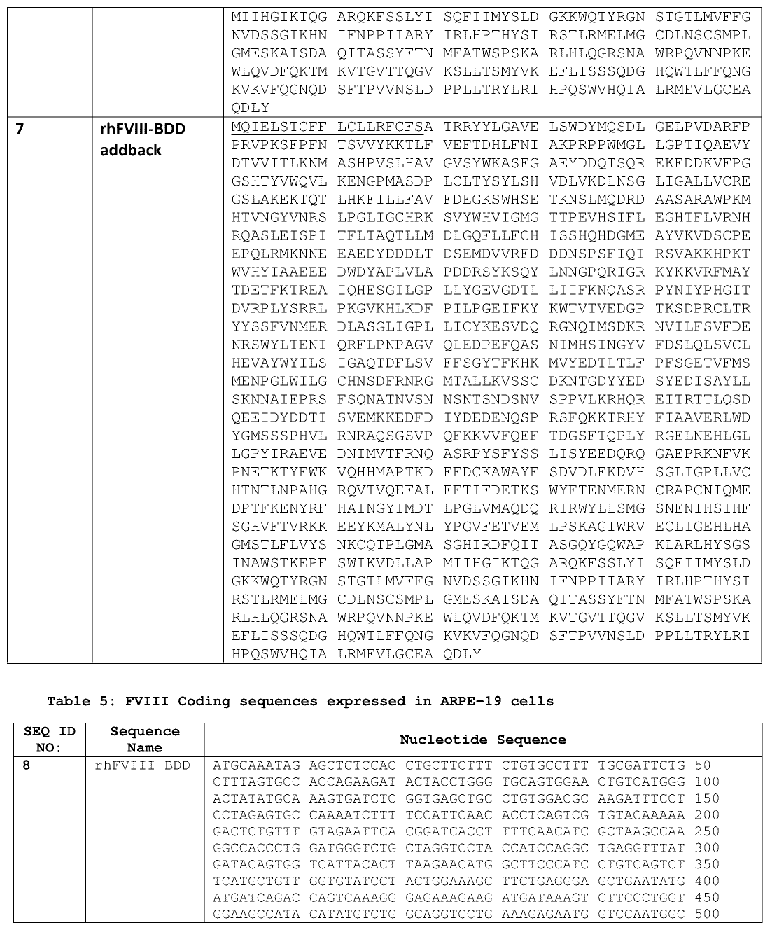

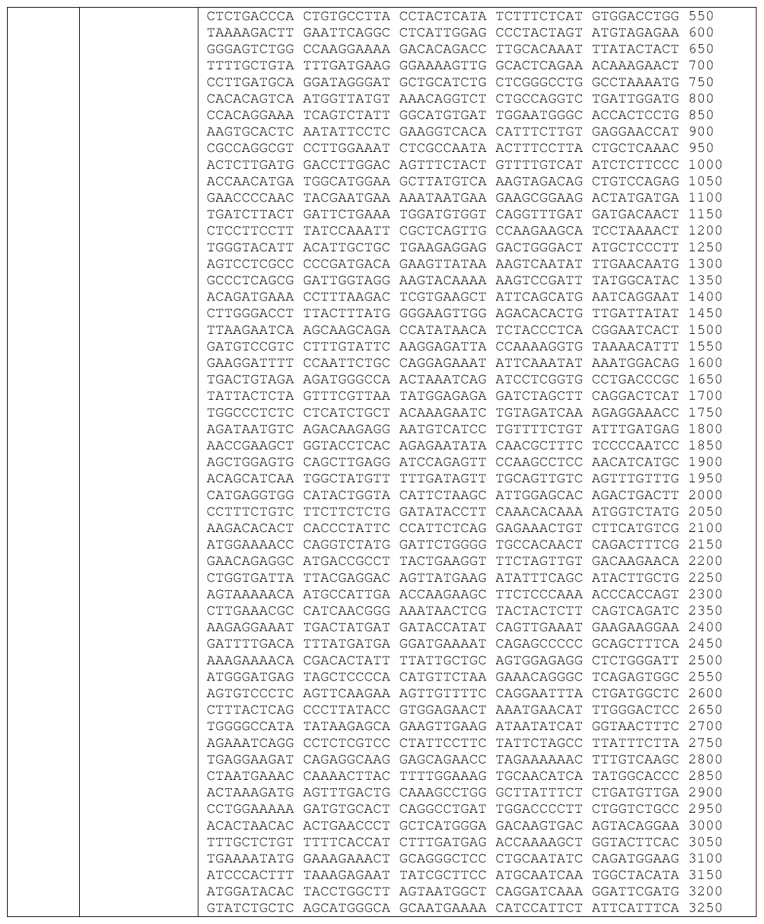

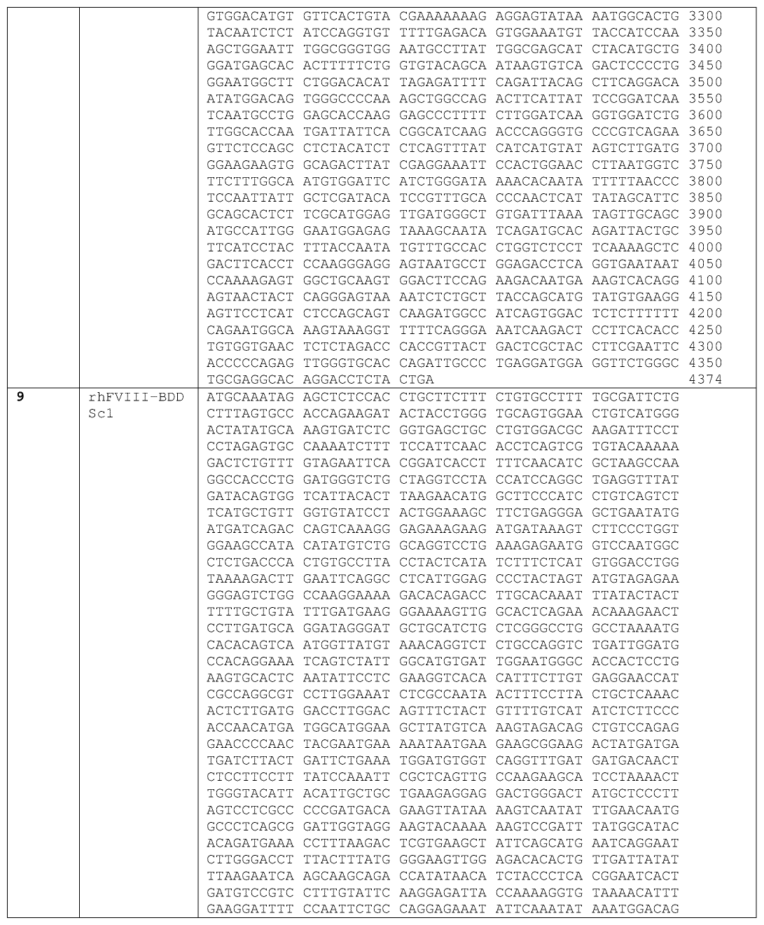

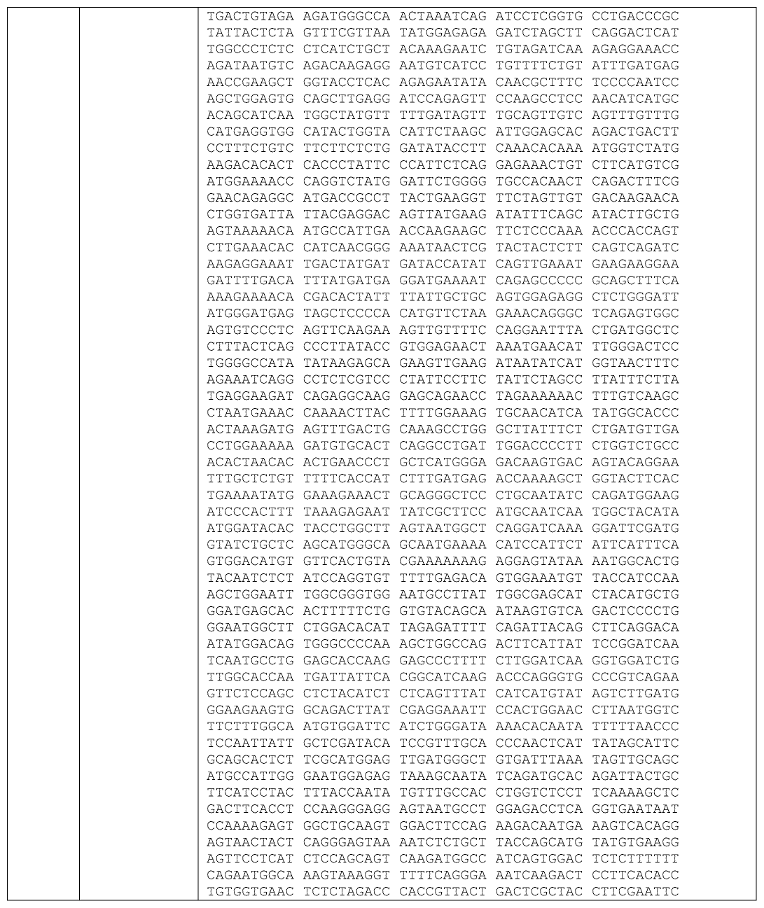

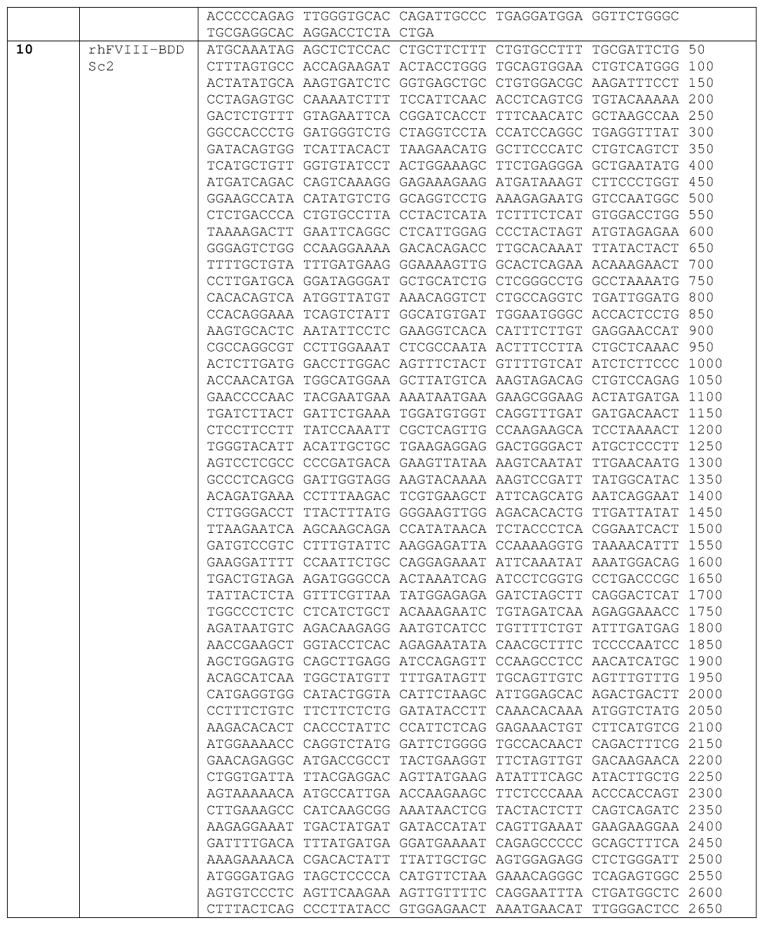

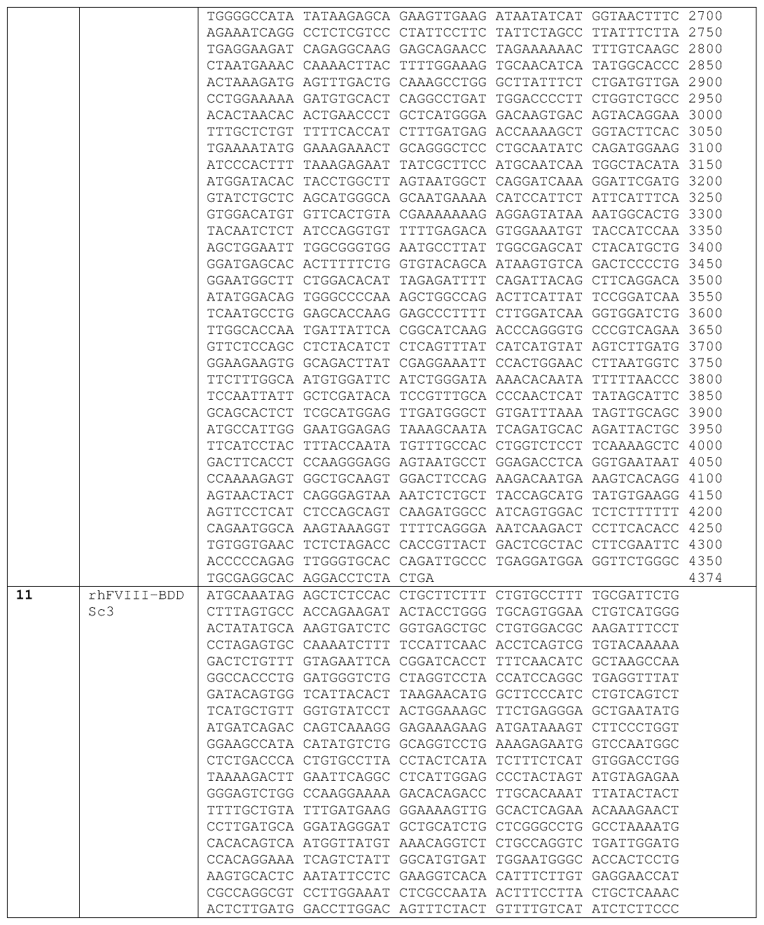

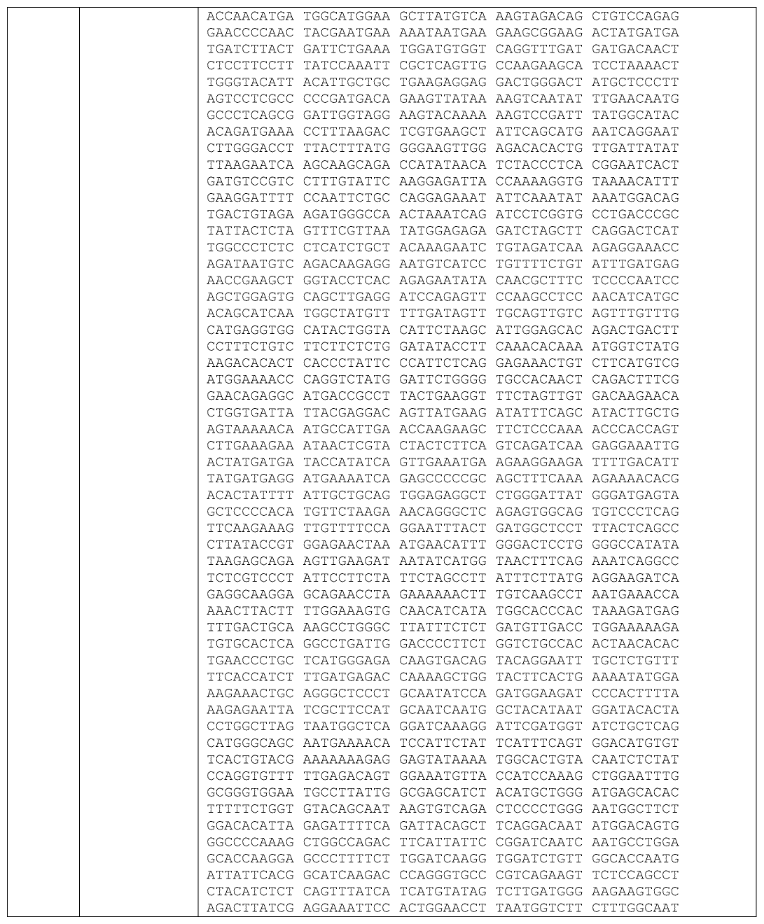

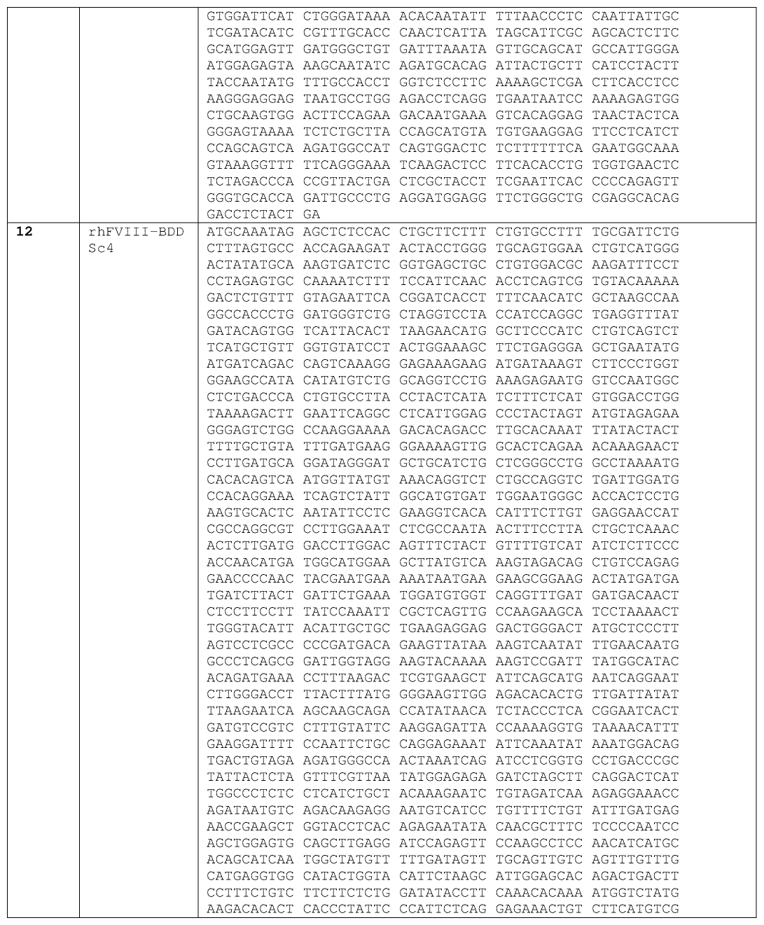

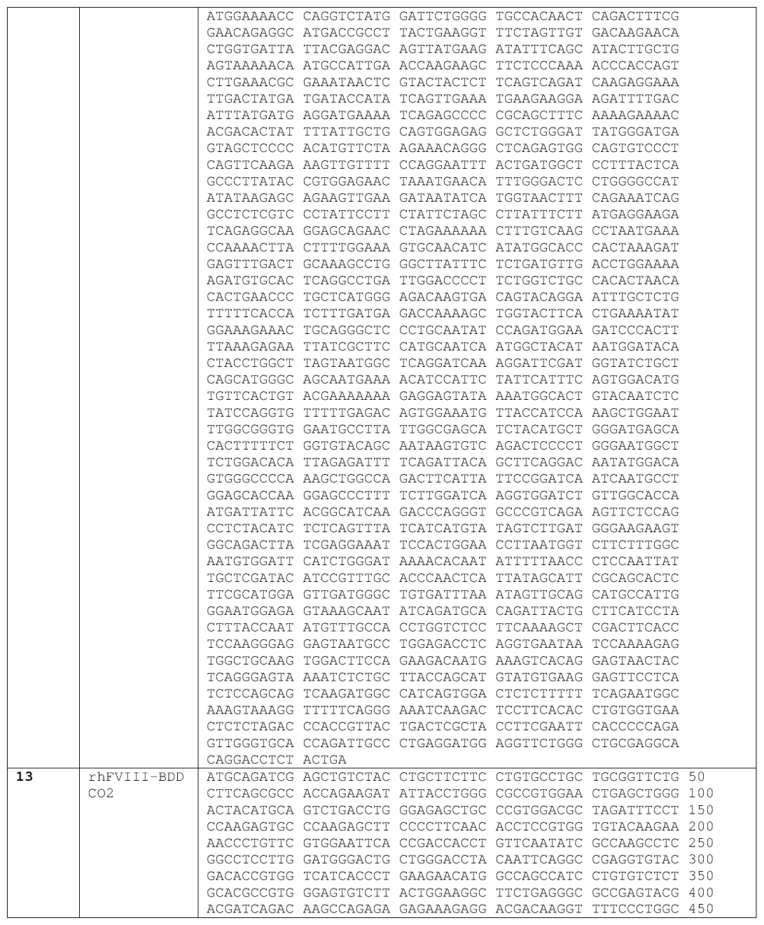

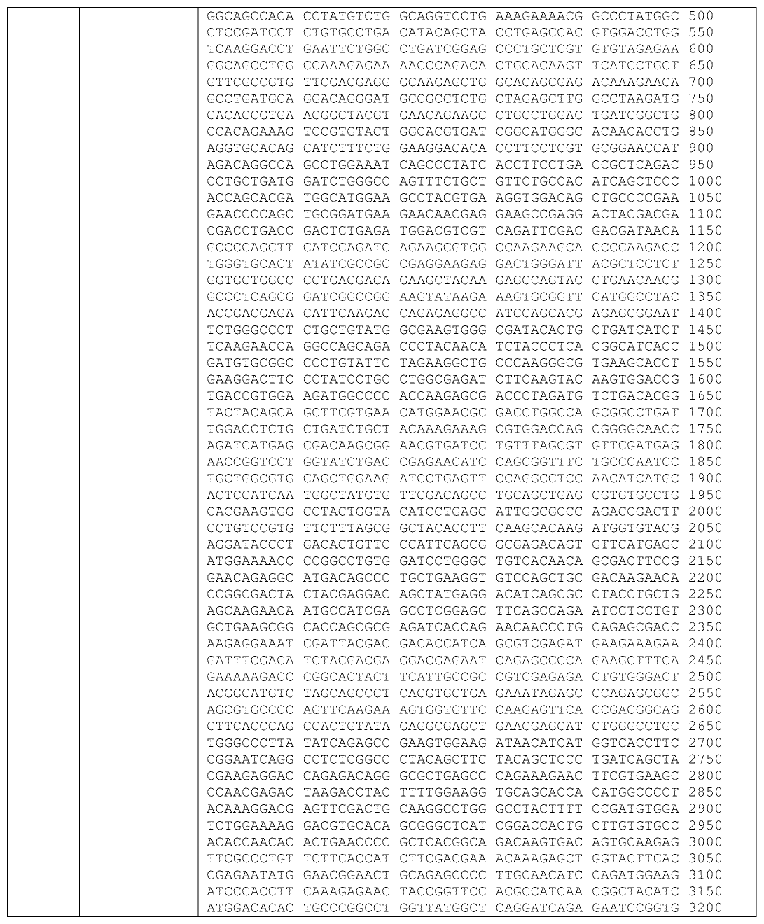

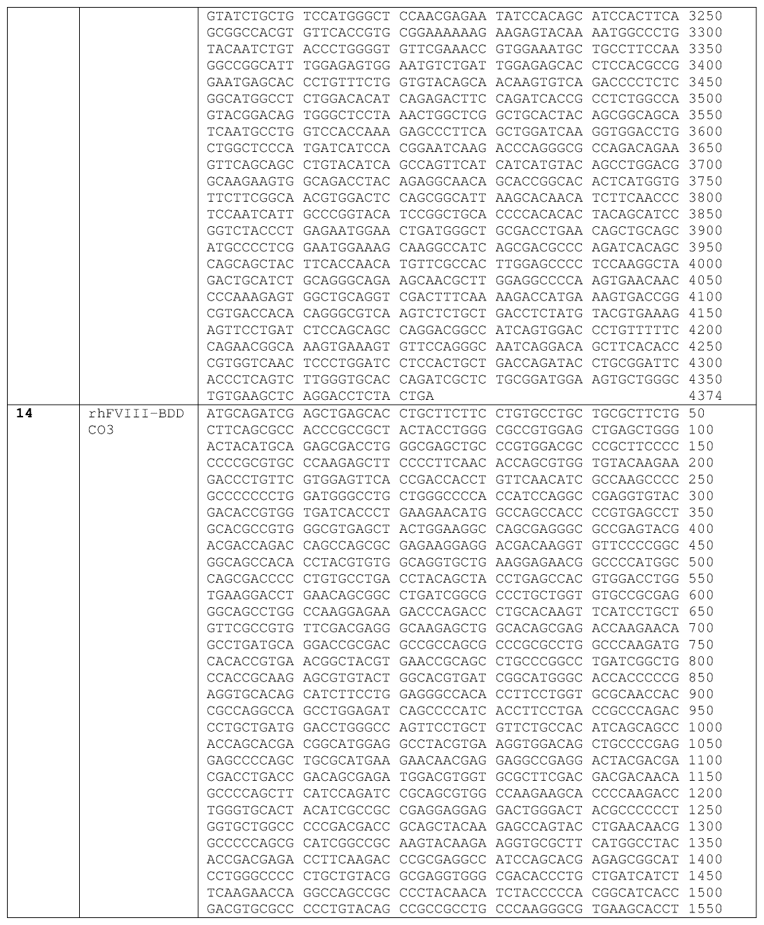

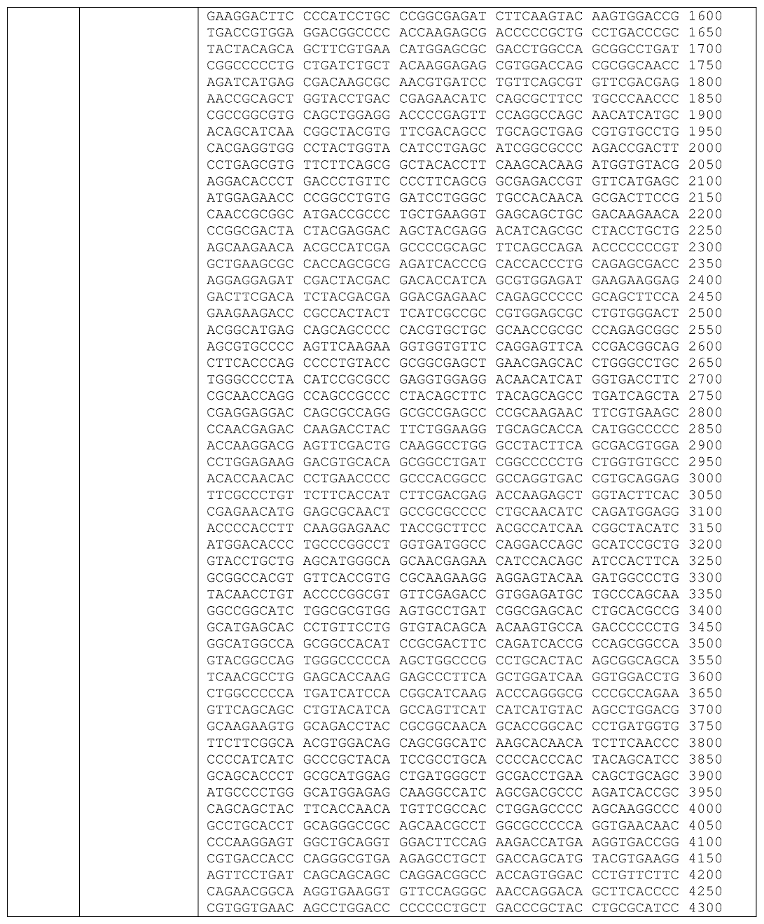

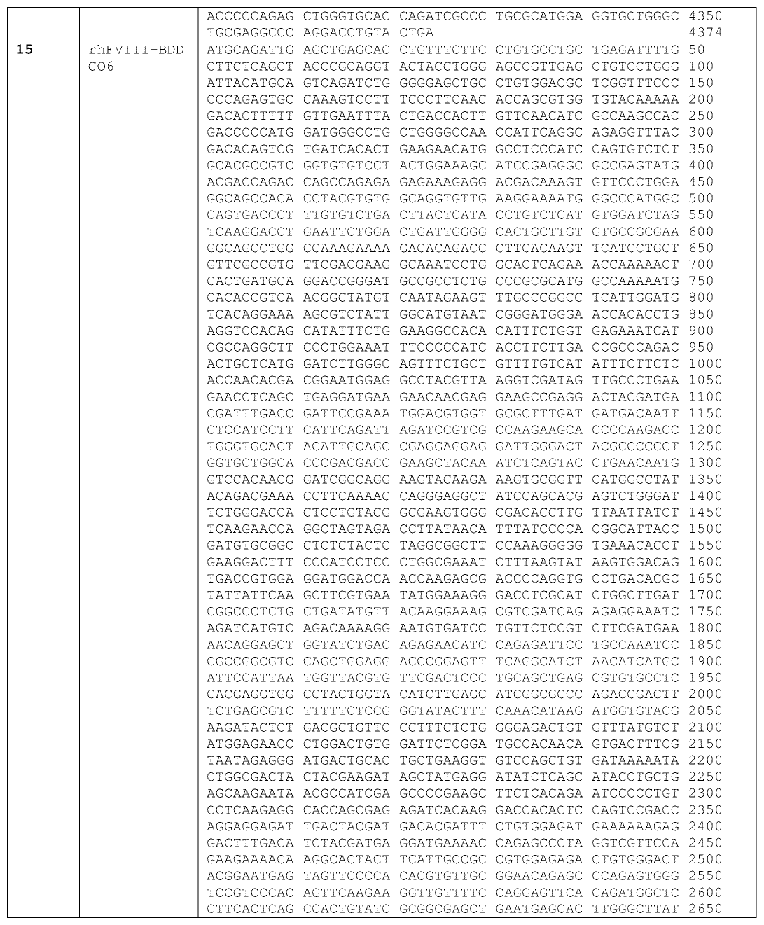

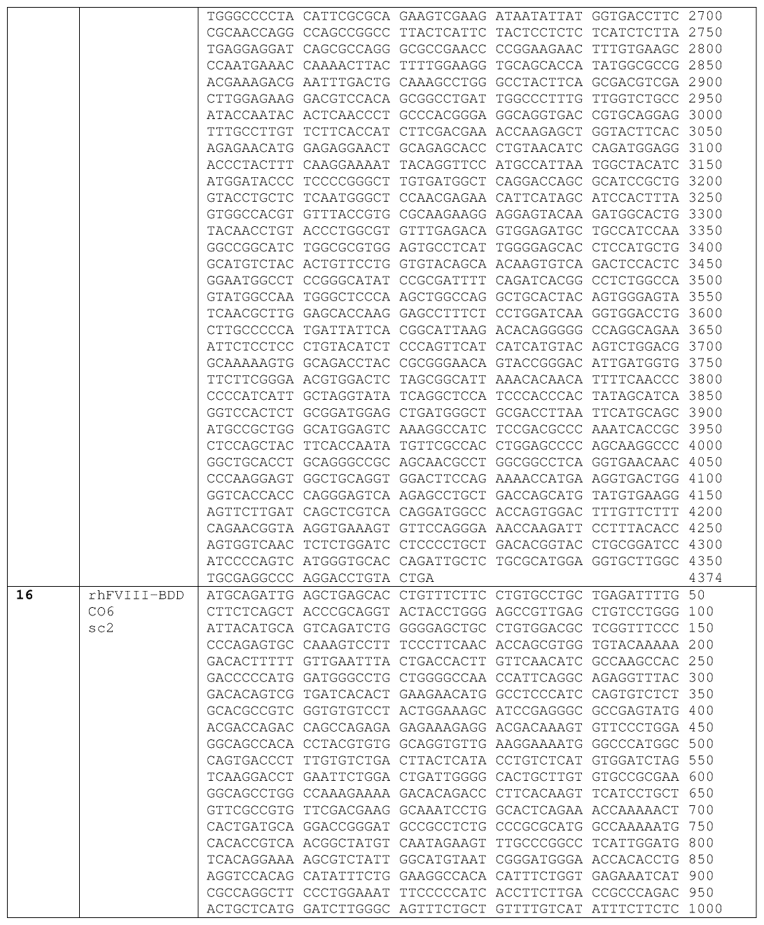

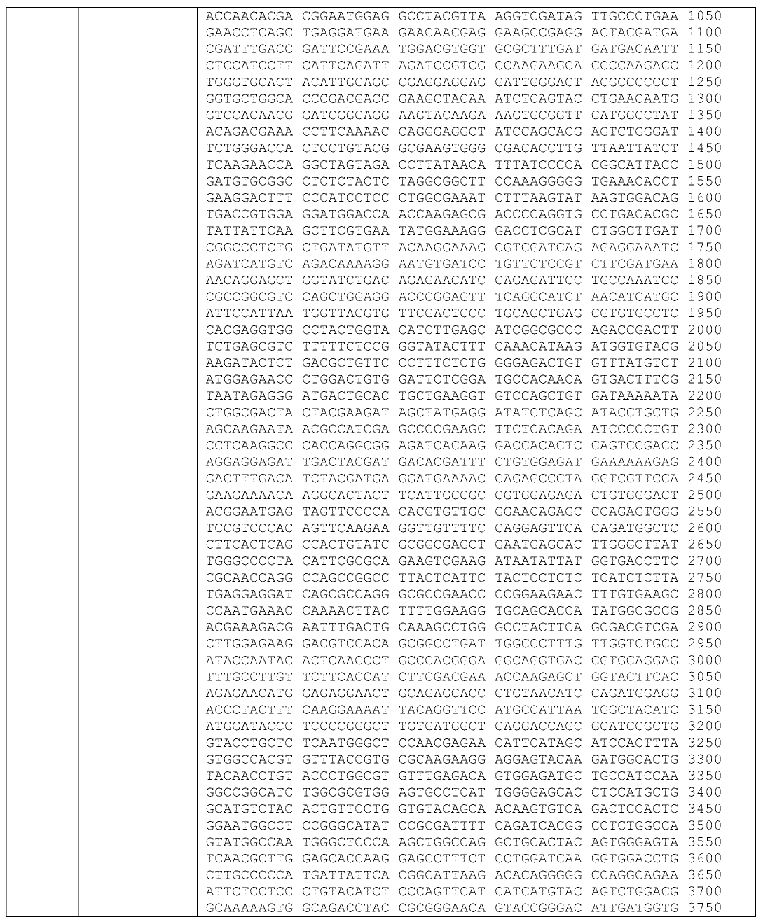

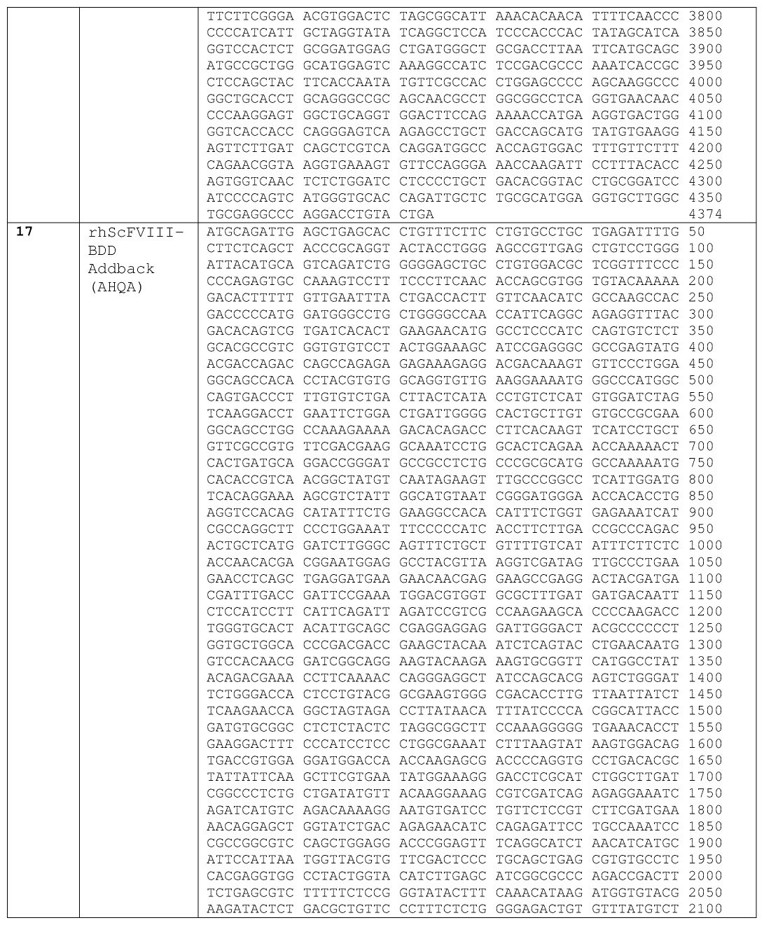

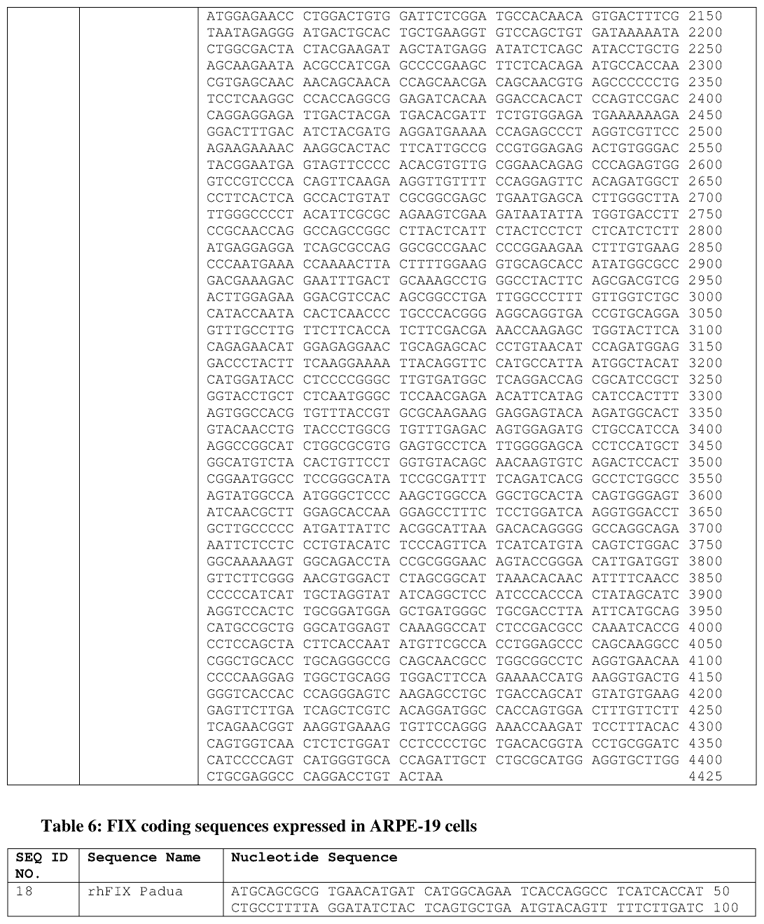

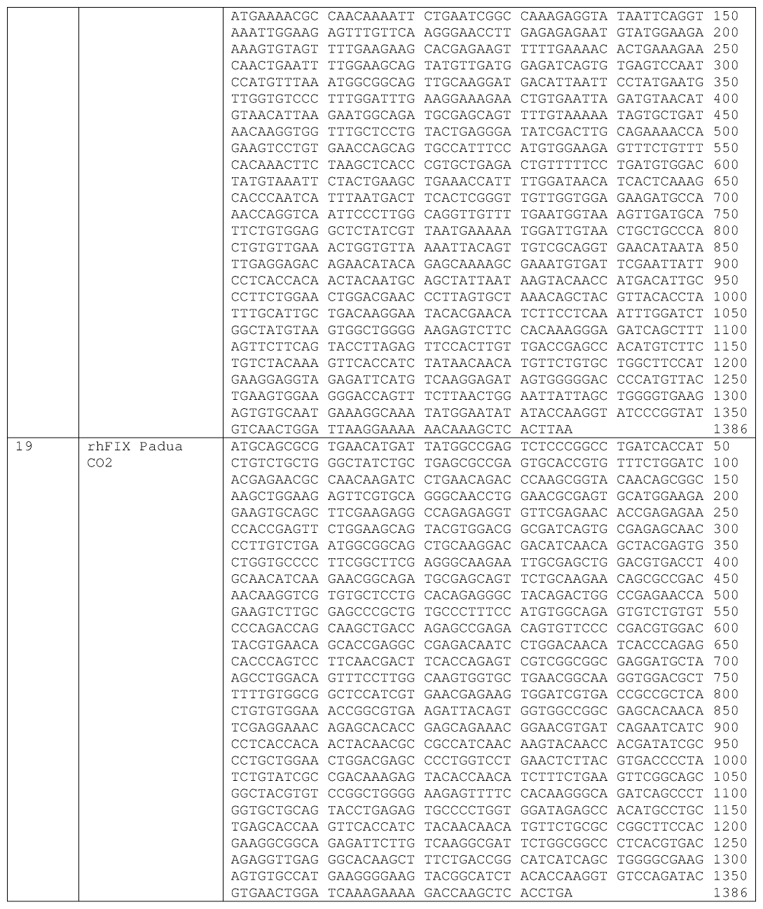

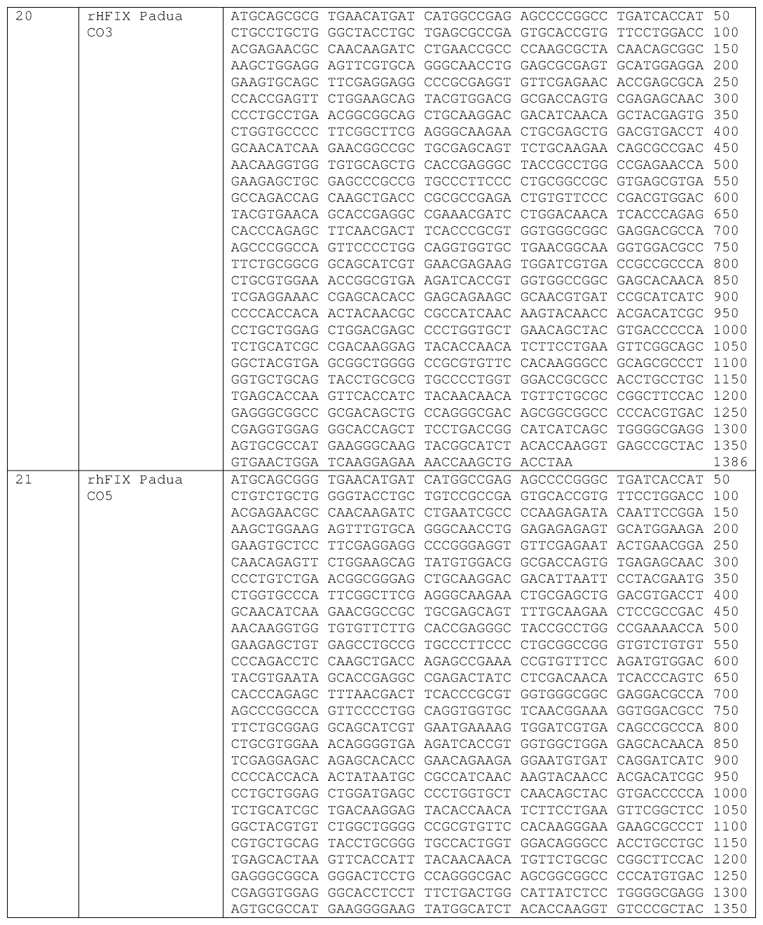

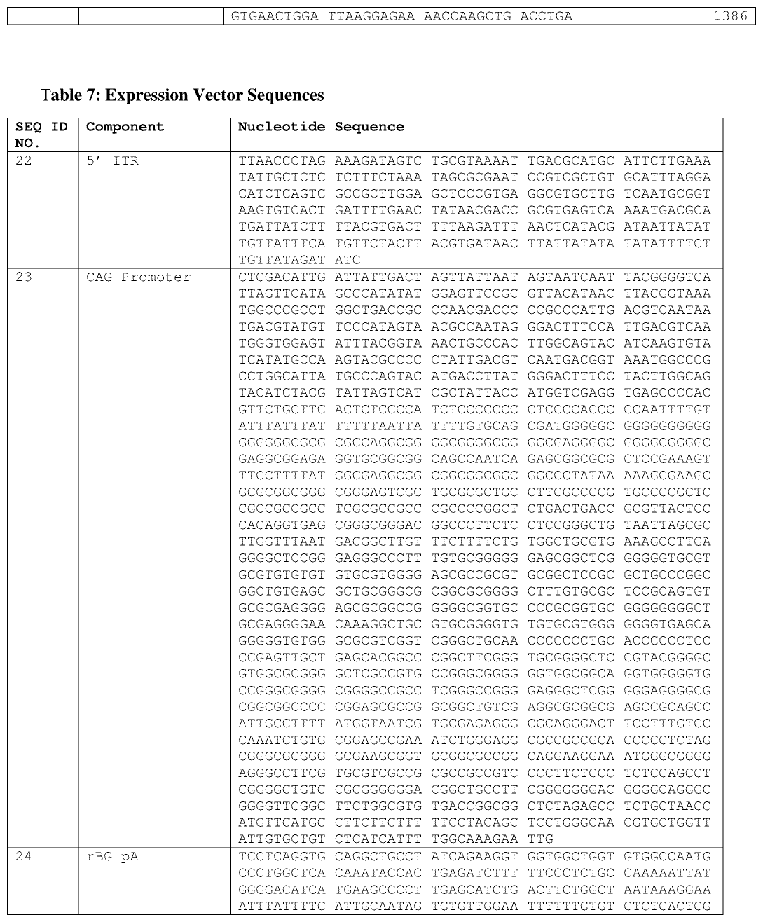

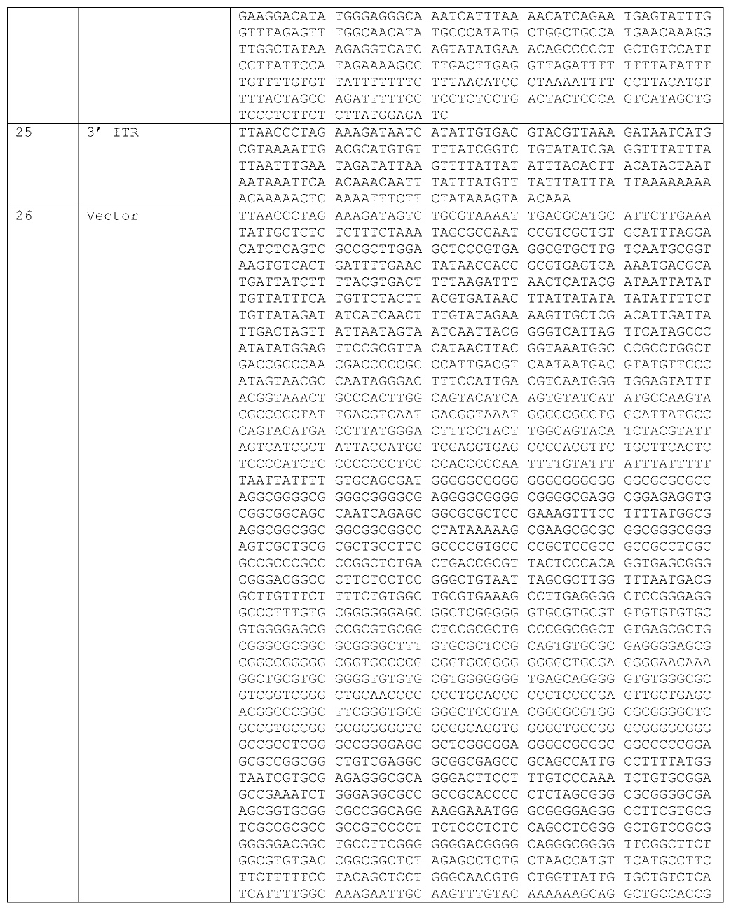

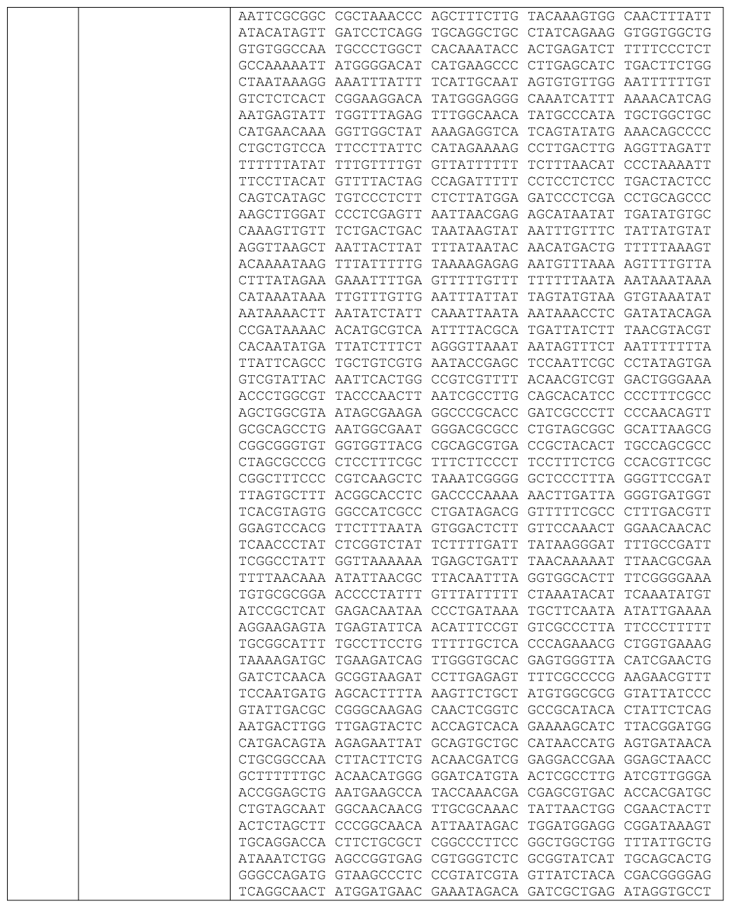

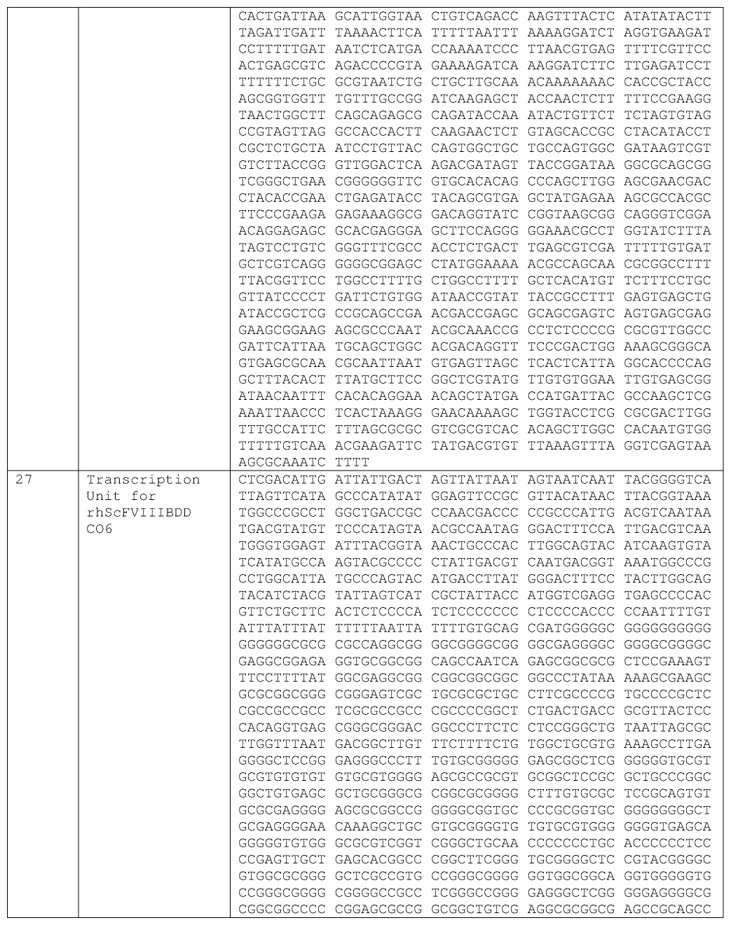

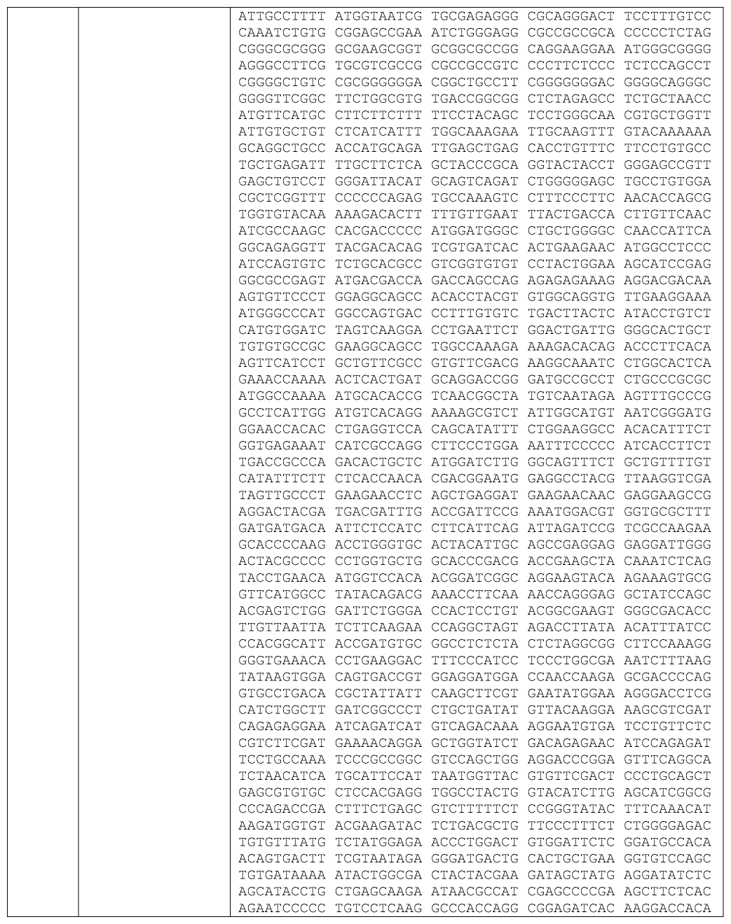

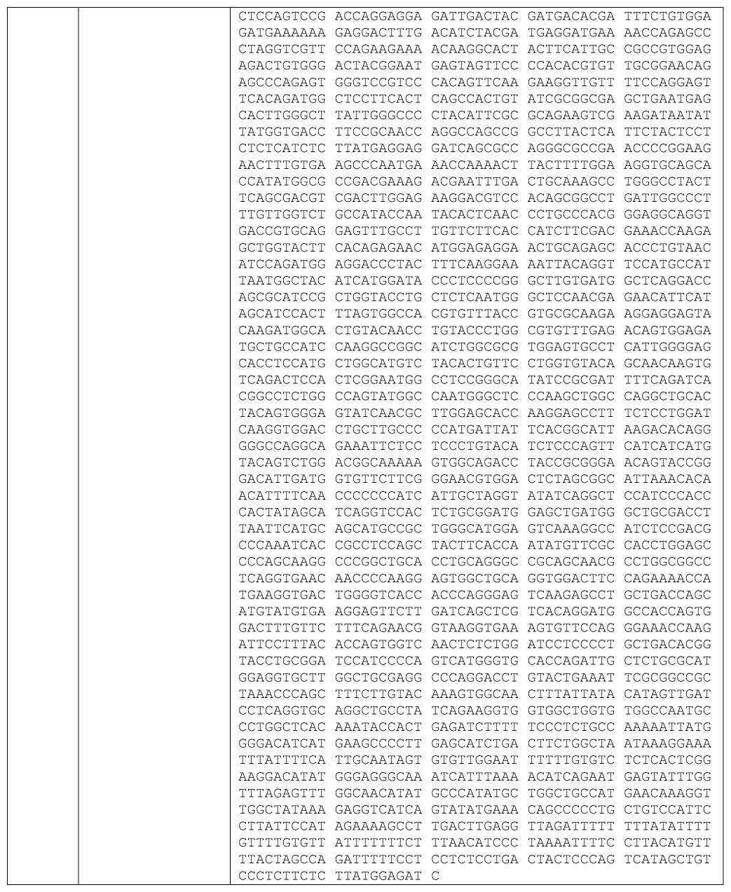

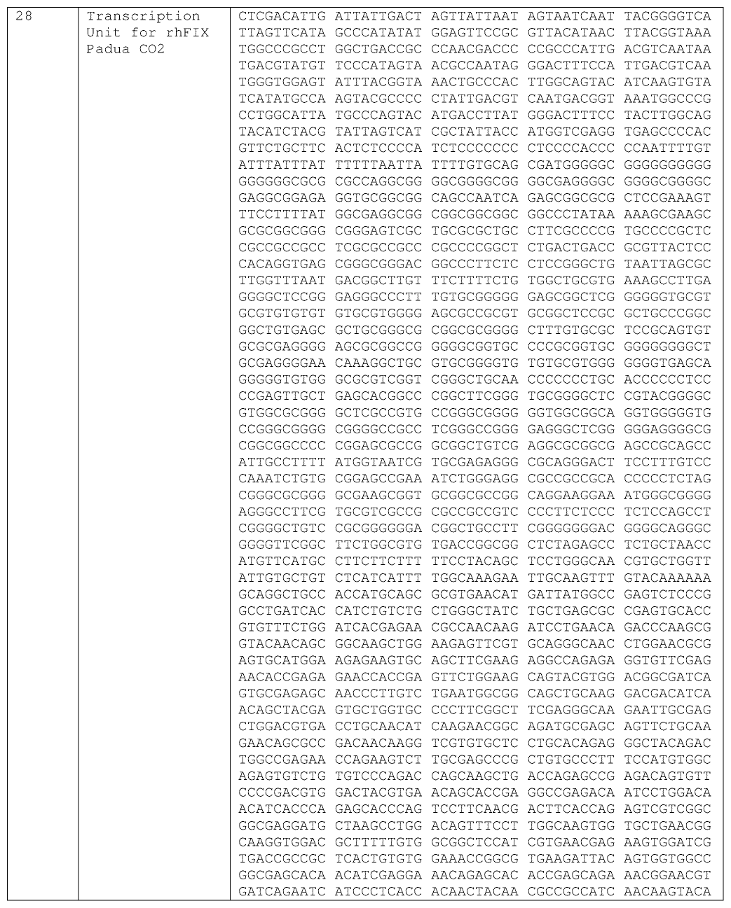

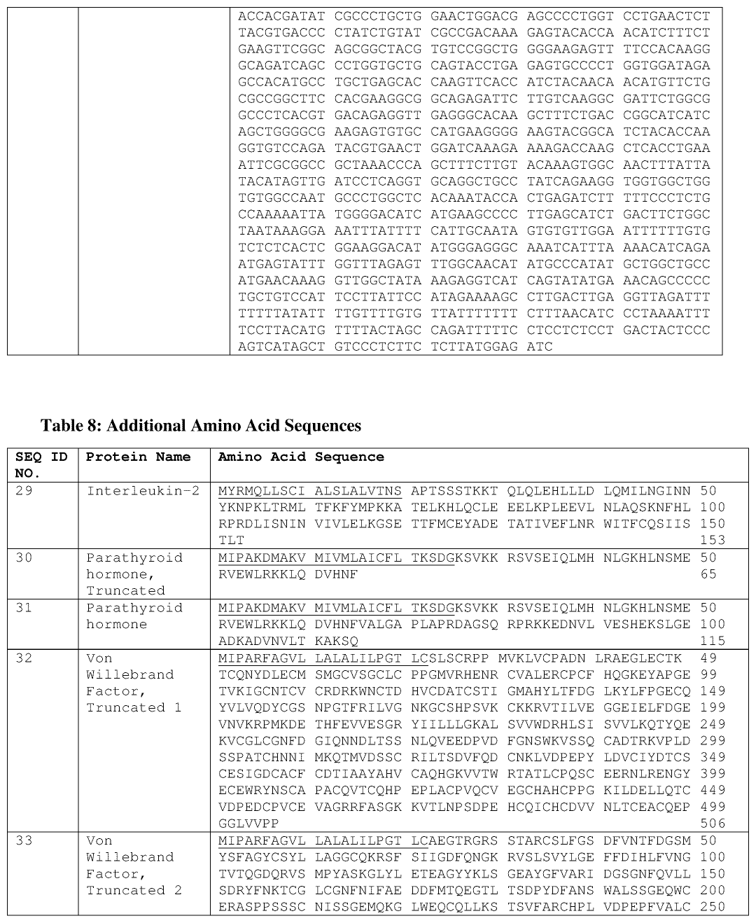

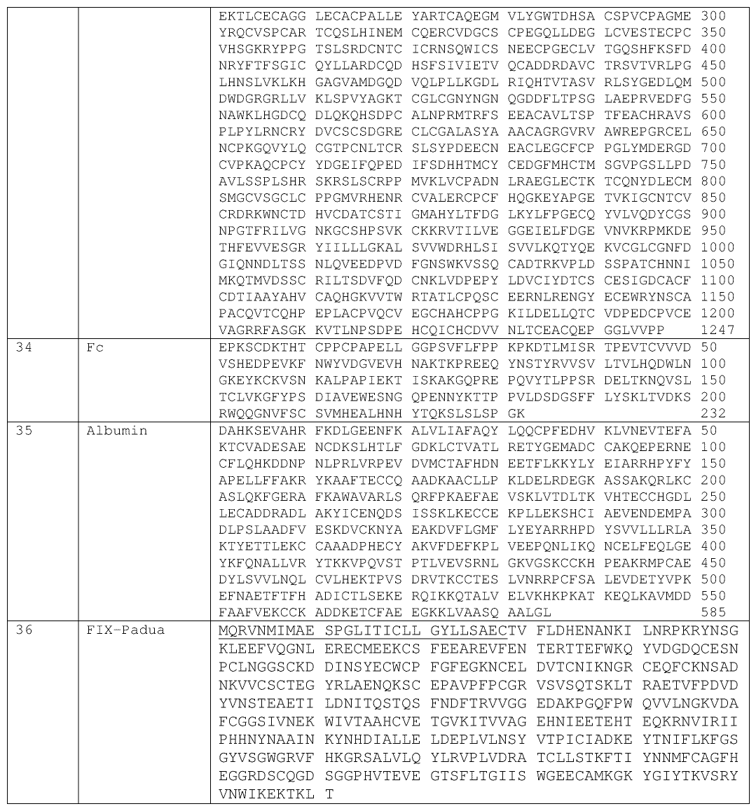

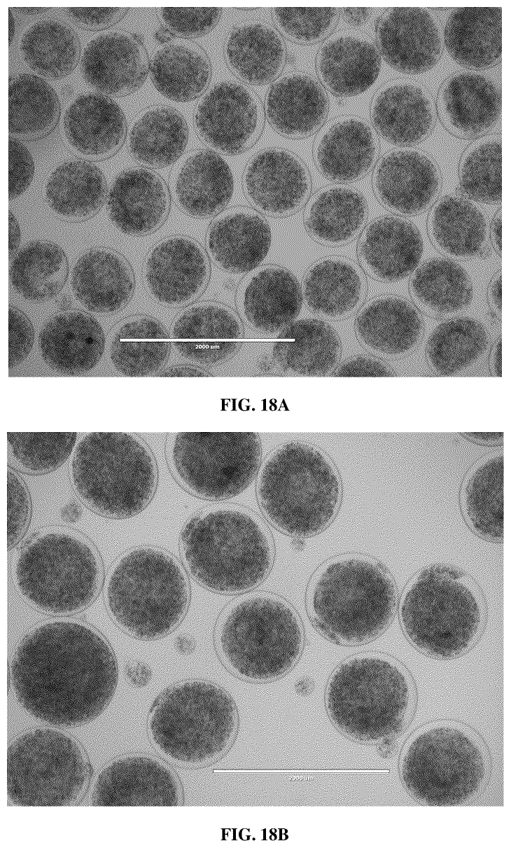

The degree of mouse macrophage adhesion in vivo over 1-4 weeks on hydrogel capsules with varying amounts (low, medium, or high) of chemical modification in the second (outer) compartment was examined as follows. Capsules were prepared as described in Example 10, and implanted in C57/BL6 mice as in Example 11, then retrieved at 1, 2, and 4 weeks post-implantation. At retrieval, particles were imaged for the presence of mouse macrophages using immunofluorescent staining (anti-F4/80). The results are shown in FIGS. 9A-9K.

After 1 week of implantation, macrophage adhesion was observed on the positive control capsules (unmodified medium molecular weight alginate) and the low conjugation capsules (FIGS. 9A-9B). Some macrophage adhesion was observed on the medium conjugation capsules (FIG. 9C), and there was minimal macrophage adhesion on the high conjugation (FIG. 9D), or empty capsules (FIG. 9E). At 2 and 4 weeks post-implantation, there was no macrophage adhesion on the empty capsules (FIGS. 9H and 9K), and macrophage adhesion was higher on the medium conjugation capsules (FIGS. 9F and 9I), compared to the high conjugation capsules (FIGS. 9G and 9J), demonstrating a dose response between the level of chemical modification in the second (outer) compartment of the capsule and macrophage adhesion (fibrosis) in vivo.

### Example 13: Effect of Varying the Level of Chemical Modification in the Second Compartment on Fibrosis

The level of chemical modification in the second (outer) compartment of exemplary particles was investigated for its impact on fibrosis using the method described below. The low, medium, and high conjugation capsules prepared in Example 10 were used. Additionally, medium-high (4.79% N) and double-high (9.00% N) conjugation capsules were prepared using methods described in Example 10, where % nitrogen is determined by combustion analysis and corresponds to the amount of small molecule conjugated to the polymer. The capsules were then implanted in C57/BL6 mice as described in Example 11, and retrieved at 2 weeks post-implantation. Fibrosis of the retrieved capsules was analyzed with brightfield imaging, where an opaque layer around the capsule indicates fibrosis. The results are shown in FIGS. 10A-10E.

Empty capsules with medium conjugation were used as a control, and showed no fibrosis (FIG. 10A). Capsules with medium conjugation or medium-high conjugation showed higher levels of fibrosis (FIGS. 10B-10C), compared with the high-conjugation capsules (FIG. 10D). Many of the double high conjugation capsules were not intact spheres after retrieval, some appeared fibrosed, or reduced in size, and there was significant capsule debris (FIG. 10E). This suggests that the second (outer) compartment containing the high levels of a compound of Formula (I) was not intact, demonstrating that there is an upper limit to the amount of a compound of Formula (I) that can effectively resist fibrosis.

### Example 14: Effect of Varying the Level of Chemical Modification in the Second Compartment on Mechanical Strength

The mechanical strength of the capsules used in Example 13 were measured by initial fracture using a texture analyzer. Each of the control, medium, medium high, high, and double high conjugated capsules were tested for mechanical strength at both pre- and post-implantation stages. The results are shown in FIG. 11.

Initial mechanical strength was higher in the control (empty) capsules compared to cell-loaded capsules. The medium, medium-high, and high conjugation capsules had similar strength at both pre- and post-implantation, with a decrease in strength observed after implantation. The capsules with the highest level of conjugation (double high) were the weakest capsules, and most of these capsules were not intact at retrieval (FIG. 11), as noted in Example 13. This further demonstrates that there is an upper limit to the amount of afibrotic small molecule conjugation that is efficacious, as very high levels of conjugation compromise mechanical strength.

### Example 15: Effect of Covalent and Non-Covalent Chemical Modification of the Second (Outer) Compartment on Fibrosis

The difference between conjugated or non-conjugated compounds of Formula (I) in the second (outer) compartment of hydrogel capsules as a means to confer resistance to fibrosis was determined using the following experiment.

Capsules with medium and high amounts of compounds of Formula (I) were prepared as in Example 10. In addition, a third type of capsule featuring non-conjugated afibrotic small molecules (denoted “amine added back”) was prepared in a similar manner to the method of Example 10, where the second (outer) compartment of the capsule was made from alginate solution containing unconjugated compounds of Formula (I). This solution was prepared by adding unconjugated small molecule to a medium conjugation alginate solution, using an amount of unconjugated small molecule necessary to achieve an overall amount of compounds of Formula (I) equivalent to the high conjugation alginate solution. The 70:30 CM-Alg:U-Alg was used for the first (inner) compartment of the “amine added back” capsules. All capsules were then implanted in vivo using C57/BL6 mice as in Example 11, and were retrieved at 2 weeks post-implantation. Retrieved capsules were imaged with brightfield microscopy to detect the presence of an opaque outer layer of adhered cells, indicating the beginning of fibrosis. The results are shown in FIGS. 12A-12C.

There was a layer of adhered cells observed on the medium conjugation capsules (FIG. 12A), while only minimal adhesion to the high conjugation capsules was observed (FIG. 12B). The “amine added back” capsules also appeared opaque, indicating a layer of fibrotic cell adhesion on the capsule (FIG. 12C). Therefore, this data demonstrates that only conjugated small molecules contribute to the afibrotic properties of the capsules.

### Example 16: Effect of Varying Level of Small Molecule Conjugation and Polymer Blend Ratio in the Second Compartment on Macrophage Adhesion

Capsules prepared from alginate solutions of two polymer blends (containing different ratios of low molecular weight alginate (CM-LMW) to unmodified high molecular weight alginate (U-HMW)), and varying amounts (medium, medium high, or high) of compounds of Formula (I) were compared in terms of their fibrosis resistance in vivo with the following experiment.

Three polymer blends were prepared at a 70% CM-LMW-Alg to 30% U-HMW-Alg ratio (70:30) as described in Example 3, with medium, medium high, or high levels of compounds of Formula (I). An additional three polymer blends were also prepared at a 60% CM-LMW-Alg to 40% U-HMW-Alg ratio (60:40), again with medium, medium high, or high levels of compounds of Formula (I). Each polymer blend was then used to form the second (outer) compartment of capsules using the method described in Example 10, providing a set of six hydrogel capsules, featuring medium, medium-high, or high-levels of conjugation from either 70:30 or 60:40 ratio polymer blends. The inner compartments of all the capsules contained the 70:30 CM-Alg:U-Alg solution. The capsules were then implanted in vivo, as described in Example 11, and were retrieved at 1 week post-implantation and analyzed via immunofluorescent staining for mouse macrophages adhesion (fibrosis) on the capsules. The results are shown in FIGS. 13A-13F

A trend of decreasing levels of adhered macrophages was observed from the medium to high conjugated capsules, across both the 70:30 (FIGS. 13A-13C) and the 60:40 ratio blends (FIGS. 13D-13F). Also, a larger amount of adhered macrophages was observed in the 60:40 ratio blend capsules, compared with the relatively lower amount of macrophage adhesion in the capsules form the 70:30 ratio blend. As the change in ratio affects the total dose of small molecules on the capsule, the results infer that the amount of small molecule conjugated to the polymer and the amount of chemically modified polymer used to prepare the capsules can independently alter macrophage adhesion and fibrosis.

### Example 17: Comparison of Capsule Architecture on the Fibrotic Response

Compositions of one-compartment or two-compartment hydrogel millicapsules encapsulating single ARPE-19 cells expressing FVIII-BDD were prepared by extruding droplets of the 70:30 CM-Alg:U-Alg solution described in Example 3 with various cell loading concentrations into a crosslinking solution. One-compartment capsules with either no cells or 5000 cells dispersed throughout the entire capsule were prepared as the controls. The two-compartment capsules had a cell-free second (outer) compartment and a first (inner) compartment containing 5000 or 2500 cells per capsule. Capsules with 2500 and 5000 cells were prepared using an outer:inner volume ratio of 50%:50%. Capsules with 2500 cells and a thicker second (outer) compartment were prepared using an outer:inner volume ratio of 75%:25%. The capsules were implanted into the IP space of C57/BL6 mice at 0.5 ml capsules/mouse. Capsules were retrieved after 14 days and imaged to observe presence or absence of fibrosis. In this mouse model, the xenogeneic ARPE-19 cells in the capsule were expected to induce a FBR.

The results of this experiment showed that all capsules encapsulating ARPE-19 cells, fabricated with either one or two compartments, were fibrosed in the C57/BL6 mouse model (data not shown). No fibrosis was observed on the empty one-compartment control capsules. Thus, in C57/BL6 mice, the configuration of capsules as one-compartment or two-compartments had no apparent effect on FBR induced by the xenogeneic RPE cells.

### Example 18: Assessing Cell Proliferation in Exemplary Particles Configured as Two-Compartment Hydrogel Capsules

HEK293F cells, which grow in suspension, were encapsulated within the inner compartment of two-compartment hydrogel millicapsules of about 1.5 mm in diameter. The capsules were prepared using the 70:30 CM-Alg:U-MW-Alg solution described in Example 3 to form both inner and outer compartments. The cell loading concentration in the inner compartment was 20 million HEK293F cells/ml (equivalent of 10 million cells/ml alginate solution in a 1.5 mm one-compartment capsule). Two-compartment capsules were fabricated using 5 ml/h inner and outer flow rates. As a control, one-compartment capsules of about 1.5 mm diameter were prepared using the same 70:30 CM-Alg:U-MW-Alg alginate solution comprising 10 million HEK293F cells/ml. Capsules were incubated at 37° C., 5% CO₂ for 7 days and then observed by microscopy.

Observation of the capsule edges revealed the presence of cells protruding from the edges of the one-compartment capsules, but not the two-compartment capsules, after one week of culture (FIGS. 14A-9B). Free floating and adhered cells were observed on the surface of the tissue culture plate containing the one-compartment capsules, demonstrating incomplete encapsulation and/or escape of cells (FIG. 14C). In contrast, no cells were observed outside of the two-compartment capsules in the culture plate after one week of culture (FIG. 14D). Similar results were obtained when one-compartment and two-compartment capsules were prepared with ARPE19 cells instead of HEK293F cells (data not shown). Thus, the two-compartment capsules are more effective than one-compartment capsules in containing cells, e.g., without cell leakage or protrusion through the capsule surface.

### Example 19: Assessing Protein Release from One- and Two-Compartment Capsules

To determine if protein molecules expressed by encapsulated cells can be released from two-compartment hydrogel capsules, engineered ARPE19 cells expressing FVIII-BDD were encapsulated in two-compartment capsules and protein secretion was measured after 24 hours.

The capsules were prepared using the 70:30 CM-alg:U-HMW-Alg solution described in Example 3 in each of the first and second syringes and 5 ml/h inner and outer flow rates. The alginate solution to prepare the inner compartment also contained ARPE19-FVIII at 20, 50 or 100 million cells/ml. Capsules were incubated for 24 h at 37° C. in a known volume of medium, and medium supernatant was collected and FVIII was detected by FVIII ELISA.

After 24 h, FVIII was detected in medium supernatant samples for each cell loading concentration. An increasing trend between cell loading concentration and amount of FVIII detected from the 2-compartment particles was observed (data not shown). These results suggest that FVIII expressed by cells encapsulated in the inner compartment of a two-compartment capsule diffuses through the second compartment and then exits the capsule.

### Example 20. Cell Concentration in Capsules can be Increased to Increase FIX Levels in Plasma and IP Fluid of Nude Mice

Compositions of two-compartment hydrogel millicapsules (1.5 mm) encapsulating single ARPE-19:FIX cells were prepared by extruding droplets of the 70:30 CM-Alg:U-Alg solution described in Example 3 with various cell loading concentrations into a crosslinking solution. Cells were encapsulated at 2, 20, 100, 300 million cells/ml alginate in the inner compartment (equivalent of 1, 10, 50, 150 million cells/ml alginate solution in a 1.5 mm one-compartment capsule). Capsules were implanted into the IP space of nude mice (0.5 ml/mouse) and were retrieved after 5 days. Levels of FIX in the plasma and IP fluid was measured by ELISA.

By varying the encapsulated cell concentration, the total number of encapsulated cells was varied from 500 to 39,000 cells/capsule as shown in FIG. 15A. At all cell concentrations, capsules were created with spherical morphology (data not shown). Some proliferation of cells was observed during implant (FIG. 15A). The levels of FIX in the plasma and IP fluid increased with increasing cell concentration, with the exception of 2 million cells/ml, which was at the lower limit of detection of the ELISA assay (FIG. 15. B, C and data not shown). There was an approximately 10-fold increase in FIX levels in the IP fluid, where the capsules are in the local environment, compared to the plasma. Capsules at all cell concentrations were retrieved intact (data not shown). Therefore, a dose response for FIX expression in both the IP and plasma was observed between 2 and 300 M cells/ml alginate solution used to form the inner compartment. In addition, the successful encapsulation of 39,000 cell/capsule is significantly greater than what has been reported in the scientific literature.

### Example 21: Optimal Cell Concentration Maximized FIX Levels and Maintained Capsule Integrity

Compositions of two-compartment hydrogel millicapsules encapsulating single ARPE-19:FIX cells were prepared as in the previous examples, with cells encapsulated at 100, 200, 300 and 646 million cells/ml of the alginate solution used to form the inner compartment (equivalent of 50, 100, 150, 323 million cells/ml alginate solution in a 1.5 mm one-compartment capsule). Capsules were implanted into the IP space of nude mice (0.5 ml/mouse) and were retrieved after 4 weeks. Levels of FIX in the IP fluid was measured by ELISA.

By varying the encapsulated cell concentration, the total number of encapsulated cells was varied from 24,000 to 54,000 cells/capsule (data not shown). At all cell concentrations, capsules were created with spherical morphology (data not shown). The levels of FIX in the IP fluid increased with increasing cell concentration to 150 million cells/ml (FIG. 16A). At the highest loading cell concentration (646 million cells/ml), the FIX levels were low and the retrieved capsules were no longer intact (FIG. 16B). The majority of the capsules prepared with 100 million cells/ml had greatest number of capsules retrieved intact (data not shown). This demonstrates that there is an optimal cell concentration where protein levels are maximized and capsules maintain their integrity.

### Example 22: Preparation of Two-Compartment Capsules with a Target Size of 0.75 mm Diameter or 1.0 mm Diameter

Two-compartment capsule compositions with a capsule target size of 1.0 mm or 0.75 mm were prepared as described in the previous examples, but with the following adjustments. The alginate solution used to form the second (outer) compartment contained CM-Alg-101 with a medium conjugation level of Compound 101 (e.g., prepared as described in Example 10) blended with U-HMW-Alg at a 70:30 ratio of CM-Alg-101 to U-HMW-Alg. The same blended alginate solution with a suspension of ARPE19 cells (50 million cells per ml) engineered to express human FVIII was used to form the first (inner) compartment. Capsule compositions with a capsule target size of 1.0 mm capsules were generated using a coaxial needle (20 G OD/26 G ID), a voltage of 7.3 kV, first and second compartment flow rates each of 5 mL/h, and a capsule droplet rate of 30-35 droplets per 10 seconds. Capsule compositions with a target size of 0.75 mm were prepared using a coaxial needle (20 G OD/26 G ID), a voltage of approximately 7.5 kV, first and second compartment flow rates each of 5 mL/h, and a capsule droplet rate of 35-40 droplets per 10 seconds.

As shown in FIGS. 18A-18B, spherical 0.75 mm and 1.0 mm two compartment capsules with cells in the inner compartment were produced. These capsules had outer compartments that completely encapsulated the cells in the inner compartment. Therefore, two-compartment capsules can be prepared at 0.75 and 1.0 mm mean diameters.

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1-44. (canceled)

45. A method of treating a subject in need of a substance comprising:
providing a particle; which comprises or has the ability to produce the substance; and
disposing the particle in the body of the subject;
wherein the particle comprises:
a) a first compartment;
b) a second compartment; and,
c) a compound of Formula (I-a): 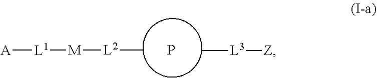
or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆-alkylene)-, —N(R^{C})C(O)(C₂-C₆-alkenylene)-, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F}), —Si(OR^{A})₂, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring, optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynl, heteroalkyl, halogen, cyano, oxo, hydroxyl, cycloalkyl, or heterocyclyl;
x is 1 or 2; and
y is 2, 3, or 4.

46. The method of claim 45, wherein the first compartment is surrounded by the second component.

47. The method of claim 45, wherein the differential volume of the second compartment is about 1%, 2%, 5%, 7.5%, 10%, 15%, 20%, 25%, 30%, 35%, 40%, 45%, 50%, 55%, 60%, 65%, or 75% less than the volume of the first compartment.

48. The method of claim 45, comprising a property selected from the following:
a) the first compartment comprises a compound of Formula (I-a);
b) the second compartment comprises a compound of Formula (I-a);
c) a compound of Formula (I-a) is disposed on the exterior surface of the particle; and/or
d) the particle comprises an interface between the first and second compartment and a compound of Formula (I-a) is disposed at the interface.

49. The method of claim 45, wherein the first compartment or the second compartment is substantially free of a compound of Formula (I-a).

50. The method of claim 45, comprising a property selected from the following:
a) the first compartment is substantially free of a compound of Formula (I-a);
b) the second compartment is substantially free of a compound of Formula (I-a);
c) the outer surface of the particle is substantially free of a compound of Formula (I-a); or
d) the particle comprises an interface between the first and second compartment and the interface is substantially free of a compound of Formula (I-a).

51. The method of claim 45, wherein the particle has a largest linear dimension (LLD) of between 1 millimeter to 5 millimeters.

52. The method of claim 45, wherein the particle comprises a cell.

53. The method of claim 45, wherein the first compartment comprises a cell.

54. The method of claim 45, wherein the first compartment comprises a cell and the second compartment does not comprise a cell.

55. The method of claim 52, wherein the particle comprises at least 5×10⁶, 10×10⁶, 15×10⁶ or 20×10⁶ cells per mL.

56. The method of claim 52, wherein:
a) one or a plurality of cells is disposed within the first compartment;
b) one or a plurality of cells is disposed within the second compartment;
c) the number or density of cells in the second compartment is at least 2, 5, 10, 10², 10³, or 10⁴ times less than the number of density of cells in the first compartment;
d) the first compartment comprises a compound of Formula (I-a); and/or,
e) the second compartment comprises a compound of Formula (I-a).

57. The method of claim 52, wherein the cell is an epithelial cell, endothelial cell, fibroblast cell, mesenchymal stem cell, or keratinocyte cell.

58. The method of claim 57, wherein the cell is an islet cell.

59. The method of claim 57, wherein the cell expresses a polypeptide.

60. The method of claim 59, wherein the polypeptide is insulin.

61. The method of claim 45, wherein the first compartment and the second compartment comprise a polymer.

62. The method of claim 61, wherein the polymer is selected from alginate, chitosan, hyaluronate, gelatin, poly(L-lactic acid) (PLLA), or poly(lactic glycolic acid) (PLGA).

63. The method of claim 45, wherein the compound of Formula (I-a) is a compound of any one of Formulas (I-b), (I-c), (I-d), (I-e), (I-f), (II), (II-a), (III), (III-a), (III-b), (III-c), or (III-d), or a pharmaceutically acceptable salt thereof.

64. The method of claim 45, wherein the compound of Formula (I-a) is selected from::

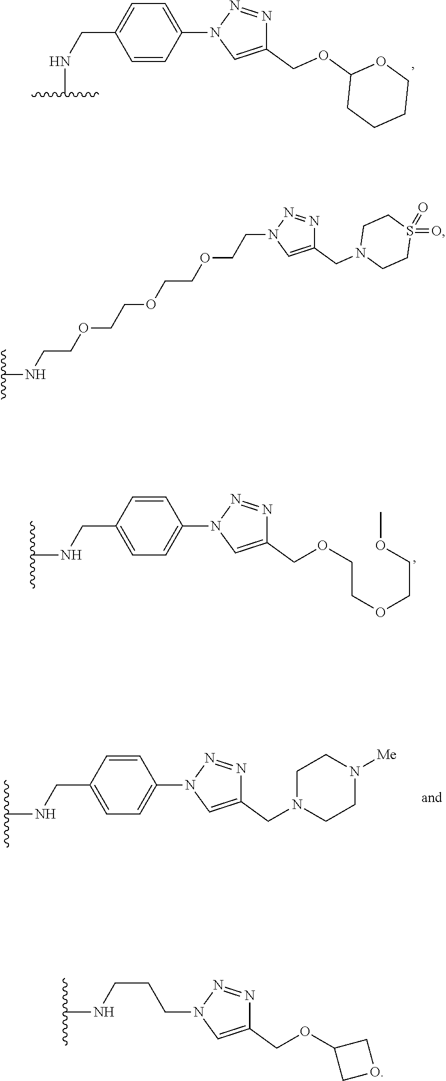 [SMILES: *NCc1ccc(-n2cc(COC3CCCCO3)nn2)cc1 ; *NCCOCCOCCOCCn1cc(CN2CCS(=O)(=O)CC2)nn1 ; *NCc1ccc(-n2cc(COCCOCCOC)nn2)cc1 ; *NCc1ccc(-n2cc(CN3CCN(C)CC3)nn2)cc1 ; *NCCCn1cc(COC2COC2)nn1]

65. The method of claim 45, wherein the method comprises a method of treating a disease, disorder, or condition in the subject.


SEQUENCE LISTING (36 sequences)

SEQ ID NO: 1 - protein, 1457 aa, synthetic construct
  REGION 1..19: note = Signal sequence
  REGION 1..1457: note = Description of Artificial Sequence: Synthetic polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 461 aa, synthetic construct
  REGION 1..28: note = Signal sequence
  REGION 1..461: note = Description of Artificial Sequence: Synthetic polypeptide
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 3 - protein, 1457 aa, synthetic construct
  REGION 1..19: note = Signal sequence
  REGION 1..1457: note = Description of Artificial Sequence: Synthetic polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKHHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - protein, 1457 aa, synthetic construct
  REGION 1..19: note = Signal sequence
  REGION 1..1457: note = Description of Artificial Sequence: Synthetic polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKAHQAEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - protein, 1453 aa, synthetic construct
  REGION 1..19: note = Signal sequence
  REGION 1..1453: note = Description of Artificial Sequence: Synthetic polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - protein, 1454 aa, synthetic construct
  REGION 1..19: note = Signal sequence
  REGION 1..1454: note = Description of Artificial Sequence: Synthetic polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 7 - protein, 1474 aa, synthetic construct [sequence omitted]

SEQ ID NO: 8 - DNA, 4374 bp, synthetic construct
  misc_feature 1..4374: note = Description of Artificial Sequence: Synthetic polynucleotide
  source 1..4374: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 9 - DNA, 4374 bp, synthetic construct
  misc_feature 1..4374: note = Description of Artificial Sequence: Synthetic polynucleotide
  source 1..4374: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaacaccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 10 - DNA, 4374 bp, synthetic construct
  misc_feature 1..4374: note = Description of Artificial Sequence: Synthetic polynucleotide
  source 1..4374: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaagcccatcaagcggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 11 - DNA, 4362 bp, synthetic construct
  misc_feature 1..4362: note = Description of Artificial Sequence: Synthetic polynucleotide
  source 1..4362: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaagaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 12 - DNA, 4365 bp, synthetic construct
  misc_feature 1..4365: note = Description of Artificial Sequence: Synthetic polynucleotide
  source 1..4365: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 13 - DNA, 4374 bp, synthetic construct
  misc_feature 1..4374: note = Description of Artificial Sequence: Synthetic polynucleotide
  source 1..4374: mol_type = other DNA
atgcagatcgagctgtctacctgcttcttcctgtgcctgctgcggttctgcttcagcgccaccagaagatattacctgggcgccgtggaactgagctgggactacatgcagtctgacctgggagagctgcccgtggacgctagatttcctccaagagtgcccaagagcttccccttcaacacctccgtggtgtacaagaaaaccctgttcgtggaattcaccgaccacctgttcaatatcgccaagcctcggcctccttggatgggactgctgggacctacaattcaggccgaggtgtacgacaccgtggtcatcaccctgaagaacatggccagccatcctgtgtctctgcacgccgtgggagtgtcttactggaaggcttctgagggcgccgagtacgacgatcagacaagccagagagagaaagaggacgacaaggttttccctggcggcagccacacctatgtctggcaggtcctgaaagaaaacggccctatggcctccgatcctctgtgcctgacatacagctacctgagccacgtggacctggtcaaggacctgaattctggcctgatcggagccctgctcgtgtgtagagaaggcagcctggccaaagagaaaacccagacactgcacaagttcatcctgctgttcgccgtgttcgacgagggcaagagctggcacagcgagacaaagaacagcctgatgcaggacagggatgccgcctctgctagagcttggcctaagatgcacaccgtgaacggctacgtgaacagaagcctgcctggactgatcggctgccacagaaagtccgtgtactggcacgtgatcggcatgggcacaacacctgaggtgcacagcatctttctggaaggacacaccttcctcgtgcggaaccatagacaggccagcctggaaatcagccctatcaccttcctgaccgctcagaccctgctgatggatctgggccagtttctgctgttctgccacatcagctcccaccagcacgatggcatggaagcctacgtgaaggtggacagctgccccgaagaaccccagctgcggatgaagaacaacgaggaagccgaggactacgacgacgacctgaccgactctgagatggacgtcgtcagattcgacgacgataacagccccagcttcatccagatcagaagcgtggccaagaagcaccccaagacctgggtgcactatatcgccgccgaggaagaggactgggattacgctcctctggtgctggcccctgacgacagaagctacaagagccagtacctgaacaacggccctcagcggatcggccggaagtataagaaagtgcggttcatggcctacaccgacgagacattcaagaccagagaggccatccagcacgagagcggaattctgggccctctgctgtatggcgaagtgggcgatacactgctgatcatcttcaagaaccaggccagcagaccctacaacatctaccctcacggcatcaccgatgtgcggcccctgtattctagaaggctgcccaagggcgtgaagcacctgaaggacttccctatcctgcctggcgagatcttcaagtacaagtggaccgtgaccgtggaagatggccccaccaagagcgaccctagatgtctgacacggtactacagcagcttcgtgaacatggaacgcgacctggccagcggcctgattggacctctgctgatctgctacaaagaaagcgtggaccagcggggcaaccagatcatgagcgacaagcggaacgtgatcctgtttagcgtgttcgatgagaaccggtcctggtatctgaccgagaacatccagcggtttctgcccaatcctgctggcgtgcagctggaagatcctgagttccaggcctccaacatcatgcactccatcaatggctatgtgttcgacagcctgcagctgagcgtgtgcctgcacgaagtggcctactggtacatcctgagcattggcgcccagaccgacttcctgtccgtgttctttagcggctacaccttcaagcacaagatggtgtacgaggataccctgacactgttcccattcagcggcgagacagtgttcatgagcatggaaaaccccggcctgtggatcctgggctgtcacaacagcgacttccggaacagaggcatgacagccctgctgaaggtgtccagctgcgacaagaacaccggcgactactacgaggacagctatgaggacatcagcgcctacctgctgagcaagaacaatgccatcgagcctcggagcttcagccagaatcctcctgtgctgaagcggcaccagcgcgagatcaccagaacaaccctgcagagcgaccaagaggaaatcgattacgacgacaccatcagcgtcgagatgaagaaagaagatttcgacatctacgacgaggacgagaatcagagccccagaagctttcagaaaaagacccggcactacttcattgccgccgtcgagagactgtgggactacggcatgtctagcagccctcacgtgctgagaaatagagcccagagcggcagcgtgccccagttcaagaaagtggtgttccaagagttcaccgacggcagcttcacccagccactgtatagaggcgagctgaacgagcatctgggcctgctgggcccttatatcagagccgaagtggaagataacatcatggtcaccttccggaatcaggcctctcggccctacagcttctacagctccctgatcagctacgaagaggaccagagacagggcgctgagcccagaaagaacttcgtgaagcccaacgagactaagacctacttttggaaggtgcagcaccacatggcccctacaaaggacgagttcgactgcaaggcctgggcctacttttccgatgtggatctggaaaaggacgtgcacagcgggctcatcggaccactgcttgtgtgccacaccaacacactgaaccccgctcacggcagacaagtgacagtgcaagagttcgccctgttcttcaccatcttcgacgaaacaaagagctggtacttcaccgagaatatggaacggaactgcagagccccttgcaacatccagatggaagatcccaccttcaaagagaactaccggttccacgccatcaacggctacatcatggacacactgcccggcctggttatggctcaggatcagagaatccggtggtatctgctgtccatgggctccaacgagaatatccacagcatccacttcagcggccacgtgttcaccgtgcggaaaaaagaagagtacaaaatggccctgtacaatctgtaccctggggtgttcgaaaccgtggaaatgctgccttccaaggccggcatttggagagtggaatgtctgattggagagcacctccacgccggaatgagcaccctgtttctggtgtacagcaacaagtgtcagacccctctcggcatggcctctggacacatcagagacttccagatcaccgcctctggccagtacggacagtgggctcctaaactggctcggctgcactacagcggcagcatcaatgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctacagaggcaacagcaccggcacactcatggtgttcttcggcaacgtggactccagcggcattaagcacaacatcttcaaccctccaatcattgcccggtacatccggctgcaccccacacactacagcatccggtctaccctgagaatggaactgatgggctgcgacctgaacagctgcagcatgcccctcggaatggaaagcaaggccatcagcgacgcccagatcacagccagcagctacttcaccaacatgttcgccacttggagcccctccaaggctagactgcatctgcagggcagaagcaacgcttggaggccccaagtgaacaaccccaaagagtggctgcaggtcgactttcaaaagaccatgaaagtgaccggcgtgaccacacagggcgtcaagtctctgctgacctctatgtacgtgaaagagttcctgatctccagcagccaggacggccatcagtggaccctgtttttccagaacggcaaagtgaaagtgttccagggcaatcaggacagcttcacacccgtggtcaactccctggatcctccactgctgaccagatacctgcggattcaccctcagtcttgggtgcaccagatcgctctgcggatggaagtgctgggctgtgaagctcaggacctctactga

SEQ ID NO: 14 - DNA, 4374 bp, synthetic construct
  misc_feature 1..4374: note = Description of Artificial Sequence: Synthetic polynucleotide
  source 1..4374: mol_type = other DNA
atgcagatcgagctgagcacctgcttcttcctgtgcctgctgcgcttctgcttcagcgccacccgccgctactacctgggcgccgtggagctgagctgggactacatgcagagcgacctgggcgagctgcccgtggacgcccgcttccccccccgcgtgcccaagagcttccccttcaacaccagcgtggtgtacaagaagaccctgttcgtggagttcaccgaccacctgttcaacatcgccaagccccgccccccctggatgggcctgctgggccccaccatccaggccgaggtgtacgacaccgtggtgatcaccctgaagaacatggccagccaccccgtgagcctgcacgccgtgggcgtgagctactggaaggccagcgagggcgccgagtacgacgaccagaccagccagcgcgagaaggaggacgacaaggtgttccccggcggcagccacacctacgtgtggcaggtgctgaaggagaacggccccatggccagcgaccccctgtgcctgacctacagctacctgagccacgtggacctggtgaaggacctgaacagcggcctgatcggcgccctgctggtgtgccgcgagggcagcctggccaaggagaagacccagaccctgcacaagttcatcctgctgttcgccgtgttcgacgagggcaagagctggcacagcgagaccaagaacagcctgatgcaggaccgcgacgccgccagcgcccgcgcctggcccaagatgcacaccgtgaacggctacgtgaaccgcagcctgcccggcctgatcggctgccaccgcaagagcgtgtactggcacgtgatcggcatgggcaccacccccgaggtgcacagcatcttcctggagggccacaccttcctggtgcgcaaccaccgccaggccagcctggagatcagccccatcaccttcctgaccgcccagaccctgctgatggacctgggccagttcctgctgttctgccacatcagcagccaccagcacgacggcatggaggcctacgtgaaggtggacagctgccccgaggagccccagctgcgcatgaagaacaacgaggaggccgaggactacgacgacgacctgaccgacagcgagatggacgtggtgcgcttcgacgacgacaacagccccagcttcatccagatccgcagcgtggccaagaagcaccccaagacctgggtgcactacatcgccgccgaggaggaggactgggactacgcccccctggtgctggcccccgacgaccgcagctacaagagccagtacctgaacaacggcccccagcgcatcggccgcaagtacaagaaggtgcgcttcatggcctacaccgacgagaccttcaagacccgcgaggccatccagcacgagagcggcatcctgggccccctgctgtacggcgaggtgggcgacaccctgctgatcatcttcaagaaccaggccagccgcccctacaacatctacccccacggcatcaccgacgtgcgccccctgtacagccgccgcctgcccaagggcgtgaagcacctgaaggacttccccatcctgcccggcgagatcttcaagtacaagtggaccgtgaccgtggaggacggccccaccaagagcgacccccgctgcctgacccgctactacagcagcttcgtgaacatggagcgcgacctggccagcggcctgatcggccccctgctgatctgctacaaggagagcgtggaccagcgcggcaaccagatcatgagcgacaagcgcaacgtgatcctgttcagcgtgttcgacgagaaccgcagctggtacctgaccgagaacatccagcgcttcctgcccaaccccgccggcgtgcagctggaggaccccgagttccaggccagcaacatcatgcacagcatcaacggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggacaccctgaccctgttccccttcagcggcgagaccgtgttcatgagcatggagaaccccggcctgtggatcctgggctgccacaacagcgacttccgcaaccgcggcatgaccgccctgctgaaggtgagcagctgcgacaagaacaccggcgactactacgaggacagctacgaggacatcagcgcctacctgctgagcaagaacaacgccatcgagccccgcagcttcagccagaacccccccgtgctgaagcgccaccagcgcgagatcacccgcaccaccctgcagagcgaccaggaggagatcgactacgacgacaccatcagcgtggagatgaagaaggaggacttcgacatctacgacgaggacgagaaccagagcccccgcagcttccagaagaagacccgccactacttcatcgccgccgtggagcgcctgtgggactacggcatgagcagcagcccccacgtgctgcgcaaccgcgcccagagcggcagcgtgccccagttcaagaaggtggtgttccaggagttcaccgacggcagcttcacccagcccctgtaccgcggcgagctgaacgagcacctgggcctgctgggcccctacatccgcgccgaggtggaggacaacatcatggtgaccttccgcaaccaggccagccgcccctacagcttctacagcagcctgatcagctacgaggaggaccagcgccagggcgccgagccccgcaagaacttcgtgaagcccaacgagaccaagacctacttctggaaggtgcagcaccacatggcccccaccaaggacgagttcgactgcaaggcctgggcctacttcagcgacgtggacctggagaaggacgtgcacagcggcctgatcggccccctgctggtgtgccacaccaacaccctgaaccccgcccacggccgccaggtgaccgtgcaggagttcgccctgttcttcaccatcttcgacgagaccaagagctggtacttcaccgagaacatggagcgcaactgccgcgccccctgcaacatccagatggaggaccccaccttcaaggagaactaccgcttccacgccatcaacggctacatcatggacaccctgcccggcctggtgatggcccaggaccagcgcatccgctggtacctgctgagcatgggcagcaacgagaacatccacagcatccacttcagcggccacgtgttcaccgtgcgcaagaaggaggagtacaagatggccctgtacaacctgtaccccggcgtgttcgagaccgtggagatgctgcccagcaaggccggcatctggcgcgtggagtgcctgatcggcgagcacctgcacgccggcatgagcaccctgttcctggtgtacagcaacaagtgccagacccccctgggcatggccagcggccacatccgcgacttccagatcaccgccagcggccagtacggccagtgggcccccaagctggcccgcctgcactacagcggcagcatcaacgcctggagcaccaaggagcccttcagctggatcaaggtggacctgctggcccccatgatcatccacggcatcaagacccagggcgcccgccagaagttcagcagcctgtacatcagccagttcatcatcatgtacagcctggacggcaagaagtggcagacctaccgcggcaacagcaccggcaccctgatggtgttcttcggcaacgtggacagcagcggcatcaagcacaacatcttcaacccccccatcatcgcccgctacatccgcctgcaccccacccactacagcatccgcagcaccctgcgcatggagctgatgggctgcgacctgaacagctgcagcatgcccctgggcatggagagcaaggccatcagcgacgcccagatcaccgccagcagctacttcaccaacatgttcgccacctggagccccagcaaggcccgcctgcacctgcagggccgcagcaacgcctggcgcccccaggtgaacaaccccaaggagtggctgcaggtggacttccagaagaccatgaaggtgaccggcgtgaccacccagggcgtgaagagcctgctgaccagcatgtacgtgaaggagttcctgatcagcagcagccaggacggccaccagtggaccctgttcttccagaacggcaaggtgaaggtgttccagggcaaccaggacagcttcacccccgtggtgaacagcctggacccccccctgctgacccgctacctgcgcatccacccccagagctgggtgcaccagatcgccctgcgcatggaggtgctgggctgcgaggcccaggacctgtactga

SEQ ID NO: 15 - DNA, 4374 bp, synthetic construct
  misc_feature 1..4374: note = Description of Artificial Sequence: Synthetic polynucleotide
  source 1..4374: mol_type = other DNA
atgcagattgagctgagcacctgtttcttcctgtgcctgctgagattttgcttctcagctacccgcaggtactacctgggagccgttgagctgtcctgggattacatgcagtcagatctgggggagctgcctgtggacgctcggtttccccccagagtgccaaagtcctttcccttcaacaccagcgtggtgtacaaaaagacactttttgttgaatttactgaccacttgttcaacatcgccaagccacgacccccatggatgggcctgctggggccaaccattcaggcagaggtttacgacacagtcgtgatcacactgaagaacatggcctcccatccagtgtctctgcacgccgtcggtgtgtcctactggaaagcatccgagggcgccgagtatgacgaccagaccagccagagagagaaagaggacgacaaagtgttccctggaggcagccacacctacgtgtggcaggtgttgaaggaaaatgggcccatggccagtgaccctttgtgtctgacttactcatacctgtctcatgtggatctagtcaaggacctgaattctggactgattggggcactgcttgtgtgccgcgaaggcagcctggccaaagaaaagacacagacccttcacaagttcatcctgctgttcgccgtgttcgacgaaggcaaatcctggcactcagaaaccaaaaactcactgatgcaggaccgggatgccgcctctgcccgcgcatggccaaaaatgcacaccgtcaacggctatgtcaatagaagtttgcccggcctcattggatgtcacaggaaaagcgtctattggcatgtaatcgggatgggaaccacacctgaggtccacagcatatttctggaaggccacacatttctggtgagaaatcatcgccaggcttccctggaaatttcccccatcaccttcttgaccgcccagacactgctcatggatcttgggcagtttctgctgttttgtcatatttcttctcaccaacacgacggaatggaggcctacgttaaggtcgatagttgccctgaagaacctcagctgaggatgaagaacaacgaggaagccgaggactacgatgacgatttgaccgattccgaaatggacgtggtgcgctttgatgatgacaattctccatccttcattcagattagatccgtcgccaagaagcaccccaagacctgggtgcactacattgcagccgaggaggaggattgggactacgcccccctggtgctggcacccgacgaccgaagctacaaatctcagtacctgaacaatggtccacaacggatcggcaggaagtacaagaaagtgcggttcatggcctatacagacgaaaccttcaaaaccagggaggctatccagcacgagtctgggattctgggaccactcctgtacggcgaagtgggcgacaccttgttaattatcttcaagaaccaggctagtagaccttataacatttatccccacggcattaccgatgtgcggcctctctactctaggcggcttccaaagggggtgaaacacctgaaggactttcccatcctccctggcgaaatctttaagtataagtggacagtgaccgtggaggatggaccaaccaagagcgaccccaggtgcctgacacgctattattcaagcttcgtgaatatggaaagggacctcgcatctggcttgatcggccctctgctgatatgttacaaggaaagcgtcgatcagagaggaaatcagatcatgtcagacaaaaggaatgtgatcctgttctccgtcttcgatgaaaacaggagctggtatctgacagagaacatccagagattcctgccaaatcccgccggcgtccagctggaggacccggagtttcaggcatctaacatcatgcattccattaatggttacgtgttcgactccctgcagctgagcgtgtgcctccacgaggtggcctactggtacatcttgagcatcggcgcccagaccgactttctgagcgtctttttctccgggtatactttcaaacataagatggtgtacgaagatactctgacgctgttccctttctctggggagactgtgtttatgtctatggagaaccctggactgtggattctcggatgccacaacagtgactttcgtaatagagggatgactgcactgctgaaggtgtccagctgtgataaaaatactggcgactactacgaagatagctatgaggatatctcagcatacctgctgagcaagaataacgccatcgagccccgaagcttctcacagaatccccctgtcctcaagaggcaccagcgagagatcacaaggaccacactccagtccgaccaggaggagattgactacgatgacacgatttctgtggagatgaaaaaagaggactttgacatctacgatgaggatgaaaaccagagccctaggtcgttccagaagaaaacaaggcactacttcattgccgccgtggagagactgtgggactacggaatgagtagttccccacacgtgttgcggaacagagcccagagtgggtccgtcccacagttcaagaaggttgttttccaggagttcacagatggctccttcactcagccactgtatcgcggcgagctgaatgagcacttgggcttattgggcccctacattcgcgcagaagtcgaagataatattatggtgaccttccgcaaccaggccagccggccttactcattctactcctctctcatctcttatgaggaggatcagcgccagggcgccgaaccccggaagaactttgtgaagcccaatgaaaccaaaacttacttttggaaggtgcagcaccatatggcgccgacgaaagacgaatttgactgcaaagcctgggcctacttcagcgacgtcgacttggagaaggacgtccacagcggcctgattggccctttgttggtctgccataccaatacactcaaccctgcccacgggaggcaggtgaccgtgcaggagtttgccttgttcttcaccatcttcgacgaaaccaagagctggtacttcacagagaacatggagaggaactgcagagcaccctgtaacatccagatggaggaccctactttcaaggaaaattacaggttccatgccattaatggctacatcatggataccctccccgggcttgtgatggctcaggaccagcgcatccgctggtacctgctctcaatgggctccaacgagaacattcatagcatccactttagtggccacgtgtttaccgtgcgcaagaaggaggagtacaagatggcactgtacaacctgtaccctggcgtgtttgagacagtggagatgctgccatccaaggccggcatctggcgcgtggagtgcctcattggggagcacctccatgctggcatgtctacactgttcctggtgtacagcaacaagtgtcagactccactcggaatggcctccgggcatatccgcgattttcagatcacggcctctggccagtatggccaatgggctcccaagctggccaggctgcactacagtgggagtatcaacgcttggagcaccaaggagcctttctcctggatcaaggtggacctgcttgcccccatgattattcacggcattaagacacagggggccaggcagaaattctcctccctgtacatctcccagttcatcatcatgtacagtctggacggcaaaaagtggcagacctaccgcgggaacagtaccgggacattgatggtgttcttcgggaacgtggactctagcggcattaaacacaacattttcaacccccccatcattgctaggtatatcaggctccatcccacccactatagcatcaggtccactctgcggatggagctgatgggctgcgaccttaattcatgcagcatgccgctgggcatggagtcaaaggccatctccgacgcccaaatcaccgcctccagctacttcaccaatatgttcgccacctggagccccagcaaggcccggctgcacctgcagggccgcagcaacgcctggcggcctcaggtgaacaaccccaaggagtggctgcaggtggacttccagaaaaccatgaaggtgactggggtcaccacccagggagtcaagagcctgctgaccagcatgtatgtgaaggagttcttgatcagctcgtcacaggatggccaccagtggactttgttctttcagaacggtaaggtgaaagtgttccagggaaaccaagattcctttacaccagtggtcaactctctggatcctcccctgctgacacggtacctgcggatccatccccagtcatgggtgcaccagattgctctgcgcatggaggtgcttggctgcgaggcccaggacctgtactga

SEQ ID NO: 16 - DNA, 4374 bp, synthetic construct
  misc_feature 1..4374: note = Description of Artificial Sequence: Synthetic polynucleotide
  source 1..4374: mol_type = other DNA
atgcagattgagctgagcacctgtttcttcctgtgcctgctgagattttgcttctcagctacccgcaggtactacctgggagccgttgagctgtcctgggattacatgcagtcagatctgggggagctgcctgtggacgctcggtttccccccagagtgccaaagtcctttcccttcaacaccagcgtggtgtacaaaaagacactttttgttgaatttactgaccacttgttcaacatcgccaagccacgacccccatggatgggcctgctggggccaaccattcaggcagaggtttacgacacagtcgtgatcacactgaagaacatggcctcccatccagtgtctctgcacgccgtcggtgtgtcctactggaaagcatccgagggcgccgagtatgacgaccagaccagccagagagagaaagaggacgacaaagtgttccctggaggcagccacacctacgtgtggcaggtgttgaaggaaaatgggcccatggccagtgaccctttgtgtctgacttactcatacctgtctcatgtggatctagtcaaggacctgaattctggactgattggggcactgcttgtgtgccgcgaaggcagcctggccaaagaaaagacacagacccttcacaagttcatcctgctgttcgccgtgttcgacgaaggcaaatcctggcactcagaaaccaaaaactcactgatgcaggaccgggatgccgcctctgcccgcgcatggccaaaaatgcacaccgtcaacggctatgtcaatagaagtttgcccggcctcattggatgtcacaggaaaagcgtctattggcatgtaatcgggatgggaaccacacctgaggtccacagcatatttctggaaggccacacatttctggtgagaaatcatcgccaggcttccctggaaatttcccccatcaccttcttgaccgcccagacactgctcatggatcttgggcagtttctgctgttttgtcatatttcttctcaccaacacgacggaatggaggcctacgttaaggtcgatagttgccctgaagaacctcagctgaggatgaagaacaacgaggaagccgaggactacgatgacgatttgaccgattccgaaatggacgtggtgcgctttgatgatgacaattctccatccttcattcagattagatccgtcgccaagaagcaccccaagacctgggtgcactacattgcagccgaggaggaggattgggactacgcccccctggtgctggcacccgacgaccgaagctacaaatctcagtacctgaacaatggtccacaacggatcggcaggaagtacaagaaagtgcggttcatggcctatacagacgaaaccttcaaaaccagggaggctatccagcacgagtctgggattctgggaccactcctgtacggcgaagtgggcgacaccttgttaattatcttcaagaaccaggctagtagaccttataacatttatccccacggcattaccgatgtgcggcctctctactctaggcggcttccaaagggggtgaaacacctgaaggactttcccatcctccctggcgaaatctttaagtataagtggacagtgaccgtggaggatggaccaaccaagagcgaccccaggtgcctgacacgctattattcaagcttcgtgaatatggaaagggacctcgcatctggcttgatcggccctctgctgatatgttacaaggaaagcgtcgatcagagaggaaatcagatcatgtcagacaaaaggaatgtgatcctgttctccgtcttcgatgaaaacaggagctggtatctgacagagaacatccagagattcctgccaaatcccgccggcgtccagctggaggacccggagtttcaggcatctaacatcatgcattccattaatggttacgtgttcgactccctgcagctgagcgtgtgcctccacgaggtggcctactggtacatcttgagcatcggcgcccagaccgactttctgagcgtctttttctccgggtatactttcaaacataagatggtgtacgaagatactctgacgctgttccctttctctggggagactgtgtttatgtctatggagaaccctggactgtggattctcggatgccacaacagtgactttcgtaatagagggatgactgcactgctgaaggtgtccagctgtgataaaaatactggcgactactacgaagatagctatgaggatatctcagcatacctgctgagcaagaataacgccatcgagccccgaagcttctcacagaatccccctgtcctcaaggcccaccaggcggagatcacaaggaccacactccagtccgaccaggaggagattgactacgatgacacgatttctgtggagatgaaaaaagaggactttgacatctacgatgaggatgaaaaccagagccctaggtcgttccagaagaaaacaaggcactacttcattgccgccgtggagagactgtgggactacggaatgagtagttccccacacgtgttgcggaacagagcccagagtgggtccgtcccacagttcaagaaggttgttttccaggagttcacagatggctccttcactcagccactgtatcgcggcgagctgaatgagcacttgggcttattgggcccctacattcgcgcagaagtcgaagataatattatggtgaccttccgcaaccaggccagccggccttactcattctactcctctctcatctcttatgaggaggatcagcgccagggcgccgaaccccggaagaactttgtgaagcccaatgaaaccaaaacttacttttggaaggtgcagcaccatatggcgccgacgaaagacgaatttgactgcaaagcctgggcctacttcagcgacgtcgacttggagaaggacgtccacagcggcctgattggccctttgttggtctgccataccaatacactcaaccctgcccacgggaggcaggtgaccgtgcaggagtttgccttgttcttcaccatcttcgacgaaaccaagagctggtacttcacagagaacatggagaggaactgcagagcaccctgtaacatccagatggaggaccctactttcaaggaaaattacaggttccatgccattaatggctacatcatggataccctccccgggcttgtgatggctcaggaccagcgcatccgctggtacctgctctcaatgggctccaacgagaacattcatagcatccactttagtggccacgtgtttaccgtgcgcaagaaggaggagtacaagatggcactgtacaacctgtaccctggcgtgtttgagacagtggagatgctgccatccaaggccggcatctggcgcgtggagtgcctcattggggagcacctccatgctggcatgtctacactgttcctggtgtacagcaacaagtgtcagactccactcggaatggcctccgggcatatccgcgattttcagatcacggcctctggccagtatggccaatgggctcccaagctggccaggctgcactacagtgggagtatcaacgcttggagcaccaaggagcctttctcctggatcaaggtggacctgcttgcccccatgattattcacggcattaagacacagggggccaggcagaaattctcctccctgtacatctcccagttcatcatcatgtacagtctggacggcaaaaagtggcagacctaccgcgggaacagtaccgggacattgatggtgttcttcgggaacgtggactctagcggcattaaacacaacattttcaacccccccatcattgctaggtatatcaggctccatcccacccactatagcatcaggtccactctgcggatggagctgatgggctgcgaccttaattcatgcagcatgccgctgggcatggagtcaaaggccatctccgacgcccaaatcaccgcctccagctacttcaccaatatgttcgccacctggagccccagcaaggcccggctgcacctgcagggccgcagcaacgcctggcggcctcaggtgaacaaccccaaggagtggctgcaggtggacttccagaaaaccatgaaggtgactggggtcaccacccagggagtcaagagcctgctgaccagcatgtatgtgaaggagttcttgatcagctcgtcacaggatggccaccagtggactttgttctttcagaacggtaaggtgaaagtgttccagggaaaccaagattcctttacaccagtggtcaactctctggatcctcccctgctgacacggtacctgcggatccatccccagtcatgggtgcaccagattgctctgcgcatggaggtgcttggctgcgaggcccaggacctgtactga

SEQ ID NO: 17 - DNA, 4425 bp, synthetic construct
  misc_feature 1..4425: note = Description of Artificial Sequence: Synthetic polynucleotide
  source 1..4425: mol_type = other DNA
atgcagattgagctgagcacctgtttcttcctgtgcctgctgagattttgcttctcagctacccgcaggtactacctgggagccgttgagctgtcctgggattacatgcagtcagatctgggggagctgcctgtggacgctcggtttccccccagagtgccaaagtcctttcccttcaacaccagcgtggtgtacaaaaagacactttttgttgaatttactgaccacttgttcaacatcgccaagccacgacccccatggatgggcctgctggggccaaccattcaggcagaggtttacgacacagtcgtgatcacactgaagaacatggcctcccatccagtgtctctgcacgccgtcggtgtgtcctactggaaagcatccgagggcgccgagtatgacgaccagaccagccagagagagaaagaggacgacaaagtgttccctggaggcagccacacctacgtgtggcaggtgttgaaggaaaatgggcccatggccagtgaccctttgtgtctgacttactcatacctgtctcatgtggatctagtcaaggacctgaattctggactgattggggcactgcttgtgtgccgcgaaggcagcctggccaaagaaaagacacagacccttcacaagttcatcctgctgttcgccgtgttcgacgaaggcaaatcctggcactcagaaaccaaaaactcactgatgcaggaccgggatgccgcctctgcccgcgcatggccaaaaatgcacaccgtcaacggctatgtcaatagaagtttgcccggcctcattggatgtcacaggaaaagcgtctattggcatgtaatcgggatgggaaccacacctgaggtccacagcatatttctggaaggccacacatttctggtgagaaatcatcgccaggcttccctggaaatttcccccatcaccttcttgaccgcccagacactgctcatggatcttgggcagtttctgctgttttgtcatatttcttctcaccaacacgacggaatggaggcctacgttaaggtcgatagttgccctgaagaacctcagctgaggatgaagaacaacgaggaagccgaggactacgatgacgatttgaccgattccgaaatggacgtggtgcgctttgatgatgacaattctccatccttcattcagattagatccgtcgccaagaagcaccccaagacctgggtgcactacattgcagccgaggaggaggattgggactacgcccccctggtgctggcacccgacgaccgaagctacaaatctcagtacctgaacaatggtccacaacggatcggcaggaagtacaagaaagtgcggttcatggcctatacagacgaaaccttcaaaaccagggaggctatccagcacgagtctgggattctgggaccactcctgtacggcgaagtgggcgacaccttgttaattatcttcaagaaccaggctagtagaccttataacatttatccccacggcattaccgatgtgcggcctctctactctaggcggcttccaaagggggtgaaacacctgaaggactttcccatcctccctggcgaaatctttaagtataagtggacagtgaccgtggaggatggaccaaccaagagcgaccccaggtgcctgacacgctattattcaagcttcgtgaatatggaaagggacctcgcatctggcttgatcggccctctgctgatatgttacaaggaaagcgtcgatcagagaggaaatcagatcatgtcagacaaaaggaatgtgatcctgttctccgtcttcgatgaaaacaggagctggtatctgacagagaacatccagagattcctgccaaatcccgccggcgtccagctggaggacccggagtttcaggcatctaacatcatgcattccattaatggttacgtgttcgactccctgcagctgagcgtgtgcctccacgaggtggcctactggtacatcttgagcatcggcgcccagaccgactttctgagcgtctttttctccgggtatactttcaaacataagatggtgtacgaagatactctgacgctgttccctttctctggggagactgtgtttatgtctatggagaaccctggactgtggattctcggatgccacaacagtgactttcgtaatagagggatgactgcactgctgaaggtgtccagctgtgataaaaatactggcgactactacgaagatagctatgaggatatctcagcatacctgctgagcaagaataacgccatcgagccccgaagcttctcacagaatgccaccaacgtgagcaacaacagcaacaccagcaacgacagcaacgtgagcccccctgtcctcaaggcccaccaggcggagatcacaaggaccacactccagtccgaccaggaggagattgactacgatgacacgatttctgtggagatgaaaaaagaggactttgacatctacgatgaggatgaaaaccagagccctaggtcgttccagaagaaaacaaggcactacttcattgccgccgtggagagactgtgggactacggaatgagtagttccccacacgtgttgcggaacagagcccagagtgggtccgtcccacagttcaagaaggttgttttccaggagttcacagatggctccttcactcagccactgtatcgcggcgagctgaatgagcacttgggcttattgggcccctacattcgcgcagaagtcgaagataatattatggtgaccttccgcaaccaggccagccggccttactcattctactcctctctcatctcttatgaggaggatcagcgccagggcgccgaaccccggaagaactttgtgaagcccaatgaaaccaaaacttacttttggaaggtgcagcaccatatggcgccgacgaaagacgaatttgactgcaaagcctgggcctacttcagcgacgtcgacttggagaaggacgtccacagcggcctgattggccctttgttggtctgccataccaatacactcaaccctgcccacgggaggcaggtgaccgtgcaggagtttgccttgttcttcaccatcttcgacgaaaccaagagctggtacttcacagagaacatggagaggaactgcagagcaccctgtaacatccagatggaggaccctactttcaaggaaaattacaggttccatgccattaatggctacatcatggataccctccccgggcttgtgatggctcaggaccagcgcatccgctggtacctgctctcaatgggctccaacgagaacattcatagcatccactttagtggccacgtgtttaccgtgcgcaagaaggaggagtacaagatggcactgtacaacctgtaccctggcgtgtttgagacagtggagatgctgccatccaaggccggcatctggcgcgtggagtgcctcattggggagcacctccatgctggcatgtctacactgttcctggtgtacagcaacaagtgtcagactccactcggaatggcctccgggcatatccgcgattttcagatcacggcctctggccagtatggccaatgggctcccaagctggccaggctgcactacagtgggagtatcaacgcttggagcaccaaggagcctttctcctggatcaaggtggacctgcttgcccccatgattattcacggcattaagacacagggggccaggcagaaattctcctccctgtacatctcccagttcatcatcatgtacagtctggacggcaaaaagtggcagacctaccgcgggaacagtaccgggacattgatggtgttcttcgggaacgtggactctagcggcattaaacacaacattttcaacccccccatcattgctaggtatatcaggctccatcccacccactatagcatcaggtccactctgcggatggagctgatgggctgcgaccttaattcatgcagcatgccgctgggcatggagtcaaaggccatctccgacgcccaaatcaccgcctccagctacttcaccaatatgttcgccacctggagccccagcaaggcccggctgcacctgcagggccgcagcaacgcctggcggcctcaggtgaacaaccccaaggagtggctgcaggtggacttccagaaaaccatgaaggtgactggggtcaccacccagggagtcaagagcctgctgaccagcatgtatgtgaaggagttcttgatcagctcgtcacaggatggccaccagtggactttgttctttcagaacggtaaggtgaaagtgttccagggaaaccaagattcctttacaccagtggtcaactctctggatcctcccctgctgacacggtacctgcggatccatccccagtcatgggtgcaccagattgctctgcgcatggaggtgcttggctgcgaggcccaggacctgtactaa

SEQ ID NO: 18 - DNA, 1386 bp, synthetic construct
  misc_feature 1..1386: note = Description of Artificial Sequence: Synthetic polynucleotide
  source 1..1386: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctgtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 19 - DNA, 1386 bp, synthetic construct
  misc_feature 1..1386: note = Description of Artificial Sequence: Synthetic polynucleotide
  source 1..1386: mol_type = other DNA
atgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaagctggaagagttcgtgcagggcaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggacgacatcaacagctacgagtgctggtgccccttcggcttcgagggcaagaattgcgagctggacgtgacctgcaacatcaagaacggcagatgcgagcagttctgcaagaacagcgccgacaacaaggtcgtgtgctcctgcacagagggctacagactggccgagaaccagaagtcttgcgagcccgctgtgccctttccatgtggcagagtgtctgtgtcccagaccagcaagctgaccagagccgagacagtgttccccgacgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggcgagcacaacatcgaggaaacagagcacaccgagcagaaacggaacgtgatcagaatcatccctcaccacaactacaacgccgccatcaacaagtacaaccacgatatcgccctgctggaactggacgagcccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcaaggcgattctggcggccctcacgtgacagaggttgagggcacaagctttctgaccggcatcatcagctggggcgaagagtgtgccatgaaggggaagtacggcatctacaccaaggtgtccagatacgtgaactggatcaaagaaaagaccaagctcacctga

SEQ ID NO: 20 - DNA, 1386 bp, synthetic construct
  misc_feature 1..1386: note = Description of Artificial Sequence: Synthetic polynucleotide
  source 1..1386: mol_type = other DNA
atgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgagactgtgttccccgacgtggactacgtgaacagcaccgaggccgaaacgatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggaaaccggcgtgaagatcaccgtggtggccggcgagcacaacatcgaggaaaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaaaaccaagctgacctaa

SEQ ID NO: 21 - DNA, 1386 bp, synthetic construct
  misc_feature 1..1386: note = Description of Artificial Sequence: Synthetic polynucleotide
  source 1..1386: mol_type = other DNA
atgcagcgggtgaacatgatcatggccgagagccccgggctgatcaccatctgtctgctggggtacctgctgtccgccgagtgcaccgtgttcctggaccacgagaacgccaacaagatcctgaatcgccccaagagatacaattccggaaagctggaagagtttgtgcagggcaacctggagagagagtgcatggaagagaagtgctccttcgaggaggcccgggaggtgttcgagaatactgaacggacaacagagttctggaagcagtatgtggacggcgaccagtgtgagagcaacccctgtctgaacggcgggagctgcaaggacgacattaattcctacgaatgctggtgcccattcggcttcgagggcaagaactgcgagctggacgtgacctgcaacatcaagaacggccgctgcgagcagttttgcaagaactccgccgacaacaaggtggtgtgttcttgcaccgagggctaccgcctggccgaaaaccagaagagctgtgagcctgccgtgcccttcccctgcggccgggtgtctgtgtcccagacctccaagctgaccagagccgaaaccgtgtttccagatgtggactacgtgaatagcaccgaggccgagactatcctcgacaacatcacccagtccacccagagctttaacgacttcacccgcgtggtgggcggcgaggacgccaagcccggccagttcccctggcaggtggtgctcaacggaaaggtggacgccttctgcggaggcagcatcgtgaatgaaaagtggatcgtgacagccgcccactgcgtggaaacaggggtgaagatcaccgtggtggctggagagcacaacatcgaggagacagagcacaccgaacagaagaggaatgtgatcaggatcatcccccaccacaactataatgccgccatcaacaagtacaaccacgacatcgccctgctggagctggatgagcccctggtgctcaacagctacgtgacccccatctgcatcgctgacaaggagtacaccaacatcttcctgaagttcggctccggctacgtgtctggctggggccgcgtgttccacaagggaagaagcgccctcgtgctgcagtacctgcgggtgccactggtggacagggccacctgcctgctgagcactaagttcaccatttacaacaacatgttctgcgccggcttccacgagggcggcagggactcctgccagggcgacagcggcggcccccatgtgaccgaggtggagggcacctcctttctgactggcattatctcctggggcgaggagtgcgccatgaaggggaagtatggcatctacaccaaggtgtcccgctacgtgaactggattaaggagaaaaccaagctgacctga

SEQ ID NO: 22 - DNA, 313 bp, synthetic construct
  misc_feature 1..313: note = Description of Artificial Sequence: Synthetic polynucleotide
  source 1..313: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 23 - DNA, 1733 bp, synthetic construct
  misc_feature 1..1733: note = Description of Artificial Sequence: Synthetic polynucleotide
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 24 - DNA, 522 bp, synthetic construct
  misc_feature 1..522: note = Description of Artificial Sequence: Synthetic polynucleotide
  source 1..522: mol_type = other DNA
tcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 25 - DNA, 235 bp, synthetic construct
  misc_feature 1..235: note = Description of Artificial Sequence: Synthetic polynucleotide
  source 1..235: mol_type = other DNA
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaa

SEQ ID NO: 26 - DNA, 6164 bp, synthetic construct
  misc_feature 1..6164: note = Description of Artificial Sequence: Synthetic polynucleotide
  source 1..6164: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccgaattcgcggccgctaaacccagctttcttgtacaaagtggcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 27 - DNA, 6721 bp, synthetic construct
  misc_feature 1..6721: note = Description of Artificial Sequence: Synthetic polynucleotide
  source 1..6721: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgcagattgagctgagcacctgtttcttcctgtgcctgctgagattttgcttctcagctacccgcaggtactacctgggagccgttgagctgtcctgggattacatgcagtcagatctgggggagctgcctgtggacgctcggtttccccccagagtgccaaagtcctttcccttcaacaccagcgtggtgtacaaaaagacactttttgttgaatttactgaccacttgttcaacatcgccaagccacgacccccatggatgggcctgctggggccaaccattcaggcagaggtttacgacacagtcgtgatcacactgaagaacatggcctcccatccagtgtctctgcacgccgtcggtgtgtcctactggaaagcatccgagggcgccgagtatgacgaccagaccagccagagagagaaagaggacgacaaagtgttccctggaggcagccacacctacgtgtggcaggtgttgaaggaaaatgggcccatggccagtgaccctttgtgtctgacttactcatacctgtctcatgtggatctagtcaaggacctgaattctggactgattggggcactgcttgtgtgccgcgaaggcagcctggccaaagaaaagacacagacccttcacaagttcatcctgctgttcgccgtgttcgacgaaggcaaatcctggcactcagaaaccaaaaactcactgatgcaggaccgggatgccgcctctgcccgcgcatggccaaaaatgcacaccgtcaacggctatgtcaatagaagtttgcccggcctcattggatgtcacaggaaaagcgtctattggcatgtaatcgggatgggaaccacacctgaggtccacagcatatttctggaaggccacacatttctggtgagaaatcatcgccaggcttccctggaaatttcccccatcaccttcttgaccgcccagacactgctcatggatcttgggcagtttctgctgttttgtcatatttcttctcaccaacacgacggaatggaggcctacgttaaggtcgatagttgccctgaagaacctcagctgaggatgaagaacaacgaggaagccgaggactacgatgacgatttgaccgattccgaaatggacgtggtgcgctttgatgatgacaattctccatccttcattcagattagatccgtcgccaagaagcaccccaagacctgggtgcactacattgcagccgaggaggaggattgggactacgcccccctggtgctggcacccgacgaccgaagctacaaatctcagtacctgaacaatggtccacaacggatcggcaggaagtacaagaaagtgcggttcatggcctatacagacgaaaccttcaaaaccagggaggctatccagcacgagtctgggattctgggaccactcctgtacggcgaagtgggcgacaccttgttaattatcttcaagaaccaggctagtagaccttataacatttatccccacggcattaccgatgtgcggcctctctactctaggcggcttccaaagggggtgaaacacctgaaggactttcccatcctccctggcgaaatctttaagtataagtggacagtgaccgtggaggatggaccaaccaagagcgaccccaggtgcctgacacgctattattcaagcttcgtgaatatggaaagggacctcgcatctggcttgatcggccctctgctgatatgttacaaggaaagcgtcgatcagagaggaaatcagatcatgtcagacaaaaggaatgtgatcctgttctccgtcttcgatgaaaacaggagctggtatctgacagagaacatccagagattcctgccaaatcccgccggcgtccagctggaggacccggagtttcaggcatctaacatcatgcattccattaatggttacgtgttcgactccctgcagctgagcgtgtgcctccacgaggtggcctactggtacatcttgagcatcggcgcccagaccgactttctgagcgtctttttctccgggtatactttcaaacataagatggtgtacgaagatactctgacgctgttccctttctctggggagactgtgtttatgtctatggagaaccctggactgtggattctcggatgccacaacagtgactttcgtaatagagggatgactgcactgctgaaggtgtccagctgtgataaaaatactggcgactactacgaagatagctatgaggatatctcagcatacctgctgagcaagaataacgccatcgagccccgaagcttctcacagaatccccctgtcctcaaggcccaccaggcggagatcacaaggaccacactccagtccgaccaggaggagattgactacgatgacacgatttctgtggagatgaaaaaagaggactttgacatctacgatgaggatgaaaaccagagccctaggtcgttccagaagaaaacaaggcactacttcattgccgccgtggagagactgtgggactacggaatgagtagttccccacacgtgttgcggaacagagcccagagtgggtccgtcccacagttcaagaaggttgttttccaggagttcacagatggctccttcactcagccactgtatcgcggcgagctgaatgagcacttgggcttattgggcccctacattcgcgcagaagtcgaagataatattatggtgaccttccgcaaccaggccagccggccttactcattctactcctctctcatctcttatgaggaggatcagcgccagggcgccgaaccccggaagaactttgtgaagcccaatgaaaccaaaacttacttttggaaggtgcagcaccatatggcgccgacgaaagacgaatttgactgcaaagcctgggcctacttcagcgacgtcgacttggagaaggacgtccacagcggcctgattggccctttgttggtctgccataccaatacactcaaccctgcccacgggaggcaggtgaccgtgcaggagtttgccttgttcttcaccatcttcgacgaaaccaagagctggtacttcacagagaacatggagaggaactgcagagcaccctgtaacatccagatggaggaccctactttcaaggaaaattacaggttccatgccattaatggctacatcatggataccctccccgggcttgtgatggctcaggaccagcgcatccgctggtacctgctctcaatgggctccaacgagaacattcatagcatccactttagtggccacgtgtttaccgtgcgcaagaaggaggagtacaagatggcactgtacaacctgtaccctggcgtgtttgagacagtggagatgctgccatccaaggccggcatctggcgcgtggagtgcctcattggggagcacctccatgctggcatgtctacactgttcctggtgtacagcaacaagtgtcagactccactcggaatggcctccgggcatatccgcgattttcagatcacggcctctggccagtatggccaatgggctcccaagctggccaggctgcactacagtgggagtatcaacgcttggagcaccaaggagcctttctcctggatcaaggtggacctgcttgcccccatgattattcacggcattaagacacagggggccaggcagaaattctcctccctgtacatctcccagttcatcatcatgtacagtctggacggcaaaaagtggcagacctaccgcgggaacagtaccgggacattgatggtgttcttcgggaacgtggactctagcggcattaaacacaacattttcaacccccccatcattgctaggtatatcaggctccatcccacccactatagcatcaggtccactctgcggatggagctgatgggctgcgaccttaattcatgcagcatgccgctgggcatggagtcaaaggccatctccgacgcccaaatcaccgcctccagctacttcaccaatatgttcgccacctggagccccagcaaggcccggctgcacctgcagggccgcagcaacgcctggcggcctcaggtgaacaaccccaaggagtggctgcaggtggacttccagaaaaccatgaaggtgactggggtcaccacccagggagtcaagagcctgctgaccagcatgtatgtgaaggagttcttgatcagctcgtcacaggatggccaccagtggactttgttctttcagaacggtaaggtgaaagtgttccagggaaaccaagattcctttacaccagtggtcaactctctggatcctcccctgctgacacggtacctgcggatccatccccagtcatgggtgcaccagattgctctgcgcatggaggtgcttggctgcgaggcccaggacctgtactgaaattcgcggccgctaaacccagctttcttgtacaaagtggcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 28 - DNA, 3733 bp, synthetic construct
  misc_feature 1..3733: note = Description of Artificial Sequence: Synthetic polynucleotide
  source 1..3733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaagctggaagagttcgtgcagggcaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggacgacatcaacagctacgagtgctggtgccccttcggcttcgagggcaagaattgcgagctggacgtgacctgcaacatcaagaacggcagatgcgagcagttctgcaagaacagcgccgacaacaaggtcgtgtgctcctgcacagagggctacagactggccgagaaccagaagtcttgcgagcccgctgtgccctttccatgtggcagagtgtctgtgtcccagaccagcaagctgaccagagccgagacagtgttccccgacgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggcgagcacaacatcgaggaaacagagcacaccgagcagaaacggaacgtgatcagaatcatccctcaccacaactacaacgccgccatcaacaagtacaaccacgatatcgccctgctggaactggacgagcccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcaaggcgattctggcggccctcacgtgacagaggttgagggcacaagctttctgaccggcatcatcagctggggcgaagagtgtgccatgaaggggaagtacggcatctacaccaaggtgtccagatacgtgaactggatcaaagaaaagaccaagctcacctgaaattcgcggccgctaaacccagctttcttgtacaaagtggcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 29 - protein, 153 aa, Homo sapiens
  REGION 1..20: note = Signal sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 30 - protein, 65 aa, synthetic construct
  REGION 1..25: note = Signal sequence
  REGION 1..65: note = Description of Artificial Sequence: Synthetic polypeptide
MIPAKDMAKVMIVMLAICFLTKSDGKSVKKRSVSEIQLMHNLGKHLNSMERVEWLRKKLQDVHNF

SEQ ID NO: 31 - protein, 115 aa, synthetic construct
  REGION 1..25: note = Signal sequence
  REGION 1..115: note = Description of Artificial Sequence: Synthetic polypeptide
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 32 - protein, 506 aa, synthetic construct
  REGION 1..22: note = Signal sequence
  REGION 1..506: note = Description of Artificial Sequence: Synthetic polypeptide
MIPARFAGVLLALALILPGTLCSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPP

SEQ ID NO: 33 - protein, 1247 aa, synthetic construct
  REGION 1..22: note = Signal sequence
  REGION 1..1247: note = Description of Artificial Sequence: Synthetic polypeptide
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPP

SEQ ID NO: 34 - protein, 232 aa, synthetic construct
  REGION 1..232: note = Description of Artificial Sequence: Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 585 aa, Homo sapiens [sequence omitted]

SEQ ID NO: 36 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT